SEQUENCE LISTING (264 sequences)

SEQ ID NO: 1 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 2 - protein, 500 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKR

SEQ ID NO: 3 - protein, 501 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 4 - protein, 502 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRT

SEQ ID NO: 5 - protein, 503 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTG

SEQ ID NO: 6 - protein, 504 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQ

SEQ ID NO: 7 - protein, 506 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 8 - protein, 505 aa, Homo sapiens
AERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 9 - protein, 507 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLC

SEQ ID NO: 10 - protein, 508 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCI

SEQ ID NO: 11 - protein, 48 aa, Homo sapiens
MAERAALEELVKLQGERVRGLKQQKASAELIEEEVAKLLKLKAQLGPD

SEQ ID NO: 12 - protein, 80 aa, Homo sapiens
MAERAALEELVKLQGERVRGLKQQKASAELIEEEVAKLLKLKAQLGPDESKQKFVLKTPKGTRDYSPRQMAVREKVFDVI

SEQ ID NO: 13 - protein, 79 aa, Homo sapiens
MAERAALEELVKLQGERVRGLKQQKASAELIEEEVAKLLKLKAQLGPDESKQKFVLKTPKGTRDYSPRQMAVREKVFDV

SEQ ID NO: 14 - protein, 78 aa, Homo sapiens
MAERAALEELVKLQGERVRGLKQQKASAELIEEEVAKLLKLKAQLGPDESKQKFVLKTPKGTRDYSPRQMAVREKVFD

SEQ ID NO: 15 - protein, 77 aa, Homo sapiens
MAERAALEELVKLQGERVRGLKQQKASAELIEEEVAKLLKLKAQLGPDESKQKFVLKTPKGTRDYSPRQMAVREKVF

SEQ ID NO: 16 - protein, 76 aa, Homo sapiens
MAERAALEELVKLQGERVRGLKQQKASAELIEEEVAKLLKLKAQLGPDESKQKFVLKTPKGTRDYSPRQMAVREKV

SEQ ID NO: 17 - protein, 75 aa, Homo sapiens
MAERAALEELVKLQGERVRGLKQQKASAELIEEEVAKLLKLKAQLGPDESKQKFVLKTPKGTRDYSPRQMAVREK

SEQ ID NO: 18 - protein, 74 aa, Homo sapiens
MAERAALEELVKLQGERVRGLKQQKASAELIEEEVAKLLKLKAQLGPDESKQKFVLKTPKGTRDYSPRQMAVRE

SEQ ID NO: 19 - protein, 73 aa, Homo sapiens
MAERAALEELVKLQGERVRGLKQQKASAELIEEEVAKLLKLKAQLGPDESKQKFVLKTPKGTRDYSPRQMAVR

SEQ ID NO: 20 - protein, 72 aa, Homo sapiens
MAERAALEELVKLQGERVRGLKQQKASAELIEEEVAKLLKLKAQLGPDESKQKFVLKTPKGTRDYSPRQMAV

SEQ ID NO: 21 - protein, 71 aa, Homo sapiens
MAERAALEELVKLQGERVRGLKQQKASAELIEEEVAKLLKLKAQLGPDESKQKFVLKTPKGTRDYSPRQMA

SEQ ID NO: 22 - protein, 70 aa, Homo sapiens
MAERAALEELVKLQGERVRGLKQQKASAELIEEEVAKLLKLKAQLGPDESKQKFVLKTPKGTRDYSPRQM

SEQ ID NO: 23 - protein, 69 aa, Homo sapiens
MAERAALEELVKLQGERVRGLKQQKASAELIEEEVAKLLKLKAQLGPDESKQKFVLKTPKGTRDYSPRQ

SEQ ID NO: 24 - protein, 68 aa, Homo sapiens
MAERAALEELVKLQGERVRGLKQQKASAELIEEEVAKLLKLKAQLGPDESKQKFVLKTPKGTRDYSPR

SEQ ID NO: 25 - protein, 67 aa, Homo sapiens
MAERAALEELVKLQGERVRGLKQQKASAELIEEEVAKLLKLKAQLGPDESKQKFVLKTPKGTRDYSP

SEQ ID NO: 26 - protein, 66 aa, Homo sapiens
MAERAALEELVKLQGERVRGLKQQKASAELIEEEVAKLLKLKAQLGPDESKQKFVLKTPKGTRDYS

SEQ ID NO: 27 - protein, 65 aa, Homo sapiens
MAERAALEELVKLQGERVRGLKQQKASAELIEEEVAKLLKLKAQLGPDESKQKFVLKTPKGTRDY

SEQ ID NO: 28 - protein, 64 aa, Homo sapiens
MAERAALEELVKLQGERVRGLKQQKASAELIEEEVAKLLKLKAQLGPDESKQKFVLKTPKGTRD

SEQ ID NO: 29 - protein, 63 aa, Homo sapiens
MAERAALEELVKLQGERVRGLKQQKASAELIEEEVAKLLKLKAQLGPDESKQKFVLKTPKGTR

SEQ ID NO: 30 - protein, 62 aa, Homo sapiens
MAERAALEELVKLQGERVRGLKQQKASAELIEEEVAKLLKLKAQLGPDESKQKFVLKTPKGT

SEQ ID NO: 31 - protein, 61 aa, Homo sapiens
MAERAALEELVKLQGERVRGLKQQKASAELIEEEVAKLLKLKAQLGPDESKQKFVLKTPKG

SEQ ID NO: 32 - protein, 60 aa, Homo sapiens
MAERAALEELVKLQGERVRGLKQQKASAELIEEEVAKLLKLKAQLGPDESKQKFVLKTPK

SEQ ID NO: 33 - protein, 59 aa, Homo sapiens
MAERAALEELVKLQGERVRGLKQQKASAELIEEEVAKLLKLKAQLGPDESKQKFVLKTP

SEQ ID NO: 34 - protein, 58 aa, Homo sapiens
MAERAALEELVKLQGERVRGLKQQKASAELIEEEVAKLLKLKAQLGPDESKQKFVLKT

SEQ ID NO: 35 - protein, 57 aa, Homo sapiens
MAERAALEELVKLQGERVRGLKQQKASAELIEEEVAKLLKLKAQLGPDESKQKFVLK

SEQ ID NO: 36 - protein, 56 aa, Homo sapiens
MAERAALEELVKLQGERVRGLKQQKASAELIEEEVAKLLKLKAQLGPDESKQKFVL

SEQ ID NO: 37 - protein, 55 aa, Homo sapiens
MAERAALEELVKLQGERVRGLKQQKASAELIEEEVAKLLKLKAQLGPDESKQKFV

SEQ ID NO: 38 - protein, 54 aa, Homo sapiens
MAERAALEELVKLQGERVRGLKQQKASAELIEEEVAKLLKLKAQLGPDESKQKF

SEQ ID NO: 39 - protein, 53 aa, Homo sapiens
MAERAALEELVKLQGERVRGLKQQKASAELIEEEVAKLLKLKAQLGPDESKQK

SEQ ID NO: 40 - protein, 52 aa, Homo sapiens
MAERAALEELVKLQGERVRGLKQQKASAELIEEEVAKLLKLKAQLGPDESKQ

SEQ ID NO: 41 - protein, 51 aa, Homo sapiens
MAERAALEELVKLQGERVRGLKQQKASAELIEEEVAKLLKLKAQLGPDESK

SEQ ID NO: 42 - protein, 50 aa, Homo sapiens
MAERAALEELVKLQGERVRGLKQQKASAELIEEEVAKLLKLKAQLGPDES

SEQ ID NO: 43 - protein, 49 aa, Homo sapiens
MAERAALEELVKLQGERVRGLKQQKASAELIEEEVAKLLKLKAQLGPDE

SEQ ID NO: 44 - protein, 48 aa, Homo sapiens; identical to SEQ ID NO: 11

SEQ ID NO: 45 - protein, 47 aa, Homo sapiens
MAERAALEELVKLQGERVRGLKQQKASAELIEEEVAKLLKLKAQLGP

SEQ ID NO: 46 - protein, 46 aa, Homo sapiens
MAERAALEELVKLQGERVRGLKQQKASAELIEEEVAKLLKLKAQLG

SEQ ID NO: 47 - protein, 45 aa, Homo sapiens
MAERAALEELVKLQGERVRGLKQQKASAELIEEEVAKLLKLKAQL

SEQ ID NO: 48 - protein, 44 aa, Homo sapiens
MAERAALEELVKLQGERVRGLKQQKASAELIEEEVAKLLKLKAQ

SEQ ID NO: 49 - protein, 43 aa, Homo sapiens
MAERAALEELVKLQGERVRGLKQQKASAELIEEEVAKLLKLKA

SEQ ID NO: 50 - protein, 42 aa, Homo sapiens
MAERAALEELVKLQGERVRGLKQQKASAELIEEEVAKLLKLK

SEQ ID NO: 51 - protein, 41 aa, Homo sapiens
MAERAALEELVKLQGERVRGLKQQKASAELIEEEVAKLLKL

SEQ ID NO: 52 - protein, 40 aa, Homo sapiens
MAERAALEELVKLQGERVRGLKQQKASAELIEEEVAKLLK

SEQ ID NO: 53 - protein, 79 aa, Homo sapiens
AERAALEELVKLQGERVRGLKQQKASAELIEEEVAKLLKLKAQLGPDESKQKFVLKTPKGTRDYSPRQMAVREKVFDVI

SEQ ID NO: 54 - protein, 78 aa, Homo sapiens
ERAALEELVKLQGERVRGLKQQKASAELIEEEVAKLLKLKAQLGPDESKQKFVLKTPKGTRDYSPRQMAVREKVFDVI

SEQ ID NO: 55 - protein, 77 aa, Homo sapiens
RAALEELVKLQGERVRGLKQQKASAELIEEEVAKLLKLKAQLGPDESKQKFVLKTPKGTRDYSPRQMAVREKVFDVI

SEQ ID NO: 56 - protein, 76 aa, Homo sapiens
AALEELVKLQGERVRGLKQQKASAELIEEEVAKLLKLKAQLGPDESKQKFVLKTPKGTRDYSPRQMAVREKVFDVI

SEQ ID NO: 57 - protein, 75 aa, Homo sapiens
ALEELVKLQGERVRGLKQQKASAELIEEEVAKLLKLKAQLGPDESKQKFVLKTPKGTRDYSPRQMAVREKVFDVI

SEQ ID NO: 58 - protein, 74 aa, Homo sapiens
LEELVKLQGERVRGLKQQKASAELIEEEVAKLLKLKAQLGPDESKQKFVLKTPKGTRDYSPRQMAVREKVFDVI

SEQ ID NO: 59 - protein, 73 aa, Homo sapiens
EELVKLQGERVRGLKQQKASAELIEEEVAKLLKLKAQLGPDESKQKFVLKTPKGTRDYSPRQMAVREKVFDVI

SEQ ID NO: 60 - protein, 72 aa, Homo sapiens
ELVKLQGERVRGLKQQKASAELIEEEVAKLLKLKAQLGPDESKQKFVLKTPKGTRDYSPRQMAVREKVFDVI

SEQ ID NO: 61 - protein, 71 aa, Homo sapiens
LVKLQGERVRGLKQQKASAELIEEEVAKLLKLKAQLGPDESKQKFVLKTPKGTRDYSPRQMAVREKVFDVI

SEQ ID NO: 62 - protein, 70 aa, Homo sapiens
VKLQGERVRGLKQQKASAELIEEEVAKLLKLKAQLGPDESKQKFVLKTPKGTRDYSPRQMAVREKVFDVI

SEQ ID NO: 63 - protein, 69 aa, Homo sapiens
KLQGERVRGLKQQKASAELIEEEVAKLLKLKAQLGPDESKQKFVLKTPKGTRDYSPRQMAVREKVFDVI

SEQ ID NO: 64 - protein, 68 aa, Homo sapiens
LQGERVRGLKQQKASAELIEEEVAKLLKLKAQLGPDESKQKFVLKTPKGTRDYSPRQMAVREKVFDVI

SEQ ID NO: 65 - protein, 67 aa, Homo sapiens
QGERVRGLKQQKASAELIEEEVAKLLKLKAQLGPDESKQKFVLKTPKGTRDYSPRQMAVREKVFDVI

SEQ ID NO: 66 - protein, 66 aa, Homo sapiens
GERVRGLKQQKASAELIEEEVAKLLKLKAQLGPDESKQKFVLKTPKGTRDYSPRQMAVREKVFDVI

SEQ ID NO: 67 - protein, 64 aa, Homo sapiens
RVRGLKQQKASAELIEEEVAKLLKLKAQLGPDESKQKFVLKTPKGTRDYSPRQMAVREKVFDVI

SEQ ID NO: 68 - protein, 63 aa, Homo sapiens
VRGLKQQKASAELIEEEVAKLLKLKAQLGPDESKQKFVLKTPKGTRDYSPRQMAVREKVFDVI

SEQ ID NO: 69 - protein, 62 aa, Homo sapiens
RGLKQQKASAELIEEEVAKLLKLKAQLGPDESKQKFVLKTPKGTRDYSPRQMAVREKVFDVI

SEQ ID NO: 70 - protein, 61 aa, Homo sapiens
GLKQQKASAELIEEEVAKLLKLKAQLGPDESKQKFVLKTPKGTRDYSPRQMAVREKVFDVI

SEQ ID NO: 71 - protein, 60 aa, Homo sapiens
LKQQKASAELIEEEVAKLLKLKAQLGPDESKQKFVLKTPKGTRDYSPRQMAVREKVFDVI

SEQ ID NO: 72 - protein, 59 aa, Homo sapiens
KQQKASAELIEEEVAKLLKLKAQLGPDESKQKFVLKTPKGTRDYSPRQMAVREKVFDVI

SEQ ID NO: 73 - protein, 58 aa, Homo sapiens
QQKASAELIEEEVAKLLKLKAQLGPDESKQKFVLKTPKGTRDYSPRQMAVREKVFDVI

SEQ ID NO: 74 - protein, 57 aa, Homo sapiens
QKASAELIEEEVAKLLKLKAQLGPDESKQKFVLKTPKGTRDYSPRQMAVREKVFDVI

SEQ ID NO: 75 - protein, 56 aa, Homo sapiens
KASAELIEEEVAKLLKLKAQLGPDESKQKFVLKTPKGTRDYSPRQMAVREKVFDVI

SEQ ID NO: 76 - protein, 55 aa, Homo sapiens
ASAELIEEEVAKLLKLKAQLGPDESKQKFVLKTPKGTRDYSPRQMAVREKVFDVI

SEQ ID NO: 77 - protein, 54 aa, Homo sapiens
SAELIEEEVAKLLKLKAQLGPDESKQKFVLKTPKGTRDYSPRQMAVREKVFDVI

SEQ ID NO: 78 - protein, 53 aa, Homo sapiens
AELIEEEVAKLLKLKAQLGPDESKQKFVLKTPKGTRDYSPRQMAVREKVFDVI

SEQ ID NO: 79 - protein, 52 aa, Homo sapiens
ELIEEEVAKLLKLKAQLGPDESKQKFVLKTPKGTRDYSPRQMAVREKVFDVI

SEQ ID NO: 80 - protein, 51 aa, Homo sapiens
LIEEEVAKLLKLKAQLGPDESKQKFVLKTPKGTRDYSPRQMAVREKVFDVI

SEQ ID NO: 81 - protein, 50 aa, Homo sapiens
IEEEVAKLLKLKAQLGPDESKQKFVLKTPKGTRDYSPRQMAVREKVFDVI

SEQ ID NO: 82 - protein, 49 aa, Homo sapiens
EEEVAKLLKLKAQLGPDESKQKFVLKTPKGTRDYSPRQMAVREKVFDVI

SEQ ID NO: 83 - protein, 48 aa, Homo sapiens
EEVAKLLKLKAQLGPDESKQKFVLKTPKGTRDYSPRQMAVREKVFDVI

SEQ ID NO: 84 - protein, 47 aa, Homo sapiens
EVAKLLKLKAQLGPDESKQKFVLKTPKGTRDYSPRQMAVREKVFDVI

SEQ ID NO: 85 - protein, 46 aa, Homo sapiens
VAKLLKLKAQLGPDESKQKFVLKTPKGTRDYSPRQMAVREKVFDVI

SEQ ID NO: 86 - protein, 45 aa, Homo sapiens
AKLLKLKAQLGPDESKQKFVLKTPKGTRDYSPRQMAVREKVFDVI

SEQ ID NO: 87 - protein, 44 aa, Homo sapiens
KLLKLKAQLGPDESKQKFVLKTPKGTRDYSPRQMAVREKVFDVI

SEQ ID NO: 88 - protein, 43 aa, Homo sapiens
LLKLKAQLGPDESKQKFVLKTPKGTRDYSPRQMAVREKVFDVI

SEQ ID NO: 89 - protein, 42 aa, Homo sapiens
LKLKAQLGPDESKQKFVLKTPKGTRDYSPRQMAVREKVFDVI

SEQ ID NO: 90 - protein, 41 aa, Homo sapiens
KLKAQLGPDESKQKFVLKTPKGTRDYSPRQMAVREKVFDVI

SEQ ID NO: 91 - protein, 40 aa, Homo sapiens
LKAQLGPDESKQKFVLKTPKGTRDYSPRQMAVREKVFDVI

SEQ ID NO: 92 - protein, 141 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAM

SEQ ID NO: 93 - protein, 408 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTE

SEQ ID NO: 94 - protein, 113 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKL

SEQ ID NO: 95 - protein, 60 aa, Homo sapiens; identical to SEQ ID NO: 32

SEQ ID NO: 96 - protein, 270 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVGYPWWNSCSRILNYPKTSRPWRAWET

SEQ ID NO: 97 - protein, 105 aa, Homo sapiens
RTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 98 - protein, 395 aa, Homo sapiens
MAERAALEELVKLQGERVRGLKQQKASAELIEEEVAKLLKLKAQLGPDESKQKFVLKTPKDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 99 - protein, 359 aa, Homo sapiens
MAERAALEELVKLQGERVRGLKQQKASAELIEEEVAKLLKLKAQLGPDESKQKFVLKTPKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 100 - protein, 399 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 101 - protein, 473 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 102 - protein, 469 aa, Homo sapiens
MAERAALEELVKLQGERVRGLKQQKASAELIEEEVAKLLKLKAQLGPDESKQKFVLKTPKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 103 - protein, 435 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 104 - protein, 171 aa, Homo sapiens
MAERAALEELVKLQGERVRGLKQQKASAELIEEEVAKLLKLKAQLGPDESKQKFVLKTPKALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 105 - protein, 211 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 106 - protein, 141 aa, Homo sapiens [sequence omitted]

SEQ ID NO: 107 - protein, 143 aa, Homo sapiens
CLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTG

SEQ ID NO: 108 - protein, 506 aa, Homo sapiens
MPLLGLLPRRAWASLLSQLLRPPCASCTGAVRCQSQVAEAVLTSQLKAHQEKPNFIIKTPKGTRDLSPQHMVVREKILDLVISCFKRHGAKGMDTPAFELKETLTEKYGEDSGLMYDLKDQGGELLSLRYDLTVPFARYLAMNKVKKMKRYHVGKVWRRESPTIVQGRYREFCQCDFDIAGQFDPMIPDAECLKIMCEILSGLQLGDFLIKVNDRRIVDGMFAVCGVPESKFRAICSSIDKLDKMAWKDVRHEMVVKKGLAPEVADRIGDYVQCHGGVSLVEQMFQDPRLSQNKQALEGLGDLKLLFEYLTLFGIADKISFDLSLARGLDYYTGVIYEAVLLQTPTQAGEEPLNVGSVAAGGRYDGLVGMFDPKGHKVPCVGLSIGVERIFYIVEQRMKTKGEKVRTTETQVFVATPQKNFLQERLKLIAELWDSGIKAEMLYKNNPKLLTQLHYCESTGIPLVVIIGEQELKEGVIKIRSVASREEVAIKRENFVAEIQKRLSES

SEQ ID NO: 109 - protein, 509 aa, Mus musculus
MADRAALEELVRLQGAHVRGLKEQKASAEQIEEEVTKLLKLKAQLGQDEGKQKFVLKTPKGTRDYSPRQMAVREKVFDVIIRCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMIPDAECLKIMCEILSSLQIGNFLVKVNDRRILDGMFAVCGVPDSKFRTICSSVDKLDKVSWEEVKNEMVGEKGLAPEVADRIGDYVQQHGGVSLVEQLLQDPKLSQNKQAVEGLGDLKLLFEYLILFGIDDKISFDLSLARGLDYYTGVIYEAVLLQMPTQAGEEPLGVGSIAAGGRYDGLVGMFDPKGRKVPCVGLSIGVERIFSIVEQRLEASEEKVRTTETQVLVASAQKKLLEERLKLVSELWDAGIKAELLYKKNPKLLNQLQYWEEAGIPLVAIIGEQELRDGVIKLRSVASREEVDVRREDLVEEIRRRTNQPLSTC

SEQ ID NO: 110 - protein, 509 aa, Canis lupus familiaris
MAERAALEELVRLQGERVRGLKQQKASAEQIEEEVAKLLKLKAQLGPDEGKQKFVLKTPKGTRDYSPRQMAVREKVFDVIISCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMIPDAECLEIMCEILRSLQIGDFLVKVNDRRILDGMFAICGVPDSKFRTICSSVDKLDKVSWEEVKNEMVGEKGLAPEVADHIGDYVQQHGGISLVEQLLQDPELSQNKQALEGLGDLKLLFEYLTLFGIADKISFDLSLARGLDYYTGVIYEAVLLQTPVQAGEEPLGVGSVAAGGRYDGLVGMFDPKGRKVPCVGLSIGVERIFSIVEQRLEATEEKVRTTETQVLVASAQKKLLEERLKLVSELWNAGIKAELLYKKNPKLLNQLQYCEEAGIPLVAIIGEQELKDGVIKLRSVASREEVDVPREDLVEEIKRRTSQPFCIC

SEQ ID NO: 111 - protein, 509 aa, Bos taurus
MADRAALEDLVRVQGERVRGLKQQKASAEQIEEEVAKLLKLKAQLGPDEGKPKFVLKTPKGTRDYSPRQMAVREKVFDVIISCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMLPDAECLKIMCEILSSLQIGDFLVKVNDRRILDGMFAICGVPDSKFRTICSSVDKLDKVSWEEVKNEMVGEKGLAPEVADRIGDYVQQHGGVSLVEQLLQDPKLSQNKQALEGLGDLKLLFEYLTLFGIADKISFDLSLARGLDYYTGVIYEAVLLQPPARAGEEPLGVGSVAAGGRYDGLVGMFDPKGRKVPCVGLSIGVERIFSIVEQRLEALEEKVRTTETQVLVASAQKKLLEERLKLISELWDAGIKAELLYKKNPKLLNQLQYCEETGIPLVAIIGEQELKDGVIKLRSVASREEVDVRREDLVEEIKRRTSQPLCIC

SEQ ID NO: 112 - protein, 508 aa, Rattus norvegicus
MADRAALEELVRLQGAHVRGLKEQKASAEQIEEEVTKLLKLKAQLGHDEGKQKFVLKTPKGTRDYSPRQMAVREKVFDVIIRCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMIPDAECLKIMCEILSSLQIGNFQVKVNDRRILDGMFAVCGVPDSKFRTICSSVDKLDKVSWEEVKNEMVGEKGLAPEVADRIGDYVQQHGGVSLVEQLLQDPKLSQNKQAVEGLGDLKLLFEYLTLFGIDDKISFDLSLARGLDYYTGVIYEAVLLQMPTQAGEEPLGVGSIAAGGRYDGLVGMFDPKGRKVPCVGLSIGVERIFSIVEQKLEASEEKVRTTETQVLVASAQKKLLEERLKLISELWDAGIKAELLYKKNPKLLNQLQYCEEAGIPLVAIIGEQELKDGVIKLRSVTSREEVDVRREDLVEEIRRRTSQPLSM

SEQ ID NO: 113 - protein, 500 aa, Gallus gallus
MADEAAVRQQAEVVRRLKQDKAEPDEIAKEVAKLLEMKAHLGGDEGKHKFVLKTPKGTRDYGPKQMAIRERVFSAIIACFKRHGAEVIDTPVFELKETLTGKYGEDSKLIYDLKDQGGELLSLRYDLTVPFARYLAMNKITNIKRYHIAKVYRRDNPAMTRGRYREFYQCDFDIAGQFDPMIPDAECLKIVQEILSDLQLGDFLIKVNDRRILDGMFAVCGVPDSKFRTICSSVDKLDKMPWEEVRNEMVGEKGLSPEAADRIGEYVQLHGGMDLIEQLLQDPKLSQNKLVKEGLGDMKLLFEYLTLFGITGKISFDLSLARGLDYYTGVIYEAVLLQQNDHGEESVSVGSVAGGGRYDGLVGMFDPKGRKVPCVGISIGIERIFSILEQRVEASEEKIRTTETQVLVASAQKKLLEERLKLISELWDAGIKAEVLYKKNPKLLNQLQYCEDTGIPLVAIVGEQELKDGVVKLRVVATGEEVNIRRESLVEEIRRRTNQL

SEQ ID NO: 114 - protein, 437 aa, Danio rerio
MAALGLVSMRLCAGLMGRRSAVRLHSLRVCSGMTISQIDEEVARLLQLKAQLGGDEGKHVFVLKTAKGTRDYNPKQMAIREKVFNIIINCFKRHGAETIDSPVFELKETLTGKYGEDSKLIYDLKDQGGELLSLRYDLTVPFARYLAMNKITNIKRYHIAKVYRRDNPAMTRGRYREFYQCDFDIAGQYDAMIPDAECLKLVYEILSELDLGDFRIKVNDRRILDGMFAICGVPDEKFRTICSTVDKLDKLAWEEVKKEMVNEKGLSEEVADRIRDYVSMQGGKDLAERLLQDPKLSQSKQACAGITDMKLLFSYLELFQITDKVVFDLSLARGLDYYTGVIYEAILTQANPAPASTPAEQNGAEDAGVSVGSVAGGGRYDGLVGMFDPKAGKCPVWGSALALRGSSPSWSRRQSCLQRRCAPLKLKCLWLQHRRTF

SEQ ID NO: 115 - protein, 435 aa, Macaca fascicularis
MAERAALEELVKLQGERVRGLKQQQASAELIEEEVGKLLKLKAQLGPDESKQKFVLKTPKGTRDYSPRQMAVREKVFDVIIRCFKRHGAEVIDTPVFELKDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKVRTTETQVLVASAQKKLLEERLKLVSELWDAGIKAELLYKKNPKLLNQLQYCEEAGIPLVAIIGEQELKDGVIKLRSVTSREEVNVRREDLVEEIKRRTGQLLRIC

SEQ ID NO: 116 - protein, 138 aa, Artificial Sequence
  Consensus HRS WHEP sequence
  MOD_RES (1)..(50): Xaa is any amino acid or absent
  misc_feature (52)..(56): Xaa can be any naturally occurring amino acid
  MOD_RES (57)..(58): Xaa is any amino acid or absent
  MOD_RES (61)..(62): Xaa is any amino acid
  MOD_RES (65)..(65): Xaa is any amino acid
  MOD_RES (68)..(69): Xaa is any amino acid
  misc_feature (72)..(78): Xaa can be any naturally occurring amino acid
  MOD_RES (79)..(80): Xaa is any amino acid or absent
  MOD_RES (82)..(83): Xaa is any amino acid
  MOD_RES (86)..(86): Xaa is any amino acid
  MOD_RES (89)..(138): Xaa is any amino acid or absent
XXXXXXXXXXXXXXXXXXXXXXXXXXXXXXXXXXXXXXXXXXXXXXXXXXLXXXXXXXQGXXVRXLKXXKAXXXXXXXXXVXXLLXLKXXXXXXXXXXXXXXXXXXXXXXXXXXXXXXXXXXXXXXXXXXXXXXXXXX

SEQ ID NO: 117 - protein, 61 aa, Artificial Sequence
  Made in Lab  cysteine modified HRS polypeptide
MCAERAALEELVKLQGERVRGLKQQKASAELIEEEVAKLLKLKAQLGPDESKQKFVLKTPK

SEQ ID NO: 118 - protein, 60 aa, Artificial Sequence
  Made in Lab  cysteine modified HRS polypeptide
MAERAALEELVKLQGERVRGLKQQKCSAELIEEEVAKLLKLKAQLGPDESKQKFVLKTPK

SEQ ID NO: 119 - protein, 61 aa, Artificial Sequence
  Made in Lab  cysteine modified HRS polypeptide
MAERAALEELVKLQGERVRGLKQQKASAELIEEEVAKLLKLKAQLGPDESKQKFVLKTPKC

SEQ ID NO: 120 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQADFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 121 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQVDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 122 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAEALKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 123 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAESLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 124 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAEVLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 125 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAISGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 126 - protein, 506 aa, Artificial Sequence
  Made in Lab modified HRS polypeptide to remove surface exposed cysteines
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTISSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 127 - protein, 19 aa, Homo sapiens
VPSTPPTPSPSTPPTPSPS

SEQ ID NO: 128 - protein, 101 aa, Homo sapiens
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 129 - protein, 131 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 130 - protein, 6 aa, Homo sapiens
VPPPPP

SEQ ID NO: 131 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 132 - protein, 131 aa, Homo sapiens; identical to SEQ ID NO: 129

SEQ ID NO: 133 - protein, 58 aa, Homo sapiens
ESPKAQASSVPTAQPQAEGSLAKATTAPATTRNTGRGGEEKKKEKEKEEQEERETKTP

SEQ ID NO: 134 - protein, 108 aa, Homo sapiens
ECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREP

SEQ ID NO: 135 - protein, 117 aa, Homo sapiens
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 136 - protein, 107 aa, Homo sapiens
VCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 137 - protein, 108 aa, Homo sapiens
DSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 138 - protein, 110 aa, Homo sapiens
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 139 - protein, 15 aa, Homo sapiens
EPKSCDKTHTCPPCP

SEQ ID NO: 140 - protein, 11 aa, Homo sapiens
SDKTHTCPPCP

SEQ ID NO: 141 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 142 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 229 aa, Homo sapiens
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 12 aa, Homo sapiens
ERKCCVECPPCP

SEQ ID NO: 145 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 146 - protein, 107 aa, Homo sapiens
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 147 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 148 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 149 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 150 - protein, 12 aa, Homo sapiens
ESKYGPPCPSCP

SEQ ID NO: 151 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 152 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 153 - protein, 112 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVP

SEQ ID NO: 154 - protein, 106 aa, Homo sapiens
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 155 - protein, 131 aa, Homo sapiens
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 156 - protein, 287 aa, Artificial Sequence
  Made in Lab HRS-Fc fusion protein
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQLGPDESKQKFVLKTPK

SEQ ID NO: 157 - protein, 288 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQLGPDESKQKFVLKTPK

SEQ ID NO: 158 - protein, 288 aa, Artificial Sequence
  Made in Lab HRS-Fc fusion protein
MAERAALEELVKLQGERVRGLKQQKASAELIEEEVAKLLKLKAQLGPDESKQKFVLKTPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 288 aa, Artificial Sequence; identical to SEQ ID NO: 157
  Made in Lab HRS-Fc fusion protein

SEQ ID NO: 160 - protein, 288 aa, Artificial Sequence; identical to SEQ ID NO: 158
  Made in Lab HRS-Fc fusion protein

SEQ ID NO: 161 - protein, 268 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLK

SEQ ID NO: 162 - protein, 273 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQL

SEQ ID NO: 163 - protein, 278 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQLGPDES

SEQ ID NO: 164 - protein, 283 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQLGPDESKQKFV

SEQ ID NO: 165 - protein, 294 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQLGPDESKQKFVLKTPKGTRDYS

SEQ ID NO: 166 - protein, 268 aa, Artificial Sequence
  Made in Lab HRS-Fc fusion protein
MAERAALEELVKLQGERVRGLKQQKASAELIEEEVAKLLKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 273 aa, Artificial Sequence
  Made in Lab HRS-Fc fusion protein
MAERAALEELVKLQGERVRGLKQQKASAELIEEEVAKLLKLKAQLSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 278 aa, Artificial Sequence
  Made in Lab HRS-Fc fusion protein
MAERAALEELVKLQGERVRGLKQQKASAELIEEEVAKLLKLKAQLGPDESSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 283 aa, Artificial Sequence
  Made in Lab HRS-Fc fusion protein
MAERAALEELVKLQGERVRGLKQQKASAELIEEEVAKLLKLKAQLGPDESKQKFVSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 294 aa, Artificial Sequence
  Made in Lab HRS-Fc fusion protein
MAERAALEELVKLQGERVRGLKQQKASAELIEEEVAKLLKLKAQLGPDESKQKFVLKTPKGTRDYSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 347 aa, Artificial Sequence
  Made in Lab HRS-Fc fusion protein
MSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAERAALEELVKLQGERVRGLKQQKASAELIEEEVAKLLKLKAQLGPDESKQKFVLKTPKAERAALEELVKLQGERVRGLKQQKASAELIEEEVAKLLKLKAQLGPDESKQKFVLKTPK

SEQ ID NO: 172 - protein, 505 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQP

SEQ ID NO: 173 - protein, 158 aa, Artificial Sequence
  Made in Lab - HRS-COMP Fusion Protein
MRAWIFFLLCLAGRALAAERAALEELVKLQGERVRGLKQQKASAELIEEEVAKLLKLKAQLGPDESKQKFVLKTPKGGGGSGGGGSSDLGPQMLRELQETNAALQDVRELLRQQVREITFLKNTVMECDACGGGGGSEQKLISEEDLNMHTGHHHHHH

SEQ ID NO: 174 - protein, 141 aa, Artificial Sequence
  Made in Lab - HRS-COMP Fusion Protein
AERAALEELVKLQGERVRGLKQQKASAELIEEEVAKLLKLKAQLGPDESKQKFVLKTPKGGGGSGGGGSSDLGPQMLRELQETNAALQDVRELLRQQVREITFLKNTVMECDACGGGGGSEQKLISEEDLNMHTGHHHHHH

SEQ ID NO: 175 - protein, 113 aa, Artificial Sequence
  Made in Lab - HRS-COMP Fusion Protein
ERAALEELVKLQGERVRGLKQQKASAELIEEEVAKLLKLKAQLGPDESKQKFVLKTPKGGGGSGGGGSSDLGPQMLRELQETNAALQDVRELLRQQVREITFLKNTVMECDAC

SEQ ID NO: 176 - protein, 931 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIRFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 177 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 178 - protein, 909 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFADEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 179 - protein, 906 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGENFKGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 180 - protein, 901 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 181 - protein, 555 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKVGCSWRPL

SEQ ID NO: 182 - protein, 904 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 183 - protein, 879 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 184 - protein, 533 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKVGCSWRPL

SEQ ID NO: 185 - protein, 114 aa, Homo sapiens
CGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEI

SEQ ID NO: 186 - protein, 117 aa, Homo sapies
CSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYY

SEQ ID NO: 187 - protein, 147 aa, Homo sapiens
PLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFG

SEQ ID NO: 188 - protein, 154 aa, Homo sapiens
LGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLG

SEQ ID NO: 189 - protein, 154 aa, Homo sapiens
PSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTD

SEQ ID NO: 190 - protein, 243 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYY

SEQ ID NO: 191 - protein, 278 aa, Homo sapiens
CSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFG

SEQ ID NO: 192 - protein, 404 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFG

SEQ ID NO: 193 - protein, 573 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 194 - protein, 451 aa, Homo sapiens
GSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 195 - protein, 320 aa, Homo sapiens
QCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 196 - protein, 1060 aa, Artificial Sequence
  Made in Lab - NRP2 v2 Fc fusion protein construct
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 678 aa, Artificial Sequence
  Made in Lab - NRP2 A2B1B2-Fc fusion protein construct
GSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 357 aa, Homo sapiens
LGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTD

SEQ ID NO: 199 - protein, 519 aa, Homo sapiens
QCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTD

SEQ ID NO: 200 - DNA, 477 bp, Artificial Sequence
  Made in Lab - optimized nucleic acid sequence of SPARC-HRS(2-60)-COMP-MycHIS construct
atgagggcctggattttctttctgctgtgcctggctggcagagctctggctgctgagagagccgccctggaggagctggtcaagctgcagggcgagagggtgaggggcctgaagcagcagaaggccagcgccgagctgatcgaggaggaggtggccaagctgctgaagctgaaggcccagctcggccctgacgagagcaagcagaagttcgtgctgaagacacccaagggaggaggcggatccggaggaggaggaagcagcgatctgggcccccagatgctgagggagctgcaggagacaaacgccgccctgcaggacgtgagagagctgctgagacagcaggtgagggagatcaccttcctgaagaacaccgtgatggagtgcgacgcctgtggaggaggaggaggcagcgagcagaagctgatcagcgaggaggacctgaacatgcacaccggccatcaccatcaccaccactga

SEQ ID NO: 201 - protein, 4 aa, Artificial Sequence
  Made in Lab peptide linker sequence
GSGS

SEQ ID NO: 202 - protein, 4 aa, Artificial Sequence
  Made in Lab peptide linker sequence
GGSG

SEQ ID NO: 203 - protein, 4 aa, Artificial Sequence
  Made in Lab peptide linker sequence
GGGS

SEQ ID NO: 204 - protein, 5 aa, Artificial Sequence
  Made in Lab peptide linker sequence
GGGGS

SEQ ID NO: 205 - protein, 4 aa, Artificial Sequence
  Made in Lab peptide linker sequence
GNGN

SEQ ID NO: 206 - protein, 4 aa, Artificial Sequence
  Made in Lab peptide linker sequence
GGNG

SEQ ID NO: 207 - protein, 4 aa, Artificial Sequence
  Made in Lab peptide linker sequence
GGGN

SEQ ID NO: 208 - protein, 5 aa, Artificial Sequence
  Made in Lab peptide linker sequence
GGGGN

SEQ ID NO: 209 - protein, 15 aa, Artificial Sequence
  Made in Lab peptide linker sequence
GGGGSGGGGSGGGGS

SEQ ID NO: 210 - protein, 22 aa, Artificial Sequence
  Made in Lab peptide linker sequence
GSGGGGSGGGGSGGGGSGGGGS

SEQ ID NO: 211 - protein, 30 aa, Artificial Sequence
  Made in Lab peptide linker sequence
GGGGSGGGGSGGGGSGGGGSGGGGSGGGGS

SEQ ID NO: 212 - protein, 25 aa, Artificial Sequence
  Made in Lab peptide linker sequence
DAAAKEAAAKDAAAREAAARDAAAK

SEQ ID NO: 213 - protein, 14 aa, Artificial Sequence
  Made in Lab peptide linker sequence
NVDHKPSNTKVDKR

SEQ ID NO: 214 - protein, 5 aa, Artificial Sequence
  Made in Lab peptide linker sequence
DGGGS

SEQ ID NO: 215 - protein, 5 aa, Artificial Sequence
  Made in Lab peptide linker sequence
TGEKP

SEQ ID NO: 216 - protein, 4 aa, Artificial Sequence
  Made in Lab peptide linker sequence
GGRR

SEQ ID NO: 217 - protein, 14 aa, Artificial Sequence
  Made in Lab peptide linker sequence
EGKSSGSGSESKVD

SEQ ID NO: 218 - protein, 18 aa, Artificial Sequence
  Made in Lab peptide linker sequence
KESGSVSSEQLAQFRSLD

SEQ ID NO: 219 - protein, 8 aa, Artificial Sequence
  Made in Lab peptide linker sequence
GGRRGGGS

SEQ ID NO: 220 - protein, 9 aa, Artificial Sequence
  Made in Lab peptide linker sequence
LRQRDGERP

SEQ ID NO: 221 - protein, 12 aa, Artificial Sequence
  Made in Lab peptide linker sequence
LRQKDGGGSERP

SEQ ID NO: 222 - protein, 16 aa, Artificial Sequence
  Made in Lab peptide linker sequence
LRQKDGGGSGGGSERP

SEQ ID NO: 223 - protein, 19 aa, Unknown
  Naturally occurring 2A or 2A-like self cleaving peptide
LLNFDLLKLAGDVESNPGP

SEQ ID NO: 224 - protein, 19 aa, Unknown
  Naturally occurring 2A or 2A-like self cleaving peptide
TLNFDLLKLAGDVESNPGP

SEQ ID NO: 225 - protein, 14 aa, Unknown
  Naturally occurring 2A or 2A-like self cleaving peptide
LLKLAGDVESNPGP

SEQ ID NO: 226 - protein, 17 aa, Unknown
  Naturally occurring 2A or 2A-like self cleaving peptide
NFDLLKLAGDVESNPGP

SEQ ID NO: 227 - protein, 20 aa, Unknown
  Naturally occurring 2A or 2A-like self cleaving peptide
QLLNFDLLKLAGDVESNPGP

SEQ ID NO: 228 - protein, 24 aa, Unknown
  Naturally occurring 2A or 2A-like self cleaving peptide
APVKQTLNFDLLKLAGDVESNPGP

SEQ ID NO: 229 - protein, 40 aa, Unknown
  Naturally occurring 2A or 2A-like self cleaving peptide
VTELLYRMKRAETYCPRPLLAIHPTEARHKQKIVAPVKQT

SEQ ID NO: 230 - protein, 18 aa, Unknown
  Naturally occurring 2A or 2A-like self cleaving peptide
LNFDLLKLAGDVESNPGP

SEQ ID NO: 231 - protein, 40 aa, Unknown
  Naturally occurring 2A or 2A-like self cleaving peptide
LLAIHPTEARHKQKIVAPVKQTLNFDLLKLAGDVESNPGP

SEQ ID NO: 232 - protein, 33 aa, Unknown
  Naturally occurring 2A or 2A-like self cleaving peptide
EARHKQKIVAPVKQTLNFDLLKLAGDVESNPGP

SEQ ID NO: 233 - protein, 7 aa, Tobacco etch virus
  VARIANT (2)..(2): Xaa = any amino acid
  VARIANT (3)..(3): Xaa = any amino acid
  VARIANT (5)..(5): Xaa = any amino acid
  VARIANT (7)..(7): Xaa is either Gly or Ser
EXXYXQX

SEQ ID NO: 234 - protein, 7 aa, Tobacco etch virus
ENLYFQG

SEQ ID NO: 235 - protein, 7 aa, Tabacco etch virus
ENLYFQS

SEQ ID NO: 236 - protein, 4 aa, Artificial Sequence
  Thrombin cleavable linker
GRGD

SEQ ID NO: 237 - protein, 6 aa, Artificial Sequence
  Thrombin cleavable linker
GRGDNP

SEQ ID NO: 238 - protein, 5 aa, Artificial Sequence
  Thrombin cleavable linker
GRGDS

SEQ ID NO: 239 - protein, 7 aa, Artificial Sequence
  Thrombin cleavable linker
GRGDSPK

SEQ ID NO: 240 - protein, 4 aa, Artificial Sequence
  Elastase cleavable linker
AAPV

SEQ ID NO: 241 - protein, 4 aa, Artificial Sequence
  Elastase cleavable linker
AAPL

SEQ ID NO: 242 - protein, 4 aa, Artificial Sequence
  Elastase cleavable linker
AAPF

SEQ ID NO: 243 - protein, 4 aa, Artificial Sequence
  Elastase cleavable linker
AAPA

SEQ ID NO: 244 - protein, 4 aa, Artificial Sequence
  Elastase cleavable linker
AYLV

SEQ ID NO: 245 - protein, 6 aa, Artificial Sequence
  Matrix metalloproteinase cleavable linker
  VARIANT (3)..(3): Xaa = Any amino acid
  VARIANT (6)..(6): Xaa = Any amino acid
GPXGPX

SEQ ID NO: 246 - protein, 4 aa, Artificial Sequence
  Matrix metalloproteinase cleavable linker
  VARIANT (4)..(4): Xaa = Any amino acid
LGPX

SEQ ID NO: 247 - protein, 6 aa, Artificial Sequence
  Matrix metalloproteinase cleavable linker
  VARIANT (6)..(6): Xaa = Any amino acid
GPIGPX

SEQ ID NO: 248 - protein, 5 aa, Artificial Sequence
  Matrix metalloproteinase cleavable linker
  VARIANT (5)..(5): Xaa = Any amino acid
APGLX

SEQ ID NO: 249 - protein, 7 aa, Artificial Sequence
  Collagenase cleavable linker
  VARIANT (7)..(7): Xaa = Any amino acid
PLGPDRX

SEQ ID NO: 250 - protein, 7 aa, Artificial Sequence
  Collagenase cleavable linker
  VARIANT (7)..(7): Xaa = Any amino acid
PLGLLGX

SEQ ID NO: 251 - protein, 7 aa, Artificial Sequence
  Collagenase cleavable linker
PQGIAGW

SEQ ID NO: 252 - protein, 5 aa, Artificial Sequence
  Collagenase cleavable linker
PLGCH

SEQ ID NO: 253 - protein, 6 aa, Artificial Sequence
  Collagenase cleavable linker
PLGLYA

SEQ ID NO: 254 - protein, 7 aa, Artificial Sequence
  Collagenase cleavable linker
PLALWAR

SEQ ID NO: 255 - protein, 7 aa, Artificial Sequence
  Collagenase cleavable linker
PLAYWAR

SEQ ID NO: 256 - protein, 7 aa, Artificial Sequence
  Stromelysin cleavable linker
PYAYYMR

SEQ ID NO: 257 - protein, 7 aa, Artificial Sequence
  Gelatinase cleavable linker
PLGMYSR

SEQ ID NO: 258 - protein, 4 aa, Artificial Sequence
  Angiotensin converting enzyme cleavable linker
GDKP

SEQ ID NO: 259 - protein, 5 aa, Artificial Sequence
  Angiotensin converting enzyme cleavable linker
GSDKP

SEQ ID NO: 260 - protein, 4 aa, Artificial Sequence
  Cathepsin B cleavable linker
ALAL

SEQ ID NO: 261 - protein, 4 aa, Artificial Sequence
  Cathepsin B cleavable linker
GFLG

SEQ ID NO: 262 - protein, 21 aa, Artificial Sequence
  Made in Lab - Myc-His tag construct
EQKLISEEDLNMHTGHHHHHH

SEQ ID NO: 263 - DNA, 36 bp, Artificial Sequence
  Primer
gctggcagagctctggctggaggaggcggatccgga

SEQ ID NO: 264 - DNA, 36 bp, Artificial Sequence
  Primer
tccggatccgcctcctccagccagagctctgccagc

(19) United States
(12) Patent Application Publication
(10) Pub. No.: US 2023/0039401 A1
(43) Pub. Date: Feb. 9, 2023

(54) COMPOSITIONS AND METHODS FOR TREATING NRP2-ASSOCIATED DISEASES

(71) Applicant: aTyr Pharma, Inc., San Diego, CA (US)

(72) Inventors: Christoph BURKART, San Diego, CA (US); Kathleen M. OGILVIE, San Diego, CA (US); Suzanne PAZ, San Marcos, CA (US); Sanna ROSENGREN, Encinitas, CA (US); Kaustubh DATTA, Omaha, NE (US); Samikshan DUTTA, Omaha, NE (US)

(21) Appl. No.: 17/841,187

(22) Filed: Jun. 15, 2022

Related U.S. Application Data
(62) Division of application No. 16/523,783, filed on Jul. 26, 2019, now abandoned.
(60) Provisional application No. 62/849,440, filed on May 17, 2019.
(60) Provisional application No. 62/800,035, filed on Feb. 1, 2019.
(60) Provisional application No. 62/776,208, filed on Dec. 6, 2018.
(60) Provisional application No. 62/703,757, filed on Jul. 26, 2018.

(51) Int. Cl. A61K 38/53; A61P 29/00

(52) U.S. Cl. CPC A61K 38/53; A61P 29/00; A61K 45/06

(57) ABSTRACT

Provided are therapies, including standalone and combination therapies, for treating neuropilin-2 (NRP2)-associated diseases and conditions, which include the use of at least one histidyl-tRNA synthetase (HRS) polypeptide.

### CROSS-REFERENCE TO RELATED APPLICATIONS

This application is a Divisional of U.S. patent application Ser. No. 16/523,783, filed Jul. 26, 2019, which claims priority under 35 U.S.C. § 119(e) to U.S. provisional application No. 62/703,757, filed Jul. 26, 2018; U.S. provisional application No. 62/776,208, filed Dec. 6, 2018; U.S. provisional application No. 62/800,035, filed Feb. 1, 2019; and U.S. provisional application No. 62/849,440, filed May 17, 2019; each of which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is ATYR_135_05US_ST25.txt. The text file is about 392 KB, was created on Jun. 15, 2022, and is being submitted electronically via EFS-Web.

### BACKGROUND

### Technical Field

Embodiments of the present disclosure relate to therapies, including standalone and combination therapies, for treating neuropilin-2 (NRP2)-associated diseases and conditions, which include the use of at least one histidyl-tRNA synthetase (HRS) polypeptide.

### Description of the Related Art

Recent research developments suggest that tRNA synthetases play important roles in cellular responses beyond their well characterized role in protein synthesis. In particular, there is a growing recognition that tRNA synthetases may participate in a range of previously unrecognized roles in responding to cellular stress, and tissue homeostasis, both within the intracellular and extracellular environments.

The Resokine family of proteins (HRS Polypeptides) are derived from the histidyl tRNA synthetase gene (HARS) via proteolysis or alternative splicing, and are important extracellular modulators of cellular activity. Extracellular HARS is readily detectable in the circulation in normal healthy volunteers, and autoantibodies to HARS (Jo-1 antibodies) have been characterized in some inflammatory myopathies (IM) and subjects with inflammatory lung disease (ILD). While the role of Jo-1 antibodies in disease progression is not yet well understood, subjects with Jo-1 antibodies tend to be less susceptible to cancer, compared to subjects with inflammatory myopathies without Jo-1 antibodies (see, e.g., Lu et al., PLOS ONE 9(4) e94128, 2014; Modan et al., Clin. Exp. Dermatol. 34(5) 561-565, 2009; and Shi et al., J. Rheum 44 (7) doi 10.3899/jrheum.161480).

Recently, significant progress has been made in elucidating the role of extracellular HARS derived proteins, including the identification of a putative cellular receptor, neuropilin-2 (NRP2, or NRP-2). Interactions of HARS with NRP2 appear to be mediated by the N-terminal region of HARS, and can lead to important changes in the cellular function of NRP2.

Accordingly, the current discovery of the Resokine/neuropilin-2 axis represents a previously unknown mechanism, which acts as a central regulator of cellular processes with direct relevance to muscular, vascular, neuronal, bone, and immune homeostasis. The deregulation of any of these processes may lead to a spectrum of diseases, which new HRS polypeptide based therapeutics may be able to address.

### BRIEF SUMMARY

Embodiments of the present disclosure include methods for treating a neuropilin-2 (NRP2) associated disease or condition in a subject in need thereof, comprising administering to the subject a therapeutic composition comprising a histidyl-tRNA synthetase (HRS) polypeptide.

In some embodiments, the NRP2-associated disease or condition is selected from one or more of cancer and diseases or pathologies associated with cancer, such as cancer cell growth, cancer initiation, cancer migration, cancer cell adhesion, cancer cell invasion, cancer cell chemoresistance, and cancer cell metastasis; diseases associated with inflammation and autoimmunity, optionally inflammatory lung diseases such as hypersensitivity pneumonitis and pulmonary inflammation, and diseases associated with inappropriate immune cell activation or migration, optionally graft versus host disease (GVHD) and rheumatoid arthritis-associated interstitial lung disease (RA-ILD); diseases associated with lymphatic development, lymphangiogenesis, and lymphatic damage, optionally edema, lymphedema, secondary lymphedema, inappropriate fat absorption and deposition, excess fat deposition, and vascular permeability; diseases associated with infections such as latent infections; diseases associated with allergic disorders and allergic responses, optionally chronic obstructive pulmonary disorder (COPD), neutrophilic asthma, antineutrophil cytoplasmic antibody (ANCA)-associated systemic vasculitis, systemic lupus erythematosus, rheumatoid arthritis, inflammasome-related disease(s), and skin-related neutrophil-mediated disease(s) such as pyoderma gangrenosum; diseases associated with granulomatous inflammatory diseases, optionally sarcoidosis, pulmonary granulomatous diseases, and non-lung granulomas; fibrotic diseases such as endometriosis, fibrosis, endothelial to mesenchymal transition (EMT), and wound healing; diseases associated with inappropriate smooth muscle contractility, vascular smooth muscle cell migration, and adhesion; diseases associated with inappropriate autophagy, phagocytosis, and efferocytosis; neuronal diseases, optionally diseases associated with peripheral nervous system remodeling and pain perception; diseases associated with bone development and/or bone remodeling; and diseases associated with inappropriate migratory cell movement.

In some embodiments, the subject has, and/or is selected for treatment based on having, increased extracellular fluid levels of a soluble NRP2 polypeptide (optionally selected from Table N1), either bound or free, relative to the levels of a healthy control or matched control standard or population of subject(s), optionally levels of about or at least about 10, 20, 30, 50, 100, 200, 300, 400, 500, 600, 700, 800, 900, 1000, 1100, 1200, 1300, 1400, 1500, 1600, 1700, 1800, 1900, 2000, 3000, 4000, 5000 pM of the soluble NRP2 polypeptide, or about 30-50, 50-100, 100-2000, 200-2000, 300-2000, 400-2000, 500-2000, 600-2000, 700-2000, 800-2000, 900-2000, 1000-2000, 2000-3000, 3000-4000, 4000-5000 pM of the soluble NRP2 polypeptide.

In some embodiments, the subject has, and/or is selected for treatment based on having, increased extracellular fluid levels of NRP2:NRP2 ligand complexes (optionally selected from Table N1 and Table N2) relative to a healthy control or matched control standard or population of subject(s), optionally levels that are about or at least about 1.5, 2, 3, 4, 5, 6, 7, 8, 9, 10, 15, 20, 30, 40, 50, 60, 70, 80, 90, 100 or more times the levels of the control or reference.

In some embodiments, the subject has, and/or is selected for treatment based on having, increased extracellular fluid levels of HRS:NRP2 complexes (optionally selected from Table H1 and Table N1) relative to a healthy control or matched control standard or population of subject(s), optionally levels that are about or at least about 1.5, 2, 3, 4, 5, 6, 7, 8, 9, 10, 15, 20, 30, 40, 50, 60, 70, 80, 90, 100 or more times the levels of the control or reference.

In some embodiments, the subject has, and/or is selected for treatment based on having, a single nucleotide polymorphism (SNP) in an NRP2 polypeptide or an NRP2 encoding polynucleotide from the subject.

In some embodiments, the subject has, and/or is selected for treatment based on having, a disease associated with increased levels or expression of NRP2a and/or NRP2b, or an altered ratio of NRP2a:NRP2b expression, relative to a healthy control or matched control standard or population of subject(s). In some embodiments, the levels of NRP2b are increased by about or at least about 10%, 20%, 30%, 40%, 50%, 100%, 200%, 300%, 400%, 500%, 600%, 700%, 800%, 900%, 1000% compared to a healthy control or matched control standard or population of subject(s).

In some embodiments, the healthy control or matched control standard or population of subject(s) comprises average ranges for age-matched samples of cancerous or non-cancerous cells or tissue of the same type as the cancer, which comprise specific characteristics such as drug resistance, metastatic potential, aggressiveness, genetic signature (optionally p53 mutation(s), PTEN deletion, IGFR expression), and/or expression patterns

In some embodiments, the subject in need thereof has, and/or is selected for treatment based on having, an infection, and optionally wherein the method further comprises administering to the subject at least one antimicrobial agent, antifungal agent, and/or antihelminthic agent. In some embodiments, (a) the HRS polypeptide and (b) the antimicrobial agent, antifungal agent, and/or antihelminthic agent, are administered together as part of the same therapeutic composition. In some embodiments, (a) the HRS polypeptide and (b) the antimicrobial agent, antifungal agent, and/or antihelminthic agent, are administered as separate therapeutic compositions. In some embodiments, the antimicrobial agent, antifungal agent, and/or antihelminthic agent is selected from one or more of aminoglycosides such as Amikacin, Gentamicin, Kanamycin, Neomycin, Netilmicin, Tobramycin, Paromomycin, Streptomycin, and Spectinomycin; carbapenems such as Ertapenem, Doripenem, Imipenem/Cilastatin, and Meropenem; cephalasporins such as Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Cefepime, Ceftaroline fosamil, and Ceftobiprole; glycopeptides such as Teicoplanin, Vancomycin, Telavancin, Dalbavancin, Oritavancin; lincosamines such as Clindamycin and Lincomycin; macrolides such as Azithromycin, Clarithromycin, Erythromycin, Roxithromycin, Telithromycin, and Spiramycin; penicillins such as Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; polypeptides such as Bacitracin, Colistin, and Polymyxin B; quinolones/fluoroquinolones such as Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; sulfonamides such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), and Sulfonamidochrysoidine; tetracyclines such as Demeclocycline, Doxycycline, Metacycline, Minocycline, Oxytetracycline, and Tetracycline; anti-mycobacterials such as Clofazimine, Dapsone, Capreomycin, Cycloserine, Ethambutol, Ethionamide, Isoniazid, Pyrazinamide, Rifampicin (Rifampin), Rifabutin, Rifapentine, and Streptomycin; Chloramphenicol; Metronidazole; Mupirocin; Tigecycline; Tinidazole; and antihelminthic agents such as Diethylcarbamazine and Albendazole.

Also included are methods selected from one or more of the following:
a method for improving or restoring lymphatic vessel function in a subject in need thereof;
a method for modulating lymphangiogenesis in a subject in need thereof;
method for treating a semaphorin signaling-associated disease or disorder in a subject in need thereof;
a method for modulating vascular endothelial growth factor C (VEGF-C) signaling in a subject in need thereof;
a method for modulating integrin signaling in a subject in need thereof;
a method for modulating TGF-β signaling in a subject in need thereof;
a method for modulating autophagy, phagocytosis, or efferocytosis in a subject in need thereof;
a method for modulating neuronal development in a subject in need thereof;
a method for reducing lymphatic endothelial cell migration or adhesion in a subject in need thereof;
a method for modulating endothelial to mesenchymal transition (EMT) in a subject in need thereof;
a method for modulating bone development in a subject in need thereof;
a method for modulating vascular permeability in a subject in need thereof;
a method for modulating binding or functional interaction between an NRP2 polypeptide and an NRP2 ligand in a subject in need thereof;
a method for inhibiting immune cell activity, migration, or adhesion in a subject in need thereof; and
a method for reducing tumor cell migration or adhesion in a subject in need thereof,

wherein the method comprises administering to the subject in need thereof a therapeutic composition comprising a histidyl-tRNA synthetase (HRS) polypeptide.

In some embodiments, the lymphangiogenesis is secondary to a cancer, a corneal injury, a dry eye disease, inflammation, lymphedema, a graft rejection, or any combination thereof. In some embodiments, the neuronal development is peripheral nerve remodeling associated with an inflammatory or autoimmune condition. In some embodiments, the NRP2 ligand is selected from VEGF-C, VEGF-D, VEGF-A145, VEGFA165, PIGF-2, Semaphorin 3B, 3C, 3D and 3F, heparin, an integrin, and TGF-beta. In some embodiments, the NRP2 ligand is selected from VEGF-C, VEGF-D, VEGF-A145, VEGFA165, and PIGF-2. In some embodiments, the NRP2 ligand is selected from Semaphorins 3B, 3C, 3D, 3F, and 3G. In some embodiments, the immune cell is selected from a myeloid derived cell, a macrophage, a neutrophil, an eosinophil, a granulocyte, a dendritic cell, a T cell, a B cell, and a natural killer (NK) cell. In some embodiments, the T cell is a T_{REG} cell, a T_{H1} cell, or a T_{H2} cell. In some embodiments, the macrophage is an M1 or M2 macrophage. In some embodiments, the methods include reducing the tumor cell migration within the lymphatic system.

In some embodiments, the subject has a neuropilin-2 (NRP2) associated disease or condition, optionally wherein the subject has, and/or is selected for treatment based on having, increased extracellular fluid levels of a soluble NRP2 polypeptide, increased extracellular fluid levels of NRP2:NRP2 ligand complexes, increased extracellular fluid levels of HRS:NRP2 complexes, and/or a single nucleotide polymorphism (SNP) in an NRP2 polypeptide or an NRP2 encoding polynucleotide from the subject.

In some embodiments, the disease is a cancer, for example, wherein the cancer expresses or overexpresses NRP2. In some instances, the cancer displays NRP2-dependent growth, NRP2-dependent adhesion, NRP2-dependent migration, NRP2-dependent chemoresistance, and/or NRP2-dependent invasion. In some embodiments, the cancer is a primary cancer. In some embodiments, the cancer is a metastatic cancer, optionally a metastatic cancer that expresses NRP2a and/or NRP2b.

In some embodiments, the cancer is chemoresistant to at least one cancer therapy, for example, a cancer immunotherapy agent, a chemotherapeutic agent, a hormonal therapeutic agent, and/or a kinase inhibitor. In some instances, the method comprises selecting a subject having a cancer that is chemoresistant to at least one cancer therapy prior to administering the HRS polypeptide.

In some embodiments, the HRS polypeptide modulates autophagy, efferocytosis, or phagocyte maturation in a cancer cell or cancer-associated macrophage. In specific embodiments, the HRS polypeptide modulates autophagy in the cancer cell.

In some embodiments, the cancer is selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, and stomach cancer.

In some embodiments, the metastatic cancer is selected from one or more of:

(a) a bladder cancer which has metastasized to the bone, liver, and/or lungs;

(b) a breast cancer which has metastasized to the bone, brain, liver, and/or lungs;

(c) a colorectal cancer which has metastasized to the liver, lungs, and/or peritoneum;

(d) a kidney cancer which has metastasized to the adrenal glands, bone, brain, liver, and/or lungs;

(e) a lung cancer which has metastasized to the adrenal glands, bone, brain, liver, and/or other lung sites;

(f) a melanoma which has metastasized to the bone, brain, liver, lung, and/or skin/muscle;

(g) a ovarian cancer which has metastasized to the liver, lung, and/or peritoneum;

(h) a pancreatic cancer which has metastasized to the liver, lung, and/or peritoneum;

(i) a prostate cancer which has metastasized to the adrenal glands, bone, liver, and/or lungs;

(j) a stomach cancer which has metastasized to the liver, lung, and/or peritoneum;

(l) a thyroid cancer which has metastasized to the bone, liver, and/or lungs; and

(m) a uterine cancer which has metastasized to the bone, liver, lung, peritoneum, and/or vagina.

Some embodiments include administering to the subject at least one additional agent selected from one or more of a cancer immunotherapy agent, a chemotherapeutic agent, a hormonal therapeutic agent, and a kinase inhibitor. In some embodiments, the at least one HRS polypeptide and the at least one agent are administered separately, as separate compositions. In some embodiments, the at least one HRS polypeptide and the at least one agent are administered together as part of the same therapeutic composition.

In some embodiments, the cancer immunotherapy agent is selected from one or more of an immune checkpoint modulatory agent, a cancer vaccine, an oncolytic virus, a cytokine, and a cell-based immunotherapies. In some embodiments, the immune checkpoint modulatory agent is a polypeptide, optionally an antibody or antigen-binding fragment thereof or a ligand, or a small molecule. In some embodiments, the immune checkpoint modulatory agent comprises

(a) an antagonist of a inhibitory immune checkpoint molecule; or

(b) an agonist of a stimulatory immune checkpoint molecule, for example, wherein the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736), optionally wherein the cancer is selected from one or more of colorectal cancer, melanoma, breast cancer, non-small-cell lung carcinoma, bladder cancer, and renal cell carcinoma;

the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab, optionally wherein the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer;

the PD-1 antagonist is pembrolizumab and the cancer is optionally selected from one or more of melanoma, non-small cell lung cancer, small cell lung cancer, head and neck cancer, and urothelial cancer;

the antagonist is a CTLA-4 antagonist optionally selected from one or more of an antibody or antigen-binding fragment or small molecule that specifically binds thereto, ipilimumab, tremelimumab, optionally wherein the cancer is selected from one or more of melanoma, prostate cancer, lung cancer, and bladder cancer;

the antagonist is an IDO antagonist optionally selected from one or more of an antibody or antigen-binding fragment or small molecule that specifically binds thereto, indoximod (NLG-8189), 1-methyl-tryptophan (IMT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor;

the antagonist is a TDO antagonist optionally selected from one or more of an antibody or antigen-binding fragment or small molecule that specifically binds thereto, 680C91, and LM10;

the antagonist is a TIM-3 antagonist optionally selected from one or more of an antibody or antigen-binding fragment or small molecule that specifically binds thereto;

the antagonist is a LAG-3 antagonist optionally selected from one or more of an antibody or antigen-binding fragment or small molecule that specifically binds thereto, and BMS-986016;

the antagonist is a VISTA antagonist optionally selected from one or more of an antibody or antigen-binding fragment or small molecule that specifically binds thereto;

the antagonist is a BTLA, CD160, and/or HVEM antagonist optionally selected from one or more of an antibody or antigen-binding fragment or small molecule that specifically binds thereto;

the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agonist is an OX40 agonist optionally selected from one or more of an antibody or antigen-binding fragment or small molecule or ligand that specifically binds thereto, OX86, Fc-OX40L, and GSK3174998;

the agonist is a CD40 agonist optionally selected from one or more of an antibody or antigen-binding fragment or small molecule or ligand that specifically binds thereto, CP-870,893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma;

the agonist is a GITR agonist optionally selected from one or more of an antibody or antigen-binding fragment or small molecule or ligand that specifically binds thereto, INCAGN01876, DTA-1, and MEDI1873;

the agonist is a CD137 agonist optionally selected from one or more of an antibody or antigen-binding fragment or small molecule or ligand that specifically binds thereto, utomilumab, and 4-1BB ligand;

the agonist is a CD27 agonist optionally selected from one or more of an antibody or antigen-binding fragment or small molecule or ligand that specifically binds thereto, varlilumab, and CDX-1127 (1F5);

the agonist is a CD28 agonist optionally selected from one or more of an antibody or antigen-binding fragment or small molecule or ligand that specifically binds thereto, and TAB08; and/or the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In some embodiments, the cytokine selected from one or more of interferon (IFN)-α, IL-2, IL-12, IL-7, IL-21, and Granulocyte-macrophage colony-stimulating factor (GM-CSF).

In some embodiments, the cell-based immunotherapy agent comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

In some embodiments, the at least one chemotherapeutic agent is selected from one or more of an alkylating agent, an anti-metabolite, a cytotoxic antibiotic, a topoisomerase inhibitor (type 1 or type II), and an anti-microtubule agent.

In some embodiments, the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);

the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);

the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;

the topoisomerase inhibitor is selected from one or more of camptothecin, irinotecan, topotecan, etoposide, doxorubicin, mitoxantrone, teniposide, novobiocin, merbarone, and aclarubicin; and/or

the anti-microtubule agent is selected from one or more of taxanes (optionally paclitaxel and docetaxel) and vinca alkaloids (optionally vinblastine, vincristine, vindesine, vinorelbine).

In some embodiments, the at least one hormonal therapeutic agent is a hormonal agonist or a hormonal antagonist. In some embodiments, the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog. In some embodiments, the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib. In some embodiments, the kinase inhibitor is a PI3 kinase inhibitor selected from one or more of alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147.

Also included are methods for treating an inflammatory lung disease in a subject in need thereof, comprising administering to the subject a therapeutic composition comprising a histidyl-tRNA synthetase (HRS) polypeptide. In some embodiments, the inflammatory lung disease is a neuropilin-2 (NRP2) associated disease or condition, for example, RA-ILD, chronic hypersensitivity pneumonitis, pulmonary inflammation, pulmonary granulomatous disease, or sarcoidosis. In some embodiments, the subject has, and/or is selected for treatment based on having, increased extracellular fluid levels of a soluble NRP2 polypeptide, increased extracellular fluid levels of NRP2:NRP2 ligand complexes, increased extracellular fluid levels of HRS:NRP2 complexes, and/or a single nucleotide polymorphism (SNP) in an NRP2 polypeptide or an NRP2 encoding polynucleotide from the subject, as described herein.

Some embodiments include methods for treating lymphedema in a subject in need thereof, comprising administering to the subject a therapeutic composition comprising a histidyl-tRNA synthetase (HRS) polypeptide. In some embodiments, the subject has, and/or is selected for treatment based on having, a Stage of lymphedema selected from Stage 1, Stage 2, Stage 3, Stage 4, Stage 5, Stage 6, and Stage 7. In some embodiments, the subject has, and/or is selected for treatment based on having, a Grade of lymphedema selected from Grade 1 (mild edema), Grade 2 (moderate edema), Grade 3a (severe edema), Grade 3b (massive edema), and Grade 4 (gigantic edema). In some embodiments, the subject has, and/or is selected for treatment based on having, lymphedema-associated fibrosis. In some embodiments, the subject has, and/or is selected for treatment based on having, lymphedema that is secondary to cancer (optionally breast cancer), surgery (optionally cancer surgery, optionally breast cancer surgery), radiation therapy, obesity, congestive heart failure, hypertension, peripheral vascular/venous disease, or any combination thereof.

Some embodiments comprise measuring a decrease in at least one symptom of lymphedema in the subject. In some embodiments, the at least one symptom of lymphedema is selected from swelling, thickness of skin, hardening of skin, feeling of fullness, aching, discomfort, restricted range of motion, and any combination thereof.

In some embodiments, the lymphedema is a neuropilin-2 (NRP2) associated disease or condition. In some embodiments, the subject has, and/or is selected for treatment based on having, increased extracellular fluid levels of a soluble NRP2 polypeptide, increased extracellular fluid levels of NRP2:NRP2 ligand complexes, increased extracellular fluid levels of HRS:NRP2 complexes, and/or a single nucleotide polymorphism (SNP) in an NRP2 polypeptide or an NRP2 encoding polynucleotide from the subject.

In some embodiments, the subject in need thereof has, and/or is selected for treatment based on having, a lymphedema-associated infection, optionally erysipelas, cellulitis, lymphangitis, and/or sepsis, and optionally wherein the method further comprises administering to the subject at least one antimicrobial agent, antifungal agent, and/or antihelminthic agent. In some embodiments, (a) the HRS polypeptide and (b) the antimicrobial agent, antifungal agent, and/or antihelminthic agent, are administered together as part of the same therapeutic composition. In some embodiments, (a) the HRS polypeptide and (b) the antimicrobial agent, antifungal agent, and/or antihelminthic agent, are administered as separate therapeutic compositions. In some embodiments, the antimicrobial agent, antifungal agent, and/or antihelminthic agent is selected from one or more of aminoglycosides such as Amikacin, Gentamicin, Kanamycin, Neomycin, Netilmicin, Tobramycin, Paromomycin, Streptomycin, and Spectinomycin; carbapenems such as Ertapenem, Doripenem, Imipenem/Cilastatin, and Meropenem; cephalasporins such as Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Cefepime, Ceftaroline fosamil, and Ceftobiprole; glycopeptides such as Teicoplanin, Vancomycin, Telavancin, Dalbavancin, Oritavancin; lincosamines such as Clindamycin and Lincomycin; macrolides such as Azithromycin, Clarithromycin, Erythromycin, Roxithromycin, Telithromycin, and Spiramycin; penicillins such as Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; polypeptides such as Bacitracin, Colistin, and Polymyxin B; quinolones/fluoroquinolones such as Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; sulfonamides such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), and Sulfonamidochrysoidine; tetracyclines such as Demeclocycline, Doxycycline, Metacycline, Minocycline, Oxytetracycline, and Tetracycline; anti-mycobacterials such as Clofazimine, Dapsone, Capreomycin, Cycloserine, Ethambutol, Ethionamide, Isoniazid, Pyrazinamide, Rifampicin (Rifampin), Rifabutin, Rifapentine, and Streptomycin; Chloramphenicol; Metronidazole; Mupirocin; Tigecycline; Tinidazole; and antihelminthic agents such as Diethylcarbamazine and Albendazole.

In some embodiments, the HRS polypeptide comprises, consists, or consists essentially of an amino acid sequence that is at least 80%, 85%, 90%, 95%, 96%, 97%, 98%, 99%, or 100% identical to SEQ ID NO:156 (Fc-HRS(2-60) or HRS^{FC1}). In some embodiments, the HRS polypeptide comprises, consists, or consists essentially of an amino acid sequence that is at least 80%, 85%, 90%, 95%, 96%, 97%, 98%, 99%, or 100% identical to a sequence selected from Table H1, Table H2, and Table H4. In some embodiments, the HRS polypeptide is 500-506 amino acids in length and is at least 90%, 95%, 96%, 97%, 98%, 99%, or 100% identical to SEQ ID NO:7 (HRS(1-506)) or 8 (HRS(2-506)) and lacks residues 507-509 of SEQ ID NO:1.

In some embodiments, the HRS polypeptide is fused to a heterologous polypeptide. In some embodiments, the heterologous polypeptide comprises an Fc region, to form an HRS-Fc fusion polypeptide, optionally wherein the HRS-Fc fusion polypeptide comprises, consists, or consists essentially of an amino acid sequence that is at least 80%, 85%, 90%, 95%, 96%, 97%, 98%, 99%, or 100% identical to a sequence selected from Table H8. In some embodiments, the heterologous polypeptide comprises a cartilage oligomeric protein (COMP) polypeptide, optionally a COMP pentameric domain polypeptide, to form an HRS-COMP fusion polypeptide, optionally wherein the HRS-COMP fusion polypeptide comprises, consists, or consists essentially of an amino acid sequence that is at least 80%, 85%, 90%, 95%, 96%, 97%, 98%, 99%, or 100% identical to a sequence selected from Table H9.

Also included are therapeutic compositions, comprising:

(a) a histidyl-tRNA synthetase (HRS) polypeptide; and

(b) at least one additional agent selected from an antimicrobial agent, an antifungal agent, an antihelminthic agent, a cancer immunotherapy agent, a chemotherapeutic agent, a hormonal therapeutic agent, and a kinase inhibitor.

In some embodiments, the HRS polypeptide comprises, consists, or consists essentially of an amino acid sequence that is at least 80%, 85%, 90%, 95%, 96%, 97%, 98%, 99%, or 100% identical to SEQ ID NO:156 (Fc-HRS(2-60) or HRS^{F}″). In some embodiments, the HRS polypeptide comprises, consists, or consists essentially of an amino acid sequence that is at least 80%, 85%, 90%, 95%, 96%, 97%, 98%, 99%, or 100% identical to a sequence selected from Table H1, Table H2, and Table H4. In some embodiments, the HRS polypeptide is 500-506 amino acids in length and is at least 90%, 95%, 96%, 97%, 98%, 99%, or 100% identical to SEQ ID NO:7 (HRS(1-506)) or 8 (HRS(2-506)) and lacks residues 507-509 of SEQ ID NO:1.

In some embodiments, the HRS polypeptide is fused to a heterologous polypeptide. In some embodiments, the heterologous polypeptide comprises an Fc region, to form an HRS-Fc fusion polypeptide, optionally wherein the HRS-Fc fusion polypeptide comprises, consists, or consists essentially of an amino acid sequence that is at least 80%, 85%, 90%, 95%, 96%, 97%, 98%, 99%, or 100% identical to a sequence selected from Table H8. In some embodiments, the heterologous polypeptide comprises a cartilage oligomeric protein (COMP) polypeptide, optionally a COMP pentameric domain polypeptide, to form an HRS-COMP fusion polypeptide, optionally wherein the HRS-COMP fusion polypeptide comprises, consists, or consists essentially of an amino acid sequence that is at least 80%, 85%, 90%, 95%, 96%, 97%, 98%, 99%, or 100% identical to a sequence selected from Table H9.

In some embodiments, the antimicrobial agent, antifungal agent, and/or antihelminthic agent is selected from one or more of aminoglycosides such as Amikacin, Gentamicin, Kanamycin, Neomycin, Netilmicin, Tobramycin, Paromomycin, Streptomycin, and Spectinomycin; carbapenems such as Ertapenem, Doripenem, Imipenem/Cilastatin, and Meropenem; cephalasporins such as Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Cefepime, Ceftaroline fosamil, and Ceftobiprole; glycopeptides such as Teicoplanin, Vancomycin, Telavancin, Dalbavancin, Oritavancin; lincosamines such as Clindamycin and Lincomycin; macrolides such as Azithromycin, Clarithromycin, Erythromycin, Roxithromycin, Telithromycin, and Spiramycin; penicillins such as Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; polypeptides such as Bacitracin, Colistin, and Polymyxin B; quinolones/fluoroquinolones such as Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; sulfonamides such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), and Sulfonamidochrysoidine; tetracyclines such as Demeclocycline, Doxycycline, Metacycline, Minocycline, Oxytetracycline, and Tetracycline; anti-mycobacterials such as Clofazimine, Dapsone, Capreomycin, Cycloserine, Ethambutol, Ethionamide, Isoniazid, Pyrazinamide, Rifampicin (Rifampin), Rifabutin, Rifapentine, and Streptomycin; Chloramphenicol; Metronidazole; Mupirocin; Tigecycline; Tinidazole; and antihelminthic agents such as Diethylcarbamazine and Albendazole

In some embodiments, the cancer immunotherapy agent is selected from one or more of an immune checkpoint modulatory agent, a cancer vaccine, an oncolytic virus, a cytokine, and a cell-based immunotherapies.

In some embodiments, the immune checkpoint modulatory agent is a polypeptide, optionally an antibody or antigen-binding fragment thereof or a ligand, or a small molecule. In some embodiments, the immune checkpoint modulatory agent comprises

(a) an antagonist of a inhibitory immune checkpoint molecule; or

(b) an agonist of a stimulatory immune checkpoint molecule, for example, wherein the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736);

the antagonist is a PD-1 antagonist optionally selected from one or more of an antibody or antigen-binding fragment or small molecule that specifically binds thereto, nivolumab, pembrolizumab, MK-3475, AMP-224, AMP-514, PDR001, and pidilizumab;

the antagonist is a CTLA-4 antagonist optionally selected from one or more of an antibody or antigen-binding fragment or small molecule that specifically binds thereto, ipilimumab, and tremelimumab;

the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat;

the antagonist is a TDO antagonist optionally selected from one or more of an antibody or antigen-binding fragment or small molecule that specifically binds thereto, 680C91, and LM10;

the antagonist is a TIM-3 antagonist optionally selected from one or more of an antibody or antigen-binding fragment or small molecule that specifically binds thereto;

the antagonist is a LAG-3 antagonist optionally selected from one or more of an antibody or antigen-binding fragment or small molecule that specifically binds thereto, and BMS-986016;

the antagonist is a VISTA antagonist optionally selected from one or more of an antibody or antigen-binding fragment or small molecule that specifically binds thereto;

the antagonist is a BTLA, CD160, and/or HVEM antagonist optionally selected from one or more of an antibody or antigen-binding fragment or small molecule that specifically binds thereto; and/or the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agonist is an OX40 agonist optionally selected from one or more of an antibody or antigen-binding fragment or small molecule or ligand that specifically binds thereto, OX86, Fc-OX40L, and GSK3174998;

the agonist is a CD40 agonist optionally selected from one or more of an antibody or antigen-binding fragment or small molecule or ligand that specifically binds thereto, CP-870,893, dacetuzumab, Chi Lob 7/4, ADC-1013, and rhCD40L;

the agonist is a GITR agonist optionally selected from one or more of an antibody or antigen-binding fragment or small molecule or ligand that specifically binds thereto, INCAGN01876, DTA-1, and MEDI1873;

the agonist is a CD137 agonist optionally selected from one or more of an antibody or antigen-binding fragment or small molecule or ligand that specifically binds thereto, utomilumab, and 4-1BB ligand;

the agonist is a CD27 agonist optionally selected from one or more of an antibody or antigen-binding fragment or small molecule or ligand that specifically binds thereto, varlilumab, and CDX-1127 (1F5);

the agonist is a CD28 agonist optionally selected from one or more of an antibody or antigen-binding fragment or small molecule or ligand that specifically binds thereto, and TAB08; and/or

the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In some embodiments, the cytokine selected from one or more of interferon (IFN)-a, IL-2, IL-12, IL-7, IL-21, and Granulocyte-macrophage colony-stimulating factor (GM-CSF).

In some embodiments, the cell-based immunotherapy agent comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

In some embodiments, the at least one chemotherapeutic agent is selected from one or more of an alkylating agent, an anti-metabolite, a cytotoxic antibiotic, a topoisomerase inhibitor (type 1 or type II), and an anti-microtubule agent.

In some embodiments, the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);

the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);

the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;

the topoisomerase inhibitor is selected from one or more of camptothecin, irinotecan, topotecan, etoposide, doxorubicin, mitoxantrone, teniposide, novobiocin, merbarone, and aclarubicin; and/or

the anti-microtubule agent is selected from one or more of taxanes (optionally paclitaxel and docetaxel) and vinca alkaloids (optionally vinblastine, vincristine, vindesine, vinorelbine).

In some embodiments, the at least one hormonal therapeutic agent is a hormonal agonist or a hormonal antagonist. In some embodiments, the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog. In some embodiments, the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib. In some embodiments, the kinase inhibitor is a PI3 kinase inhibitor selected from one or more of alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147.

In some embodiments, the therapeutic composition has a purity of at least about 80%, 85%, 90%, 95%, 98%, or 99% on a protein basis with respect to the HRS polypeptide, and is substantially aggregate-free. In some embodiments, the therapeutic composition is substantially endotoxin-free. In some embodiments, the therapeutic composition is a sterile, injectable solution, optionally suitable for intravenous, intramuscular, subcutaneous, or intraperitoneal administration.

Also included are patient care kits, comprising:

(a) a histidyl-tRNA synthetase (HRS) polypeptide, as described herein; and

(b) at least one additional agent selected from an antimicrobial agent, an antifungal agent, an antihelminthic agent, a cancer immunotherapy agent, a chemotherapeutic agent, a hormonal therapeutic agent, and a kinase inhibitor, as described herein.

In some embodiments, (a) and (b) are in separate therapeutic compositions. In some embodiments, (a) and (b) are in the same therapeutic composition.

Certain embodiments include an isolated fusion protein, comprising a histidyl-tRNA synthetase polypeptide fused to a cartilage oligomeric protein (COMP) polypeptide, optionally a COMP pentameric domain polypeptide, to form an HRS-COMP fusion polypeptide, optionally wherein the HRS-COMP fusion polypeptide comprises, consists, or consists essentially of an amino acid sequence that is at least 80%, 85%, 90%, 95%, 96%, 97%, 98%, 99%, or 100% identical to a sequence selected from Table H9.

Some embodiments include a therapeutic composition, comprising a HRS-COMP fusion protein described herein. In some embodiments, the therapeutic composition has a purity of at least about 80%, 85%, 90%, 95%, 98%, or 99% on a protein basis with respect to the HRS-COMP fusion polypeptide, and is substantially aggregate-free. In particular embodiments, the therapeutic composition is substantially endotoxin-free. In some embodiments, the therapeutic composition is a sterile, injectable solution, optionally suitable for intravenous, intramuscular, subcutaneous, or intraperitoneal administration

### BRIEF DESCRIPTION OF THE DRAWINGS

FIGS. 1A-1B illustrate the general domain structure of neuropilins (1A) and exemplary neuropilin co-receptor functions (1B).

FIG. 2 illustrates the domain structure of certain NRP2 isoforms and NRP2 ligand binding domains.

FIG. 3 shows binding of human NRP2 to Fc-HRS(2-60) on an SPR chip. 50 nM of NRP2 (solid black line), NRP1 (solid gray line) and mouse Plexin A1 (dotted line) were flowed as analytes over an SPR chip coated with immobilized Fc-HRS(2-60).

FIGS. 4A-4B shows binding of NRP2 from human, mouse, and rat to Fc-HRS(2-60) and not a truncated form of Fc-HRS(2-11). 50 nM of human NRP2 (solid black line), mouse NRP2 (dashed line), rat NRP2 (solid gray line), or NRP1 (dotted line) were flowed as analytes over an SPR chip coated with immobilized full length Fc-HRS(2-60) (4A), or a truncated form of Fc-HRS(2-11) missing 49 amino acids at the C-terminus (4B).

FIGS. 5A-5D show binding of human NRP2 to Fc-HRS(2-60) and t-RNA synthetases comprising domains that share homology with the WHEP domain of Fc-HRS(2-60). 20 nM of NRP2 was flowed as an analyte over SPR chip surfaces coated with immobilized Fc-HRS(2-60) (5A), GARS Fc-WHEP (5B), MARS Fc-WHEP (5C), or WARS WHEP (5D).

FIGS. 6A-6B show the binding of human NRP2 to Fc-HRS(2-60) on an SPR chip coated with immobilized Fc-HRS(2-60) in the presence and absence of divalent cations. The running buffer in this experiment was 50 mM HEPES, 300 mM NaCl, 0.005% Tween20, pH 7.4. For each analyte, 20 nM NRP2 was prepared in running buffer supplemented with 5 mM of either CaCl₂), EDTA (6A) or MgCl₂, MgCl₂+CaCl₂) or ZnCl₂ (6B).

FIGS. 7A-7B show binding of a pre-formed complex of Fc-HRS(2-60) and NRP2 to 4D4 monoclonal antibody but not to the 1C8 monoclonal antibody. Monoclonal antibodies against Fc-HRS(2-60) (monoclonal antibody clones 1C8 (7A) and 4D4 (7B)) were immobilized on an SPR chip. Analytes consisted of 200 nM NRP2 (dotted line), 100 nM Fc-HRS(2-60) (solid black line), a mixture of 100 nM Fc-HRS(2-60) and 200 nM NRP2 (solid gray line), or a mixture of 100 nM Fc-HRS(2-60) and 200 nM 1C8 mAb (dashed line).

FIGS. 8A-8D show binding of NRP2 to Fc-HRS(2-60) captured by some monoclonal antibodies against Fc-HRS(2-60) but not others. Monoclonal antibodies against Fc-HRS(2-60) (Monoclonal antibody clones 12H6 (8A), 1C8 (8B), 4D4 (8C) and 13E9 (8D) were immobilized on an SPR chip. Co-injections were then carried out where one analyte is injected, immediately followed by a second analyte. Timing of the two injections is indicated by arrowheads. In each of the panels above, 2000 nM Fc-HRS(2-60) was injected as the first analyte to saturate the antibody surfaces, followed by either additional Fc-HRS(2-60) (solid gray line), or 200 nM NRP2 (solid black line). To rule out non-specific binding of NRP2 to the antibody surfaces, co-injection of buffer followed by 200 nM NRP2 were also performed (dotted line).

FIGS. 9A-9B show dose-dependent binding of Fc-HRS (2-60) to cells expressing a NRP2a-GFP fusion protein. Quantification of the staining intensity (9A) and staining intensity CV (9B) of Fc-HRS (2-60)/anti-Fc-PE complex on HEK293T cells overexpressing NRP2v2-GFP. Intensity values are from cells gated on high NRP2 expression (GFP Bright). Fc-HRS (2-60) was titrated in 2 fold steps and then combined with 87.5 nM of anti-Fc-PE. As a control for specificity, 175 nM Fc-HRS (2-11)/anti-Fc-PE was included.

FIG. 10 shows binding inhibition of Fc-HRS (2-60) to cells expressing a NRP2a-GFP fusion protein in the presence of anti-HRS antibody clone 1C8. Quantification of the staining intensity of Fc-HRS (2-60)/anti-Fc-PE complex pre-incubated with either an isotype antibody control or anti-HRS (WHEP) clone 1C8 on HEK293T cells overexpressing NRP2v2-GFP. Intensity values are from cells gated on high NRP2 expression (GFP Bright). 175 nM of Fc-HRS (2-60)/anti-Fc-PE was used. As a control for specificity, 175 nM Fc-HRS (2-11)/anti-Fc-PE was included.

FIGS. 11A-11B show that anti-HRS antibodies from the KL31 series blocked binding of Fc-HRS(2-60) to NRP2 in a concentration-dependent manner, whereas other antibodies of the AB04 and AB13 series did not demonstrate significant blocking characteristics in this assay. Quantification of the staining of stably expressing Expi293-NRP2 cells with biotinylated Fc-HRS—streptavidin-PE using flow cytometry in the presence of various concentrations of anti-HRS antibodies. Data are from two experiments using different antibodies. FIG. 11A shows control human IgG1 (filled circles), KL31-467 (filled triangles), KL31-356 (partially filled triangles), mouse clone 13C8 (crosses), and 11B shows control human IgG1 (filled circles), AB04-425 (open triangles), AB13-288 (partially filled squares), and KL31-478 (filled triangles), which are shown as the percentage of streptavidin-PE+/NRP2+ cells in the viable singlet gate.

FIGS. 12A-12B show binding inhibition of Fc-HRS (2-60) to cells expressing a NRP2a-GFP fusion protein in the presence of VEGF-C. Quantification of the staining intensity of Fc-HRS (2-60)/anti-Fc-PE complex pre-incubated with different doses of VEGF-C on HEK293T cells overexpressing NRP2v2-GFP. Intensity values are from cells gated on high NRP2 expression (GFP Bright). 175 nM of Fc-HRS (2-60)/anti-Fc-PE was used. As a control for specificity, 175 nM Fc-HRS (2-11)/anti-Fc-PE was included.

FIG. 13 shows quantification of circulating NRP2 levels in serum and plasma from normal healthy donors. Normal healthy volunteer (n=72) serum and plasma was isolated and quantified for circulating levels of NRP-2. Serum (black circles) and plasma (open squares) samples were tested in an ELISA specific for human NRP-2. Mean levels for serum (16.3 μM) and plasma (15.6 μM) were shown for all 72 samples. The limit of quantification for the NRP2 ELISA was 1.5 μM.

FIG. 14 shows a comparison of circulating HRS and NRP2 levels. Serum HRS (black circles) levels show a broad range in circulation within 72 normal healthy volunteers tested. Matching serum NRP2 levels from the identical donors were overlaid on the same axes. Donors with low HRS levels show low to undetectable levels of soluble NRP2 (Limit of quantification=1.5 μM). Those donors with elevated HRS levels generally have corresponding increased levels of circulating NRP2.

FIG. 15 shows HRS N-terminal interference in human serum from healthy donors. Normal serum from healthy donors was assayed in two separate HARS ELISAs. Samples were assayed in an ELISA to detect full length HARS (HARS_FL; black circles) as well as an ELISA directed against specifically the N-terminus (HARS_NT; open squares). The lack of correlation between these two ELISAs, as full length HARS levels increased, is referred to as N-terminal interference and may represent the presence of a cofactor, binding partner or soluble receptor to HRS.

FIG. 16 shows a correlation between HARS N-terminal assay interference and soluble NRP2 levels. Normal healthy serum was analyzed for differences in detection with two HARS ELISAs and compared to circulating NRP2 levels. The difference in levels detected between the full length HARS ELISA and the N-terminal HARS ELISA was termed HARS N-terminal Interference Units. These interference units were plotted versus soluble NRP2 levels. The results show a relationship between increased N-terminal interference and soluble NRP2 in normal serum.

FIG. 17 shows detection of an endogenous HRS & NRP2 soluble complex. Serum samples from normal healthy donors were analyzed in multiple HRS & NRP-2 complex ELISAs. These assay formats utilized capture of circulating HRS (HARS_NT or HARS_CT) and detection with an NRP2 monoclonal antibody. Similarly the reverse format was also used whereby circulating NRP2 was captured and detection was observed with anti-HRS antibodies. In both formats, signals were elevated in the high interference samples as compared to low interference serum samples.

FIG. 18 shows that complexed HRS and NRP2 in high interference samples blocks detection with a site-specific HRS antibody. Serum from low and high HRS N-terminal interference samples was assayed in a HRS and NRP2 complex ELISA. Serum samples were captured with an NRP2 monoclonal antibody and detected with either of two unique HRS N-terminal monoclonal antibodies. Samples with high interference showed complex formation when detected with HRS NT (black bars) but this signal was completely blocked with an N-terminal anti-HRS antibody (HRS blocking antibody; gray bars).

FIGS. 19A-19C shows the activity of Fc-HRS(2-60) on skin markers in a murine model of sclerodermatous chronic Graft vs. Host Disease. FIG. 19A shows the impact of treatment with Vehicle, Fc-HRS(2-60) or nintedanib beginning 7 (7 D) or 21 (21 D) days after allogenic transplantation on dermal thickness. FIG. 19B shows the impact of treatment with Vehicle, Fc-HRS(2-60) or nintedanib beginning 7 (7 D) or 21 (21 D) days after allogenic transplantation on the number of myofibroblasts counted in skin sections. FIG. 19C shows the impact of treatment with Vehicle, Fc-HRS(2-60) or nintedanib beginning 7 (7 D) or 21 (21 D) days after allogenic transplantation on hydroxyproline content (an indicator or collagen content) in the skin. ****p<0.0001, ***p<0.001, **p<0.01, *p<0.05 Kruskal-Wallis non-parametric ANOVA followed by Dunn's multiple comparison test to Vehicle animals euthanized 8 weeks after allogenic transplant (3^{rd} group from the left).

FIGS. 20A-20D show the activity of Fc-HRS(2-60) on lung markers in a murine model of sclerodermatous chronic Graft vs. Host Disease. FIG. 20A shows the impact of treatment with Vehicle, Fc-HRS(2-60) or nintedanib beginning 7 (7 D) or 21 (21 D) days after allogenic transplantation on Ashcroft score. FIG. 20B shows the impact of treatment with Vehicle, Fc-HRS(2-60) or nintedanib beginning 7 (7 D) or 21 (21 D) days after allogenic transplantation on the percentage of each section occupied by tissue that stains with picrosirius red, a stain that specific stains collagen fibers. FIG. 20C shows the impact of treatment with Vehicle, Fc-HRS(2-60) or nintedanib beginning 7 (7 D) or 21 (21 D) days after allogenic transplantation on the number of myofibroblasts counted in lung sections. FIG. 20D shows the impact of treatment with Vehicle, Fc-HRS(2-60) or nintedanib beginning 7 (7 D) or 21 (21 D) days after allogenic transplantation on hydroxyproline content (an indicator or collagen content) in the skin. ****p<0.0001, ***p<0.001, **p<0.01, *p<0.05 Kruskal-Wallis non-parametric ANOVA followed by Dunn's multiple comparison test to Vehicle animals euthanized 8 weeks after allogenic transplant (3^{rd} group from the left).

FIGS. 21A-21C show the effects of HRS(2-60) containing proteins on LPS-induced leukocyte infiltration into the mouse lung. Mice were treated intravenously with Fc-HRS(2-60) or HRS(2-60)-COMP at the doses indicated. The following day, saline or LPS (10 μg/mouse) was instilled into the airways via oropharyngeal administration, and infiltrating cells were collected 24 hours later via bronchoalveolar lavage and analyzed by flow cytometry. Groups receiving LPS are demarcated by a horizontal bar. Data from individual animals (circles) along with mean and SEM are shown. Asterisk indicates significant difference (p<0.05) from the LPS/Vehicle group by ANOVA followed by Dunnett's post-hoc test.

FIGS. 22A-22B show that incubation of bone marrow derived macrophages with either 100 nM or 200 nM Fc-HRS(2-60) (Imod), but not the Fc control compound N15, for 5 days during monocyte differentiation resulted in a profound inhibition of phagocyte maturation, as revealed by the dramatically diminished spectral shift reported by the pH sensitive fluorescent dye pH Rhodo™.

FIGS. 23A-23B show that incubation of bone marrow derived macrophages with 100 nM HRS(2-60)-COMP), but not the control compound COMP, for 5 days during monocyte differentiation resulted in a profound inhibition of efferocytosis, as revealed by the dramatically diminished spectral shift reported by the pH sensitive fluorescent dye pH Rhodo™.

FIGS. 24A-24B show histological confirmation of model induction by presence of increased inflammation (H&E) and fibrosis (Masson's Trichrome) in mice that received P. acnes (group 3 and 4) as compared to mice that did not receive P. acnes (group 2).

FIGS. 25A-25B show measurements of lung inflammation (25A) and fibrosis (25B) at study termination.

FIGS. 26A-26H show that several profibrotic cytokines in the lung were reduced in response to Fc-HRS(2-60) treatment at 3 mg/kg, as indicated.

FIG. 27A shows that mice exposed to S. rectivirgula in control groups (Group 3 and Group 4) had a robust and consistent multifocal chronic pneumonia compared to mice exposed to PBS in Group 2.

FIG. 27B shows histopathology scores as determined by a veterinary pathologist. FIG. 27C shows a reduction of individual BALT area in the Fc-HRS(2-60) 3 mg/kg group following and in-depth analysis of the H&E stained lung tissue sections using the HALO platform.

FIGS. 28A-28G show that several pro-inflammatory cytokines and chemokines were significantly reduced in presence of Fc-HRS(2-60) at both 0.4 and 3 mg/kg.

FIGS. 29A-29E show that matrix metalloproteases (MMPs) were significantly reduced in presence of Fc-HRS(2-60) at both 0.4 and 3 mg/kg.

FIGS. 30A-30H show that inflammatory arthritis was successfully induced in SKG mice upon administration of 5 mg of zymosan, and that Fc-HRS(2-60) can lower the number of specific immune cells in the lungs of SKG mice; most notably B cells and T cells.

FIGS. 31A-31D show the overall clinical arthritis scores at days 35, 42, 49, and 56.

### DETAILED DESCRIPTION

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3rd Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5th Ed. Hoboken N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols) for modification of polypeptides” in Polyethylene Glycol Chemistry: Biotechnical and Biomedical Applications.

### Definitions

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

An “antagonist” or “inhibitor” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

The term “anergy” refers to the functional inactivation of a T cell, or B cell response to re-stimulation by antigen.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

The term “binding” refers to a direct association between two molecules, due to, for example, covalent, electrostatic, hydrophobic, and ionic and/or hydrogen-bond interactions, including interactions such as salt bridges and water bridges.

The term “clonal deletion” refers to the deletion (e.g., loss, or death) of auto-reactive T-cells. Clonal deletion can be achieved centrally in the thymus, or in the periphery, or both.

The term “chemoresistance” refers to the change in therapeutic sensitivity of a cancer cell population over time following exposure to cancer therapy, including resistance to at least one of a cancer immunotherapy agent, a chemotherapeutic agent, a hormonal therapeutic agent, and/or a kinase inhibitor. Eventually, chemoresistance leads to the relapse and/or metastasis of the cancer, and challenges the improvement of clinical outcome for the cancer patients. It remains the main obstacle to long term successful cancer therapy. For example, approximately 30 percent of women diagnosed with early-stage breast cancer develop chemoresistance and eventually progress to metastatic breast cancer. The molecular mechanisms of chemoresistance include the induction of transporter pumps, oncogenes, tumor suppressor genes, mitochondrial alteration, DNA repair, autophagy, epithelial-mesenchymal transition (EMT), cancer stemness, and exosome production. These mechanisms may operate via distinct mechanisms, but ultimately coordinate to prevent cell death in response to a chemotherapeutic agent. For example, the encoding proteins of oncogenes (EGFR-Akt-NF-κB) could modulate the expression of apoptosis-related genes, and thereby contribute to EMT, cell stemness, and autophagy. Autophagic cells are characterized by anti-apoptosis during chemoresistance. Accordingly, agents which reduce chemoresistance, including those that modulate autophagy, endosome maturation, phagocytosis, and/or efferocytosis, could find utility in the treatment or reduction of chemoresistant cancers.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

As used herein, the terms “contacting a cell”, “introducing” or “delivering” include delivery of the agents described herein (e.g., polypeptide agents, polynucleotide agents) into a cell by methods routine in the art, e.g., transfection (e.g., liposome, calcium-phosphate, polyethyleneimine), electroporation (e.g., nucleofection), microinjection) or administration to a subject.

The terms “cell penetrating peptide” (CPP) or “a peptide moiety which enhances cellular uptake” are used interchangeably and refer to cationic cell penetrating peptides, also called “transport peptides”, “carrier peptides”, or “peptide transduction domains.” In some embodiments, the peptides have the capability of inducing cell (e.g., muscle cell) penetration within about or at least about 30%, 40%, 50%, 60%, 70%, 80%, 90%, or 100% of cells of a given cell culture population and allow macromolecular translocation within multiple tissues (e.g., muscle tissues) in vivo upon systemic or other form of administration. In some embodiments, the CPPs are of the formula —[(C(O)CHR′NH)ₘ]R″ wherein R′ is a side chain of a naturally occurring amino acid or a one- or two-carbon homolog thereof, R″ is selected from Hydrogen or acyl, and m is an integer up to 50. Additional CPPs are well-known in the art and are disclosed, for example, in U.S. Application No. 2010/0016215, which is incorporated by reference in its entirety. In some embodiments, m is an integer selected from 1 to 50 where, when m is 1, the moiety is a single amino acid or derivative thereof. Any of the polynucleotide agents (e.g., antisense, RNAi agents) described herein can be conjugated to a CPP, for example, to improve uptake into target cells, e.g., muscle cells.

The term “half maximal effective concentration” or “EC50” refers to the concentration of an agent (e.g., HRS polypeptide, or other agent) as described herein at which it induces a response halfway between the baseline and maximum after some specified exposure time; the EC50 of a graded dose response curve therefore represents the concentration of a compound at which 50% of its maximal effect is observed. EC50 also represents the plasma concentration required for obtaining 50% of a maximum effect in vivo. Similarly, the “EC90” refers to the concentration of an agent or composition at which 90% of its maximal effect is observed. The “EC90” can be calculated from the “EC50” and the Hill slope, or it can be determined from the data directly, using routine knowledge in the art. In some embodiments, the EC50 of an agent is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, a biotherapeutic composition will have an EC50 value of about 1 nM or less.

“Homology” refers to the percentage number of amino acids that are identical or constitute conservative substitutions. Homology may be determined using sequence comparison programs such as GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The term “innate immune response” refers to the responses of immune cells (including myeloid derived cells, such as macrophages, neutrophils, eosinophils, granulocytes, and natural killer (NK) cells) and the associated mechanisms of modulating cytokine expression and release (e.g., interferons and interferon-signaling), inducing cell death, and inhibiting protein synthesis, which defend the host from infection by pathogens.

By “isolated” is meant material that is substantially or essentially free from components that normally accompany it in its native state. For example, an “isolated polynucleotide,” “isolated oligonucleotide,” or “isolated oligonucleotide” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

The term “migratory cells” refers to cells that are capable of movement from one place to another in response to a stimulus. Exemplary migratory cells include immune cells such as monocytes, Natural Killer (NK) cells, dendritic cells (immature or mature), subsets of dendritic cells including myeloid, plasmacytoid (also called lymphoid) and Langerhans cells, macrophages such as histiocytes, tissue resident macrophages such as Kupffer's cells, microglia cells in the CNS, alveolar macrophages, and peritoneal macrophages, macrophage subtypes such as M0, M1, Mox, M2a, M2b, and M2c macrophages, neutrophils, eosinophils, mast cells, basophils, B cells including plasma B cells, memory B cells, B-1 cells, and B-2 cells, CD45RO (naive T) cells, CD45RA (memory T) cells, CD4 Helper T Cells including Th1, Th2, and Tr1/Th3 cells, CD8 Cytotoxic T Cells, Regulatory T Cells, Gamma Delta T Cells, and thymocytes. Additional examples of migratory cells include fibroblasts, fibrocytes, tumor cells, and stem cells. Accordingly the term “cell migration”, refers to the movement of migratory cells, and the term “modulation of cell migration” refers to the modulation of the movement of any such migratory cells.

The terms “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or more agents or compositions to produce or cause a greater physiological response (i.e., downstream effects) in a cell or a subject relative to the response caused by either no agent/compound or a control compound. Relevant physiological or cellular responses (in vivo or in vitro) will be apparent to persons skilled in the art, and may include increases in skeletal muscle mass in a tissue or subject in need thereof. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7, 1.8), the amount produced by no agent/compound (the absence of an agent) or a control compound. The term “reduce” or “inhibit” may relate generally to the ability of one or more agents or compositions to “decrease” a relevant physiological or cellular response, such as expression of a target gene or a symptom of a disease or condition described herein, as measured according to routine techniques in the diagnostic art. Relevant physiological or cellular responses (in vivo or in vitro) will be apparent to persons skilled in the art, and may include reductions or improvements in the symptoms or pathology of lung inflammation or an ILD, as described herein. A “decrease” in a response may be “statistically significant” as compared to the response produced by no agent or composition or a control agent or composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

In certain embodiments, the “purity” of any given agent in a composition may be specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high performance liquid chromatography (HPLC), a well-known form of column chromatography used frequently in biochemistry and analytical chemistry to separate, identify, and quantify compounds.

A “lipid nanoparticle” or “solid lipid nanoparticle” refers to one or more spherical nanoparticles with an average diameter of between about 10 to about 1000 nanometers, and which comprise a solid lipid core matrix that can solubilize lipophilic molecules. The lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, which are incorporated by reference in their entireties.

The terms or “Neuropilin 2-associated disease” or “NRP2-associated disease” refer to diseases and conditions in which NRP2 activity, expression, and/or spatial distribution plays a role in the pathophysiology of that disease or condition. In some instances, NRP2 associated diseases are modulated by the HRS polypeptides of the present disclosure, for example, by altering the interaction of NRP2 with at least one NRP2 ligand, to impact NRP2 activity, signaling, expression, and/or spatial distribution. Exemplary NRP2-associated diseases and conditions include, without limitation, cancer and diseases or pathologies associated with cancer, including cancer cell growth, cancer initiation, cancer migration, cancer cell adhesion, cancer cell invasion, cancer cell chemoresistance, and cancer cell metastasis. Also included are diseases associated with inflammation and autoimmunity, including inflammatory lung diseases such as chronic hypersensitivity pneumonitis, pulmonary inflammation, and related inflammatory diseases. Also included are diseases associated with inappropriate immune cell activation or migration such as graft versus host disease (GVHD) and rheumatoid arthritis-associated interstitial lung disease (RA-ILD). Additional examples include diseases associated with lymphatic development, lymphangiogenesis, and lymphatic damage, including edema, lymphedema, secondary lymphedema, inappropriate fat absorption and deposition, excess fat deposition, and vascular permeability. Also included are diseases associated with infections, including latent infections, and diseases associated with allergic disorders/diseases and allergic responses, including chronic obstructive pulmonary disorder (COPD), neutrophilic asthma, antineutrophil cytoplasmic antibody (ANCA)-associated systemic vasculitis, systemic lupus erythematosus, rheumatoid arthritis, inflammasome-related disease(s), and skin-related neutrophil-mediated disease(s) such as pyoderma gangrenosum. Additional examples include diseases associated with granulomatous inflammatory diseases, including sarcoidosis and other pulmonary granulomatous diseased, and non-lung granulomas. Also included are fibrotic diseases such endometriosis, fibrosis, endothelial to mesenchymal transition (EMT), and wound healing, among others. Also included are diseases associated with inappropriate smooth muscle contractility and vascular smooth muscle cell migration and/or adhesion, and diseases associated with inappropriate autophagy, phagocytosis, and efferocytosis. Additional examples include neuronal diseases, including diseases associated with peripheral nervous system remodeling and pain perception. Also included are diseases associated with bone development and/or bone remodeling, and diseases associated with inappropriate migratory cell movement.

As used herein, “nucleobase” (Nu), “base pairing moiety” or “base” are used interchangeably to refer to a purine or pyrimidine base found in native DNA or RNA (uracil, thymine, adenine, cytosine, and guanine), as well as analogs of the naturally occurring purines and pyrimidines, that confer improved properties, such as binding affinity to the oligonucleotide. Exemplary analogs include hypoxanthine (the base component of the nucleoside inosine); 2,6-diaminopurine; 5-methyl cytosine; C5-propynyl-modified pyrimidines; 9-(aminoethoxy)phenoxazine (G-clamp) and the like.

Further examples of base pairing moieties include, but are not limited to, uracil, thymine, adenine, cytosine, guanine and hypoxanthine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). The modified nucleobases disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313, are also contemplated.

Further examples of base pairing moieties include, but are not limited to, expanded-size nucleobases in which one or more benzene rings has been added. Nucleic base replacements described in the Glen Research catalog (www.glenresearch.com); Krueger A T et al, Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, are contemplated as useful for the synthesis of the oligonucleotides described herein. Examples of expanded-size nucleobases are shown below:

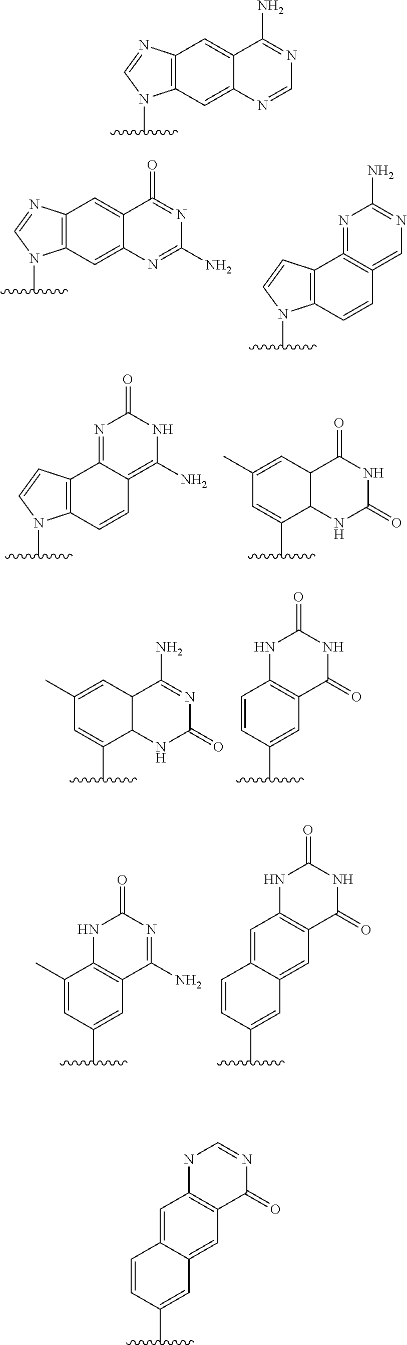

A nucleobase covalently linked to a ribose, sugar analog or morpholino comprises a nucleoside. “Nucleotides” are composed of a nucleoside together with one phosphate group. The phosphate groups covalently link adjacent nucleotides to one another to form an oligonucleotide.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

“Expression control sequences” include regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, which have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types), and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include the Tet system, (U.S. Pat. Nos. 5,464,758 and 5,814,618), the Ecdysone inducible system (No et al., Proc. Natl. Acad. Sci. (1996) 93 (8): 3346-3351; the T-REx™ system (Invitrogen Carlsbad, Calif.), LacSwitch® (Stratagene, (San Diego, Calif.) and the Cre-ERT tamoxifen inducible recombinase system (Indra et al. Nuc. Acid. Res. (1999) 27 (22): 4324-4327; Nuc. Acid. Res. (2000) 28 (23): e99; U.S. Pat. No. 7,112,715; and Kramer & Fussenegger, Methods Mol. Biol. (2005) 308: 123-144) or any promoter known in the art suitable for expression in the desired cells.

An “expressible polynucleotide” includes a cDNA, RNA, mRNA or other polynucleotide that comprises at least one coding sequence and optionally at least one expression control sequence, for example, a transcriptional and/or translational regulatory element, and which can express an encoded polypeptide (for example, an HRS polypeptide) upon introduction into a cell, for example, a cell in a subject.

In some embodiments, the expressible polynucleotide is a modified RNA or modified mRNA polynucleotide, for example, a non-naturally occurring RNA analog. In certain embodiments, the modified RNA or mRNA polypeptide comprises one or more modified or non-natural bases, for example, a nucleotide base other than adenine (A), guanine (G), cytosine (C), thymine (T), and/or uracil (U). In some embodiments, the modified mRNA comprises one or more modified or non-natural internucleotide linkages. Expressible RNA polynucleotides for delivering an encoded therapeutic polypeptide are described, for example, in Kormann et al., Nat Biotechnol. 29:154-7, 2011; and U.S. Application Nos. 2015/0111248; 2014/0243399; 2014/0147454; and 2013/0245104, which are incorporated by reference in their entireties.

In some embodiments, various viral vectors that can be utilized to deliver an expressible polynucleotide include adenoviral vectors, herpes virus vectors, vaccinia virus vectors, adeno-associated virus (AAV) vectors, and retroviral vectors. In some instances, the retroviral vector is a derivative of a murine or avian retrovirus, or is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated. By inserting a polypeptide sequence of interest into the viral vector, along with another gene that encodes the ligand for a receptor on a specific target cell, for example, the vector may be made target specific. Retroviral vectors can be made target specific by inserting, for example, a polynucleotide encoding a protein. Illustrative targeting may be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector.

In certain instances, the expressible polynucleotides described herein are engineered for localization within a cell, potentially within a specific compartment such as the nucleus, or are engineered for secretion from the cell or translocation to the plasma membrane of the cell. In exemplary embodiments, the expressible polynucleotides are engineered for nuclear localization.

Also included are biologically active “variants” and “fragments” of the polypeptides described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or polynucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60,70, 80, 90, 100, 110, 120, 130, 140, 150 or more amino acids or nucleotides and which substantially retain the activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

The term “solubility” refers to the property of an agent provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, or pH 7.4. In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” includes a mammalian subject such as a human subject.

“Substantially” or “essentially” means nearly totally or completely, for instance, 95% or greater of some given quantity.

“Therapeutic response” refers to improvement of symptoms (whether or not sustained) based on the administration of the therapeutic response.

As used herein, the term “target” refers to a RNA region, and specifically, to a RNA region of a target gene described herein. The target can include coding and non-coding sequences, 5′ upstream sequences, 3′ downstream sequences, and other RNA sequences described herein.

The term “target sequence” refers to a portion of the target RNA against which the antisense or RNAi agent is directed, for example, the sequence to which the antisense oligonucleotide will hybridize by Watson-Crick base pairing of a complementary sequence, or the sequence that corresponds to the sense strand of the RNAi agent.

As used herein, the term “quantifying”, “quantification” or other related words refer to determining the quantity, mass, or concentration in a unit volume, of a nucleic acid, polynucleotide, oligonucleotide, peptide, polypeptide, or protein.

As used herein, the terms “therapeutically effective amount”, “therapeutic dose,” “prophylactically effective amount,” or “diagnostically effective amount” is the amount of an agent needed to elicit the desired biological response following administration. Similarly the term “antisense therapy” or “RNAi therapy” includes a therapy that maintains the average steady state concentration of an antisense or RNAi agent in the patient's plasma or other tissue compartment (e.g., muscle tissue) above the minimum effective therapeutic level.

As used herein, “treatment” of a subject (e.g. a mammal, such as a human) or a cell is any type of intervention used in an attempt to alter the natural course of the subject or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

### Histidyl-tRNA Synthetase (HRS) Polypeptides and Polynucleotides

Certain embodiments include histidyl-tRNA synthetase polypeptides (“HRS” or “HisRS” polypeptides), including conjugates (e.g., fusion proteins, Fc conjugates, Fc fusion proteins, COMP conjugates, COMP fusion proteins), variants, and fragments thereof, and expressible polynucleotides that encode the HRS polypeptides. Histidyl-tRNA synthetases belong to the class II tRNA synthetase family, which has three highly conserved sequence motifs. Class I and II tRNA synthetases are widely recognized as being responsible for the specific attachment of an amino acid to its cognate tRNA in a two-step reaction: the amino acid (AA) is first activated by ATP to form AA-AMP and then transferred to the acceptor end of the tRNA. The full-length histidyl-tRNA synthetases typically exist either as a cytosolic homodimer, or an alternatively spliced mitochondrial form.

Certain biological fragments or alternatively spliced isoforms of eukaryotic histidyl-tRNA synthetases, or in some contexts the intact full-length synthetase, modulate certain therapeutically relevant cell-signaling pathways, bind to one or more neuropilin polypeptides (see, for example, Table N1), and/or have anti-inflammatory properties. These activities, which are distinct from the classical role of tRNA synthetases in protein synthesis, are referred to herein as “non-canonical activities.” For example, as provided herein, HRS polypeptides such as the N-terminal region of histidyl-tRNA synthetase (e.g., HRS 1-48, HRS 1-60) are capable, inter alia, of binding to a neuropilin polypeptide, and thereby modulating the migration, activation, and/or differentiation of inflammatory cells or migratory cells, and treating neuropilin-associated disease. In addition, certain fragments, splice variants, mutations, and/or deletions (e.g., HRS 1-60) relative to the full-length HRS polypeptide sequence confer increased activities and/or improved pharmacological properties. The sequences of certain exemplary HRS polypeptides are provided in Table H1 below.

TABLE H1
Exemplary HRS polypeptides

| | | | SEQ ID |
|---|---|---|---|
| Name | Residues | Sequence | NO: |
| FL | 1-509 | MAERAALEELVKLQGERVRGLKQQKASAELIEEEVAKLLK | 1 |
| cytosolic | | LKAQLGPDESKQKFVLKTPKGTRDYSPRQMAVREKVFDVI | |
| wild type | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGI KAEL | |
| | | LYKKNPKLLNQLQYCEEAGIPLVAIIGEQELKDGVIKLRS | |
| | | VTSREEVDVRREDLVEEIKRRTGQPLCIC | |
| HRS(1-500) | 1-500 | MAERAALEELVKLQGERVRGLKQQKASAELIEEEVAKLLK | 2 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKR | |
| HRS(1-501) | 1-501 | MAERAALEELVKLQGERVRGLKQQKASAELIEEEVAKLLK | 3 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRR | |
| HRS(1-502) | 1-502 | MAERAALEELVKLQGERVRGLKQQKASAELIEEEVAKLLK | 4 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRT | |
| HRS(1-503) | 1-503 | MAERAALEELVKLQGERVRGLKQQKASAELIEEEVAKLLK | 5 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTG | |
| HRS(1-504) | 1-504 | MAERAALEELVKLQGERVRGLKQQKASAELIEEEVAKLLK | 6 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTGQ | |
| HRS(1-505) | 1-505 | MAERAALEELVKLQGERVRGLKQQKASAELIEEEVAKLLK | 172 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTGQP | |
| HisRS1^{N8} | 1-506 | MAERAALEELVKLQGERVRGLKQQKASAELIEEEVAKLLK | 7 |
| HRS(1-506) | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTGQPL | |
| HRS(2-506) | 2-506 | AERAALEELVKLQGERVRGLKQQKASAELIEEEVAKLLKL | 8 |
| | | KAQLGPDESKQKFVLKTPKGTRDYSPRQMAVREKVFDVII | |
| | | RCFKRHGAEVIDTPVFELKETLMGKYGEDSKLIYDLKDQG | |
| | | GELLSLRYDLTVPFARYLAMNKLTNIKRYHIAKVYRRDNP | |
| | | AMTRGRYREFYQCDFDIAGNFDPMIPDAECLKIMCEILSS | |
| | | LQIGDFLVKVNDRRILDGMFAICGVSDSKFRTICSSVDKL | |
| | | DKVSWEEVKNEMVGEKGLAPEVADRIGDYVQQHGGVSLVE | |
| | | QLLQDPKLSQNKQALEGLGDLKLLFEYLTLFGIDDKISFD | |
| | | LSLARGLDYYTGVIYEAVLLQTPAQAGEEPLGVGSVAAGG | |
| | | RYDGLVGMFDPKGRKVPCVGLSIGVERIFSIVEQRLEALE | |
| | | EKIRTTETQVLVASAQKKLLEERLKLVSELWDAGIKAELL | |
| | | YKKNPKLLNQLQYCEEAGIPLVAIIGEQELKDGVIKLRSV | |
| | | TSREEVDVRREDLVEEIKRRTGQPL | |
| HRS(1-507) | 1-507 | MAERAALEELVKLQGERVRGLKQQKASAELIEEEVAKLLK | 9 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTGQPLC | |
| HRS(1-508) | 1-508 | MAERAALEELVKLQGERVRGLKQQKASAELIEEEVAKLLK | 10 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTGQPLCI | |
| HisRS1^{N6} | 1-48 | MAERAALEELVKLQGERVRGLKQQKASAELIEEEVAKLLK | 11 |
| HRS(1-48) | | LKAQLGPD | |
| | 1-80 | MAERAALEELVKLQGERVRGLKQQKASAELIEEEVAKLLK | 12 |
| | | LKAQLGPDESKQKFVLKTPKGTRDYSPRQMAVREKVFDVI | |
| | 1-79 | MAERAALEELVKLQGERVRGLKQQKASAELIEEEVAKLLK | 13 |
| | | LKAQLGPDESKQKFVLKTPKGTRDYSPRQMAVREKVFDV | |
| | 1-78 | MAERAALEELVKLQGERVRGLKQQKASAELIEEEVAKLLK | 14 |
| | | LKAQLGPDESKQKFVLKTPKGTRDYSPRQMAVREKVFD | |
| | 1-77 | MAERAALEELVKLQGERVRGLKQQKASAELIEEEVAKLLK | 15 |
| | | LKAQLGPDESKQKFVLKTPKGTRDYSPRQMAVREKVE | |
| | 1-76 | MAERAALEELVKLQGERVRGLKQQKASAELIEEEVAKLLK | 16 |
| | | LKAQLGPDESKQKFVLKTPKGTRDYSPRQMAVREKV | |
| | 1-75 | MAERAALEELVKLQGERVRGLKQQKASAELIEEEVAKLLK | 17 |
| | | LKAQLGPDESKQKFVLKTPKGTRDYSPRQMAVREK | |
| | 1-74 | MAERAALEELVKLQGERVRGLKQQKASAELIEEEVAKLLK | 18 |
| | | LKAQLGPDESKQKFVLKTPKGTRDYSPRQMAVRE | |
| | 1-73 | MAERAALEELVKLQGERVRGLKQQKASAELIEEEVAKLLK | 19 |
| | | LKAQLGPDESKQKFVLKTPKGTRDYSPRQMAVR | |
| | 1-72 | MAERAALEELVKLQGERVRGLKQQKASAELIEEEVAKLLK | 20 |
| | | LKAQLGPDESKQKFVLKTPKGTRDYSPRQMAV | |
| | 1-71 | MAERAALEELVKLQGERVRGLKQQKASAELIEEEVAKLLK | 21 |
| | | LKAQLGPDESKQKFVLKTPKGTRDYSPRQMA | |
| | 1-70 | MAERAALEELVKLQGERVRGLKQQKASAELIEEEVAKLLK | 22 |
| | | LKAQLGPDESKQKFVLKTPKGTRDYSPRQM | |
| | 1-69 | MAERAALEELVKLQGERVRGLKQQKASAELIEEEVAKLLK | 23 |
| | | LKAQLGPDESKQKFVLKTPKGTRDYSPRQ | |
| | 1-68 | MAERAALEELVKLQGERVRGLKQQKASAELIEEEVAKLLK | 24 |
| | | LKAQLGPDESKQKFVLKTPKGTRDYSPR | |
| | 1-67 | MAERAALEELVKLQGERVRGLKQQKASAELIEEEVAKLLK | 25 |
| | | LKAQLGPDESKQKFVLKTPKGTRDYSP | |
| | 1-66 | MAERAALEELVKLQGERVRGLKQQKASAELIEEEVAKLLK | 26 |
| | | LKAQLGPDESKQKFVLKTPKGTRDYS | |
| | 1-65 | MAERAALEELVKLQGERVRGLKQQKASAELIEEEVAKLLK | 27 |
| | | LKAQLGPDESKQKFVLKTPKGTRDY | |
| | 1-64 | MAERAALEELVKLQGERVRGLKQQKASAELIEEEVAKLLK | 28 |
| | | LKAQLGPDESKQKFVLKTPKGTRD | |
| | 1-63 | MAERAALEELVKLQGERVRGLKQQKASAELIEEEVAKLLK | 29 |
| | | LKAQLGPDESKQKFVLKTPKGTR | |
| | 1-62 | MAERAALEELVKLQGERVRGLKQQKASAELIEEEVAKLLK | 30 |
| | | LKAQLGPDESKQKFVLKTPKGT | |
| | 1-61 | MAERAALEELVKLQGERVRGLKQQKASAELIEEEVAKLLK | 31 |
| | | LKAQLGPDESKQKFVLKTPKG | |
| | 1-60 | MAERAALEELVKLQGERVRGLKQQKASAELIEEEVAKLLK | 32 |
| | | LKAQLGPDESKQKFVLKTPK | |
| | 1-59 | MAERAALEELVKLQGERVRGLKQQKASAELIEEEVAKLLK | 33 |
| | | LKAQLGPDESKQKFVLKTP | |
| | 1-58 | MAERAALEELVKLQGERVRGLKQQKASAELIEEEVAKLLK | 34 |
| | | LKAQLGPDESKQKFVLKT | |
| | 1-57 | MAERAALEELVKLQGERVRGLKQQKASAELIEEEVAKLLK | 35 |
| | | LKAQLGPDESKQKFVLK | |
| | 1-56 | MAERAALEELVKLQGERVRGLKQQKASAELIEEEVAKLLK | 36 |
| | | LKAQLGPDESKQKFVL | |
| | 1-55 | MAERAALEELVKLQGERVRGLKQQKASAELIEEEVAKLLK | 37 |
| | | LKAQLGPDESKQKFV | |
| | 1-54 | MAERAALEELVKLQGERVRGLKQQKASAELIEEEVAKLLK | 38 |
| | | LKAQLGPDESKQKF | |
| | 1-53 | MAERAALEELVKLQGERVRGLKQQKASAELIEEEVAKLLK | 39 |
| | | LKAQLGPDESKQK | |
| | 1-52 | MAERAALEELVKLQGERVRGLKQQKASAELIEEEVAKLLK | 40 |
| | | LKAQLGPDESKQ | |
| | 1-51 | MAERAALEELVKLQGERVRGLKQQKASAELIEEEVAKLLK | 41 |
| | | LKAQLGPDESK | |
| | 1-50 | MAERAALEELVKLQGERVRGLKQQKASAELIEEEVAKLLK | 42 |
| | | LKAQLGPDES | |
| | 1-49 | MAERAALEELVKLQGERVRGLKQQKASAELIEEEVAKLLK | 43 |
| | | LKAQLGPDE | |
| | 1-48 | MAERAALEELVKLQGERVRGLKQQKASAELIEEEVAKLLK | 44 |
| | | LKAQLGPD | |
| | 1-47 | MAERAALEELVKLQGERVRGLKQQKASAELIEEEVAKLLK | 45 |
| | | LKAQLGP | |
| | 1-46 | MAERAALEELVKLQGERVRGLKQQKASAELIEEEVAKLLK | 46 |
| | | LKAQLG | |
| | 1-45 | MAERAALEELVKLQGERVRGLKQQKASAELIEEEVAKLLK | 47 |
| | | LKAQL | |
| | 1-44 | MAERAALEELVKLQGERVRGLKQQKASAELIEEEVAKLLK | 48 |
| | | LKAQ | |
| | 1-43 | MAERAALEELVKLQGERVRGLKQQKASAELIEEEVAKLLK | 49 |
| | | LKA | |
| | 1-42 | MAERAALEELVKLQGERVRGLKQQKASAELIEEEVAKLLK | 50 |
| | | LK | |
| | 1-41 | MAERAALEELVKLQGERVRGLKQQKASAELIEEEVAKLLK | 51 |
| | | L | |
| | 1-40 | MAERAALEELVKLQGERVRGLKQQKASAELIEEEVAKLLK | 52 |
| | 2-80 | AERAALEELVKLQGERVRGLKQQKASAELIEEEVAKLLKL | 53 |
| | | KAQLGPDESKQKFVLKTPKGTRDYSPRQMAVREKVFDVI | |
| | 3-80 | ERAALEELVKLQGERVRGLKQQKASAELIEEEVAKLLKLK | 54 |
| | | AQLGPDESKQKFVLKTPKGTRDYSPRQMAVREKVFDVI | |
| | 4-80 | RAALEELVKLQGERVRGLKQQKASAELIEEEVAKLLKLKA | 55 |
| | | QLGPDESKQKFVLKTPKGTRDYSPRQMAVREKVFDVI | |
| | 5-80 | AALEELVKLQGERVRGLKQQKASAELIEEEVAKLLKLKAQ | 56 |
| | | LGPDESKQKFVLKTPKGTRDYSPRQMAVREKVFDVI | |
| | 6-80 | ALEELVKLQGERVRGLKQQKASAELIEEEVAKLLKLKAQL | 57 |
| | | GPDESKQKFVLKTPKGTRDYSPRQMAVREKVFDVI | |
| | 7-80 | LEELVKLQGERVRGLKQQKASAELIEEEVAKLLKLKAQLG | 58 |
| | | PDESKQKFVLKTPKGTRDYSPRQMAVREKVFDVI | |
| | 8-80 | EELVKLQGERVRGLKQQKASAELIEEEVAKLLKLKAQLGP | 59 |
| | | DESKQKFVLKTPKGTRDYSPRQMAVREKVFDVI | |
| | 9-80 | ELVKLQGERVRGLKQQKASAELIEEEVAKLLKLKAQLGPD | 60 |
| | | ESKQKFVLKTPKGTRDYSPRQMAVREKVFDVI | |
| | 10-80 | LVKLQGERVRGLKQQKASAELIEEEVAKLLKLKAQLGPDE | 61 |
| | | SKQKFVLKTPKGTRDYSPRQMAVREKVFDVI | |
| | 11-80 | VKLQGERVRGLKQQKASAELIEEEVAKLLKLKAQLGPDES | 62 |
| | | KQKFVLKTPKGTRDYSPRQMAVREKVFDVI | |
| | 12-80 | KLQGERVRGLKQQKASAELIEEEVAKLLKLKAQLGPDESK | 63 |
| | | QKFVLKTPKGTRDYSPRQMAVREKVFDVI | |
| | 13-80 | LQGERVRGLKQQKASAELIEEEVAKLLKLKAQLGPDESKQ | 64 |
| | | KFVLKTPKGTRDYSPRQMAVREKVFDVI | |
| | 14-80 | QGERVRGLKQQKASAELIEEEVAKLLKLKAQLGPDESKQK | 65 |
| | | FVLKTPKGTRDYSPRQMAVREKVFDVI | |
| | 15-80 | GERVRGLKQQKASAELIEEEVAKLLKLKAQLGPDESKQKF | 66 |
| | | VLKTPKGTRDYSPRQMAVREKVFDVI | |
| | 16-80 | RVRGLKQQKASAELIEEEVAKLLKLKAQLGPDESKQKFVL | 67 |
| | | KTPKGTRDYSPRQMAVREKVFDVI | |
| | 17-80 | VRGLKQQKASAELIEEEVAKLLKLKAQLGPDESKQKFVLK | 68 |
| | | TPKGTRDYSPRQMAVREKVFDVI | |
| | 18-80 | RGLKQQKASAELIEEEVAKLLKLKAQLGPDESKQKFVLKT | 69 |
| | | PKGTRDYSPRQMAVREKVFDVI | |
| | 19-80 | GLKQQKASAELIEEEVAKLLKLKAQLGPDESKQKFVLKTP | 70 |
| | | KGTRDYSPRQMAVREKVFDVI | |
| | 20-80 | LKQQKASAELIEEEVAKLLKLKAQLGPDESKQKFVLKTPK | 71 |
| | | GTRDYSPRQMAVREKVFDVI | |
| | 21-80 | KQQKASAELIEEEVAKLLKLKAQLGPDESKQKFVLKTPKG | 72 |
| | | TRDYSPRQMAVREKVFDVI | |
| | 22-80 | QQKASAELIEEEVAKLLKLKAQLGPDESKQKFVLKTPKGT | 73 |
| | | RDYSPRQMAVREKVFDVI | |
| | 23-80 | QKASAELIEEEVAKLLKLKAQLGPDESKQKFVLKTPKGTR | 74 |
| | | DYSPRQMAVREKVFDVI | |
| | 24-80 | KASAELIEEEVAKLLKLKAQLGPDESKQKFVLKTPKGTRD | 75 |
| | | YSPRQMAVREKVFDVI | |
| | 25-80 | ASAELIEEEVAKLLKLKAQLGPDESKQKFVLKTPKGTRDY | 76 |
| | | SPRQMAVREKVFDVI | |
| | 26-80 | SAELIEEEVAKLLKLKAQLGPDESKQKFVLKTPKGTRDYS | 77 |
| | | PRQMAVREKVFDVI | |
| | 27-80 | AELIEEEVAKLLKLKAQLGPDESKQKFVLKTPKGTRDYSP | 78 |
| | | RQMAVREKVFDVI | |
| | 28-80 | ELIEEEVAKLLKLKAQLGPDESKQKFVLKTPKGTRDYSPR | 79 |
| | | QMAVREKVFDVI | |
| | 29-80 | LIEEEVAKLLKLKAQLGPDESKQKFVLKTPKGTRDYSPRQ | 80 |
| | | MAVREKVFDVI | |
| | 30-80 | IEEEVAKLLKLKAQLGPDESKQKFVLKTPKGTRDYSPRQM | 81 |
| | | AVREKVFDVI | |
| | 31-80 | EEEVAKLLKLKAQLGPDESKQKFVLKTPKGTRDYSPRQMA | 82 |
| | | VREKVFDVI | |
| | 32-80 | EEVAKLLKLKAQLGPDESKQKFVLKTPKGTRDYSPRQMAV | 83 |
| | | REKVFDVI | |
| | 33-80 | EVAKLLKLKAQLGPDESKQKFVLKTPKGTRDYSPRQMAVR | 84 |
| | | EKVFDVI | |
| | 34-80 | VAKLLKLKAQLGPDESKQKFVLKTPKGTRDYSPRQMAVRE | 85 |
| | | KVFDVI | |
| | 35-80 | AKLLKLKAQLGPDESKQKFVLKTPKGTRDYSPRQMAVREK | 86 |
| | | VFDVI | |
| | 36-80 | KLLKLKAQLGPDESKQKFVLKTPKGTRDYSPRQMAVREKV | 87 |
| | | FDVI | |
| | 37-80 | LLKLKAQLGPDESKQKFVLKTPKGTRDYSPRQMAVREKVF | 88 |
| | | DVI | |
| | 38-80 | LK | 89 |
| | | LKAQLGPDESKQKFVLKTPKGTRDYSPRQMAVREKVFDVI | |
| | 39-80 | K | 90 |
| | | LKAQLGPDESKQKFVLKTPKGTRDYSPRQMAVREKVFDVI | |
| | 40-80 | LKAQLGPDESKQKFVLKTPKGTRDYSPRQMAVREKVFDVI | 91 |
| HisRS1^{N1} | 1-141 | MAERAALEELVKLQGERVRGLKQQKASAELIEEEVAKLLK | 92 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAM | |
| HisRS1^{N2} | 1-408 | MAERAALEELVKLQGERVRGLKQQKASAELIEEEVAKLLK | 93 |
| | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTE | |
| HisRS1^{N3} | 1-113 | MAERAALEELVKLQGERVRGLKQQKASAELIEEEVAKLLK | 94 |
| | | LKAQLGPDESKQKFVLKTPKGTRDYSPRQMAVREKVFDVI | |
| | | IRCFKRHGAEVIDTPVFELKETLMGKYGEDSKL | |
| HisRS1^{N4} | 1-60 | MAERAALEELVKLQGERVRGLKQQKASAELIEEEVAKLLK | 95 |
| | | LKAQLGPDESKQKFVLKTPK | |
| HisRS1^{N5} | 1-243 + | MAERAALEELVKLQGERVRGLKQQKASAELIEEEVAKLLK | 96 |
| | 27aa | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVGYPWWNSCSRILNYPKTSRPWRAWET | |
| HisRS1^{C1} | 405-509 | RTTETQVLVASAQKKLLEERLKLVSELWDAGIKAELLYKK | 97 |
| | | NPKLLNQLQYCEEAGIPLVAIIGEQELKDGVIKLRSVTSR | |
| | | EEVDVRREDLVEEIKRRTGQPLCIC | |
| HisRS1^{C2} | 1-60 + | MAERAALEELVKLQGERVRGLKQQKASAELIEEEVAKLLK | 98 |
| | 175-509 | LKAQLGPDESKQKFVLKTPKDFDIAGNFDPMIPDAECLKI | |
| | | MCEILSSLQIGDFLVKVNDRRILDGMFAICGVSDSKFRTI | |
| | | CSSVDKLDKVSWEEVKNEMVGEKGLAPEVADRIGDYVQQH | |
| | | GGVSLVEQLLQDPKLSQNKQALEGLGDLKLLFEYLTLFGI | |
| | | DDKISFDLSLARGLDYYTGVIYEAVLLQTPAQAGEEPLGV | |
| | | GSVAAGGRYDGLVGMFDPKGRKVPCVGLSIGVERIFSIVE | |
| | | QRLEALEEKIRTTETQVLVASAQKKLLEERLKLVSELWDA | |
| | | GIKAELLYKKNPKLLNQLQYCEEAGIPLVAIIGEQELKDG | |
| | | VIKLRSVTSREEVDVRREDLVEEIKRRTGQPLCIC | |
| HisRS1^{C3} | 1-60 + | MAERAALEELVKLQGERVRGLKQQKASAELIEEEVAKLLK | 99 |
| | 211-509 | LKAQLGPDESKQKFVLKTPKVNDRRILDGMFAICGVSDSK | |
| | | FRTICS SVDKLDKVSWEEVKNEMVGEKGLAPEVADRI GDY | |
| | | VQQHGGVSLVEQLLQDPKLSQNKQALEGLGDLKLLFEYLT | |
| | | LFGIDDKISFDLSLARGLDYYTGVIYEAVLLQTPAQAGEE | |
| | | PLGVGSVAAGGRYDGLVGMFDPKGRKVPCVGLSIGVERIF | |
| | | SIVEQRLEALEEKIRTTETQVLVASAQKKLLEERLKLVSE | |
| | | LWDAGIKAELLYKKNPKLLNQLQYCEEAGIPLVAIIGEQE | |
| | | LKDGVIKLRSVTSREEVDVRREDLVEEIKRRTGQPLCIC | |
| HisRS1^{C4} | 1-100 + | MAERAALEELVKLQGERVRGLKQQKASAELIEEEVAKLLK | 100 |
| | 211-509 | LKAQLGPDESKQKFVLKTPKGTRDYSPRQMAVREKVFDVI | |
| | | IRCFKRHGAEVIDTPVFELKVNDRRILDGMFAICGVSDSK | |
| | | FRTICS SVDKLDKVSWEEVKNEMVGEKGLAPEVADRIGDY | |
| | | VQQHGGVSLVEQLLQDPKLSQNKQALEGLGDLKLLFEYLT | |
| | | LFGIDDKISFDLSLARGLDYYTGVIYEAVLLQTPAQAGEE | |
| | | PLGVGSVAAGGRYDGLVGMFDPKGRKVPCVGLSIGVERIF | |
| | | SIVEQRLEALEEKIRTTETQVLVASAQKKLLEERLKLVSE | |
| | | LWDAGIKAELLYKKNPKLLNQLQYCEEAGIPLVAIIGEQE | |
| | | LKDGVIKLRSVTSREEVDVRREDLVEEIKRRTGQPLCIC | |
| HisRS1^{C5} | 1-174 + | MAERAALEELVKLQGERVRGLKQQKASAELIEEEVAKLLK | 101 |
| | 211-509 | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRYDLTVPFARYLAMNKLTNIKRYHIAKVYRRDN | |
| | | PAMTRGRYREFYQCVNDRRILDGMFAICGVSDSKFRTICS | |
| | | SVDKLDKVSWEEVKNEMVGEKGLAPEVADRIGDYVQQHGG | |
| | | VSLVEQLLQDPKLSQNKQALEGLGDLKLLFEYLTLFGIDD | |
| | | KISFDLSLARGLDYYTGVIYEAVLLQTPAQAGEEPLGVGS | |
| | | VAAGGRYDGLVGMFDPKGRKVPCVGLSIGVERIFSIVEQR | |
| | | LEALEEKIRTTETQVLVASAQKKLLEERLKLVSELWDAGI | |
| | | KAELLYKKNPKLLNQLQYCEEAGIPLVAIIGEQELKDGVI | |
| | | KLRSVTSREEVDVRREDLVEEIKRRTGQPLCIC | |
| HisRS1^{C6} | 1-60 + | MAERAALEELVKLQGERVRGLKQQKASAELIEEEVAKLLK | 102 |
| | 101-509 | LKAQLGPDESKQKFVLKTPKETLMGKYGEDSKLIYDLKDQ | |
| | | GGELLSLRYDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIDDKISF | |
| | | DLSLARGLDYYTGVIYEAVLLQTPAQAGEEPLGVGSVAAG | |
| | | GRYDGLVGMFDPKGRKVPCVGLSIGVERIFSIVEQRLEAL | |
| | | EEKIRTTETQVLVASAQKKLLEERLKLVSELWDAGIKAEL | |
| | | LYKKNPKLLNQLQYCEEAGIPLVAIIGEQELKDGVIKLRS | |
| | | VTSREEVDVRREDLVEEIKRRTGQPLCIC | |
| HisRS1^{C7} | P1-100 + | MAERAALEELVKLQGERVRGLKQQKASAELIEEEVAKLLK | 103 |
| | 175-509 | LKAQLGPDESKQKFVLKTPKGTRDYSPRQMAVREKVFDVI | |
| | | IRCFKRHGAEVIDTPVFELKDFDIAGNFDPMIPDAECLKI | |
| | | MCEILSSLQIGDFLVKVNDRRILDGMFAICGVSDSKFRTI | |
| | | CSSVDKLDKVSWEEVKNEMVGEKGLAPEVADRIGDYVQQH | |
| | | GGVSLVEQLLQDPKLSQNKQALEGLGDLKLLFEYLTLFGI | |
| | | DDKISFDLSLARGLDYYTGVIYEAVLLQTPAQAGEEPLGV | |
| | | GSVAAGGRYDGLVGMFDPKGRKVPCVGLSIGVERIFSIVE | |
| | | QRLEALEEKIRTTETQVLVASAQKKLLEERLKLVSELWDA | |
| | | GIKAELLYKKNPKLLNQLQYCEEAGIPLVAIIGEQELKDG | |
| | | VIKLRSVTSREEVDVRREDLVEEIKRRTGQPLCIC | |
| HisRS1^{C8} | 1-60 + | MAERAALEELVKLQGERVRGLKQQKASAELIEEEVAKLLK | 104 |
| | 399-509 | LKAQLGPDESKQKFVLKTPKALEEKIRTTETQVLVASAQK | |
| | | KLLEERLKLVSELWDAGIKAELLYKKNPKLLNQLQYCEEA | |
| | | GIPLVAIIGEQELKDGVIKLRSVTSREEVDVRREDLVEEI | |
| | | KRRTGQPLCIC | |
| HisRS1^{C9} | 1-100 + | MAERAALEELVKLQGERVRGLKQQKASAELIEEEVAKLLK | 105 |
| | 399-509 | LKAQLGPDESKQKFVLKTPKGTRDYSPRQMAVREKVFDVI | |
| | | IRCFKRHGAEVIDTPVFELKALEEKIRTTETQVLVASAQK | |
| | | KLLEERLKLVSELWDAGIKAELLYKKNPKLLNQLQYCEEA | |
| | | GIPLVAIIGEQELKDGVIKLRSVTSREEVDVRREDLVEEI | |
| | | KRRTGQPLCIC | |
| HisRS1^{C10} | 369-509 | MFDPKGRKVPCVGLSIGVERIFSIVEQRLEALEEKIRTTE | 106 |
| | | TQVLVASAQKKLLEERLKLVSELWDAGIKAELLYKKNPKL | |
| | | LNQLQYCEEAGIPLVAIIGEQELKDGVIKLRSVTSREEVD | |
| | | VRREDLVEEIKRRTGQPLCIC | |
| HisRS1¹¹ | 191-333 | CLKIMCEILSSLQIGDFLVKVNDRRILDGMFAICGVSDSK | 107 |
| | | FRTICSSVDKLDKVSWEEVKNEMVGEKGLAPEVADRIGDY | |
| | | VQQHGGVSLVEQLLQDPKLSQNKQALEGLGDLKLLFEYLT | |
| | | LFGIDDKISFDLSLARGLDYYTG | |
| FL mito. | 1-506 | MPLLGLLPRRAWASLLSQLLRPPCASCTGAVRCQSQVAEA | 108 |
| wild type | | VLTSQLKAHQEKPNFIIKTPKGTRDLSPQHMVVREKILDL | |
| | | VISCFKRHGAKGMDTPAFELKETLTEKYGEDSGLMYDLKD | |
| | | QGGELLSLRYDLTVPFARYLAMNKVKKMKRYHVGKVWRRE | |
| | | SPTIVQGRYREFCQCDFDIAGQFDPMIPDAECLKIMCEIL | |
| | | SGLQLGDFLIKVNDRRIVDGMFAVCGVPESKFRAICSSID | |
| | | KLDKMAWKDVRHEMVVKKGLAPEVADRIGDYVQCHGGVSL | |
| | | VEQMFQDPRLSQNKQALEGLGDLKLLFEYLTLFGIADKIS | |
| | | FDLSLARGLDYYTGVIYEAVLLQTPTQAGEEPLNVGSVAA | |
| | | GGRYDGLVGMFDPKGHKVPCVGLSIGVERIFYIVEQRMKT | |
| | | KGEKVRTTETQVFVATPQKNFLQERLKLIAELWDSGIKAE | |
| | | MLYKNNPKLLTQLHYCESTGIPLVVIIGEQELKEGVIKIR | |
| | | SVASREEVAIKRENFVAEIQKRLSES | |
| Mus | FL | MADRAALEELVRLQGAHVRGLKEQKASAEQIEEEVTKLLK | 109 |
| musculus | | LKAQLGQDEGKQKFVLKTPKGTRDYSPRQMAVREKVFDVI | |
| | | IRCFKRHGAEVIDTPVFELKETLTGKYGEDSKLIYDLKDQ | |
| | | GGELLSLRYDLTVPFARYLAMNKLTNIKRYHIAKVYRRDN | |
| | | PAMTRGRYREFYQCDFDIAGQFDPMIPDAECLKIMCEILS | |
| | | SLQIGNFLVKVNDRRILDGMFAVCGVPDSKFRTICSSVDK | |
| | | LDKVSWEEVKNEMVGEKGLAPEVADRIGDYVQQHGGVSLV | |
| | | EQLLQDPKLSQNKQAVEGLGDLKLLFEYLILFGIDDKISF | |
| | | DLSLARGLDYYTGVIYEAVLLQMPTQAGEEPLGVGSIAAG | |
| | | GRYDGLVGMFDPKGRKVPCVGLSIGVERIFSIVEQRLEAS | |
| | | EEKVRTTETQVLVASAQKKLLEERLKLVSELWDAGIKAEL | |
| | | LYKKNPKLLNQLQYWEEAGIPLVAIIGEQELRDGVIKLRS | |
| | | VASREEVDVRREDLVEEIRRRTNQPLSTC | |
| Canis | FL | MAERAALEELVRLQGERVRGLKQQKASAEQIEEEVAKLLK | 110 |
| lupus | | LKAQLGPDEGKQKFVLKTPKGTRDYSPRQMAVREKVFDVI | |
| familiaris | | ISCFKRHGAEVIDTPVFELKETLTGKYGEDSKLIYDLKDQ | |
| | | GGELLSLRYDLTVPFARYLAMNKLTNIKRYHIAKVYRRDN | |
| | | PAMTRGRYREFYQCDFDIAGQFDPMIPDAECLEIMCEILR | |
| | | SLQIGDFLVKVNDRRILDGMFAICGVPDSKFRTICSSVDK | |
| | | LDKVSWEEVKNEMVGEKGLAPEVADHIGDYVQQHGGISLV | |
| | | EQLLQDPELSQNKQALEGLGDLKLLFEYLTLFGIADKISF | |
| | | DLSLARGLDYYTGVIYEAVLLQTPVQAGEEPLGVGSVAAG | |
| | | GRYDGLVGMFDPKGRKVPCVGLSIGVERIFSIVEQRLEAT | |
| | | EEKVRTTETQVLVASAQKKLLEERLKLVSELWNAGIKAEL | |
| | | LYKKNPKLLNQLQYCEEAGIPLVAIIGEQELKDGVIKLRS | |
| | | VASREEVDVPREDLVEEIKRRTSQPFCIC | |
| Bos taurus | FL | MADRAALEDLVRVQGERVRGLKQQKASAEQIEEEVAKLLK | ill |
| | | LKAQLGPDEGKPKFVLKTPKGTRDYSPRQMAVREKVFDVI | |
| | | ISCFKRHGAEVIDTPVFELKETLTGKYGEDSKLIYDLKDQ | |
| | | GGELLSLRYDLTVPFARYLAMNKLTNIKRYHIAKVYRRDN | |
| | | PAMTRGRYREFYQCDFDIAGQFDPMLPDAECLKIMCEILS | |
| | | SLQIGDFLVKVNDRRILDGMFAICGVPDSKFRTICSSVDK | |
| | | LDKVSWEEVKNEMVGEKGLAPEVADRIGDYVQQHGGVSLV | |
| | | EQLLQDPKLSQNKQALEGLGDLKLLFEYLTLFGIADKISF | |
| | | DLSLARGLDYYTGVIYEAVLLQPPARAGEEPLGVGSVAAG | |
| | | GRYDGLVGMFDPKGRKVPCVGLSIGVERIFSIVEQRLEAL | |
| | | EEKVRTTETQVLVASAQKKLLEERLKLISELWDAGIKAEL | |
| | | LYKKNPKLLNQLQYCEETGIPLVAIIGEQELKDGVIKLRS | |
| | | VASREEVDVRREDLVEEIKRRTSQPLCIC | |
| Rattus | FL | MADRAALEELVRLQGAHVRGLKEQKASAEQIEEEVTKLLK | 112 |
| norvegicus | | LKAQLGHDEGKQKFVLKTPKGTRDYSPRQMAVREKVFDVI | |
| | | IRCFKRHGAEVIDTPVFELKETLTGKYGEDSKLIYDLKDQ | |
| | | GGELLSLRYDLTVPFARYLAMNKLTNIKRYHIAKVYRRDN | |
| | | PAMTRGRYREFYQCDFDIAGQFDPMIPDAECLKIMCEILS | |
| | | SLQIGNFQVKVNDRRILDGMFAVCGVPDSKFRTICSSVDK | |
| | | LDKVSWEEVKNEMVGEKGLAPEVADRIGDYVQQHGGVSLV | |
| | | EQLLQDPKLSQNKQAVEGLGDLKLLFEYLTLFGIDDKISF | |
| | | DLSLARGLDYYTGVIYEAVLLQMPTQAGEEPLGVGSIAAG | |
| | | GRYDGLVGMFDPKGRKVPCVGLSIGVERIFSIVEQKLEAS | |
| | | EEKVRTTETQVLVASAQKKLLEERLKLISELWDAGIKAEL | |
| | | LYKKNPKLLNQLQYCEEAGI PLVAIIGEQE | |
| | | LKDGVIKLRSVTSREEVDVR REDLVEEIRR RTSQPLSM | |
| Gallus | FL | MADEAAVRQQAEVVRRLKQDKAEPDEIAKEVAKLLEMKAH | 113 |
| gallus | | LGGDEGKHKFVLKTPKGTRDYGPKQMAIRERVFSAIIACF | |
| | | KRHGAEVIDTPVFELKETLTGKYGEDSKLIYDLKDQGGEL | |
| | | LSLRYDLTVPFARYLAMNKITNIKRYHIAKVYRRDNPAMT | |
| | | RGRYREFYQCDFDIAGQFDPMIPDAECLKIVQEILSDLQL | |
| | | GDFLIKVNDRRILDGMFAVCGVPDSKFRTICSSVDKLDKM | |
| | | PWEEVRNEMVGEKGLSPEAADRIGEYVQLHGGMDLIEQLL | |
| | | QDPKLSQNKLVKEGLGDMKLLFEYLTLFGITGKISFDLSL | |
| | | ARGLDYYTGVIYEAVLLQQNDHGEESVSVGSVAGGGRYDG | |
| | | LVGMFDPKGR | |
| | | KVPCVGISIGIERIFSILEQRVEASEEKIR | |
| | | TTETQVLVASAQKKLLEERLKLISELWDAGIKAEVLYKKN | |
| | | PKLLNQLQYCEDTGIPLVAIVGEQELKDGVVKLRVVATGE | |
| | | EVNIRRESLVEEIRRRTNQL | |
| Danio | FL | MAALGLVSMRLCAGLMGRRSAVRLHSLRVCSGMTISQIDE | 114 |
| rerio | | EVARLLQLKAQLGGDEGKHVFVLKTAKGTRDYNPKQMAIR | |
| | | EKVFNIIINCFKRHGAETIDSPVFELKETLTGKYGEDSKL | |
| | | IYDLKDQGGELLSLRYDLTVPFARYLAMNKITNIKRYHIA | |
| | | KVYRRDNPAMTRGRYREFYQCDFDIAGQYDAMIPDAECLK | |
| | | LVYEILSELDLGDFRIKVNDRRILDGMFAICGVPDEKFRT | |
| | | ICSTVDKLDKLAWEEVKKEMVNEKGLSEEVADRIRDYVSM | |
| | | QGGKDLAERLLQDPKLSQSKQACAGITDMKLLFSYLELFQ | |
| | | ITDKVVFDLSLARGLDYYTGVIYEAILTQANPAPASTPAE | |
| | | QNGAEDAGVSVGSVAGGGRYDGLVGMFDPKAGKCPVWGSA | |
| | | LALRGSSPSWSRRQSCLQRRCAPLKLKCLWLQHRRTF | |
| Macaca | FL | MAERAALEELVKLQGERVRGLKQQKASAELIEEEVGKLLK | 115 |
| fascicularis | | LKAQLGPDESKQKFVLKTPKGTRDYSPRQMAVREKVFDVI | |
| | | IRCFKRHGAEVIDTPVFELKETLMGKYGEDSKLIYDLKDQ | |
| | | GGELLSLRHDLTVPFARYLAMNKLTNIKRYHIAKVYRRDN | |
| | | PAMTRGRYREFYQCDFDIAGNFDPMIPDAECLKIMCEILS | |
| | | SLQIGDFLVKVNDRRILDGMFAICGVSDSKFRTICSSVDK | |
| | | LDKVSWEEVKNEAVLLQTPAQAGEEPLGVGSVAAGGRYDG | |
| | | LVGMFDPKGRKVPCVGLSIGVERIFSIVEQ | |
| | | RLEALEEKVRTTETQVLVASAQKKLLEERLKLVSELWDAG | |
| | | IKAELLYKKNPKLLNQLQYCEEAGIPLVAIIGEQELKDGV | |
| | | IKLRSVTSREEVNVRREDLKTGQNGDFNFYYGYFIDYYWQ | |
| | | KWPDTTPFSYKALGA | |
| HRS WHEP | | X_{A}-L-X_{B}-Q-G-X-X-V-R-X-L-K-X-X-K-A-X_{C}-V-X- | 116 |
| consensus | | x-L-L-X-L-K-X_{D} | |
| | | Where: | |
| | | X is any amino acid | |
| | | X_{A} is 0-50 amino acids | |
| | | X_{B} is about 5-7 amino acids, | |
| | | preferably 6 amino acids | |
| | | X_{c} is about 7-9 amino acids, | |
| | | preferably 8 amino acids | |
| | | X_{D} is 0-50 amino acids | |

Accordingly, in certain embodiments, the HRS polypeptide comprises, consists, or consists essentially of a mammalian HRS amino acid sequence in Table H1 (e.g., SEQ ID NOs:1-116 and 172), or an active variant or fragment thereof. In some embodiments, the HRS polypeptide comprises, consists, or consists essentially of a human HRS amino acid sequence in Table H1 (e.g., SEQ ID NOs:1-108 and 172), or an active variant or fragment thereof. In some embodiments, the expressible polynucleotide encodes an HRS polypeptide that comprises consists, or consists essentially of an amino acid sequence in Table H1 (e.g., SEQ ID NO:1-116 and 172), for example, a human HRS sequence in Table H1 SEQ ID NOs:1-108 and 172), or an active variant or fragment thereof.

As noted herein, a HRS polypeptide may be altered in various ways including amino acid substitutions, deletions, truncations, additions, and insertions. Methods for such manipulations are generally known in the art. For example, amino acid sequence variants of a HRS reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Biologically active truncated and/or variant HRS polypeptides may contain conservative amino acid substitutions at various locations along their sequence, relative to a reference HRS amino acid residue. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices are known in the art (see e.g., PAM120 matrix and PAM250 matrix as disclosed for example by Dayhoff et al., 1978, A model of evolutionary change in proteins). Matrices for determining distance relationships In M. O. Dayhoff, (ed.), Atlas of protein sequence and structure, Vol. 5, pp. 345-358, National Biomedical Research Foundation, Washington D.C.; and by Gonnet et al., (Science, 256: 14430-1445, 1992), however, include proline in the same group as glycine, serine, alanine and threonine. Accordingly, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table A.

TABLE A

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, Threonine |
| Polar/large | Asparagine, Glutamine |
| Hydrophobic | Tyrosine, Valine, Isoleucine, Leucine, Methionine, Phenylalanine, |
| | Tryptophan |
| Aromatic | Tryptophan, Tyrosine, Phenylalanine |
| Residues that influence chain | Glycine and Proline |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant HRS polypeptide can readily be determined by assaying its non-canonical activity, as described herein. Conservative substitutions are shown in Table B under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, (c) the bulk of the side chain, or (d) the biological function. After the substitutions are introduced, the variants are screened for biological activity.

TABLE B

| Original Residue | Exemplary Substitutions | Preferred Substitutions |
|---|---|---|
| Ala | Val, Leu, He | Val |
| Arg | Lys, Gin, Asn | Lys |
| Asn | Gin, His, Lys, Arg | Gin |
| Asp | Glu | Glu |
| Cys | Ser, Ala, Leu, Val | Ser, Ala |
| Gin | Asn, His, Lys, | Asn |
| Glu | Asp, Lys | Asp |
| Gly | Pro | Pro |
| His | Asn, Gin, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gin, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

In some embodiments, HRS polypeptides have one or more cysteine insertions or substitutions, for example, where one or more non-cysteine residues are substituted with a cysteine residue (e.g., to alter stability, to facilitate thiol-based conjugation of an Fc fragment, to facilitate thiol-based attachment of PEG or other molecules). In some embodiments, the one or more cysteine substitutions are near the N-terminus and/or C-terminus of the HRS polypeptide, or other surface exposed regions of a HRS polypeptide. Particular embodiments include where one or more of residues within 1, 2, 3, 4, 5, 6, 7, 8, 9, 10, 11, 12, 13, 14, 15, 16, 17, 18, 19, 20, 21, 22, 23, 24, or 25 amino acids relative to the N-terminus and/or C-terminus of an HRS polypeptide are substituted with a cysteine residue. In some embodiments, cysteine residues may be added to the HRS polypeptide through the creation of N, or C-terminal fusion proteins. Such fusion proteins may be of any length, but will typically be about 1-5, or about 5-10, about 10 to 20, or about 20 to 30 amino acids in length.

Specific examples of cysteine modified proteins are shown in Table H2, which are based on the HRS polypeptide HRS(1-60). This approach can be applied to the HRS polypeptides of Table H1 and other HRS polypeptides described herein.

TABLE H2
Exemplary cysteine modified HRS polypeptides

| | | SEQ ID |
|---|---|---|
| Name | Protein Sequences | NO: |
| HRS | MCAERAALEELVKLQGERVRGLKQQKASAEL | 117 |
| (1-60)- | IEEEVAKLLKLKAQLGPDESKQKFVLKTPK | |
| M1MC- | | |
| HRS | MAERAALEELVKLQGERVRGLKQQKCSAELI | 118 |
| (1-60)- | EEEVAKLLKLKAQLGPDESKQKFVLKTPK | |
| A26C- | | |
| HRS | MAERAALEELVKLQGERVRGLKQQKASAELI | 119 |
| (1-60)- | EEEVAKLLKLKAQLGPDESKQKFVLKTPKC | |
| C61 | | |

Accordingly, in certain embodiments, the HRS polypeptide comprises, consists, or consists essentially of an amino acid sequence in Table H2 (SEQ ID NO:117-119) or an active variant or fragment thereof. In some embodiments, the expressible polynucleotide encodes an HRS polypeptide that comprises consists, or consists essentially of an amino acid sequence in Table H2 (e.g., SEQ ID NO:117-119) or an active variant or fragment thereof.

In some embodiments, the HRS polypeptide have mutations in which the endogenous or naturally-occurring cysteine residues are mutated to alternative amino acids, or deleted. In some embodiments, the insertion or substitution of cysteine residue(s) into the HRS polypeptide is combined with the elimination of other surface exposed reactive cysteine residues. Accordingly, in some embodiments, an HRS polypeptide comprises one or more substitutions and/or deletions at any one or more of Cys83, Cys174, Cys191, Cys196, Cys224, Cys235, Cys379, Cys455, Cys507, and/or Cys509 (as defined by SEQ ID NO:1), for instance, to remove naturally-occurring cysteine residues, including combinations thereof.

Specific embodiments include an HRS polypeptide of Table H1 having a mutation or deletion of any one or more of Cys83, Cys174, Cys191, Cys196, Cys224, Cys235, Cys379, Cys455, or the deletion of Cys507 and Cys509, for instance, by the deletion of the C-terminal 3 amino acids (4507-509). Exemplary mutations at these positions include for example the mutation of cysteine to serine, alanine, leucine, valine or glycine. In certain embodiments, amino acid residues for specific cysteine substitutions can be selected from naturally-occurring substitutions that are found in HRS orthologs from other species and organisms. Exemplary substitutions of this type are presented in Table H3.

TABLE H3
Naturally-occurring sequence variation at positions occupied by cysteine residues in human HRS

| H. | | | | | | | | | | | | |
|---|---|---|---|---|---|---|---|---|---|---|---|---|
| sapiens | | | | | | | | | | | | |
| cysteine | P. | M. | B. | M. | R. | G. | X. | D. | D. | C. | S. | E. |
| residue # | troglodyte | mulatta | taurus | musculus | norvegicus | gallus | laevis | rerio | melanogaster | elegans | cerevisiae | coli |
| 83 | C | C | C | C | C | C | C | C | V | T | L | V |
| 174 | C | C | C | C | C | C | C | C | C | C | C | L |
| 191 | C | C | C | C | C | C | C | C | C | V | C | A/L |
| 196 | C | C | C | C | C | Q | H | Y | S | M | V | L/A |
| 224 | C | C | C | C | C | C | C | C | C | S | A | A |
| 235 | C | C | C | C | C | C | C | C | C | C | S | E |
| 379 | C | C | C | C | C | C | C | V | C | C | C | A |
| 455 | C | C | C | C | C | C | C | — | C | C | A | A |
| 507 | C | R | C | S | S | — | — | — | — | S/Q | S/E | — |
| 509 | C | C | C | C | — | — | — | — | — | I | I/G | — |

In some embodiments, the naturally-occurring cysteines selected for mutagenesis are selected based on their surface exposure. Accordingly, in one aspect the cysteine residues selected for substitution are selected from Cys224, Cys235, Cys507 and Cys509. In some embodiments, the last three (C-terminal) residues of SEQ ID NO:1 are deleted so as to delete residues 507 to 509. In some embodiments, the cysteines are selected for mutation or deletion so as to eliminate an intramolecular cysteine pair, for example Cys174 and Cys191.

Specific examples of cysteine mutations/substitutions (indicated in bold underline) to reduce surface exposed cysteine residues include those listed below in Table H4.

TABLE H4
Exemplary HRS polypeptides with substitutions to remove surface
exposed cysteines

| | | SEQ ID |
|---|---|---|
| Name | Protein Sequence | NO: |
| HRS(1-506) | MAERAALEELVKLQGERVRGLKQQKASAELIEEEVAKLLKLKAQLGPDESK | 120 |
| C174A | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQADFDIAGNFDPMIPDAECLKIMCEILSSLQI | |
| | GDFLVKVNDRRILDGMFAICGVSDSKFRTICSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |
| HRS(1-506) | MAERAALEELVKLQGERVRGLKQQKASAELIEEEVAKLLKLKAQLGPDESK | 121 |
| C174V | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQVDFDIAGNFDPMIPDAECLKIMCEILSSLQI | |
| | GDFLVKVNDRRILDGMFAICGVSDSKFRTICSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |
| HRS(1-506) | MAERAALEELVKLQGERVRGLKQQKASAELIEEEVAKLLKLKAQLGPDESK | 122 |
| C191A | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQCDFDIAGNFDPMIPDAEALKIMCEILSSLQI | |
| | GDFLVKVNDRRILDGMFAICGVSDSKFRTICSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |
| HRS(1-506) | MAERAALEELVKLQGERVRGLKQQKASAELIEEEVAKLLKLKAQLGPDESK | 123 |
| C191S | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQCDFDIAGNFDPMIPDAESLKIMCEILSSLQI | |
| | GDFLVKVNDRRILDGMFAICGVSDSKFRTICSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |
| HRS(1-506) | MAERAALEELVKLQGERVRGLKQQKASAELIEEEVAKLLKLKAQLGPDESK | 124 |
| C191V | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQCDFDIAGNFDPMIPDAEVLKIMCEILSSLQI | |
| | GDFLVKVNDRRILDGMFAICGVSDSKFRTICSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |
| HRS(1-506) | MAERAALEELVKLQGERVRGLKQQKASAELIEEEVAKLLKLKAQLGPDESK | 125 |
| C224S | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQCDFDIAGNFDPMIPDAECLKIMCEILSSLQI | |
| | GDFLVKVNDRRILDGMFAISGVSDSKFRTICSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |
| HRS(1-506) | MAERAALEELVKLQGERVRGLKQQKASAELIEEEVAKLLKLKAQLGPDESK | 126 |
| C235S | QKFVLKTPKGTRDYSPRQMAVREKVFDVIIRCFKRHGAEVIDTPVFELKET | |
| | LMGKYGEDSKLIYDLKDQGGELLSLRYDLTVPFARYLAMNKLTNIKRYHIA | |
| | KVYRRDNPAMTRGRYREFYQCDFDIAGNFDPMIPDAECLKIMCEILSSLQI | |
| | GDFLVKVNDRRILDGMFAICGVSDSKFRTISSSVDKLDKVSWEEVKNEMVG | |
| | EKGLAPEVADRIGDYVQQHGGVSLVEQLLQDPKLSQNKQALEGLGDLKLLF | |
| | EYLTLFGIDDKISFDLSLARGLDYYTGVIYEAVLLQTPAQAGEEPLGVGSV | |
| | AAGGRYDGLVGMFDPKGRKVPCVGLSIGVERIFSIVEQRLEALEEKIRTTE | |
| | TQVLVASAQKKLLEERLKLVSELWDAGIKAELLYKKNPKLLNQLQYCEEAG | |
| | IPLVAIIGEQELKDGVIKLRSVTSREEVDVRREDLVEEIKRRTGQPL | |

Accordingly, in certain embodiments, the HRS polypeptide comprises, consists, or consists essentially of an amino acid sequence in Table H4 (SEQ ID NO:120-126) or an active variant or fragment thereof. In some embodiments, the expressible polynucleotide encodes an HRS polypeptide that comprises consists, or consists essentially of an amino acid sequence in Table H4 (e.g., SEQ ID NO:120-126) or an active variant or fragment thereof.

In some embodiments, such cysteine substituted mutants are modified to engineer-in, insert, or otherwise introduce a new surface exposed cysteine residue at a defined surface exposed position, where the introduced residue does not substantially interfere with the non-canonical activity of the HRS polypeptide. Specific examples include for example the insertion (or re-insertion back) of additional cysteine residues at the N- or C-terminus of any of the reduced cysteine HRS polypeptides described above. In some embodiments, the insertion of such N- or C-terminal surface exposed cysteines involves the re-insertion of the last 1, last 2, or last 3 naturally occurring C-terminal amino acids of the full length human HRS to a reduced cysteine variant of a HRS polypeptide e.g., the re-insertion of all or part of the sequence CIC (Cys Ile Cys). Exemplary reduced cysteine mutants include for example any combination of mutations (or the deletion of) at residues Cys174, Cys191, Cys224, and Cys235, and or the deletion or substitution of Cys507 and Cys509 (based on the numbering of full length human cytosolic HRS (SEQ ID NO:1) in any of the HRS polypeptides of Table H1.

For some types of site-specific conjugation or attachment to heterologous molecules such as Fc regions or PEG or other heterologous molecules, HRS polypeptides may have one or more glutamine substitutions, where one or more naturally-occurring (non-glutamine) residues are substituted with glutamine, for example, to facilitate transglutaminase-catalyzed attachment of the molecule(s) to the glutamine's amide group. In some embodiments, glutamine substitutions are introduced near the N-terminus and/or C-terminus of the HRS polypeptide. Particular embodiments include where one or more of residues within 1, 2, 3, 4, 5, 6, 7, 8, 9, 10, 11, 12, 13, 14, 15, 16, 17, 18, 19, 20, 21, 22, 23, 24 or 25 amino acids relative to the N-terminus and/or C-terminus of an HRS polypeptide are substituted with a glutamine residue. These and related HRS polypeptides can also include substitutions (e.g., conservative substitutions) to remove any naturally-occurring glutamine residues, if desired, and thereby regulate the degree of site-specific conjugation or attachment.

For certain types of site-specific conjugation or attachment to heterologous molecules such as Fc regions or PEG or other heterologous molecules, HRS polypeptides may have one or more lysine substitutions, where one or more naturally-occurring (non-lysine) residues are substituted with lysine, for example, to facilitate acylation or alkylation-based attachment of molecule(s) to the lysine's amino group. These methods also typically result in attachment of molecule(s) to the N-terminal residue. In some embodiments, lysine substations are near the N-terminus and/or C-terminus of the HRS polypeptide. Particular embodiments include where one or more of residues within 1, 2, 3, 4, 5, 6, 7, 8, 9, 10, 11, 12, 13, 14, 15, 16, 17, 18, 19, 20, 21, 22, 23, 24 or 25 amino acids to the N-terminus and/or C-terminus of an HRS polypeptide are substituted with a lysine residue. These and related HRS polypeptides can also include substitutions (e.g., conservative substitutions) to remove any naturally-occurring lysine residues, if desired, and thereby regulate the degree of site-specific conjugation or attachment.

Site-specific conjugation to HRS polypeptides may also be performed by substituting one or more solvent accessible surface amino acids of a HRS polypeptide. For example, suitable solvent accessible amino acids may be determined based on the predicted solvent accessibility using the SPIDDER server (http://sppider.cchmc.org/) using the published crystal structure of an exemplary HRS polypeptide (see Xu et al., Structure. 20:1470-7, 2012; and U.S. Application No. 61/674,639). Based on this analysis several amino acids on the surface may potentially be used as mutation sites to introduce functional groups suitable for conjugation or attachment. The surface accessibility score of amino acids based on the crystal structure can be calculated, where the higher scores represent better accessibility. In particular embodiments, higher scores (for example, >40) are preferred. Accordingly in some embodiments an amino acid position have a surface accessibility score of greater than 40 may be used to introduce a cysteine, lysine, glutamine, or other non-naturally-occurring amino acid.

In particular embodiments, a solvent accessible surface amino acid is selected from the group consisting of: alanine, glycine, and serine, and can be substituted with naturally occurring amino acids including, but not limited to, cysteine, glutamine, or lysine, or a non-naturally occurring amino acid that is optimized for site specific conjugation or attachment.

Certain embodiments include site-specific conjugation or attachment to an HRS polypeptide at any amino acid position by virtue of substituting a non-naturally-occurring amino acid comprising a functional group that will form a covalent bond with the functional group attached to a heterologous molecules such as an Fc region or PEG or other heterologous molecule. Non-natural amino acids can be inserted or substituted at, for example, one or more of residues within 1, 2, 3, 4, 5, 6, 7, 8, 9, 10, 11, 12, 13, 14, 15, 16, 17, 18, 19, 20, 21, 22, 23, 24 or 25 amino acids relative to the N-terminus and/or C-terminus, at the N-terminus and/or C-terminus, or at a solvent accessible surface amino acid residue of an HRS polypeptide described herein.

In particular embodiments, non-naturally occurring amino acids include, without limitation, any amino acid, modified amino acid, or amino acid analogue other than selenocysteine and the following twenty genetically encoded alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine. The generic structure of an alpha-amino acid is illustrated by the following formula:

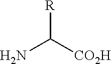

A non-natural amino acid is typically any structure having the foregoing formula wherein the R group is any substituent other than one used in the twenty natural amino acids. See, e.g., biochemistry texts such as Biochemistry by L. Stryer, 3rd ed. 1988, Freeman and Company, New York, for structures of the twenty natural amino acids. Note that the non-natural amino acids disclosed herein may be naturally occurring compounds other than the twenty alpha-amino acids above. Because the non-natural amino acids disclosed herein typically differ from the natural amino acids in side chain only, the non-natural amino acids form amide bonds with other amino acids, e.g., natural or non-natural, in the same manner in which they are formed in naturally occurring proteins. However, the non-natural amino acids have side chain groups that distinguish them from the natural amino acids. For example, R in foregoing formula optionally comprises an alkyl-, aryl-, aryl halide, vinyl halide, alkyl halide, acetyl, ketone, aziridine, nitrile, nitro, halide, acyl-, keto-, azido-, hydroxyl-, hydrazine, cyano-, halo-, hydrazide, alkenyl, alkynyl, ether, thio ether, epoxide, sulfone, boronic acid, boronate ester, borane, phenylboronic acid, thiol, seleno-, sulfonyl-, borate, boronate, phospho, phosphono, phosphine, heterocyclic-, pyridyl, naphthyl, benzophenone, a constrained ring such as a cyclooctyne, thio ester, enone, imine, aldehyde, ester, thioacid, hydroxylamine, amino, carboxylic acid, alpha-keto carboxylic acid, alpha or beta unsaturated acids and amides, glyoxyl amide, or organosilane group, or the like or any combination thereof.

Specific examples of unnatural amino acids include, but are not limited to, p-acetyl-L-phenylalanine, O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, β-O-GlcNAc-L-serine, a tri-O-acetyl-GalNAc-α-threonine, an α-GalNAc-L-threonine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, those listed below, or elsewhere herein, and the like.

Accordingly, one may select a non-naturally occurring amino acid comprising a functional group that forms a covalent bond with any preferred functional group of a desired molecule (e.g., Fc region, PEG). Non-natural amino acids, once selected, can either be purchased from vendors, or chemically synthesized. Any number of non-natural amino acids may be incorporated into the target molecule and may vary according to the number of desired molecules that are to be attached. The molecules may be attached to all or only some of the non-natural amino acids. Further, the same or different non-natural amino acids may be incorporated into a HRS polypeptide, depending on the desired outcome. In certain embodiments, about 1, 2, 3, 4, 5, 6, 7, 8, 9, 10 or more non-natural amino acids are incorporated into a HRS polypeptide any or all of which may be conjugated to a molecule comprising a desired functional group.

In certain aspects, the use of non-natural amino acids can be utilized to modify (e.g., increase) a selected non-canonical activity of an HRS polypeptide, or to alter the in vivo or in vitro half-life of the protein. Non-natural amino acids can also be used to facilitate (selective) chemical modifications (e.g., pegylation) of an HRS polypeptide, as described herein. For instance, certain non-natural amino acids allow selective attachment of polymers such as an Fc region or PEG to a given protein, and thereby improve their pharmacokinetic properties.

Specific examples of amino acid analogs and mimetics can be found described in, for example, Roberts and Vellaccio, The Peptides: Analysis, Synthesis, Biology, Eds. Gross and Meinhofer, Vol. 5, p. 341, Academic Press, Inc., New York, N.Y. (1983), the entire volume of which is incorporated herein by reference. Other examples include peralkylated amino acids, particularly permethylated amino acids. See, for example, Combinatorial Chemistry, Eds. Wilson and Czarnik, Ch. 11, p. 235, John Wiley & Sons Inc., New York, N.Y. (1997), the entire book of which is incorporated herein by reference. Yet other examples include amino acids whose amide portion (and, therefore, the amide backbone of the resulting peptide) has been replaced, for example, by a sugar ring, steroid, benzodiazepine or carbo cycle. See, for instance, Burger's Medicinal Chemistry and Drug Discovery, Ed. Manfred E. Wolff, Ch. 15, pp. 619-620, John Wiley & Sons Inc., New York, N.Y. (1995), the entire book of which is incorporated herein by reference. Methods for synthesizing peptides, polypeptides, peptidomimetics and proteins are well known in the art (see, for example, U.S. Pat. No. 5,420,109; M. Bodanzsky, Principles of Peptide Synthesis (1st ed. & 2d rev. ed.), Springer-Verlag, New York, N.Y. (1984 & 1993), see Chapter 7; Stewart and Young, Solid Phase Peptide Synthesis, (2d ed.), Pierce Chemical Co., Rockford, Ill. (1984), each of which is incorporated herein by reference). Accordingly, the HRS polypeptides can be composed of naturally occurring and non-naturally occurring amino acids as well as amino acid analogs and mimetics.

In certain embodiments, an HRS polypeptide comprises, consists, or consists essentially of the minimal active fragment of a full-length HRS polypeptide capable of modulating an anti-inflammatory activity in vivo or having neuropilin polypeptide binding activity. In some embodiments, such a minimal active fragment comprises, consists, or consists essentially of the WHEP domain (e.g., about amino acids 1-43 of SEQ ID NO:1) or an active variant or fragment thereof.

In certain embodiments, the HRS polypeptide is about, at least about, and/or up to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 501, 502, 503, 504, 505, 506, 507, 508, or 509 amino acids in length, including all integers ranges in between, and comprises, consists, or consists essentially of an amino acid sequence in Table H1, Table H2, or Table H4.

In certain embodiments, the HRS polypeptide possesses at least one non-canonical activity, for example, an anti-inflammatory activity or binding to a neuropilin polypeptide, examples of which are described herein. Assays to determine anti-inflammatory activity, or neuropilin polypeptide or receptor binding, including routine measurements of cytokine release from in vitro cell based, and animal studies are well established in the art (see, for example, Wittmann et al., J Vis Exp. (65):e4203. doi: 10.3791/4203, 2012; Feldman et al., Mol Cell. 47:585-95, 2012; Clutterbuck et al., J Proteomics. 74:704-15, 2011, Giddings and Maitra, J Biomol Screen. 15:1204-10, 2010; Wijnhoven et al., Glycoconj J. 25:177-85, 2008; and Frow et al., Med Res Rev. 24:276-98, 2004) and can be readily used to profile and optimize anti-inflammatory activity. An exemplary in vivo experimental system is also described in the accompanying Examples.

It will be appreciated that in any of the HRS polypeptides, the N-terminal acid of the HRS polypeptide (for example, the N-terminal Met) may be deleted or replaced with a different amino acid.

In some embodiments, fusion proteins of HRS polypeptide to other (non HARS) proteins (e.g. heterologous proteins or polypeptides) are also included, and these fusion proteins may modulate the HRS polypeptide's biological activity, secretion, antigenicity, targeting, biological life, ability to penetrate cellular membranes, or the blood brain barrier, or pharmacokinetic properties. Examples of fusion proteins that improve pharmacokinetic properties (“PK modifiers”) include without limitation, fusions to human albumin (Osborn et al.: Eur. J. Pharmacol. 456(1-3): 149-158, (2002)), antibody Fc domains, poly Glu or poly Asp sequences, and transferrin. Additionally, fusion with conformationally disordered polypeptide sequences composed of the amino acids Pro, Ala, and Ser (‘PASylation’) or hydroxyethyl starch (sold under the trademark HESYLATION®) provides a simple way to increase the hydrodynamic volume of the HRS polypeptide. This additional extension adopts a bulky random structure, which significantly increases the size of the resulting fusion protein. By this means the typically rapid clearance of smaller HRS polypeptides via kidney filtration is retarded by several orders of magnitude. Additionally use of Ig G fusion proteins has also been shown to enable some fusion protein proteins to penetrate the blood brain barrier (Fu et al., (2010) Brain Res. 1352:208-13).

Examples of fusion proteins that modulate the antigenicity, or other properties of the HRS polypeptide, include fusions to T cell binding ligands, including for example, MHC Class I and II proteins, b-2 microglobulin, portions of LFA-3, portions of the Fc region of the heavy chain, and conjugates and derivatives thereof. Examples of such fusion proteins are described EP 1 964 854; U.S. Pat. Nos. 5,468,481; 5,130,297; 5,635,363; and 6,451,314; and U.S. Application No. 2009/0280135.

In some embodiments, the HRS polypeptide can include synthetic, or naturally occurring secretion signal sequences, derived from other well characterized secreted proteins. In some embodiments such proteins, may be processed by proteolytic cleavage to form the HRS polypeptide in situ. In some embodiments the HRS polypeptide can comprise heterologous proteolytic cleavage sites, to enable the in situ expression, and production of the HRS polypeptide either at an intracellular, or an extracellular location. Other fusions proteins may also include for example fusions of HRS polypeptide to ubiquitin to provide a new N-terminal amino acid, or the use of a secretion signal to mediate high level secretion of the HRS polypeptide into the extracellular medium, or N, or C-terminal epitope tags to improve purification or detection, and fusions to cell penetrating peptides.

In certain aspects, the use of non-natural amino acids can be utilized to modify (e.g., increase) a selected non-canonical activity of a HRS polypeptide, or to alter the in vivo or in vitro half-life of the protein. Non-natural amino acids can also be used to facilitate (selective) chemical modifications (e.g., pegylation) of a HRS protein, as described elsewhere herein. For instance, certain non-natural amino acids allow selective attachment of polymers such as PEG to a given protein, and thereby improve their pharmacokinetic properties.

Certain embodiments include HRS-Fc conjugates, which comprise at least one Fc region that is covalently attached to one or more HRS polypeptides. Examples of HRS-Fc conjugates include fusion proteins and various forms of chemically cross-linked proteins. A wide variety of Fc region sequences may be employed in the HRS-Fc conjugates, including wild-type sequences from any number of species, as well as variants, fragments, hybrids, and chemically modified forms thereof. The HRS-Fc polypeptides may also (optionally) comprise one or more linkers, which typically separate the Fc region(s) from the HRS polypeptide(s), including peptide linkers and chemical linkers, as described herein and known in the art. It will be appreciated that in any of these HRS-Fc conjugates the native N or C terminal amino acid of the HRS polypeptides, or native N or C-amino acid in the Fc domain, may be deleted and/or replaced with non-native amino acid(s), for example, to facilitate expression and or cloning or to serve as a linker sequence between the two proteins.

HRS-Fc conjugate polypeptides can provide a variety of advantages relative to un-conjugated or unmodified HRS polypeptides, e.g., corresponding HRS polypeptides of the same or similar sequence having no Fc region(s) attached thereto. Merely by way of illustration, the covalent attachment of one or more Fc regions can alter (e.g., increase, decrease) the HRS polypeptide's solubility, half-life (e.g., in serum, in a selected tissue, in a test tube under storage conditions, for example, at room temperature or under refrigeration), dimerization or multimerization properties, biological activity or activities, for instance, by providing Fc-region-associated effector functions (e.g., activation of the classical complement cascade, interaction with immune effector cells via the Fc receptor (FcR), compartmentalization of immunoglobulins), cellular uptake, intracellular transport, tissue distribution, and/or bioavailability, relative to an unmodified HRS polypeptide having the same or similar sequence. In certain aspects, Fc regions can confer effector functions relating to complement-dependent cytotoxicity (CDC), antibody-dependent cell-mediated cytotoxicity (ADCC), and/or antibody-dependent cell-mediated phagocytocis (ADCP), which are believed to play a role in clearing specific target cells such as tumor cells and infected cells.

Certain embodiments employ HRS-Fc fusion proteins. “Fusion proteins” are defined elsewhere herein and well known in the art, as are methods of making fusion proteins (see, e.g., U.S. Pat. Nos. 5,116,964; 5,428,130; 5,455,165; 5,514,582; 6,406,697; 6,291,212; and 6,300,099 for general disclosure and methods related to Fc fusion proteins). In a HRS-Fc fusion protein, the Fc region can be fused to the N-terminus of the HRS polypeptide, the C-terminus, or both. In some embodiments, one or more Fc regions can be fused internally relative to HRS sequences, for instance, by placing an Fc region between a first HRS sequence (e.g., domain) and a second HRS sequence (e.g., domain), where the first HRS sequence is fused to the N-terminus of the Fc region and the second HRS sequence is fused to the C-terminus of the Fc region. In specific embodiments, the first and second HRS sequences are identical. In some embodiments, the first and second HRS sequences are different (e.g., they include different functional domains of the HRS polypeptide). Certain HRS-Fc fusion proteins can also include additional heterologous protein sequences, that is, non-Fc region and non-HRS polypeptide sequences.

The term “HRS-Fc” can indicate, but does not necessarily indicate, the N-terminal or C-terminal attachment of the Fc region to the HRS polypeptide. For instance, in certain instances the term “Fc-HRS” indicates fusion of the Fc region to the N-terminus of the HRS polypeptide, and the term “HRS-Fc” indicates fusion of the Fc region to the C-terminus of the HRS polypeptide. However, either term can be used more generally to refer to any fusion protein or conjugate of an Fc region and a HRS polypeptide.

In some embodiments the HRS-Fc fusion proteins may comprise tandemly repeated copies of the HRS polypeptide coupled to a single Fc domain, optionally separated by linker peptides. Exemplary tandemly repeated HRS-Fc fusion proteins are provided in Table H5. The preparation and sequences for specific tandemly repeated HRS-Fc conjugates are illustrated in the Examples.

TABLE H5
Exemplary Tandem HRS-Fc conjugates

HRS polypeptide-L-HRS-polypeptide-L-Fc
HRS-polypeptide-L-HRS-polypeptide-L-HRS-polypeptide-L-Fc
HRS-polypeptide-L-HRS-polypeptide-L-HRS-polypeptide-L-
HRS-polypeptide-L-Fc
Fc-L-HRS-polypeptide-L-HRS-polypeptide
Fc-L-HRS-polypeptide-L-HRS-L-HRS-polypeptide
Fc-L-HRS-polypeptide-L-HRS-L-HRS-L-HRS-polypeptide
Where:
“Fc” is an Fc domain as described herein
″HRS-polypeptide″ is an HRS polypeptide as described herein
“L” is an optional peptide linker

Certain embodiments relate to HRS-Fc conjugates, where, for instance, one or more Fc regions are chemically conjugated or cross-linked to the HRS polypeptide(s). In these and related aspects, the Fc region can be conjugated to the HRS polypeptide at the N-terminal region (e.g., within the first 10, 20, 30, 40, 50, 60, 70, 80, 90, 100 or so amino acids), the internal region (between the N-terminal and C-terminal regions), and/or the C-terminal region (e.g., within the last 10, 20, 30, 40, 50, 60, 70, 80, 90, 100 or so amino acids). Polypeptides can be conjugated or cross-linked to other polypeptides according to a variety of routine techniques in the art. For instance, certain techniques employ the carboxyl-reactive carbodiimide crosslinker EDC (or EDAC), which covalently attaches via D, E, and C-terminal carboxyl groups. Other techniques employ activated EDC, which covalently attaches via K and N-terminal amino groups). Still other techniques employ m-maleimidobenzoyl-N-hydroxysuccinimide ester (MBS) or Sulfo-MBS, which covalently attach via the thiol group of a cysteine residue (see also U.S. Application No. 2007/0092940 for cysteine engineered Ig regions that can be used for thiol conjugation). Such cross-linked proteins can also comprise linkers, including cleavable or otherwise releasable linkers (e.g., enzymatically cleavable linkers, hydrolysable linkers), and non-cleavable linkers (i.e., physiologically-stable linkers). Certain embodiments may employ non-peptide polymers (e.g., PEG polymers; HRS-N-PEG-N-Fc conjugate) as a cross-linker between the Fc region(s) and the HRS polypeptide(s), as described, for example, in U.S. Application No. 2006/0269553. See also US Application No. 2007/0269369 for exemplary descriptions of Fc region conjugation sites.

In certain embodiments, discussed in greater detail below, variant or otherwise modified Fc regions can be employed, including those having altered properties or biological activities relative to wild-type Fc region(s). Examples of modified Fc regions include those having mutated sequences, for instance, by substitution, insertion, deletion, or truncation of one or more amino acids relative to a wild-type sequence, hybrid Fc polypeptides composed of domains from different immunoglobulin classes/subclasses, Fc polypeptides having altered glycosylation/sialylation patterns, and Fc polypeptides that are modified or derivatized, for example, by biotinylation (see, e.g., US Application No. 2010/0209424), phosphorylation, sulfation, etc., or any combination of the foregoing. Such modifications can be employed to alter (e.g., increase, decrease) the binding properties of the Fc region to one or more particular FcRs (e.g., FcγRI, FcγRIIa, FcγRIIb, FcγRIIc, FcγRIIIa, FcγRIIIb, FcRn), its pharmacokinetic properties (e.g., stability or half-life, bioavailability, tissue distribution, volume of distribution, concentration, elimination rate constant, elimination rate, area under the curve (AUC), clearance, Cₘₐₓ, tₘₐₓ, Cₘᵢₙ, fluctuation), its immunogenicity, its complement fixation or activation, and/or the CDC/ADCC/ADCP-related activities of the Fc region, among other properties described herein, relative to a corresponding wild-type Fc sequence.

The “Fc region” of a HRS-Fc conjugate provided herein is usually derived from the heavy chain of an immunoglobulin (Ig) molecule. A typical Ig molecule is composed of two heavy chains and two light chains. The heavy chains can be divided into at least three functional regions: the Fd region, the Fc region (fragment crystallizable region), and the hinge region, the latter being found only in IgG, IgA, and IgD immunoglobulins. The Fd region comprises the variable (V_{H}) and constant (CH₁) domains of the heavy chains, and together with the variable (VL) and constant (CL) domains of the light chains forms the antigen-binding fragment or Fab region.

The Fc region of IgG, IgA, and IgD immunoglobulins comprises the heavy chain constant domains 2 and 3, designated respectively as CH₂ and CH₃ regions; and the Fc region of IgE and IgM immunoglobulins comprises the heavy chain constant domains 2, 3, and 4, designated respectively as CH₂, CH₃, and CH₄ regions. The Fc region is mainly responsible for the immunoglobulin effector functions, which include, for example, complement fixation and binding to cognate Fc receptors of effector cells.

The hinge region (found in IgG, IgA, and IgD) acts as a flexible spacer that allows the Fab portion to move freely in space relative to the Fc region. In contrast to the constant regions, the hinge regions are structurally diverse, varying in both sequence and length among immunoglobulin classes and subclasses. The hinge region may also contain one or more glycosylation site(s), which include a number of structurally distinct types of sites for carbohydrate attachment. For example, IgA 1 contains five glycosylation sites within a 17 amino acid segment of the hinge region, conferring significant resistance of the hinge region polypeptide to intestinal proteases. Residues in the hinge proximal region of the CH₂ domain can also influence the specificity of the interaction between an immunoglobulin and its respective Fc receptor(s) (see, e.g., Shin et al., Intern. Rev. Immunol. 10:177-186, 1993).

The term “Fc region” or “Fc fragment” or “Fc” as used herein, thus refers to a protein that contains one or more of a CH₂ region, a CH₃ region, and/or a CH₄ region from one or more selected immunoglobulin(s), including fragments and variants and combinations thereof. An “Fc region” may also include one or more hinge region(s) of the heavy chain constant region of an immunoglobulin. In certain embodiments, the Fc region does not contain one or more of the CH₁, C_{L}, V_{L}, and/or V_{H} regions of an immunoglobulin.

The Fc region can be derived from the CH₂ region, CH₃ region, CH₄ region, and/or hinge region(s) of any one or more immunoglobulin classes, including but not limited to IgA, IgD, IgE, IgG, IgM, including subclasses and combinations thereof. In some embodiments, the Fc region is derived from an IgA immunoglobulin, including subclasses IgA1 and/or IgA2. In certain embodiments, the Fc region is derived from an IgD immunoglobulin. In particular embodiments, the Fc region is derived from an IgE immunoglobulin. In some embodiments, the Fc region is derived from an IgG immunoglobulin, including subclasses IgG1, IgG2, IgG2, IgG3, and/or IgG4. In certain embodiments, the Fc region is derived from an IgM immunoglobulin.

Certain Fc regions demonstrate specific binding for one or more Fc-receptors (FcRs). Examples of classes of Fc receptors include Fcγ receptors (FcγR), Fcα receptors (FcαR), Fcε receptors (FcεR), and the neonatal Fc receptor (FcRn). For instance, certain Fc regions have increased binding to (or affinity for) one or more FcγRs, relative to FcαRs, FcεRs, and/or FcRn. In some embodiments, Fc regions have increased binding to FcαRs, relative to one or more FcγRs, FcεRs, and/or FcRn. In some embodiments, Fc regions have increased binding to FcεRs (e.g., FcαRI), relative to one or more FcγRs, FcαRs, and/or FcRn. In particular embodiments, Fc regions have increased binding to FcRn, relative to one or more FcγRs, FcαRs, and/or FcεRs. In certain embodiments, the binding (or affinity) of an Fc region to one or more selected FcR(s) is increased relative to its binding to (or affinity for) one or more different FcR(s), typically by about 1.5×, 2×, 2.5×, 3×, 3.5×, 4×, 4.5×, 5×, 6×, 7×, 8×, 9×, 10×, 15×, 20×, 25×, 30×, 40×, 50×, 60×, 70×, 80×, 90×, 100×, 200×, 300×, 400×, 500×, 600×, 700×, 800×, 900×, 1000× or more (including all integers in between).

Examples of FcγRs include FcγRI, FcγRIIa, FcγRIIb, FcγRIIc, FcγRIIIa, and FcγRIIIb. FcγRI (CD64) is expressed on macrophages and dendritic cells and plays a role in phagocytosis, respiratory burst, cytokine stimulation, and dendritic cell endocytic transport. Expression of FcγRI is upregulated by both GM-CSF and γ-interferon (γ-IFN) and downregulated by interleukin-4 (IL-4). FcγRIIa is expressed on polymorphonuclear leukocytes (PMN), macrophages, dendritic cells, and mast cells. FcγRIIa plays a role in phagocytosis, respiratory burst, and cytokine stimulation. Expression of FcγRIIa is upregulated by GM-CSF and γ-IFN, and decreased by IL-4. FcγIIb is expressed on B cells, PMN, macrophages, and mast cells. FcγIIb inhibits immunoreceptor tyrosine-based activation motif (ITAM) mediated responses, and is thus an inhibitory receptor. Expression of FcγRIIc is upregulated by intravenous immunoglobulin (IVIG) and IL-4 and decreased by γ-IFN. FcγRIIc is expressed on NK cells. FcγRIIIa is expressed on natural killer (NK) cells, macrophages, mast cells, and platelets. This receptor participates in phagocytosis, respiratory burst, cytokine stimulation, platelet aggregation and degranulation, and NK-mediated ADCC. Expression of FcγRIII is upregulated by C5a, TGF-β, and γ-IFN and downregulated by IL-4. Fc γ RIIIb is a GPI-linked receptor expressed on PMN.

Certain Fc regions have increased binding to FcγRI, relative to FcγRIIa, FcγRIIb, FcγRIIc, FcγRIIIa, and/or FcγRIIIb. Some embodiments have increased binding to FcγRIIa, relative to FcγRI, FcγRIIb, FcγRIIc, FcγRIIIa, and/or FcγRIIIb. Particular Fc regions have increased binding to FcγRIIb, relative to FcγRI, FcγRIIa, FcγRIIc, FcγRIIIa, and/or FcγRIIIb. Certain Fc regions have increased binding to FcγRIIc, relative to FcγRI, FcγRIIa, FcγRIIb, FcγRIIIa, and/or FcγRIIIb. Some Fc regions have increased binding to FcγRIIIa, relative to FcγRI, FcγRIIa, FcγRIIb, FcγRIIc, and/or FcγRIIIb. Specific Fc regions have increased binding to FcγRIIIb, relative to FcγRI, FcγRIIa, FcγRIIb, FcγRIIc, and/or FcγRIIIa.

FcαRs include FcαRI (CD89). FcαRI is found on the surface of neutrophils, eosinophils, monocytes, certain macrophages (e.g., Kupffer cells), and certain dendritic cells. FcαRI is composed of two extracellular Ig-like domains, is a member of both the immunoglobulin superfamily and the multi-chain immune recognition receptor (MIRR) family, and signals by associating with two FcRγ signaling chains.

FcεRs include FcεRI and FcεRII. The high-affinity receptor FcεRI is a member of the immunoglobulin superfamily, is expressed on epidermal Langerhans cells, eosinophils, mast cells and basophils, and plays a major role in controlling allergic responses. FcεRI is also expressed on antigen-presenting cells, and regulates the production pro-inflammatory cytokines. The low-affinity receptor FcεRII (CD23) is a C-type lectin that can function as a membrane-bound or soluble receptor. FcεRII regulates B cell growth and differentiation, and blocks IgE-binding of eosinophils, monocytes, and basophils. Certain Fc regions have increased binding to FcεRI, relative to FcεRII. Other Fc regions have increased binding to FcεRII, relative to FcεRI.

Table H6 below summarizes the characteristics of certain FcRs.

TABLE H6
Exemplary Fc-Receptors

| | Primary | | | |
|---|---|---|---|---|
| | Antibody | | Cell | Exemplary Effects Following |
| Receptor | Ligand | Ligand Affinity | Distribution | Binding to Fc Ligand |
| FcγRI | IgG1 and | High (Kd~10⁻⁹ | Macrophages | Phagocytosis |
| (CD64) | IgG3 | M) | Neutrophils | Cell activation |
| | | | Eosinophils | Activation of respiratory burst |
| | | | Dendritic cells | Induction of microbe killing |
| FcγRIIa | IgG | Low (Kd > 10⁻⁷ | Macrophages | Phagocytosis |
| (CD32) | | M) | Neutrophils | Degranulation (eosinophils) |
| | | | Eosinophils | |
| | | | Platelets | |
| | | | Langerhans cells | |
| FcγRIIb1 | IgG | Low (Kd > 10⁻⁷ | B Cells | No phagocytosis |
| (CD32) | | M) | Mast cells | Inhibition of cell activity |
| FcγRIIb2 | IgG | Low (Kd > 10⁻⁷ | Macrophages | Phagocytosis |
| (CD32) | | M) | Neutrophils | Inhibition of cell activity |
| | | | Eosinophils | |
| FcγRIlIa | IgG | Low (Kd > 10⁻⁶ | NK cells | Induction of antibody- |
| (CD16a) | | M) | Macrophages | dependent cell-mediated |
| | | | (certain tissues) | cytotoxicity (ADCC) |
| | | | | Induction of cytokine release |
| | | | | by macrophages |
| FcγRIIIb | IgG | Low (Kd > 10⁻⁶ | Eosinophils | Induction of microbe killing |
| (CD16b) | | M) | Macrophages | |
| | | | Neutrophils | |
| | | | Mast cells | |
| | | | Follicular | |
| | | | dendritic cells | |
| FcεRI | IgE | High (Kd ~ 10⁻¹⁰ | Mast cells | Degranulation |
| | | M) | Eosinophils | |
| | | | Basophils | |
| | | | Langerhans cells | |
| FcεRII | IgE | Low (Kd > 10⁻⁷ | B cells | Possible adhesion molecule |
| (CD23) | | M) | Eosinophils | |
| | | | Langerhans cells | |
| FcαRI | IgA | Low (Kd > 10⁻⁶ | Monocytes | Phagocytosis |
| (CD89) | | M) | Macrophages | Induction of microbe killing |
| | | | Neutrophils | |
| | | | Eosinophils | |
| Fcα/uR | IgA and | High for IgM, | B cells | Endocytosis |
| | IgM | Moderate for IgA | Mesangial cells | Induction of microbe killing |
| | | | Macrophages | |
| FeRn | IgG | | Monocytes | Transfers IgG from a mother |
| | | | Macrophages | to fetus through the placenta |
| | | | Dendrite cells | Transfers IgG from a mother |
| | | | Epithelial cells | to infant in milk |
| | | | Endothelial cells | Protects IgG from degradation |
| | | | Hepatocytes | |

Fc regions can be derived from the immunoglobulin molecules of any animal, including vertebrates such as mammals such cows, goats, swine, dogs, mice, rabbits, hamsters, rats, guinea pigs, non-human primates, and humans. The amino acid sequences of CH₂, CH₃, CH₄, and hinge regions from exemplary, wild-type human IgA1, IgA2, IgD, IgE, IgG1, IgG2, IgG3, IgG4, and IgM immunoglobulins are shown in Table H7.

TABLE H7
Exemplary Fc sequences

| | | SEQ ID |
|---|---|---|
| Name | Sequence | NO: |
| IgA1 hinge | VPSTPPTPSPSTPPTPSPS | 127 |
| IgA1 CH2 | CCHPRLSLHRPALEDLLLGSEANLTCTLTGLRDASGVTFTWTPSSGKSAVQ | 128 |
| | GPPERDLCGCYSVSSVLPGCAEPWNHGKTFTCTAAYPESKTPLTATLSKS | |
| IgA1 CH 3 | GNTFRPEVHLLPPPSEELALNELVTLTCLARGFSPKDVLVRWLQGSQELPR | 129 |
| | EKYLTWASRQEPSQGTTTFAVTSILRVAAEDWKKGDTFSCMVGHEALPLAF | |
| | TQKTIDRLAGKPTHVNVSVVMAEVDGTCY | |
| IgA2 hinge | VPPPPP | 130 |
| IgA2 CH2 | CCHPRLSLHRPALEDLLLGSEANLTCTLTGLRDASGATFTWTPSSGKSAVQ | 131 |
| | GPPERDLCGCYSVSSVLPGCAQPWNHGETFTCTAAHPELKTPLTANITKS | |
| IgA2 CH3 | GNTFRPEVHLLPPPSEELALNELVTLTCLARGFSPKDVLVRWLQGSQELPR | 132 |
| | EKYLTWASRQEPSQGTTTFAVTSILRVAAEDWKKGDTFSCMVGHEALPLAF | |
| | TQKTIDRLAGKPTHVNVSVVMAEVDGTCY | |
| IgD hinge | ESPKAQASSVPTAQPQAEGSLAKATTAPATTRNTGRGGEEKKKEKEKEEQE | 133 |
| | ERETKTP | |
| IgD CH2 | ECPSHTQPLGVYLLTPAVQDLWLRDKATFTCFVVGSDLKDAHLTWEVAGKV | 134 |
| | PTGGVEEGLLERHSNGSQSQHSRLTLPRSLWNAGTSVTCTLNHPSLPPQRL | |
| | MALREP | |
| IgD CH3 | AAQAPVKLSLNLLASSDPPEAASWLLCEVSGFSPPNILLMWLEDQREVNTS | 135 |
| | GFAPARPPPQPRSTTFWAWSVLRVPAPPSPQPATYTCVVSHEDSRTLLNAS | |
| | RSLEVSYVTDHGPMK | |
| IgE CH2 | VCSRDFTPPTVKILQSSCDGGGHFPPTIQLLCLVSGYTPGTINITWLEDGQ | 136 |
| | VMDVDLSTASTTQEGELASTQSELTLSQKHWLSDRTYTCQVTYQGHTFEDS | |
| | TKKCA | |
| IgE CH3 | DSNPRGVSAYLSRPSPFDLFIRKSPTITCLVVDLAPSKGTVNLTWSRASGK | 137 |
| | PVNHSTRKEEKQRNGTLTVTSTLPVGTRDWIEGETYQCRVTHPHLPRALMR | |
| | STTKTS | |
| IgE CH4 | GPRAAPEVYAFATPEWPGSRDKRTLACLIQNFMPEDISVQWLHNEVQLPDA | 138 |
| | RHSTTQPRKTKGSGFFVFSRLEVTRAEWEQKDEFICRAVHEAASPSQTVQR | |
| | AVSVNPGK | |
| IgGI hinge | EPKSCDKTHTCPPCP | 139 |
| modified | SDKTHTCPPCP | 140 |
| human IgG1 | | |
| hinge | | |
| IgG1 CH2 | APELLGGPSVFLFPPKPKDTLMISRTPEVTCVVVDVSHEDPEVKFNWYVDG | 141 |
| | VEVHNAKTKPREEQYNSTYRVVSVLTVLHQDWLNGKEYKCKVSNKALPAPI | |
| | EKTISKAK | |
| IgG1 CH3 | GQPREPQVYTLPPSRDELTKNQVSLTCLVKGFYPSDIAVEWESNGQPENNY | 142 |
| | KTTPPVLDSDGSFFLYSKLTVDKSRWQQGNVFYPSDIAVEWESNGQPENNY | |
| | LSPGK | |
| IgG1 heavy | MSDKTHTCPPCPAPELLGGPSVFLFPPKPKDTLMISRTPEVTCVVVDVSHE | 143 |
| chain | DPEVKFNWYVDGVEVHNAKTKPREEQYNSTYRVVSVLTVLHQDWLNGKEYK | |
| | CKVSNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQGNV | |
| | FSCSVMHEALHNHYTQKSLSLSPGK | |
| IgG2 hinge | ERKCCVECPPCP | 144 |
| IgG2 CH2 | APPVAGPSVFLFPPKPKDTLMISRTPEVTCVVVDVSHEDPEVQFNWYVDGV | 145 |
| | EVHNAKTKPREEQFNSTFRVVSVLTVVHQDWLNGKEYKCKVSNKGLPAPIE | |
| | KTISKTK | |
| IgG2 CH3 | GQPREPQVYTLPPSREEMTKNQVSLTCLVKGFYPSDIAVEWESNGQPENNY | 146 |
| | KTTPPMLDSDGSFFLYSKLTVDKSRWQQGNVFSCSVMHEALHNHYTQKSLS | |
| | LSPGK | |
| IgG3 hinge | ELKTPLGDTTHTCPRCPEPKSCDTPPPCPRCPEPKSCDTPPPCPRCPEPKS | 147 |
| | CDTPPPCPRCP | |
| IgG3 CH2 | APELLGGPSVFLFPPKPKDTLMISRTPEVTCVVVDVSHEDPEVQFKWYVDG | 148 |
| | VEVHNAKTKPREEQYNSTFRVVSVLTVLHQDWLNGKEYKCKVSNKALPAPI | |
| | EKTISKTK | |
| IgG3 CH3 | GQPREPQVYTLPPSREEMTKNQVSLTCLVKGFYPSDIAVEWESSGQPENNY | 149 |
| | NTTPPMLDSDGSFFLYSKLTVDKSRWQQGNIFSCSVMHEALHNRFTQKSLS | |
| | LSPGK | |
| IgG4 hinge | ESKYGPPCPSCP | 150 |
| IgG4 CH2 | APEFLGGPSVFLFPPKPKDTLMISRTPEVTCVVVDVSQEDPEVQFNWYVDG | 151 |
| | VEVHNAKTKPREEQFNSTYRVVSVLTVLHQDWLNGKEYKCKVSNKGLPSSI | |
| | EKTISKAK | |
| IgG4 CH3 | GQPREPQVYTLPPSQEEMTKNQVSLTCLVKGFYPSDIAVEWESNGQPENNY | 152 |
| | KTTPPVLDSDGSFFLYSRLTVDKSRWQEGNVFSCSVMHEALHNHYTQKSLS | |
| | LSLGK | |
| IgM CH2 | VIAELPPKVSVFVPPRDGFFGNPRKSKLICQATGFSPRQIQVSWLREGKQV | 153 |
| | GSGVTTDQVQAEAKESGPTTYKVTSTLTIKESDWLGQSMFTCRVDHRGLTF | |
| | QQNASSMCVP | |
| IgM CH3 | DQDTAIRVFAIPPSFASIFLTKSTKLTCLVTDLTTYDSVTISWTRQNGEAV | 154 |
| | KTHTNISESHPNATFSAVGEASICEDDWNSGERFTCTVTHTDLPSPLKQTI | |
| | SRPK | |
| IgM CH4 | GVALHRPDVYLLPPAREQLNLRESATITCLVTGFSPADVFVQWMQRGQPLS | 155 |
| | PEKYVTSAPMPEPQAPGRYFAHSILTVSEEEWNTGETYTCVVAHEALPNRV | |
| | TERTVDKSTGKPTLYNVSLVMSDTAGTCY | |

An Fc region of an HRS-Fc conjugate can thus comprise, consist of, or consist essentially of one or more of the human Fc region amino acid sequences of Table H7, including variants, fragments, homologs, orthologs, paralogs, and combinations thereof. Certain illustrative embodiments comprise an Fc region that ranges in size from about 20-50, 20-100, 20-150, 20-200, 20-250, 20-300, 20-400, 50-100, 50-150, 50-200, 50-250, 50-300, 50-400, 100-150, 100-200, 100-250, 100-300, 100-350, 100-400, 200-250, 200-300, 200-350, or 200-400 amino acids in length, and optionally comprises, consists of, or consists essentially of any one or more of the sequences in Table H7. Certain embodiments comprise an Fc region of up to about 50, 60, 70, 80, 90, 100, 110, 120, 130, 140, 150, 160, 170, 180, 190, 200, 210, 220, 230, 240, 250, 300, 350, 400 or more amino acids, which optionally comprises, consists of, or consists essentially of any one or more of the amino acid sequences of Table H7.

Certain Fc regions comprise, consist of, or consist essentially of human IgA1 sequences of Table H7, in any order reading from N-terminus to C-terminus, including combinations thereof, and variants and fragments thereof. Certain Fc regions comprise, consist of, or consist essentially of human the IgA1 sequence of Table H7. Certain Fc regions comprise, consist of, or consist essentially of the human IgA1 sequence of Table H7. Certain Fc regions comprise, consist of, or consist essentially of the human IgA1 sequence of Table H7.

Some Fc regions comprise, consist of, or consist essentially of human IgA2 sequences of Table H7, in any order reading from N-terminus to C-terminus, including combinations thereof, and variants and fragments thereof. Certain Fc regions comprise, consist of, or consist essentially of human the IgA2 sequence of Table H7. Certain Fc regions comprise, consist of, or consist essentially of the human IgA2 sequence of Table H7. Certain Fc regions comprise, consist of, or consist essentially of the human IgA2 sequence of Table H7.

Certain Fc regions comprise, consist of, or consist essentially of human IgD sequences of Table H7, in any order reading from N-terminus to C-terminus, including combinations thereof, and variants and fragments of these sequences and combinations. Certain Fc regions comprise, consist of, or consist essentially of human IgE sequences of Table H7, in any order reading from N-terminus to C-terminus, including combinations thereof, and variants and fragments of these sequences and combinations. Certain Fc regions comprise, consist of, or consist essentially of human IgG1 sequences of Table H7, in any order reading from N-terminus to C-terminus, including combinations thereof, and variants and fragments of these sequences and combinations. Certain Fc regions comprise, consist of, or consist essentially of human IgG2 sequences of Table H7, in any order reading from N-terminus to C-terminus, including combinations thereof. Certain Fc regions comprise, consist of, or consist essentially of human IgG3 sequences of Table H7, in any order reading from N-terminus to C-terminus, including combinations thereof. Certain Fc regions comprise, consist of, or consist essentially of human IgG4 sequences of Table H7, in any order reading from N-terminus to C-terminus, including combinations thereof. Certain Fc regions comprise, consist of, or consist essentially of human IgM sequences of Table H7, in any order reading from N-terminus to C-terminus, including combinations thereof, and variants and fragments of these sequences and combinations.

Exemplary HRS-Fc fusion conjugates are provided in Table H8 below.

TABLE H8
Exemplary HRS-Fc fusion proteins

| | | SEQ ID |
|---|---|---|
| Name | Sequence | NO: |
| Fc-HRS(2-60) | MDKTHTCPPCPAPELLGGPSVFLFPPKPKDTLMISRTPEVTCVVVDVS | 156 |
| HRS^{FC1} | HEDPEVKFNWYVDGVEVHNAKTKPREEQYNSTYRVVSVLTVLHQDWLN | |
| | GKEYKCKVSNKALPAPIEKTISKAKGQPREPQVYTLPPSRDELTKNQV | |
| | SLTCLVKGFYPSDIAVEWESNGQPENNYKTTPPVLDSDGSFFLYSKLT | |
| | VDKSRWQQGNVFSCSVMHEALHNHYTQKSLSLSPGKAERAALEELVKL | |
| | QGERVRGLKQQKASAELIEEEVAKLLKLKAQLGPDESKQKFVLKTPK | |
| Fc-HRS(2-60) | MSDKTHTCPPCPAPELLGGPSVFLFPPKPKDTLMISRTPEVTCVVVDV | 157 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQLGPDESKQKFVLKTPK | |
| HRS(1-60)-Fc | MAERAALEELVKLQGERVRGLKQQKASAELIEEEVAKLLKLKAQLGPD | 158 |
| | ESKQKFVLKTPKSDKTHTCPPCPAPELLGGPSVFLFPPKPKDTLMISR | |
| | TPEVTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVYTL | |
| | PPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| Fc-HRS(2-60) | MSDKTHTCPPCPAPELLGGPSVFLFPPKPKDTLMISRTPEVTCVVVDV | 159 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQLGPDESKQKFVLKTPK | |
| HRS(1-60)-Fc | MAERAALEELVKLQGERVRGLKQQKASAELIEEEVAKLLKLKAQLGPD | 160 |
| | ESKQKFVLKTPKSDKTHTCPPCPAPELLGGPSVFLFPPKPKDTLMISR | |
| | TPEVTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVYTL | |
| | PPSREEMTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGK | |
| Fc-HRS (2-40) | MSDKTHTCPPCPAPELLGGPSVFLFPPKPKDTLMISRTPEVTCVVVDV | 161 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLK | |
| Fc-HRS (2-45) | MSDKTHTCPPCPAPELLGGPSVFLFPPKPKDTLMISRTPEVTCVVVDV | 162 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQL | |
| Fc-HRS (2-50) | MSDKTHTCPPCPAPELLGGPSVFLFPPKPKDTLMISRTPEVTCVVVDV | 163 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQLGPDES | |
| Fc-HRS (2-55) | MSDKTHTCPPCPAPELLGGPSVFLFPPKPKDTLMISRTPEVTCVVVDV | 164 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQLGPDESKQKFV | |
| Fc-HRS (2-66) | MSDKTHTCPPCPAPELLGGPSVFLFPPKPKDTLMISRTPEVTCVVVDV | 165 |
| | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQLGPDESKQKFVLKTPK | |
| | GTRDYS | |
| HRS(1-40)-Fc | MAERAALEELVKLQGERVRGLKQQKASAELIEEEVAKLLKSDKTHTCP | 166 |
| | PCPAPELLGGPSVFLFPPKPKDTLMISRTPEVTCVVVDVSHEDPEVKF | |
| | NWYVDGVEVHNAKTKPREEQYNSTYRVVSVLTVLHQDWLNGKEYKCKV | |
| | SNKALPAPIEKTISKAKGQPREPQVYTLPPSREEMTKNQVSLTCLVKG | |
| | FYPSDIAVEWESNGQPENNYKTTPPVLDSDGSFFLYSKLTVDKSRWQQ | |
| | GNVFSCSVMHEALHNHYTQKSLSLSPGK | |
| HRS(1-45)-Fc | MAERAALEELVKLQGERVRGLKQQKASAELIEEEVAKLLKLKAQLSDK | 167 |
| | THTCPPCPAPELLGGPSVFLFPPKPKDTLMISRTPEVTCVVVDVSHED | |
| | PEVKFNWYVDGVEVHNAKTKPREEQYNSTYRVVSVLTVLHQDWLNGKE | |
| | YKCKVSNKALPAPIEKTISKAKGQPREPQVYTLPPSREEMTKNQVSLT | |
| | CLVKGFYPSDIAVEWESNGQPENNYKTTPPVLDSDGSFFLYSKLTVDK | |
| | SRWQQGNVFSCSVMHEALHNHYTQKSLSLSPGK | |
| HRS(1-50)-Fc | MAERAALEELVKLQGERVRGLKQQKASAELIEEEVAKLLKLKAQLGPD | 167 |
| | ESSDKTHTCPPCPAPELLGGPSVFLFPPKPKDTLMISRTPEVTCVVVD | |
| | VSHEDPEVKFNWYVDGVEVHNAKTKPREEQYNSTYRWSVLTVLHQDW | |
| | LNGKEYKCKVSNKALPAPIEKTISKAKGQPREPQVYTLPPSREEMTKN | |
| | QVSLTCLVKGFYPSDIAVEWESNGQPENNYKTTPPVLDSDGSFFLYSK | |
| | LTVDKSRWQQGNVFSCSVMHEALHNHYTQKSLSLSPGK | |
| HRS(1-55)-Fc | MAERAALEELVKLQGERVRGLKQQKASAELIEEEVAKLLKLKAQLGPD | 169 |
| | ESKQKFVSDKTHTCPPCPAPELLGGPSVFLFPPKPKDTLMISRTPEVT | |
| | CVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRE | |
| | EMTKNQVSLTCLVKGFYPSDIAVEWESNGQPENNYKTTPPVLDSDGSF | |
| | FLYSKLTVDKSRWQQGNVFSCSVMHEALHNHYTQKSLSLSPGK | |
| HRS(1-66)-Fc | MAERAALEELVKLQGERVRGLKQQKASAELIEEEVAKLLKLKAQLGPD | 170 |
| | ESKQKFVLKTPKGTRDYSSDKTHTCPPCPAPELLGGPSVFLFPPKPKD | |
| | TLMISRTPEVTCVVVDVSHEDPEVKFNWYVDGVEVHNAKTKPREEQYN | |
| | STYRVVSVLTVLHQDWLNGKEYKCKVSNKALPAPIEKTISKAKGQPRE | |
| | PQVYTLPPSREEMTKNQVSLTCLVKGFYPSDIAVEWESNGQPENNYKT | |
| | TPPVLDSDGSFFLYSKLTVDKSRWQQGNVFSCSVMHEALHNHYTQKSL | |
| | SLSPGK | |
| Fc-HRS(2-60) | MSDKTHTCPPCPAPELLGGPSVFLFPPKPKDTLMISRTPEVTCVVVDV | 171 |
| HRS(2-60) | SHEDPEVKFNWYVDGVEVHNAKTKPREEQYNSTYRVVSVLTVLHQDWL | |
| | NGKEYKCKVSNKALPAPIEKTISKAKGQPREPQVYTLPPSREEMTKNQ | |
| | VSLTCLVKGFYPSDIAVEWESNGQPENNYKTTPPVLDSDGSFFLYSKL | |
| | TVDKSRWQQGNVFSCSVMHEALHNHYTQKSLSLSPGKAERAALEELVK | |
| | LQGERVRGLKQQKASAELIEEEVAKLLKLKAQLGPDESKQKFVLKTPK | |
| | AERAALEELVKLQGERVRGLKQQKASAELIEEEVAKLLKLKAQLGPDE | |
| | SKQKFVLKTPK | |

Accordingly, in certain embodiments, the HRS polypeptide is fused or otherwise conjugated to an Fc region and comprises, consists, or consists essentially of an amino acid sequence in Table H8 (SEQ ID NO:156-171) or an active variant or fragment thereof. In some embodiments, the expressible polynucleotide encodes an HRS polypeptide that comprises consists, or consists essentially of an amino acid sequence in Table H8 (e.g., SEQ ID NO: 156-171) or an active variant or fragment thereof.

As noted above, certain embodiments employ variants, fragments, hybrids, and/or otherwise modified forms an Fc region described herein and known in the art. Included are variants having one or more amino acid substitutions, insertions, deletions, and/or truncations relative to a reference sequence, such as any one or more of the reference sequences of Table H7 or Table H8. Polypeptide and polynucleotide variants are described elsewhere herein.

Also included are hybrid Fc regions, for example, Fc regions that comprise a combination of Fc domains (e.g., hinge, CH₂, CH₃, CH₄) from immunoglobulins of different species, different Ig classes, and/or different Ig subclasses. General examples include hybrid Fc regions that comprise, consist of, or consist essentially of the following combination of CH₂/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgE/IgA1, IgE/IgA2, IgE/IgD, IgE/IgE, IgE/IgG1, IgE/IgG2, IgE/IgG3, IgE/IgG4, IgE/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM, IgM/IgA1, IgM/IgA2, IgM/IgD, IgM/IgE, IgM/IgG1, IgM/IgG2, IgM/IgG3, IgM/IgG4, IgM/IgM (or fragments or variants thereof), and optionally include a hinge from one or more of IgA1, IgA2, IgD, IgG1, IgG2, IgG3, or IgG4, and/or a CH₄ domain from IgE and/or IgM. In specific embodiments, the hinge, CH₂, CH₃, and CH₄ domains are from human Ig.

Additional examples include hybrid Fc regions that comprise, consist of, or consist essentially of the following combination of CH₂/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₃ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Certain examples include hybrid Fc regions that comprise, consist of, or consist essentially of the following combination of CH₃/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₂ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Particular examples include hybrid Fc regions that comprise, consist of, or consist essentially of the following combination of hinge/CH₂ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₃ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Certain examples include hybrid Fc regions that comprise, consist of, or consist essentially of the following combination of hinge/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₂ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Some examples include hybrid Fc regions that comprise, consist of, or consist essentially of the following combination of hinge/CH₄ domains: IgA1/IgE, IgA1/IgM, IgA2/IgE, IgA2/IgM, IgD/IgE, IgD/IgM, IgG1/IgE, IgG1/IgM, IgG2/IgE, IgG2/IgM, IgG3/IgE, IgG3/IgM, IgG4/IgE, IgG4/IgM (or fragments or variants thereof), and optionally include a CH₂ domain from one or more of IgA1, IgA2, IgD, IgE, IgG1, IgG2, IgG3, IgG4, or IgM, and/or a CH₃ domain from one or more of IgA1, IgA2, IgD, IgE, IgG1, IgG2, IgG3, IgG4, or IgM.

Specific examples of hybrid Fc regions can be found, for example, in WO 2008/147143, which are derived from combinations of IgG subclasses or combinations of human IgD and IgG.

Also included are derivatized or otherwise modified Fc regions. In certain aspects, the Fc region may be modified by phosphorylation, sulfation, acrylation, glycosylation, methylation, farnesylation, acetylation, amidation, and the like, for instance, relative to a wild-type or naturally-occurring Fc region. In certain embodiments, the Fc region may comprise wild-type or native glycosylation patterns, or alternatively, it may comprise increased glycosylation relative to a native form, decreased glycosylation relative to a native form, or it may be entirely deglycosylated. As one example of a modified Fc glycoform, decreased glycosylation of an Fc region reduces binding to the C1q region of the first complement component C1, a decrease in ADCC-related activity, and/or a decrease in CDC-related activity. Certain embodiments thus employ a deglycosylated or aglycosylated Fc region. See, e.g., WO 2005/047337 for the production of exemplary aglycosylated Fc regions. Another example of an Fc region glycoform can be generated by substituting the Q295 position with a cysteine residue (see, e.g., U.S. Application No. 2010/0080794), according to the Kabat et al. numbering system. Certain embodiments may include Fc regions where about 80-100% of the glycoprotein in Fc region comprises a mature core carbohydrate structure that lacks fructose (see, e.g., U.S. Application No. 2010/0255013). Some embodiments may include Fc regions that are optimized by substitution or deletion to reduce the level of fucosylation, for instance, to increase affinity for FcγRI, FcγRIa, or FcγRIIIa, and/or to improve phagocytosis by FcγRIIa-expressing cells (see U.S. Application Nos. 2010/0249382 and 2007/0148170).

As another example of a modified Fc glycoform, an Fc region may comprise oligomannose-type N-glycans, and optionally have one or more of the following: increased ADCC activity, increased binding affinity for FcγRIIIA (and certain other FcRs), similar or increased binding specificity for the target of the HRS polypeptide, similar or higher binding affinity for the target of the HRS polypeptide, and/or similar or lower binding affinity for mannose receptor, relative to a corresponding Fc region or HRS-Fc conjugate that contains complex-type N-glycans (see, e.g., U.S. Application No. 2007/0092521 and U.S. Pat. No. 7,700,321). As another example, enhanced affinity of Fc regions for FcγRs has been achieved using engineered glycoforms generated by expression of antibodies in engineered or variant cell lines (see, e.g., Umana et al., Nat Biotechnol. 17:176-180, 1999; Davies et al., Biotechnol Bioeng. 74:288-294, 2001; Shields et al., J Biol Chem. 277:26733-26740, 2002; Shinkawa et al., 2003, J Biol Chem. 278:3466-3473, 2003; and U.S. Application No. 2007/0111281). Certain Fc region glycoforms comprise an increased proportion of N-glycoside bond type complex sugar chains, which do not have the 1-position of fucose bound to the 6-position of N-acetylglucosamine at the reducing end of the sugar chain (see, e.g., U.S. Application No. 2010/0092997). Particular embodiments may include IgG Fc region that is glycosylated with at least one galactose moiety connected to a respective terminal sialic acid moiety by an α-2,6 linkage, optionally where the Fc region has a higher anti-inflammatory activity relative to a corresponding, wild-type Fc region (see U.S. Application No. 2008/0206246). Certain of these and related altered glycosylation approaches have generated substantial enhancements of the capacity of Fc regions to selectively bind FcRs such as FcγRIII, to mediate ADCC, and to alter other properties of Fc regions, as described herein.

Certain variant, fragment, hybrid, or otherwise modified Fc regions may have altered binding to one or more FcRs, relative to a corresponding, wild-type Fc sequence (e.g., same species, same Ig class, same Ig subclass). For instance, such Fc regions may have increased binding to one or more of Fcγ receptors, Fcα receptors, Fcε receptors, and/or the neonatal Fc receptor, relative to a corresponding, wild-type Fc sequence. In some embodiments, variant, fragment, hybrid, or modified Fc regions may have decreased binding to one or more of Fcγ receptors, Fcα receptors, Fcε receptors, and/or the neonatal Fc receptor, relative to a corresponding, wild-type Fc sequence. Specific FcRs are described elsewhere herein.

Specific examples of Fc variants having altered (e.g., increased, decreased) FcR binding can be found, for example, in U.S. Pat. Nos. 5,624,821 and 7,425,619; U.S. Application Nos. 2009/0017023, 2009/0010921, and 2010/0203046; and WO 2000/42072 and WO 2004/016750. Certain examples include human Fc regions having a one or more substitutions at position 298, 333, and/or 334, for example, S298A, E333A, and/or K334A (based on the numbering of the EU index of Kabat et al.), which have been shown to increase binding to the activating receptor FcγRIIIa and reduce binding to the inhibitory receptor FcγRIIb. These mutations can be combined to obtain double and triple mutation variants that have further improvements in binding to FcRs. Certain embodiments include a S298A/E333A/K334A triple mutant, which has increased binding to FcγRIIIa, decreased binding to FcγRIIb, and increased ADCC (see, e.g., Shields et al., J Biol Chem. 276:6591-6604, 2001; and Presta et al., Biochem Soc Trans. 30:487-490, 2002). See also engineered Fc glycoforms that have increased binding to FcRs, as disclosed in Umana et al., supra; and U.S. Pat. No. 7,662,925. Some embodiments include Fc regions that comprise one or more substitutions selected from 434S, 252Y/428L, 252Y/434S, and 428L/434S (see U.S. Application Nos. 2009/0163699 and 20060173170), based on the EU index of Kabat et al.

Certain variant, fragment, hybrid, or modified Fc regions may have altered effector functions, relative to a corresponding, wild-type Fc sequence. For example, such Fc regions may have increased complement fixation or activation, increased C1q binding affinity, increased CDC-related activity, increased ADCC-related activity, and/or increased ADCP-related activity, relative to a corresponding, wild-type Fc sequence. In some embodiments, such Fc regions may have decreased complement fixation or activation, decreased C1q binding affinity, decreased CDC-related activity, decreased ADCC-related activity, and/or decreased ADCP-related activity, relative to a corresponding, wild-type Fc sequence. As merely one illustrative example, an Fc region may comprise a deletion or substitution in a complement-binding site, such as a C1q-binding site, and/or a deletion or substitution in an ADCC site. Examples of such deletions/substitutions are described, for example, in U.S. Pat. No. 7,030,226. Many Fc effector functions, such as ADCC, can be assayed according to routine techniques in the art. (see, e.g., Zuckerman et al., CRC Crit Rev Microbiol. 7:1-26, 1978). Useful effector cells for such assays includes, but are not limited to, natural killer (NK) cells, macrophages, and other peripheral blood mononuclear cells (PBMC). Alternatively, or additionally, certain Fc effector functions may be assessed in vivo, for example, by employing an animal model described in Clynes et al. PNAS. 95:652-656, 1998.

Certain variant hybrid, or modified Fc regions may have altered stability or half-life relative to a corresponding, wild-type Fc sequence. In certain embodiments, such Fc regions may have increased half-life relative to a corresponding, wild-type Fc sequence. In some embodiments, variant hybrid, or modified Fc regions may have decreased half-life relative to a corresponding, wild-type Fc sequence. Half-life can be measured in vitro (e.g., under physiological conditions) or in vivo, according to routine techniques in the art, such as radiolabeling, ELISA, or other methods. In vivo measurements of stability or half-life can be measured in one or more bodily fluids, including blood, serum, plasma, urine, or cerebrospinal fluid, or a given tissue, such as the liver, kidneys, muscle, central nervous system tissues, bone, etc. As one example, modifications to an Fc region that alter its ability to bind the FcRn can alter its half-life in vivo. Assays for measuring the in vivo pharmacokinetic properties (e.g., in vivo mean elimination half-life) and non-limiting examples of Fc modifications that alter its binding to the FcRn are described, for example, in U.S. Pat. Nos. 7,217,797 and 7,732,570; and U.S. Application Nos. US 2010/0143254 and 2010/0143254.

Additional non-limiting examples of modifications to alter stability or half-life include substitutions/deletions at one or more of amino acid residues selected from 251-256, 285-290, and 308-314 in the CH₂ domain, and 385-389 and 428-436 in the CH₃ domain, according to the numbering system of Kabat et al. See U.S. Application No. 2003/0190311. Specific examples include substitution with leucine at position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, and/or substitution with histidine, tyrosine, arginine or threonine at position 436, including any combination thereof. Such modifications optionally increase affinity of the Fc region for the FcRn and thereby increase half-life, relative to a corresponding, wild-type Fc region.

Certain variant hybrid, or modified Fc regions may have altered solubility relative to a corresponding, wild-type Fc sequence. In certain embodiments, such Fc regions may have increased solubility relative to a corresponding, wild-type Fc sequence. In some embodiments, variant hybrid, or modified Fc regions may have decreased solubility relative to a corresponding, wild-type Fc sequence. Solubility can be measured, for example, in vitro (e.g., under physiological conditions) according to routine techniques in the art. Exemplary solubility measurements are described elsewhere herein.

Additional examples of variants include IgG Fc regions having conservative or non-conservative substitutions (as described elsewhere herein) at one or more of positions 250, 314, or 428 of the heavy chain, or in any combination thereof, such as at positions 250 and 428, or at positions 250 and 314, or at positions 314 and 428, or at positions 250, 314, and 428 (see, e.g., U.S. Application No. 2011/0183412). In specific embodiments, the residue at position 250 is substituted with glutamic acid or glutamine, and/or the residue at position 428 is substituted with leucine or phenylalanine. As another illustrative example of an IgG Fc variant, any one or more of the amino acid residues at positions 214 to 238, 297 to 299, 318 to 322, and/or 327 to 331 may be used as a suitable target for modification (e.g., conservative or non-conservative substitution, deletion). In particular embodiments, the IgG Fc variant CH₂ domain contains amino acid substitutions at positions 228, 234, 235, and/or 331 (e.g., human IgG4 with Ser228Pro and Leu235Ala mutations) to attenuate the effector functions of the Fc region (see U.S. Pat. No. 7,030,226). Here, the numbering of the residues in the heavy chain is that of the EU index (see Kabat et al., “Sequences of Proteins of Immunological Interest,” 5^{th} Ed., National Institutes of Health, Bethesda, Md. (1991)). Certain of these and related embodiments have altered (e.g., increased, decreased) FcRn binding and/or serum half-life, optionally without reduced effector functions such as ADCC or CDC-related activities.

Additional examples include variant Fc regions that comprise one or more amino acid substitutions at positions 279, 341, 343 or 373 of a wild-type Fc region, or any combination thereof (see, e.g., U.S. Application No. 2007/0224188). The wild-type amino acid residues at these positions for human IgG are valine (279), glycine (341), proline (343) and tyrosine (373). The substation(s) can be conservative or non-conservative, or can include non-naturally occurring amino acids or mimetics, as described herein. Alone or in combination with these substitutions, certain embodiments may also employ a variant Fc region that comprises at least 1, 2, 3, 4, 5, 6, 7, 8, 9, 10 or more amino acid substitutions selected from the following: 235G, 235R, 236F, 236R, 236Y, 237K, 237N, 237R, 238E, 238G, 238H, 238I, 238L, 238V, 238W, 238Y, 244L, 245R, 247A, 247D, 247E, 247F, 247M, 247N, 247Q, 247R, 247S, 247T, 247W, 247Y, 248F, 248P, 248Q, 248W, 249L, 249M, 249N, 249P, 249Y, 251H, 251I, 251W, 254D, 254E, 254F, 254G, 254H, 254I, 254K, 254L, 254M, 254N, 254P, 254Q, 254R, 254V, 254W, 254Y, 255K, 255N, 256H, 256I, 256K, 256L, 256V, 256W, 256Y, 257A, 257I, 257M, 257N, 257S, 258D, 260S, 262L, 264S, 265K, 265S, 267H, 267I, 267K, 268K, 269N, 269Q, 271T, 272H, 272K, 272L, 272R, 279A, 279D, 279F, 279G, 279H, 279I, 279K, 279L, 279M, 279N, 279Q, 279R, 279S, 279T, 279W, 279Y, 280T, 283F, 283G, 283H, 283I, 283K, 283L, 283M, 283P, 283R, 283T, 283W, 283Y, 285N, 286F, 288N, 288P, 292E, 292F, 292G, 292I, 292L, 293S, 293V, 301W, 304E, 307E, 307M, 312P, 315F, 315K, 315L, 315P, 315R, 316F, 316K, 317P, 317T, 318N, 318P, 318T, 332F, 332G, 332L, 332M, 332S, 332V, 332W, 339D, 339E, 339F, 339G, 339H, 339I, 339K, 339L, 339M, 339N, 339Q, 339R, 339S, 339W, 339Y, 341D, 341E, 341F, 341H, 341I, 341K, 341L, 341M, 341N, 341P, 341Q, 341R, 341S, 341T, 341V, 341W, 341Y, 343A, 343D, 343E, 343F, 343G, 343H, 343I, 343K, 343L, 343M, 343N, 343Q, 343R, 343S, 343T, 343V, 343W, 343Y, 373D, 373E, 373F, 373G, 373H, 373I, 373K, 373L, 373M, 373N, 373Q, 373R, 373S, 373T, 373V, 373W, 375R, 376E, 376F, 376G, 376H, 376I, 376L, 376M, 376N, 376P, 376Q, 376R, 376S, 376T, 376V, 376W, 376Y, 377G, 377K, 377P, 378N, 379N, 379Q, 379S, 379T, 380D, 380N, 380S, 380T, 382D, 382F, 382H, 382I, 382K, 382L, 382M, 382N, 382P, 382Q, 382R, 382S, 382T, 382V, 382W, 382Y, 385E, 385P, 386K, 423N, 424H, 424M, 424V, 426D, 426L, 427N, 429A, 429F, 429M, 430A, 430D, 430F, 430G, 430H, 430I, 430K, 430L, 430M, 430N, 430P, 430Q, 430R, 430S, 430T, 430V, 430W, 430Y, 431H, 431K, 431P, 432R, 432S, 438G, 438K, 438L, 438T, 438W, 439E, 439H, 439Q, 440D, 440E, 440F, 440G, 440H, 440I, 440K, 440L, 440M, 440Q, 440T, 440V or 442K. As above, the numbering of the residues in the heavy chain is that of the EU index (see Kabat et al., supra). Such variant Fc regions typically confer an altered effector function or altered serum half-life upon HRS polypeptide to which the variant Fc region is operably attached. Preferably the altered effector function is an increase in ADCC, a decrease in ADCC, an increase in CDC, a decrease in CDC, an increase in C1q binding affinity, a decrease in C1q binding affinity, an increase in FcR (preferably FcRn) binding affinity or a decrease in FcR (preferably FcRn) binding affinity as compared to a corresponding Fc region that lacks such amino acid substitution(s).

Additional examples include variant Fc regions that comprise an amino acid substitution at one or more of position(s) 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 293, 294, 295, 296, 297, 298, 299, 300, 302, 313, 317, 318, 320, 322, 323, 324, 325, 326, 327, 328, 329, 330, 331, 332, 333, 334, 335 336 and/or 428 (see, e.g., U.S. Pat. No. 7,662,925). In specific embodiments, the variant Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, 1332E, I332N, I332Q, T335D, T335R, and T335Y. In other specific embodiments, the variant Fc region comprises at least one amino acid substitution selected from the group consisting of: V264I, F243L/V264I, L328M, 1332E, L328M/I332E, V264I/I332E, S298A/I332E, S239E/I332E, S239Q/I332E, S239E, A330Y, I332D, L328I/I332E, L328Q/I332E, V264T, V240I, V266I, S239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, S324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, In more specific embodiments, the variant Fc region comprises a series of substitutions selected from the group consisting of: N297D/I332E, F241Y/F243Y/V262T/V264T/N297D/I332E, S239D/N297D/I332E, S239E/N297D/I332E, S239D/D265Y/N297D/I332E, S239D/D265H/N297D/I332E, V264E/N297D/I332E, Y296N/N297D/I332E, N297D/A330Y/I332E, S239D/D265V/N297D/I332E, S239D/D265I/N297D/I332E, and N297D/S298A/A330Y/I332E. In specific embodiments, the variant Fc region comprises an amino acid substitution at position 332 (using the numbering of the EU index, Kabat et al., supra). Examples of substitutions include 332A, 332D, 332E, 332F, 332G, 332H, 332K, 332L, 332M, 332N, 332P, 332Q, 332R, 332S, 332T, 332V, 332W and 332Y. The numbering of the residues in the Fc region is that of the EU index of Kabat et al. Among other properties described herein, such variant Fc regions may have increased affinity for an FcγR, increased stability, and/or increased solubility, relative to a corresponding, wild-type Fc region.

Further examples include variant Fc regions that comprise one or more of the following amino acid substitutions: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, optionally where the variant has altered recognition of an Fc ligand and/or altered effector function compared with a parent Fc polypeptide, and wherein the numbering of the residues is that of the EU index as in Kabat et al. Specific examples of these and related embodiments include variant Fc regions that comprise or consist of the following sets of substitutions: (1) N276D, R292Q, V305A, I377V, T394A, V412A and K439E; (2) P244L, K246E, D399G and K409R; (3) S304G, K320R, S324T, K326E and M358T; (4) F243S, P247L, D265V, V266A, S383N and T411I; (5) H224N, F243L, T393A and H433P; (6) V240A, S267G, G341E and E356G; (7) M252T, P291L, P352A, R355W, N390D, S408G, S426F and A431S; (8) P228L, T289A, L365Q, N389S and S440G; (9) F241L, V273A, K340Q and L441F; (10) F241L, T299A, I332T and M428T; (11) E269K, Y300H, Q342R, V422I and G446A; (12) T225A, R301c, S304G, D312N, N315D, L351S and N421S; (13) S254T, L306I, K326R and Q362L; (14) H224Y, P230S, V323A, E333D, K338R and S364C; (15) T335I, K414M and P445R; (16) T335I and K414M; (17) P247A, E258K, D280N, K288R, N297D, T299A, K322E, Q342R, S354A and L365P; (18) H268N, V279M, A339T, N361D and S426P; (19) C261Y, K290E, L306F, Q311R, E333G and Q438L; (20) E283G, N315K, E333G, R344Q, L365P and S442T; (21) Q347R, N361Y and K439R; (22) S239P, S254P, S267N, H285R, N315S, F372L, A378T, N390D, Y391C, F404S, E430K, L432P and K447E; and (23) E269G, Y278H, N325S and K370R, wherein the numbering of the residues is that of the EU index as in Kabat et al. (see, e.g., U.S. Application No. 2010/0184959).

Another specific example of an Fc variant comprises an Fc sequence of Table H7, wherein Xaa at position 1 is Ala or absent; Xaa at position 16 is Pro or Glu; Xaa at position 17 is Phe, Val, or Ala; Xaa at position 18 is Leu, Glu, or Ala; Xaa at position 80 is Asn or Ala; and/or Xaa at position 230 is Lys or is absent (see, e.g., U.S. Application No. 2007/0253966). Certain of these Fc regions, and related HRS-Fc conjugates, have increased half-life, reduced effector activity, and/or are significantly less immunogenic than wild-type Fc sequences.

Variant Fc regions can also have one or more mutated hinge regions, as described, for example, in U.S. Application No. 2003/0118592. For instance, one or more cysteines in a hinge region can be deleted or substituted with a different amino acid. The mutated hinge region can comprise no cysteine residues, or it can comprise 1, 2, or 3 fewer cysteine residues than a corresponding, wild-type hinge region. In some embodiments, an Fc region having a mutated hinge region of this type exhibits a reduced ability to dimerize, relative to a wild-type Ig hinge region.

As noted above, HRS-Fc conjugates such as HRS-Fc fusion proteins typically have altered (e.g., improved, increased, decreased) pharmacokinetic properties relative to corresponding HRS polypeptides. Examples of pharmacokinetic properties include stability or half-life, bioavailability (the fraction of a drug that is absorbed), tissue distribution, volume of distribution (apparent volume in which a drug is distributed immediately after it has been injected intravenously and equilibrated between plasma and the surrounding tissues), concentration (initial or steady-state concentration of drug in plasma), elimination rate constant (rate at which drugs are removed from the body), elimination rate (rate of infusion required to balance elimination), area under the curve (AUC or exposure; integral of the concentration-time curve, after a single dose or in steady state), clearance (volume of plasma cleared of the drug per unit time), Cₘₐₓ (peak plasma concentration of a drug after oral administration), tₘₐₓ (time to reach Cₘₐₓ), Cₘᵢₙ (lowest concentration that a drug reaches before the next dose is administered), and fluctuation (peak trough fluctuation within one dosing interval at steady state). In some aspects, these improved properties are achieved without significantly altering the secondary structure and/or reducing the non-canonical biological activity of the HRS polypeptide. Indeed, some HRS-Fc conjugates have increased non-canonical biological activity.

Hence, in some embodiments, the HRS-Fc conjugate or HRS-Fc fusion polypeptide has a plasma or sera pharmacokinetic AUC profile at least 2, 3, 4, 5, 6, 7, 8, 9, 10, 11, 12, 13, 14, 15, 16, 17, 18, 19, 20, 50, 100, 200, 300, 400, or 500-fold greater than a corresponding unmodified or differently modified HRS polypeptide when administered to a mammal under the same or comparable conditions. In certain embodiments, the HRS-Fc conjugate or HRS-Fc fusion polypeptide has a stability (e.g., as measured by half-life) which is at least 10%, 20%, 30%, 40%, 50%, 60%, 70%, 80%, 90%, 100%, 200%, 300%, 400%, or 500% greater than a corresponding unmodified or differently modified HRS polypeptide when compared under similar conditions at room temperature, for example, in PBS at pH 7.4 for about 1, 2, 3, 4, 5, 6, 7, 8, 9, 10, 11, 12, 13, 14 days, or 1, 2, 3, 4 weeks or so.

In particular embodiments, a HRS-Fc conjugate or HRS-Fc fusion polypeptide has a biological half life at pH 7.4, 25° C., e.g., a physiological pH, human body temperature (e.g., in vivo, in serum, in a given tissue, in a given species such as rat, mouse, monkey, or human), of about or at least about 30 minutes, about 1 hour, about 2 hour, about 3 hours, about 4 hours, about 5 hours, about 6 hours, about 12 hours, about 18 hours, about 20 hours, about 24 hours, about 30 hours, about 36 hours, about 40 hours, about 48 hours, about 50 hours, about 60 hours, about 70 hours, about 72 hours, about 80 hours, about 84 hours, about 90 hours, about 96 hours, about 120 hours, or about 144 hours or more or any intervening half-life.

In certain embodiments, the HRS-Fc conjugate or HRS-Fc fusion polypeptide has greater bioavailability after subcutaneous (SC) administration compared to a corresponding unmodified HRS-polypeptide. In certain embodiments, the HRS-Fc conjugate or HRS-Fc fusion polypeptide has at least about 20%, at least about 30%, at least about 40%, at least about 50%, at least about 60%, at least about 70%, at least about 80%, at least about 90%, or at least about 100%, or more bioavailability compared to the corresponding unmodified HRS polypeptide.

In certain embodiments, the HRS-Fc fusion polypeptide has substantially the same secondary structure as a corresponding unmodified or differently modified HRS polypeptide, as determined via UV circular dichroism analysis. In certain embodiments, the HRS-Fc fusion polypeptide has substantially the same activity of a corresponding unmodified or differently modified HRS polypeptide in an assay of anti-inflammatory activity. In some embodiments, the HRS-Fc fusion polypeptide has greater than 2, 3, 4, 5, 6, 7, 8, 9, 10, 11, 12, 13, 14, 15, 16, 17, 18, 19, or 20-fold the activity of a corresponding unmodified or differently modified HRS polypeptide in an assay of anti-inflammatory activity.

Examples of HRS fusion proteins that modulate the oligomerization of the HRS polypeptide include fusions of HRS to the cartilage oligomeric matrix protein (COMP). Thus, certain embodiments include fusion proteins comprising an HRS polypeptide described herein fused to COMP, for example, the pentamerization domain from COMP, which is composed of approximately residues 28-73 of COMP (see, for example, Prodeus et al., JCI Insight. 2017; 2(18):e94308; and Kim et al., Biochim Biophys Acta. 2009 May; 1793(5):772-80). Exemplary HRS-COMP fusion proteins are provided in Table H9 below.

TABLE H9
Exemplary HRS-COMP fusion proteins

| | | SEQ |
|---|---|---|
| | | ID |
| Name | Sequence | NO: |
| SPARC- | MRAWIFFLLCLAGRALAAERAALEELVKLQGERVR | 173 |
| HRS(2-60)- | GLKQQKASAELIEEEVAKLLKLKAQLGPDESKQKF | |
| COMP- | VLKTPKGGGGSGGGGSSDLGPQMLRELQETNAALQ | |
| MycHIS | DVRELLRQQVREITFLKNTVMECDACGGGGGSEQK | |
| | LISEEDLNMHTGHHHHHH | |
| HRS(2-60)- | AERAALEELVKLQGERVRGLKQQKASAELIEEEVA | 174 |
| COMP- | KLLKLKAQLGPDESKQKFVLKTPKGGGGSGGGGSS | |
| MycHIS | DLGPQMLRELQETNAALQDVRELLRQQVREITFLK | |
| | NTVMECDACGGGGGSEQKLISEEDLNMHTGHHHHHH | |
| HRS(2-60)- | ERAALEELVKLQGERVRGLKQQKASAELIEEEVAK | 175 |
| COMP | LLKLKAQLGPDESKQKFVLKTPKGGGGSGGGGSSD | |
| | LGPQMLRELQETNAALQDVRELLRQQVREITFLKN | |
| | TVMECDAC | |

Underlined text represents the signal peptide.

Accordingly, in certain embodiments, the HRS polypeptide is fused or otherwise conjugated to COMP or a COMP pentamerization domain and comprises, consists, or consists essentially of an amino acid sequence in Table H9 or an active variant or fragment thereof. In some embodiments, the expressible polynucleotide encodes an HRS polypeptide that comprises consists, or consists essentially of an amino acid sequence in Table H9 (e.g., SEQ ID NO: 173-175) or an active variant or fragment thereof.

In certain embodiments, a peptide linker sequence may be employed to separate the HRS polypeptide(s) and the Fc region(s) or PEG(s) or other fusion partner(s) by a distance sufficient to ensure that each polypeptide folds into its desired secondary and tertiary structures. Such a peptide linker sequence can be incorporated into the conjugate or fusion protein using standard techniques well known in the art.

Certain peptide linker sequences may be chosen based on the following exemplary factors: (1) their ability to adopt a flexible extended conformation; (2) their inability to adopt a secondary structure that could interact with functional epitopes on the first and second polypeptides; (3) their physiological stability; and (4) the lack of hydrophobic or charged residues that might react with the polypeptide functional epitopes, or other features. See, e.g., George and Heringa, J Protein Eng. 15:871-879, 2002.

The linker sequence may generally be from 1 to about 200 amino acids in length. Particular linkers can have an overall amino acid length of about 1-200 amino acids, 1-150 amino acids, 1-100 amino acids, 1-90 amino acids, 1-80 amino acids, 1-70 amino acids, 1-60 amino acids, 1-50 amino acids, 1-40 amino acids, 1-30 amino acids, 1-20 amino acids, 1-10 amino acids, 1-5 amino acids, 1-4 amino acids, 1-3 amino acids, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more amino acids.

A peptide linker may employ any one or more naturally-occurring amino acids, non-naturally occurring amino acid(s), amino acid analogs, and/or amino acid mimetics as described elsewhere herein and known in the art. Certain amino acid sequences which may be usefully employed as linkers include those disclosed in Maratea et al., Gene 40:39-46, 1985; Murphy et al., PNAS USA. 83:8258-8262, 1986; U.S. Pat. Nos. 4,935,233 and 4,751,180. Particular peptide linker sequences contain Gly, Ser, and/or Asn residues. Other near neutral amino acids, such as Thr and Ala may also be employed in the peptide linker sequence, if desired.

Certain exemplary linkers include Gly, Ser and/or Asn-containing linkers, as follows: [G]ₓ, [S]ₓ, [N]ₓ, [GS]ₓ, [GGS]ₓ, [GSS]ₓ, [GSGS]ₓ (SEQ ID NO:201), [GGSG]ₓ (SEQ ID NO: 202), [GGGS]ₓ (SEQ ID NO: 203), [GGGGS]ₓ (SEQ ID NO: 204), [GN]ₓ, [GGN]ₓ, [GNN]ₓ, [GNGN]ₓ (SEQ ID NO: 205), [GGNG]ₓ (SEQ ID NO: 206), [GGGN]ₓ (SEQ ID NO: 207), [GGGGN]ₓ (SEQ ID NO: 208) linkers, where ₓ is 1, 2, 3, 4, 5, 6, 7, 8, 9, 10, 11, 12, 13, 14, 15, 16, 17, 18, 19, or 20 or more. Other combinations of these and related amino acids will be apparent to persons skilled in the art.

Additional examples of linker peptides include, but are not limited to the following amino acid sequences: Gly-Gly-Gly-Gly-Ser-Gly-Gly-Gly-Gly-Ser-Gly-Gly-Gly-Gly-Ser-(SEQ ID NO: 209); Gly-Ser-Gly-Gly-Gly-Gly-Ser-Gly-Gly-Gly-Gly-Ser-Gly-Gly-Gly-Gly-Ser-Gly-Gly-Gly-Gly-Ser-(SEQ ID NO: 210); Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-Gly-Gly-Gly-Gly-Ser-(SEQ ID NO: 211); Asp-Ala-Ala-Ala-Lys-Glu-Ala-Ala-Ala-Lys-Asp-Ala-Ala-Ala-Arg-Glu-Ala-Ala-Ala-Arg-Asp-Ala-Ala-Ala-Lys-(SEQ ID NO: 212); and Asn-Val-Asp-His-Lys-Pro-Ser-Asn-Thr-Lys-Val-Asp-Lys-Arg-(SEQ ID NO: 213).

Further non-limiting examples of linker peptides include DGGGS (SEQ ID NO: 214); TGEKP (SEQ ID NO: 215) (see, e.g., Liu et al., PNAS. 94:5525-5530, 1997); GGRR (SEQ ID NO: 216) (Pomerantz et al. 1995); (GGGGS)ₙ (SEQ ID NO: 204) (Kim et al., PNAS. 93:1156-1160, 1996); EGKSSGSGSESKVD (SEQ ID NO: 217) (Chaudhary et al., PNAS. 87:1066-1070, 1990); KESGSVSSEQLAQFRSLD (SEQ ID NO: 218) (Bird et al., Science. 242:423-426, 1988), GGRRGGGS (SEQ ID NO: 219); LRQRDGERP (SEQ ID NO: 220); LRQKDGGGSERP (SEQ ID NO: 221); LRQKd(GGGS)₂ ERP (SEQ ID NO: 222). In specific embodiments, the linker sequence comprises a Gly3 linker sequence, which includes three glycine residues. In particular embodiments, flexible linkers can be rationally designed using a computer program capable of modeling both DNA-binding sites and the peptides themselves (Desjarlais & Berg, PNAS. 90:2256-2260, 1993; and PNAS. 91:11099-11103, 1994) or by phage display methods.

The peptide linkers may be physiologically stable or may include a releasable linker such as a physiologically degradable or enzymatically cleavable linker (e.g., proteolytically cleavable linker). In certain embodiments, one or more releasable linkers can result in a shorter half-life and more rapid clearance of the conjugate. These and related embodiments can be used, for example, to enhance the solubility and blood circulation lifetime of HRS polypeptides in the bloodstream, while also delivering a HRS polypeptide into the bloodstream that, subsequent to linker degradation, is substantially free of the Fc region(s). These aspects are especially useful in those cases where HRS polypeptides, when permanently conjugated to an Fc region, demonstrate reduced activity. By using the linkers as provided herein, such HRS polypeptides can maintain their therapeutic activity when in conjugated form. As another example, a large and relatively inert HRS-Fc conjugate polypeptide may be administered, which is then degraded in vivo (via the degradable linker) to generate a bioactive HRS polypeptide possessing a portion of the Fc region or lacking the Fc region entirely. In these and other ways, the properties of the HRS-Fc conjugate polypeptide can be more effectively tailored to balance the bioactivity and circulating half-life of the HRS polypeptide over time.

In particular embodiments, the linker peptide comprises an autocatalytic or self-cleaving peptide cleavage site. In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus. In certain embodiments, the self-cleaving polypeptide site comprises a 2A or 2A-like site, sequence or domain (Donnelly et al., J. Gen. Vivol. 82:1027-1041, 2001). Exemplary 2A sites include the following sequences: LLNFDLLKLAGDVESNPGP (SEQ ID NO: 223); TLNFDLLKLAGDVESNPGP (SEQ ID NO: 224); LLKLAGDVESNPGP (SEQ ID NO: 225); NFDLLKLAGDVESNPGP (SEQ ID NO: 226); QLLNFDLLKLAGDVESNPGP (SEQ ID NO: 227); APVKQTLNFDLLKLAGDVESNPGP (SEQ ID NO: 228); VTELLYRMKRAETYCPRPLLAIHPTEARHKQKIVAPVKQT (SEQ ID NO: 229); LNFDLLKLAGDVESNPGP (SEQ ID NO: 230); LLAIHPTEARHKQKIVAPVKQTLNFDLLKLAGDVESNPGP (SEQ ID NO: 231); and EARHKQKIVAPVKQTLNFDLLKLAGDVESNPGP (SEQ ID NO: 232). In some embodiments, the autocatalytic peptide cleavage site comprises a translational 2A signal sequence, such as, e.g., the 2A region of the aphthovirus foot-and-mouth disease virus (FMDV) polyprotein, which is an18 amino acid sequence. Additional examples of 2A-like sequences that may be used include insect virus polyproteins, the NS34 protein of type C rotaviruses, and repeated sequences in Trypanosoma spp., as described, for example, in Donnelly et al., Journal of General Virology. 82:1027-1041, 2001.

Suitable protease cleavages sites and self-cleaving peptides are known to the skilled person (see, e.g., Ryan et al., J Gener. Virol. 78:699-722, 1997; and Scymczak et al., Nature Biotech. 5:589-594, 2004). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are included in some embodiments, e.g., EXXYXQ(G/S) (SEQ ID NO: 233), for example, ENLYFQG (SEQ ID NO: 234) and ENLYFQS (SEQ ID NO: 235), wherein X represents any amino acid (cleavage by TEV occurs between Q and G or Q and S).

Further examples of enzymatically degradable linkers suitable for use in particular embodiments include, but are not limited to: an amino acid sequence cleaved by a serine protease such as thrombin, chymotrypsin, trypsin, elastase, kallikrein, or substilisin. Illustrative examples of thrombin-cleavable amino acid sequences include, but are not limited to: -Gly-Arg-Gly-Asp-(SEQ ID NO: 236), -Gly-Gly-Arg-, -Gly-Arg-Gly-Asp-Asn-Pro-(SEQ ID NO: 237), -Gly-Arg-Gly-Asp-Ser-(SEQ ID NO: 238), -Gly-Arg-Gly-Asp-Ser-Pro-Lys-(SEQ ID NO: 239), -Gly-Pro-Arg-, -Val-Pro-Arg-, and -Phe-Val-Arg-. Illustrative examples of elastase-cleavable amino acid sequences include, but are not limited to: -Ala-Ala-Ala-, -Ala-Ala-Pro-Val-(SEQ ID NO: 240), -Ala-Ala-Pro-Leu-(SEQ ID NO: 241), -Ala-Ala-Pro-Phe-(SEQ ID NO:242), -Ala-Ala-Pro-Ala-(SEQ ID NO: 243), and -Ala-Tyr-Leu-Val-(SEQ ID NO: 244).

Enzymatically degradable linkers also include amino acid sequences that can be cleaved by a matrix metalloproteinase such as collagenase, stromelysin, and gelatinase. Illustrative examples of matrix metalloproteinase-cleavable amino acid sequences include, but are not limited to: -Gly-Pro-Y-Gly-Pro-Z-(SEQ ID NO: 245), -Gly-Pro-, Leu-Gly-Pro-Z-(SEQ ID NO: 246), -Gly-Pro-Ile-Gly-Pro-Z-(SEQ ID NO: 247), and -Ala-Pro-Gly-Leu-Z-(SEQ ID NO: 248), where Y and Z are amino acids. Illustrative examples of collagenase-cleavable amino acid sequences include, but are not limited to: -Pro-Leu-Gly-Pro-D-Arg-Z-(SEQ ID NO: 249), -Pro-Leu-Gly-Leu-Leu-Gly-Z-(SEQ ID NO: 250), -Pro-Gln-Gly-Ile-Ala-Gly-Trp-(SEQ ID NO: 251), -Pro-Leu-Gly-Cys(Me)-His-(SEQ ID NO: 252), -Pro-Leu-Gly-Leu-Tyr-Ala-(SEQ ID NO: 253), -Pro-Leu-Ala-Leu-Trp-Ala-Arg-(SEQ ID NO: 254), and -Pro-Leu-Ala-Tyr-Trp-Ala-Arg-(SEQ ID NO: 255), where Z is an amino acid. An illustrative example of a stromelysin-cleavable amino acid sequence is -Pro-Tyr-Ala-Tyr-Tyr-Met-Arg-(SEQ ID NO: 256); and an example of a gelatinase-cleavable amino acid sequence is -Pro-Leu-Gly-Met-Tyr-Ser-Arg-(SEQ ID NO: 257).

Enzymatically degradable linkers suitable for use in particular embodiments include amino acid sequences that can be cleaved by an angiotensin converting enzyme, such as, for example, -Asp-Lys-Pro-, -Gly-Asp-Lys-Pro-(SEQ ID NO: 258), and -Gly-Ser-Asp-Lys-Pro-(SEQ ID NO: 259).

Enzymatically degradable linkers suitable for use in particular embodiments include amino acid sequences that can be degraded by cathepsin B, such as, for example, Val-Cit, Ala-Leu-Ala-Leu-(SEQ ID NO: 260), Gly-Phe-Leu-Gly-(SEQ ID NO: 261) and Phe-Lys.

In particular embodiments, a releasable linker has a half life at pH 7.4, 25° C., e.g., a physiological pH, human body temperature (e.g., in vivo, in serum, in a given tissue), of about 30 minutes, about 1 hour, about 2 hour, about 3 hours, about 4 hours, about 5 hours, about 6 hours, about 12 hours, about 18 hours, about 24 hours, about 36 hours, about 48 hours, about 72 hours, or about 96 hours or more or any intervening half-life. One having skill in the art would appreciate that the half life of a HRS-Fc conjugate polypeptide can be finely tailored by using a particular releasable linker.

In certain embodiments, however, any one or more of the peptide linkers are optional. For instance, linker sequences may not required when the first and second polypeptides have non-essential N-terminal and/or C-terminal amino acid regions that can be used to separate the functional domains and prevent steric interference.

The HRS polypeptides and polynucleotides, for example, expressible polynucleotides, can be used in any of the compositions, methods, and/or kits described herein.

### Neuropilin-2 Polypeptides and NRP2 Ligands

Embodiments of the present disclosure relate to the discovery that human histidyl-tRNA synthetase (HRS) polypeptides have unexpected biological properties which are relevant to treating a broad range of diseases and conditions, and that certain of these properties relate to the interactions between HRS and human neuropilin 2 (NRP2). Accordingly, HRS polypeptides can be used as standalone therapies in the treatment of diseases, for example, NRP2-associated disease, and/or in combination with other agents to address a spectrum of diseases and conditions associated with neuropilin-2 biology.

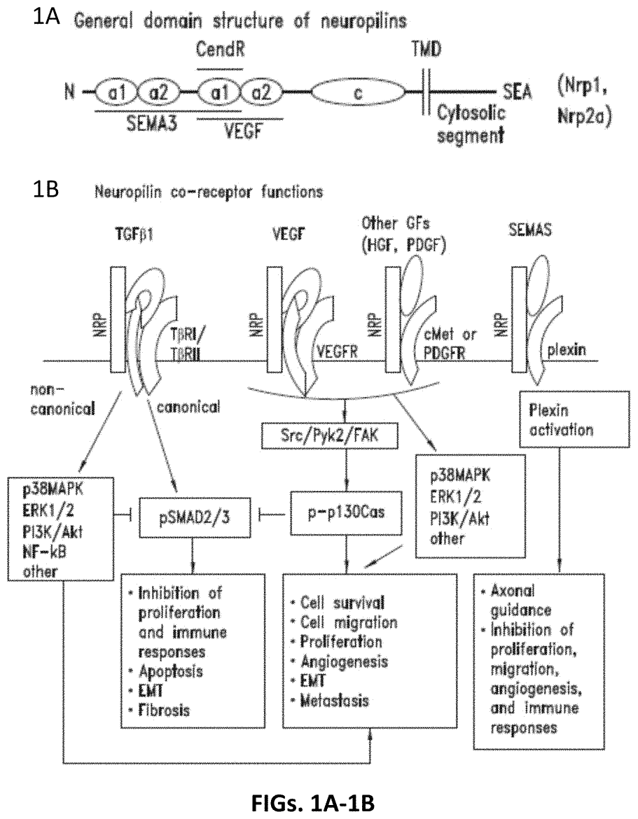

NRP2 is a single transmembrane receptor with a predominant extracellular region containing two CUB domains (al/a2 combined domain), two Factor V/VIII homology domains (b1/b2 combined domain), and a MAM domain (c domain) (see FIGS. 1A-1B). The ala2 combined domain interacts with the sema region of the semaphorins, and the b1 domain interacts with the semaphorin PSI and Ig-like domains. NRP2 has a higher affinity for SEMA3F and 3G; in contrast, SEMAs 3A, 3B and 3E preferentially interact with NRP1. Both NRP1 and NRP2 have similar affinity for SEMA 3C. The b1b2 combined domain interacts with several growth factors containing heparin-binding domains, including VEGF C & D, placenta growth factor (PIGF)-2, fibroblast growth factor (FGF), galectin, hepatocyte growth factor (HGF), platelet derived growth factor (PDGF), and transforming growth factor (TGF)-beta (see, e.g., Prud'homme et al., Oncotarget 3:921-939, 2012). NRP2 also interacts with various growth factor-specific receptors, and interactions with these receptors occur independently of binding to SEMAs. In this context, integrins and growth factor receptors like VEGF receptor, TGF-beta receptor, c-Met, EGFR, FGFR, and PDGFR have been shown to interact with NRPs and in general appear to increase the affinity of each ligand for its receptor and to modulate downstream signaling. The c domain (Mam) domain does not appear to be required for ligand binding, but appears essential for signaling.

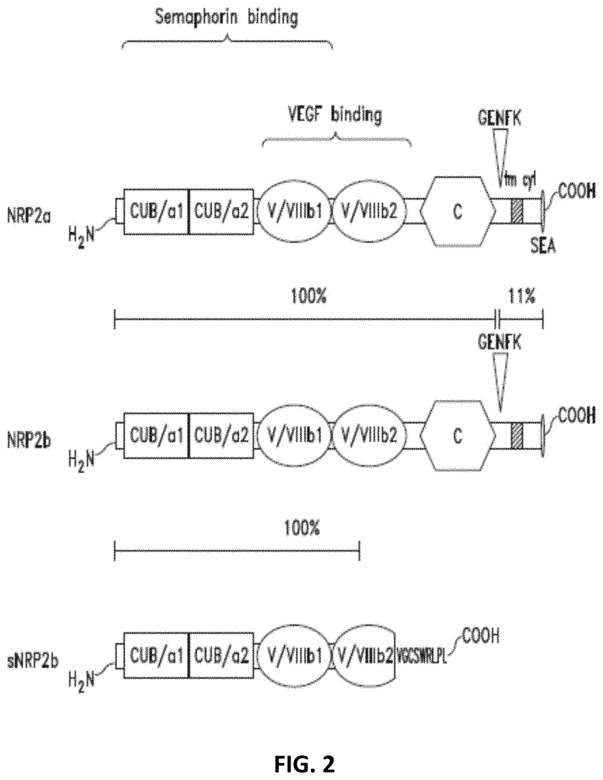

NRP2 can form homodimers as well as heterodimers with other cell surface receptors and co-receptors, and is heavily glycosylated. NRP2 also exists in the form of different splice variants which are between about 551 and 926 amino acids long. Two major variants for NRP2 are categorized as NRP2a and NRP2b. These differ in their intracellular C terminal part (see FIGS. 1A-1B) in which for NRP2a, the c-terminal domain comprises 42 amino acids and a PDZ-binding domain with the C-terminal SEA amino acid sequence. By contrast, NRP2b comprises a 46 amino acid C terminal domain which shares about 11% of the intracellular and transmembrane sequence of NRP2a. Between the MAM domain and the transmembrane domain, additional splicing can occur and 5 additional amino acids (GENFK) can be added to either the NRP2a, or NRP2b forms—these variants are named based on the number additional amino acids added through alternative splicing. Thus the two additional variants of NRP2 are named NRP2a(17) and NRP2a(22) and the two different transmembrane variants for NRP2b are named NRP2b(0) and NRP2b(5). Additionally, a soluble form called sNRP2b can be generated (see FIG. 2). The terms “Neuropilin-2”, or “NRP2” or “NRP2 polypeptides” refers to all isoforms, splice variants and naturally occurring fragments of NRP2, unless the context clearly points to a different specific meaning. Exemplary NRP2 polypeptide sequences are provided in Table N1 below.

TABLE N1
Exemplary Human NRP2 Polypeptides

| | | | SEQ ID |
|---|---|---|---|
| Name | Residues | Sequence | NO: |
| Human full | 1-931 | MDMFPLTWVFLALYFSRHQVRGQPDPPCGGRLNSKDAGYITS | 176 |
| length | | PGYPQDYPSHQNCEWIVYAPEPNQKIVLNFNPHFEIEKHDCK | |
| NRP2 | | YDFIEIRDGDSESADLLGKHCGNIAPPTIISSGSMLYIRFTS | |
| Variant 1 | | DYARQGAGFSLRYEIFKTGSEDCSKNFTSPNGTIESPGFPEK | |
| NRP2a(22) | | YPHNLDCTFTILAKPKMEIILQFLIFDLEHDPLQVGEGDCKY | |
| | | DWLDIWDGIPHVGPLIGKYCGTKTPSELRSSTGILSLTFHTD | |
| | | MAVAKDGFSARYYLVHQEPLENFQCNVPLGMESGRIANEQIS | |
| | | ASSTYSDGRWTPQQSRLHGDDNGWTPNLDSNKEYLQVDLRFL | |
| | | TMLTAIATQGAISRETQNGYYVKSYKLEVSTNGEDWMVYRHG | |
| | | KNHKVFQANNDATEWLNKLHAPLLTRFVRIRPQTWHSGIAL | |
| | | RLELFGCRVTDAPCSNMLGMLSGLIADSQISASSTQEYLWSP | |
| | | SAARLVSSRSGWFPRIPQAQPGEEWLQVDLGTPKTVKGVIIQ | |
| | | GARGGDSITAVEARAFVRKFKVSYSLNGKDWEYIQDPRTQQP | |
| | | KLFEGNMHYDTPDIRRFDPIPAQYVRVYPERWSPAGIGMRLE | |
| | | VLGCDWTDSKPTVETLGPTVKSEETTTPYPTEEEATECGENC | |
| | | SFEDDKDLQLPSGFNCNFDFLEEPCGWMYDHAKWLRTTWASS | |
| | | SSPNDRTFPDDRNFLRLQSDSQREGQYARLISPPVHLPRSPV | |
| | | CMEFQYQATGGRGVALQWREASQESKLLWVIREDQGGEWKH | |
| | | GRIILPSYDMEYQIVFEGVIGKGRSGEIAIDDIRISTDVPLE | |
| | | NCMEPISAFAGENFKVDIPEIHEREGYEDEIDDEYEVDWSNS | |
| | | SSATSGSGAPSTDKEKSWLYTLDPILITIIAMSSLGVLLGAT | |
| | | CAGLLLYCTCSYSGLSSRSCTTLENYNFELYDGLKHKVKMNH | |
| | | QKCCSEA | |
| Human NRP2 | 1-926 | MDMFPLTWVFLALYFSRHQVRGQPDPPCGGRLNSKDAGYITS | 177 |
| Variant 2 | | PGYPQDYPSHQNCEWIVYAPEPNQKIVLNFNPHFEIEKHDCK | |
| precursor | | YDFIEIRDGDSESADLLGKHCGNIAPPTIISSGSMLYIKFTS | |
| NRP2a(17) | | DYARQGAGFSLRYEIFKTGSEDCSKNFTSPNGTIESPGFPEK | |
| | | YPHNLDCTFTILAKPKMEIILQFLIFDLEHDPLQVGEGDCKY | |
| | | DWLDIWDGIPHVGPLIGKYCGTKTPSELRSSTGILSLTFHTD | |
| | | MAVAKDGFSARYYLVHQEPLENFQCNVPLGMESGRIANEQIS | |
| | | ASSTYSDGRWTPQQSRLHGDDNGWTPNLDSNKEYLQVDLRFL | |
| | | TMLTAIATQGAISRETQNGYYVKSYKLEVSTNGEDWMVYRHG | |
| | | KNHKVFQANNDATEWLNKLHAPLLTRFVRIRPQTWHSGIAL | |
| | | RLELFGCRVTDAPCSNMLGMLSGLIADSQISASSTQEYLWSP | |
| | | SAARLVSSRSGWFPRIPQAQPGEEWLQVDLGTPKTVKGVIIQ | |
| | | GARGGDSITAVEARAFVRKFKVSYSLNGKDWEYIQDPRTQQP | |
| | | KLFEGNMHYDTPDIRRFDPIPAQYVRVYPERWSPAGIGMRLE | |
| | | VLGCDWTDSKPTVETLGPTVKSEETTTPYPTEEEATECGENC | |
| | | SFEDDKDLQLPSGFNCNFDFLEEPCGWMYDHAKWLRTTWASS | |
| | | SSPNDRTFPDDRNFLRLQSDSQREGQYARLISPPVHLPRSPV | |
| | | CMEFQYQATGGRGVALQWREASQESKLLWVIREDQGGEWKH | |
| | | GRIILPSYDMEYQIVFEGVIGKGRSGEIAIDDIRISTDVPLE | |
| | | NCMEPISAFAVDIPEIHEREGYEDEIDDEYEVDWSNSSSATS | |
| | | GSGAPSTDKEKSWLYTLDPILITIIAMSSLGVLLGATCAGLL | |
| | | LYCTCSYSGLSSRSCTTLENYNFELYDGLKHKVKMNHQKCCS | |
| | | EA | |
| Human NRP2 | 1-909 | MDMFPLTWVFLALYFSRHQVRGQPDPPCGGRLNSKDAGYI | 178 |
| Variant 3 | | TSPGYPQDYPSHQNCEWIVYAPEPNQKIVLNFNPHFEIEK | |
| precursor | | HDGKYDFIEIRDGDSESADLLGKHCGNIAPPTIISSGSMLYI | |
| NRP2a(0) | | KFTSDYARQGAGFSLRYEIFKTGSEDCSKNFTSPNGTIESPG | |
| | | FPEKYPHNLDCTFTILAKPKMEIILQFLIFDLEHDPLQVGEG | |
| | | DCKYDWLDIWDGIPHVGPLIGKYCGTKTPSELRSSTGILSLT | |
| | | FHTDMAVAKDGFSARYYLVHQEPLENFQCNVPLGMESGRIAN | |
| | | EQISASSTYSDGRWTPQQSRLHGDDNGWTPNLDSNKEYLQVD | |
| | | LRFLTMLTAIATQGAISRETQNGYYVKSYKLEVSTNGEDWMV | |
| | | YRHGKNHKVFQANNDATEvvLNKLHAPLLTRFVRIRPQTWHS | |
| | | GIALRLELFGCRVTDAPCSNMLGMLSGLIADSQISASSTQEY | |
| | | LWSPSAARLVSSRSGWFPRIPQAQPGEEWLQVDLGTPKTVKG | |
| | | VIIQGARGGD | |
| | | SITAVEARAFVRKFKVSYSLNGKDWEYIQDPRTQQPKLFEGN | |
| | | MHYDTPDIRRFDPIPAQYVRVYPERWSPAGIGMRLEVLGCDW | |
| | | TDSKPTVETLGPTVKSEETTTPYPTEEEATECGENCSFEDDK | |
| | | DLQLPSGFNCNFDFLEEPCGWMYDHAKWLRTTWASSSSPNDR | |
| | | TFPDDRNFLRLQSDSQREGQYARLISPPVHLPRSPVCMEFQY | |
| | | QATGGRGVALQvvREASQESKLLWVIREDQGGEWKHGRIILP | |
| | | SYDMEYQIVFEGVIGKGRSGEIAIDDIRISTDVPLENCMEPI | |
| | | SAFADEYEVDWSNSSSATSGSGAPSTDKEKSWLYTLDPILIT | |
| | | IIAMSSLGVLLGATCAGLLLYCTCSYSGLSSRSCTTLENYNF | |
| | | ELYDGLKHKVKMNHQKCCSEA | |
| Human NRP2 | 1-906 | MDMFPLTWVFLALYFSRHQVRGQPDPPCGGRLNSKDAGYITS | 179 |
| Variant 4 | | PGYPQDYPSHQNCEWIVYAPEPNQKIVLNFNPHFEIEKHDCK | |
| precursor | | YDFIEIRDGDSESADLLGKHCGNIAPPTIISSGSMLYIKFTS | |
| NRP2b(5) | | DYARQGAGFSLRYEIFKTGSEDCSKNFTSPNGTIESPGFPEK | |
| | | YPHNLDCTFTILAKPKMEIILQFLIFDLEHDPLQVGEGDCKY | |
| | | DWLDIWDGIPHVGPLIGKYCGTKTPSELRSSTGILSLTFHTD | |
| | | MAVAKDGFSARYYLVHQEPLENFQCNVPLGMESGRIANEQIS | |
| | | ASSTYSDGRWTPQQSRLHGDDNGWTPNLDSNKEYLQVDLRFL | |
| | | TMLTAIATQGAISRETQNGYYVKSYKLEVSTNGEDWMVYRHG | |
| | | KNHKVFQANNDATEvvLNKLHAPLLTRFVRIRPQTWHSGIAL | |
| | | RLELFGCRVTDAPCSNMLGMLSGLIADSQISASSTQEYLWSP | |
| | | SAARLVSSRSGWFPRIPQAQPGEEWLQVDLGTPKTVKGVIIQ | |
| | | GARGGDSITAVEARAFVRKFKVSYSLNGKDWEYIQDPRTQQP | |
| | | KLFEGNMHYDTPDIRRFDPIPAQYVRVYPERWSPAGIGMRLE | |
| | | VLGCDWTDSKPTVETLGPTVKSEETTTPYPTEEEATECGENC | |
| | | SFEDDKDLQLPSGFNCNFDFLEEPCGWMYDHAKWLRTTWASS | |
| | | SSPNDRTFPDDRNFLRLQSDSQREGQYARLISPPVHLPRSPV | |
| | | CMEFQYQATGGRGVALQVVREASQESKLLWVIREDQGGEWKH | |
| | | GRIILPSYDMEYQIVFEGVIGKGRSGEIAIDDIRISTDVPLE | |
| | | NCMEPISAFAGENFKGGTLLPGTEPTVDTVPMQPIPAYWYYV | |
| | | MAAGGAVLVLVSVALALVLHYHRFRYAAKKTDHSITYKTSHY | |
| | | TNGAPLAVEPTLTIKLEQDRGSHC | |
| Human NRP2 | 1-901 | MDMFPLTWVFLALYFSRHQVRGQPDPPCGGRLNSKDAGYI | 180 |
| Variant 5 | | TSPGYPQDYPSHQNCEWIVYAPEPNQKIVLNFNPHFEIEK | |
| precursor | | HDCKYDFIEIRDGDSESADLLGKHCGNIAPPTIISSGSMLYI | |
| NRP2b(0) | | KFTSDYARQGAGFSLRYEIFKTGSEDCSKNFTSPNGTIESPG | |
| | | FPEKYPHNLDCTFTILAKPKMEIILQFLIFDLEHDPLQVGEG | |
| | | DCKYDWLDIWDGIPHVGPLIGKYCGTKTPSELRSSTGILSLT | |
| | | FHTDMAVAKDGFSARYYLVHQEPLENFQCNVPLGMESGRIAN | |
| | | EQISASSTYSDGRWTPQQSRLHGDDNGWTPNLDSNKEYLQVD | |
| | | LRFLTMLTAIATQGAISRETQNGYYVKSYKLEVSTNGEDWMV | |
| | | YRHGKNHKVFQANNDATEVVLNKLHAPLLTRFVRIRPQTWHS | |
| | | GIALRLELFGCRVTDAPCSNMLGMLSGLIADSQISASSTQEY | |
| | | LWSPSAARLVSSRSGWFPRIPQAQPGEEWLQVDLGTPKTVKG | |
| | | VIIQGARGGD | |
| | | SITAVEARAFVRKFKVSYSLNGKDWEYIQDPRTQQPKLFEGN | |
| | | MHYDTPDIRRFDPIPAQYVRVYPERWSPAGIGMRLEVLGCDW | |
| | | TDSKPTVETLGPTVKSEETTTPYPTEEEATECGENCSFEDDK | |
| | | DLQLPSGFNCNFDFLEEPCGWMYDHAKWLRTTWASSSSPNDR | |
| | | TFPDDRNFLRLQSDSQREGQYARLISPPVHLPRSPVCMEFQY | |
| | | QATGGRGVALQvvREASQESKLLWVIREDQGGEWKHGRIILP | |
| | | SYDMEYQIVFEGVIGKGRSGEIAIDDIRISTDVPLENCMEPI | |
| | | SAFAGGTLLPGTEPTVDTVPMQPIPAYWYYVMAAGGAVLVLV | |
| | | SVALALVLHYHRFRYAAKKTDHSITYKTSHYTNGAPLAVEPT | |
| | | LTIKLEQDRGSHC | |
| Human NRP2 | 1-555 | MDMFPLTWVFLALYFSRHQVRGQPDPPCGGRLNSKDAGYITS | 181 |
| Variant 6 | | PGYPQDYPSHQNCEWIVYAPEPNQKIVLNFNPHFEIEKHDCK | |
| precursor | | YDFIEIRDGDSESADLLGKHCGNIAPPTIISSGSMLYIKFTS | |
| S9NRP2b | | DYARQGAGFSLRYEIFKTGSEDCSKNFTSPNGTIESPGFPEK | |
| Soluble | | YPHNLDCTFTILAKPKMEIILQFLIFDLEHDPLQVGEGDCKY | |
| NRP2 | | DWLDIWDGIPHVGPLIGKYCGTKTPSELRSSTGILSLTFHTD | |
| | | MAVAKDGFSARYYLVHQEPLENFQCNVPLGMESGRIANEQIS | |
| | | ASSTYSDGRWTPQQSRLHGDDNGWTPNLDSNKEYLQVDLRFL | |
| | | TMLTAIATQGAISRETQNGYYVKSYKLEVSTNGEDWMVYRHG | |
| | | KNHKVFQANNDATEvvLNKLHAPLLTRFVRIRPQTWHSGIAL | |
| | | RLELFGCRVTDAPCSNMLGMLSGLIADSQISASSTQEYLWSP | |
| | | SAARLVSSRSGWFPRIPQAQPGEEWLQVDLGTPKTVKGVIIQ | |
| | | GARGGDSITAVEARAFVRKFKVSYSLNGKDWEYIQDPRTQQP | |
| | | KVGCSWRPL | |
| Human NRP2 | 23-926 | QPDPPCGGRLNSKDAGYITSPGYPQDYPSHQNCEWIVYAPEP | 182 |
| Variant 2 | | NQKIVLNFNPHFEIEKHDCKYDFIEIRDGDSESADLLGKHCG | |
| NRP2a(17) | | NIAPPTIISSGSMLYIKFTSDYARQGAGFSLRYEIFKTGSED | |
| | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| | | FQCNVPLGMESGRIANEQISASSTYSDGRWTPQQSRLHGDDN | |
| | | GWTPNLDSNKEYLQVDLRFLTMLTAIATQGAISRETQNGYYV | |
| | | KSYKLEVSTNGEDWMVYRHGKNHKVFQANNDATEvvLNKLHA | |
| | | PLLTRFVRIRPQTWHSGIALRLELFGCRVTDAPCSNMLGMLS | |
| | | GLIADSQISASSTQEYLWSPSAARLVSSRSGWFPRIPQAQPG | |
| | | EEWLQVDLGTPKTVKGVIIQGARGGDSITAVEARAFVRKFKV | |
| | | SYSLNGKDWEYIQDPRTQQPKLFEGNMHYDTPDIRRFDPIPA | |
| | | QYVRVYPERWSPAGIGMRLEVLGCDWTDSKPTVETLGPTVKS | |
| | | eetttpypteeeatecgencsfeddkdlqlpsgfncnfdfle | |
| | | EPCGWMYDHAKWLRTTWASSSSPNDRTFPDDRNFLRLQSDSQ | |
| | | REGQYARLISPPVHLPRSPVCMEFQYQATGGRGVALQVVREA | |
| | | SQESKLLWVIREDQGGEWKHGRIILPSYDMEYQIVFEGVIGK | |
| | | GRSGEIAIDDIRISTDVPLENCMEPISAFAVDIPEIHEREGY | |
| | | EDEIDDEYEVDWSNSSSATSGSGAPSTDKEKSWLYTLDPILI | |
| | | TIIAMSSLGVLLGATCAGLLLYCTCSYSGLSSRSCTTLENYN | |
| | | FELYDGLKHKVKMNHQKCCSEA | |
| NRP2 | 23-901 | QPDPPCGGRLNSKDAGYITSPGYPQDYPSHQNCEWIVYAPEP | 183 |
| splice | | NQKIVLNFNPHFEIEKHDCKYDFIEIRDGDSESADLLGKHCG | |
| variant 5 | | NIAPPTIISSGSMLYIKFTSDYARQGAGFSLRYEIFKTGSED | |
| NRP2b(0) | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| | | FQCNVPLGMESGRIANEQISASSTYSDGRWTPQQSRLHGDDN | |
| | | GWTPNLDSNKEYLQVDLRFLTMLTAIATQGAISRETQNGYYV | |
| | | KSYKLEVSTNGEDWMVYRHGKNHKVFQANNDATEVVLNKLHA | |
| | | PLLTRFVRIRPQTWHSGIALRLELFGCRVTDAPCSNMLGMLS | |
| | | GLIADSQISASSTQEYLWSPSAARLVSSRSGWFPRIPQAQPG | |
| | | EEWLQVDLGTPKTVKGVIIQGARGGDSITAVEARAFVRKFKV | |
| | | SYSLNGKDWEYIQDPRTQQPKLFEGNMHYDTPDIRRFDPIPA | |
| | | QYVRVYPERWSPAGIGMRLEVLGCDWTDSKPTVETLGPTVKS | |
| | | eetttpypteeeatecgencsfeddkdlqlpsgfncnfdfle | |
| | | EPCGWMYDHAKWLRTTWASSSSPNDRTFPDDRNFLRLQSDSQ | |
| | | REGQYARLISPPVHLPRSPVCMEFQYQATGGRGVALQVVREA | |
| | | SQESKLLWVIREDQGGEWKHGRIILPSYDMEYQIVFEGVIGK | |
| | | GRSGEIAIDDIRISTDVPLENCMEPISAFAGGTLLPGTEPTV | |
| | | DTVPMQPIPAYWYYVMAAGGAVLVLVSVALALVLHYHRFRYA | |
| | | AKKTDHSITYKTSHYTNGAPLAVEPTLTIKLEQDRGSHC | |
| Soluble | 23-555 | QPDPPCGGRLNSKDAGYITSPGYPQDYPSHQNCEWIVYAPEP | 184 |
| NRP2 | | NQKIVLNFNPHFEIEKHDCKYDFIEIRDGDSESADLLGKHCG | |
| S9Nrp-2b | | NIAPPTIISSGSMLYIKFTSDYARQGAGFSLRYEIFKTGSED | |
| | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| | | FQCNVPLGMESGRIANEQISASSTYSDGRWTPQQSRLHGDDN | |
| | | GWTPNLDSNKEYLQVDLRFLTMLTAIATQGAISRETQNGYYV | |
| | | KSYKLEVSTNGEDWMVYRHGKNHKVFQANNDATEvvLNKLHA | |
| | | PLLTRFVRIRPQTWHSGIALRLELFGCRVTDAPCSNMLGMLS | |
| | | GLIADSQISASSTQEYLWSPSAARLVSSRSGWFPRIPQAQPG | |
| | | EEWLQVDLGTPKTVKGVIIQGARGGDSITAVEARAFVRKFKV | |
| | | SYSLNGKDWEYIQDPRTQQPKVGCSWRPL | |
| NRP2 Al | 28-141 | CGGRLNSKDAGYITSPGYPQDYPSHQNCEWIVYAPEPNQKIV | 185 |
| domain | | LNFNPHFEIEKHDCKYDFIEIRDGDSESADLLGKHCGNIAPP | |
| | | TIISSGSMLYIKFTSDYARQGAGFSLRYEI | |
| NRP2 A2 | 149-265 | CSKNFTSPNGTIESPGFPEKYPHNLDCTFTILAKPKMEIILQ | 186 |
| domain | | FLIFDLEHDPLQVGEGDCKYDWLDIWDGIPHVGPLIGKYCGT | |
| | | KTPSELRSSTGILSLTFHTDMAVAKDGFSARYY | |
| NRP2 Bl | 280-426 | PLGMESGRIANEQISASSTYSDGRWTPQQSRLHGDDNGWTPN | 187 |
| domain | | LDSNKEYLQVDLRFLTMLTAIATQGAISRETQNGYYVKSYKL | |
| | | EVSTNGEDWMVYRHGKNHKVFQANNDATEVVLNKLHAPLLTR | |
| | | FVRIRPQTWHSGIALRLELFG | |
| NRP2 B2 | 438-591 | LGMLSGLIADSQISASSTQEYLWSPSAARLVSSRSGWFPRIP | 188 |
| domain | | QAQPGEEWLQVDLGTPKTVKGVIIQGARGGDSITAVEARAFV | |
| | | RKFKVSYSLNGKDWEYIQDPRTQQPKLFEGNMHYDTPDIRRF | |
| | | DPIPAQYVRVYPERWSPAGIGMRLEVLG | |
| NRP2 C | 641-794 | PSGFNCNFDFLEEPCGWMYDHAKWLRTTWASSSSPNDRTFPD | 189 |
| domain | | DRNFLRLQSDSQREGQYARLISPPVHLPRSPVCMEFQYQATG | |
| | | GRGVALQVVREASQESKLLWVIREDQGGEWKHGRIILPSYDM | |
| | | EYQIVFEGVIGKGRSGEIAIDDIRISTD | |
| NRP2 | 23-265 | QPDPPCGGRLNSKDAGYITSPGYPQDYPSHQNCEWIVYAPEP | 190 |
| combined | | NQKIVLNFNPHFEIEKHDCKYDFIEIRDGDSESADLLGKHCG | |
| A1A2 | | NIAPPTIISSGSMLYIKFTSDYARQGAGFSLRYEIFKTGSED | |
| domains | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYY | |
| NRP2 | 149-426 | CSKNFTSPNGTIESPGFPEKYPHNLDCTFTILAKPKMEIILQ | 191 |
| combined | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| A2B1 | | FQCNVPLGMESGRIANEQISASSTYSDGRWTPQQSRLHGDDN | |
| domains | | GWTPNLDSNKEYLQVDLRFLTMLTAIATQGAISRETQNGYYV | |
| | | KSYKLEVSTNGEDWMVYRHGKNHKVFQANNDATEVVLNKLHA | |
| | | PLLTRFVRIRPQTWHSGIALRLELFG | |
| NRP2 | 23-426 | QPDPPCGGRLNSKDAGYITSPGYPQDYPSHQNCEWIVYAPEP | 192 |
| combined | | NQKIVLNFNPHFEIEKHDCKYDFIEIRDGDSESADLLGKHCG | |
| A1A2B1 | | NIAPPTIISSGSMLYIKFTSDYARQGAGFSLRYEIFKTGSED | |
| domains | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| | | FQCNVPLGMESGRIANEQISASSTYSDGRWTPQQSRLHGDDN | |
| | | GWTPNLDSNKEYLQVDLRFLTMLTAIATQGAISRETQNGYYV | |
| | | KSYKLEVSTNGEDWMVYRHGKNHKVFQANNDATEVVLNKLHA | |
| | | PLLTRFVRIRPQTWHSGIALRLELFG | |
| NRP2 | 23-595 | QPDPPCGGRLNSKDAGYITSPGYPQDYPSHQNCEWIVYAPEP | 193 |
| combined | | NQKIVLNFNPHFEIEKHDCKYDFIEIRDGDSESADLLGKHCG | |
| A1A2B1B2 | | NIAPPTIISSGSMLYIKFTSDYARQGAGFSLRYEIFKTGSED | |
| domains | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| | | FQCNVPLGMESGRIANEQISASSTYSDGRWTPQQSRLHGDDN | |
| | | GWTPNLDSNKEYLQVDLRFLTMLTAIATQGAISRETQNGYYV | |
| | | KSYKLEVSTNGEDWMVYRHGKNHKVFQANNDATEvvLNKLHA | |
| | | PLLTRFVRIRPQTWHSGIALRLELFGCRVTDAPCSNMLGMLS | |
| | | GLIADSQISASSTQEYLWSPSAARLVSSRSGWFPRIPQAQPG | |
| | | EEWLQVDLGTPKTVKGVIIQGARGGDSITAVEARAFVRKFKV | |
| | | SYSLNGKDWEYIQDPRTQQPKLFEGNMHYDTPDIRRFDPIPA | |
| | | QYVRVYPERWSPAGIGMRLEVLGCDWT | |
| NRP2 | 145-595 | GSEDCSKNFTSPNGTIESPGFPEKYPHNLDCTFTILAKPKME | 194 |
| combined | | IILQFLIFDLEHDPLQVGEGDCKYDWLDIWDGIPHVGPLIGK | |
| A2B1B2 | | YCGTKTPSELRSSTGILSLTFHTDMAVAKDGFSARYYLVHQE | |
| domains | | PLENFQCNVPLGMESGRIANEQISASSTYSDGRWTPQQSRLH | |
| | | GDDNGWTPNLDSNKEYLQVDLRFLTMLTAIATQGAISRETQN | |
| | | GYYVKSYKLEVSTNGEDWMVYRHGKNHKVFQANNDATEvvLN | |
| | | KLHAPLLTRFVRIRPQTWHSGIALRLELFGCRVTDAPCSNML | |
| | | GMLSGLIADSQISASSTQEYLWSPSAARLVSSRSGWFPRIPQ | |
| | | AQPGEEWLQVDLGTPKTVKGVIIQGARGGDSITAVEARAFVR | |
| | | KFKVSYSLNGKDWEYIQDPRTQQPKLFEGNMHYDTPDIRRFD | |
| | | PIPAQYVRVYPERWSPAGIGMRLEVLGCDWT | |
| NRP2 | 276-595 | QCNVPLGMESGRIANEQISASSTYSDGRWTPQQSRLHGDDNG | 195 |
| combined | | WTPNLDSNKEYLQVDLRFLTMLTAIATQGAISRETQNGYYVK | |
| B1B2 | | SYKLEVSTNGEDWMVYRHGKNHKVFQANNDATEVVLNKLHAP | |
| domains | | LLTRFVRIRPQTWHSGIALRLELFGCRVTDAPCSNMLGMLSG | |
| | | LIADSQISASSTQEYLWSPSAARLVSSRSGWFPRIPQAQPGE | |
| | | EWLQVDLGTPKTVKGVIIQGARGGDSITAVEARAFVRKFKVS | |
| | | YSLNGKDWEYIQDPRTQQPKLFEGNMHYDTPDIRRFDPIPAQ | |
| | | YVRVYPERWSPAGIGMRLEVLGCDWT | |
| NRP2 v2- | 23-855 | QPDPPCGGRLNSKDAGYITSPGYPQDYPSHQNCEWIVYAPEP | 196 |
| Fc fusion | | NQKIVLNFNPHFEIEKHDCKYDFIEIRDGDSESADLLGKHCG | |
| protein | | NIAPPTIISSGSMLYIKFTSDYARQGAGFSLRYEIFKTGSED | |
| | | CSKNFTSPNGTIESPGFPEKYPHNLDCTFTILAKPKMEIILQ | |
| | | FLIFDLEHDPLQVGEGDCKYDWLDIWDGIPHVGPLIGKYCGT | |
| | | KTPSELRSSTGILSLTFHTDMAVAKDGFSARYYLVHQEPLEN | |
| | | FQCNVPLGMESGRIANEQISASSTYSDGRWTPQQSRLHGDDN | |
| | | GWTPNLDSNKEYLQVDLRFLTMLTAIATQGAISRETQNGYYV | |
| | | KSYKLEVSTNGEDWMVYRHGKNHKVFQANNDATEVVLNKLHA | |
| | | PLLTRFVRIRPQTWHSGIALRLELFGCRVTDAPCSNMLGMLS | |
| | | GLIADSQISASSTQEYLWSPSAARLVSSRSGWFPRIPQAQPG | |
| | | EEWLQVDLGTPKTVKGVIIQGARGGDSITAVEARAFVRKFKV | |
| | | SYSLNGKDWEYIQDPRTQQPKLFEGNMHYDTPDIRRFDPIPA | |
| | | QYVRVYPERWSPAGIGMRLEVLGCDWTDSKPTVETLGPTVKS | |
| | | EETTTPYPTEEEATECGENCSFEDDKDLQLPSGFNCNFDFLE | |
| | | EPCGWMYDHAKWLRTTWASSSSPNDRTFPDDRNFLRLQSDSQ | |
| | | REGQYARLISPPVHLPRSPVCMEFQYQATGGRGVALQVVREA | |
| | | SQESKLLWVIREDQGGEWKHGRIILPSYDMEYQIVFEGVIGK | |
| | | GRSGEIAIDDIRISTDVPLENCMEPISAFAVDIPEIHEREGY | |
| | | EDEIDDEYEVDWSNSSSATSGSGAPSTDKEKSWLYDKTHTCP | |
| | | PCPAPELLGGPSVFLFPPKPKDTLMISRTPEVTCVVVDVSHE | |
| | | DPEVKFNWYVDGVEVHNAKTKPREEQYNSTYRVVSVLTVLHQ | |
| | | DWLNGKEYKCKVSNKALPAPIEKTISKAKGQPREPQVYTLPP | |
| | | SRDELTKNQVSLTCLVKGFYPSDIAVEWESNGQPENNYKTTP | |
| | | PVLDSDGSFFLYSKLTVDKSRWQQGNVFSCSVMHEALHNHYT | |
| | | QKSLSLSPGK | |
| NRP2 | 145-595 | GSEDCSKNFTSPNGTIESPGFPEKYPHNLDCTFTILAKPKME | 197 |
| A2B1B2-FC | | IILQFLIFDLEHDPLQVGEGDCKYDWLDIWDGIPHVGPLIGK | |
| | | YCGTKTPSELRSSTGILSLTFHTDMAVAKDGFSARYYLVHQE | |
| | | PLENFQCNVPLGMESGRIANEQISASSTYSDGRWTPQQSRLH | |
| | | GDDNGWTPNLDSNKEYLQVDLRFLTMLTAIATQGAISRETQN | |
| | | GYYVKSYKLEVSTNGEDWMVYRHGKNHKVFQANNDATEVVLN | |
| | | KLHAPLLTRFVRIRPQTWHSGIALRLELFGCRVTDAPCSNML | |
| | | GMLSGLIADSQISASSTQEYLWSPSAARLVSSRSGWFPRIPQ | |
| | | AQPGEEWLQVDLGTPKTVKGVIIQGARGGDSITAVEARAFVR | |
| | | KFKVSYSLNGKDWEYIQDPRTQQPKLFEGNMHYDTPDIRRFD | |
| | | PIPAQYVRVYPERWSPAGIGMRLEVLGCDWTDKTHTCPPCPA | |
| | | PELLGGPSVFLFPPKPKDTLMISRTPEVTCVVVDVSHEDPEV | |
| | | KFNWYVDGVEVHNAKTKPREEQYNSTYRVVSVLTVLHQDWLN | |
| | | GKEYKCKVSNKALPAPIEKTISKAKGQPREPQVYTLPPSRDE | |
| | | LTKNQVSLTCLVKGFYPSDIAVEWESNGQPENNYKTTPPVLD | |
| | | SDGSFFLYSKLTVDKSRWQQGNVFSCSVMHEALHNHYTQKSL | |
| | | SLSPGK | |
| NRP2 | 438-794 | LGMLSGLIADSQISASSTQEYLWSPSAARLVSSRSGWFPRIP | 198 |
| combined | | QAQPGEEWLQVDLGTPKTVKGVIIQGARGGDSITAVEARAFV | |
| B2C | | RKFKVSYSLNGKDWEYIQDPRTQQPKLFEGNMHYDTPDIRRF | |
| domains | | DPIPAQYVRVYPERWSPAGIGMRLEVLGCDWTDSKPTVETLG | |
| | | PTVKSEETTTPYPTEEEATECGENCSFEDDKDLQLPSGFNCN | |
| | | FDFLEEPCGWMYDHAKWLRTTWASSSSPNDRTFPDDRNFLRL | |
| | | QSDSQREGQYARLISPPVHLPRSPVCMEFQYQATGGRGVALQ | |
| | | VVREASQESKLLWVIREDQGGEWKHGRIILPSYDMEYQIVFE | |
| | | GVIGKGRSGEIAIDDIRISTD | |
| NRP2 | 276-794 | QCNVPLGMESGRIANEQISASSTYSDGRWTPQQSRLHGDDNG | 199 |
| combined | | WTPNLDSNKEYLQVDLRFLTMLTAIATQGAISRETQNGYYVK | |
| B1B2C | | SYKLEVSTNGEDWMVYRHGKNHKVFQANNDATEVVLNKLHAP | |
| domains | | LLTRFVRIRPQTWHSGIALRLELFGCRVTDAPCSNMLGMLSG | |
| | | LIADSQISASSTQEYLWSPSAARLVSSRSGWFPRIPQAQPGE | |
| | | EWLQVDLGTPKTVKGVIIQGARGGDSITAVEARAFVRKFKVS | |
| | | YSLNGKDWEYIQDPRTQQPKLFEGNMHYDTPDIRRFDPIPAQ | |
| | | YVRVYPERWSPAGIGMRLEVLGCDWTDSKPTVETLGPTVKSE | |
| | | etttpypteeeatecgencsfeddkdlqlpsgfncnfdflee | |
| | | PCGWMYDHAKWLRTTWASSSSPNDRTFPDDRNFLRLQSDSQR | |
| | | EGQYARLISPPVHLPRSPVCMEFQYQATGGRGVALQVVREAS | |
| | | QESKLLWVIREDQGGEWKHGRIILPSYDMEYQIVFEGVIGKG | |
| | | RSGEIAIDDIRISTD | |

Neuropilin-2 can respond to multiple ligands through the recruitment of diverse co-receptors to modulate a broad range of cellular functions through its roles both as an essential cell surface receptor, and co-receptor for a variety of ligands (see, e.g., Guo and Vander Kooi, J. Cell. Biol. 290 No 49: 29120-29126. 2015; Prud'homme et al., Oncotarget 3:921-939, 2012). For instance, NRP2 functions during epithelial to mesenchymal transition (EMT), for example, by promoting TGF-β1-mediated EMT in colorectal and other cancer cells (see, e.g., Grandclement et al., PLoS ONE 6(7) e20444, 2011) as well as mediating EMT, or endo-EMT in fibroblasts, myofibroblasts and endothelial cells to promote fibrosis formation (See, e.g., Pardali et al., Int. J. Mol. Sci. 18 2157 2017).

Neuropilin-2 expression also promotes lymphangiogenesis, and modulates vascular permeability (see, e.g., Doci et al., Cancer Res. 75(14) 2937-2948, 2015; Mucka, et al., Am. J. Path 186 (11) 2803-2812 2016), and single nucleotide polymorphisms (SNPs) in NRP2 are associated with lymphedema (see, e.g., Miaskowski et al., PLoS ONE 8(4) e60164, 2013). NRP2 also regulates smooth muscle contractility (see, e.g., Bielenberg et al., Amer. J. Path. 181:548-559, 2012], and is highly expressed in developing skeletal muscle (see, e.g. Meye, et al., PLOS ONE DOI.10.1371/journal.pone.0139520 2015).

Neuropilins are also multifunctional co-receptors involved in tumor initiation, growth, metastasis, lymphangiogenesis, lymphatic metastasis and tumor immuno-surveillance, thereby directly contributing to tumor initiation, survival, and metastasis (see, e.g., Goel et al., EMBO Mol. Med. 5:488-508, 2013; Cao et al., Can. Res. 73(14) 4579-4590 2013; Tu, et al., Oncol. Lett. 12 4224-4230, 2016), Samuel et al., PLoS ONE 6(10) e23208, 2011).

Neuropilin-2 is expressed in various cells of the immune system such as B cells, T cells, NK cells, neutrophils, dendritic cells and macrophages, including for example, alveolar macrophages, and plays an important role in the regulation of immune cell activation and migration (see, e.g., Mendes-da-Cruz et al., PLoS ONE 9(7) e103405, 2014) including endosome maturation, the modulation of autophagy and efferocytosis, (see, e.g., Stanton et al., Cancer Res. 73:160-171, 2013, Schellenburg et al., Mol. Imm 90:239-244, 2017, Wang et al., Cancer Lett. 418 176-184 2018).

NRP2 is also expressed in endothelial and epithelial cells in the lung, and other tissues and cell types including bone osteoclasts and muscle cells [see, e.g., Bielenberg et al., Amer. J. Path. 181:548-559, 2012; Aung, et al., PLoS ONE 11(2) e0147358, 2016; and Wild et al., Int. J. Exp. Path. 93:81-103, 2012).

Neuropilin-2 also plays an active role both in neuronal development, and in the adult NRP2 is actively involved in peripheral nerve growth and remodeling, and plays a role in pain perception in inflammatory conditions such as arthritis, osteoarthritis and rheumatoid arthritis (see e.g. Hamilton, J et al., J. Bone & MM. Res. 2016 31(5) 911-924; Bannerman, P., et al., J. Neurosci. Res. 2008 86(14) 3163-3169; Malykhina, A., et al., BMC Physiology 2012, 12, 15).

It is becoming increasingly apparent that Neuropilin-2 also plays a key role in endosome development and regulates late endosomal maturation. These processes play important roles in phagocytosis and efferocytosis, which respectively play key roles in the clearance of infections and apoptotic cells (See, e.g. Diaz-Vera et al., J. Cell. Sci. 130, 697-711 2017; Dutta et al., Cancer Res. 76(2) 418-428 2016).

Neuropilin-2 is known to be a key player in the pathophysiology of many diseases (“NRP2-associated disease”) and interacts with a broad array of soluble ligands including, for example, semaphorin 3F, VEGF-C and D, and TGF-beta, and including an array of cellular receptors and co-factors (“NRP2 ligands”) (see, for example, Tables N2, N3 below), and FIGS. 1A-1B).

TABLE N2
Exemplary NRP2 Ligands

| Ligand | NRP1 | NRP2 |
|---|---|---|
| VEGF-A121 | + | |
| VEGF-A145 | | + |
| VEGF-A165 | + | + |
| VEGF-B167 | + | |
| VEGF-C | + | + |
| VEGF-D | + | + |
| VEGF-E | + | |
| PIGF-2 | + | + |
| VEGFR | +R1 and R2 | +R1, R2, R3 |
| Heparin | + | + |
| SEMA 3A | + | |
| SEMA 3B, 3C, 3D, 3F, 3G | + | + |
| Plexins A1, A2, A3, A4, & D1 | + | + |
| TGF-B1, 2, 3, and LAP | + | + |
| TbRI and TbRII | + | + |
| FGF-1, 2, 4, 7 | + | + |
| FGF receptor 1 | + | + |
| Integrins (see Table N3) | + | + |
| Fibronectin | + | |
| Galectin-1 | + | + |
| Li-CAM | + | + |
| Glat-1 | + | |
| HRS polypeptides | | + |

TABLE N3
Vertebrate integrins as NRP2 ligands

| Name | Synonyms | Distribution | Ligands |
|---|---|---|---|
| α₁β₁ | VLA-1 | Many | Collagens, laminins |
| α₂β₁ | VLA-2 | Many | Collagens, laminins |
| α₃β₁ | VLA-3 | Many | Laminin-5 |
| α₄β₁ | VLA-4 | Hematopoietic cells | Fibronectin, VCAM-1 |
| α₅β₁ | VLA-5; | widespread | fibronectin and proteinases |
| | fibronectin | | |
| | receptor | | |
| α₆β₁ | VLA-6; | widespread | laminins |
| | laminin | | |
| | receptor | | |
| α₇β₁ | | muscle, glioma | laminins |
| α_{L}β₂ | LFA-1 | T-lymphocytes | ICAM-1, ICAM-2 |
| α_{M}β₂ | Mac-1, CR3 | Neutrophils and monocytes | Serum proteins, ICAM-1 |
| α_{IIb}β₃ | Fibrinogen | Platelets | fibrinogen, fibronectin¹²⁴¹ |
| | receptor; | | |
| | gpllbllla | | |
| α_{V}β₁ | | ocular melanoma; neurological | vitronectin; fibrinogen |
| | | tumors | |
| α_{V}β₃ | vitronectin | activated endothelial cells, | vitronectin, fibronectin, |
| | receptor | melanoma, glioblastoma | fibrinogen, osteopontin, Cyr61, thyroxine, |
| | | | TETRAC |
| α_{V}β₅ | | widespread, esp. fibroblasts, | vitronectin and adenovirus |
| | | epithelial cells | |
| α_{V}β₆ | | proliferating epithelia, esp. | fibronectin; TGFβ1 + 3 |
| | | lung and mammary gland | |
| α_{V}β₈ | | neural tissue; peripheral nerve | fibronectin; TGFβ1 + 3 |
| α₆β₄ | | Epithelial cells | Laminin |

NRP2 is also polysialyated on dendritic cells, and actively interacts with the chemokine CCL21 to mediate immune cell migration via the CCR7 receptor, and for which single nucleotide polymorphisms in NRP2 are associated with ILD and RA have been described (see, e.g., Rey-Gallardo et al., Glycobiology 20:1139-1146, 2010., Stahl et al., Nat. Genet. 42:508-514, 2013., Miller et al., Arthritis Rheum. 65:3239-3247). Additionally, soluble, circulating forms of NRP2 are known (see, e.g., Parker et al., Structure 23(4) 677-687, 2015), and internal studies have confirmed the existence of circulating complexes of HRS polypeptides and NRP2 polypeptides in serum, and other fluids.

Given the central role played by NRP2 in pathophysiology, it is evident that interactions between NRP2 and HRS polypeptides provide potential for the treatment of diseases, including NRP2 associated diseases. Accordingly, the HRS polypeptides described herein, by selectively modulating the interactions of NRP2 with one or more of the ligands listed in Table N2 and Table N3, may be used to treat a wide range of diseases and conditions as described herein.

### Additional Therapeutic Agents and Compositions

Immunotherapy Agents. Certain embodiments employ one or more cancer immunotherapy agents. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and a cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In particular embodiments, the immune checkpoint modulatory agent is a polypeptide or peptide. The terms “peptide” and “polypeptide” are used interchangeably herein, however, in certain instances, the term “peptide” can refer to shorter polypeptides, for example, polypeptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Polypeptides and peptides can be composed of naturally-occurring amino acids and/or non-naturally occurring amino acids, as described herein

Antibodies are also included as polypeptides. Thus, in some embodiments, the immune checkpoint modulatory polypeptide agent is an antibody or “antigen-binding fragment thereof”, as described elsewhere herein.

In some embodiments, the agent is or comprises a “ligand,” for example, a natural ligand, of the immune checkpoint molecule. A “ligand” refers generally to a substance or molecule that forms a complex with a target molecule (e.g., biomolecule) to serve a biological purpose, and includes a “protein ligand,” which generally produces a signal by binding to a site on a target molecule or target protein. Thus, certain agents are protein ligands that, in nature, bind to an immune checkpoint molecule and produce a signal. Also included are “modified ligands,” for example, protein ligands that are fused to a pharmacokinetic modifier, for example, an Fc region derived from an immunoglobulin.

The binding properties of polypeptides can be quantified using methods well known in the art (see Davies et al., Annual Rev. Biochem. 59:439-473, 1990). In some embodiments, a polypeptide specifically binds to a target molecule, for example, an immune checkpoint molecule or an epitope thereof, with an equilibrium dissociation constant that is about or ranges from about ≤10-7 to about 10-8 M. In some embodiments, the equilibrium dissociation constant is about or ranges from about ≤10-9 M to about ≤10-10 M. In certain illustrative embodiments, the polypeptide has an affinity (Kd or EC50) for a target described herein (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the agent is a “small molecule,” which refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of about or less than about 1000-2000 Daltons, typically between about 300 and 700 Daltons, and including about or less than about 50, 100, 150, 200, 250, 300, 350, 400, 450, 500, 550, 500, 650, 600, 750, 700, 850, 800, 950, 1000 or 2000 Daltons.

Certain small molecules can have the “specific binding” characteristics described for herein polypeptides such as antibodies. For instance, in some embodiments a small molecule specifically binds to a target, for example, an immune checkpoint molecule, with a binding affinity (Kd or EC₅₀) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun. 33: 970-9, 2003; and Workman et al., Journal of Immun. 172: 5450-5, 2004), and has been reported to play a role in Treg suppressive function (see, e.g., Huang et al., Immunity. 21: 503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a VISTA antagonist or inhibitor. V-domain Ig suppressor of T cell activation (VISTA) is primarily expressed on hematopoietic cells and is an inhibitory immune checkpoint regulator that suppresses T-cell activation, induces Foxp3 expression, and is highly expressed within the tumor microenvironment where it suppresses anti-tumor T cell responses (see, e.g., Lines et al., Cancer Res. 74:1924-32, 2014). General examples of VISTA antagonists or inhibitors include an antibody or antigen-binding fragment or small molecule that specifically binds to VISTA and reduces one or more of its immune-suppressive activities.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derré et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a co-inhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014; 3:e27255. doi:10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the cancer immunotherapy agent is a cancer vaccine. Exemplary cancer vaccines include Oncophage, human papillomavirus HPV vaccines such Gardasil or Cervarix, hepatitis B vaccines such as Engerix-B, Recombivax HB, or Twinrix, and sipuleucel-T (Provenge). In some embodiments, the cancer vaccine comprises or utilizes one or more cancer antigens, or cancer-associate d antigens. Exemplary cancer antigens include, without limitation, human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, VEGR-3, NRP2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In certain embodiments, the cancer immunotherapy agent is an oncolytic virus. An oncolytic virus is a virus that preferentially infects and kills cancer cells. Included are naturally-occurring and man-made or engineered oncolytic viruses. Most oncolytic viruses are engineered for tumor selectivity, although there are naturally-occurring examples such as Reovirus and the SVV-001 Seneca Valley virus. General examples of oncolytic viruses include VSV, Poliovirus, Reovirus, Senecavirus, and RIGVIR, and engineered versions thereof. Non-limiting examples of oncolytic viruses include herpes simplex virus (HSV) and engineered version thereof, talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401, among others.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a T-cell based adoptive immunotherapy. In some embodiments, the cell-based immunotherapy comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015).

In certain instances, the cancer to be treated associates with the cancer antigen, that is, the cancer antigen-specific T-cells are targeted against or enriched for at least one antigen that is known to associate with the cancer to be treated. In some embodiments, the cancer antigen is selected from one or more of CD19, human Her2/neu, Her1/EGF receptor (EGFR), Her3, A33 antigen, B7H3, CD5, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

Additional exemplary cancer antigens include 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R1 71, HLA-A11/m, HLA-A2/m, FINE, homeobox NKX3.1, HOM-TES-14/SCP-1, HOM-TES-85, HPV-E6, HPV-E7, HSP70-2M, HST-2, hTERT, iCE, IGF-1 R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MCI R, M-CSF, ME1/m, mesothelin, MG50/PXDN, MMP11, MN/CA IX-antigen, MRP-3, MUC-1, MUC-2, MUM-1/m, MUM-2/m, MUM-3/m, myosin class 1/m, NA88-A, N-acetylglucosaminyltransferase-V, Neo-PAP, Neo-PAP/m, NFYC/m, NGEP, NMP22, NPM/ALK, N-Ras/m, NSE, NY-ESO-B, NY-ESO-1, OA1, OFA-iLRP, OGT, OGT/m, OS-9, OS-9/m, osteocalcin, osteopontin, pi 5, p190 minor bcr-abl, p53, p53/m, PAGE-4, PAI-1, PAI-2, PAP, PART-1, PATE, PDEF, Pim-1 Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGF-beta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, and WT1. Certain preferred antigens include p53, CA125, EGFR, Her2/neu, hTERT, PAP, MAGE-A1, MAGE-A3, Mesothelin, MUC-1, GP100, MART-1, Tyrosinase, PSA, PSCA, PSMA, STEAP-1, Ras, CEA and WT1, and more preferably PAP, MAGE-A3, WT1, and MUC-1.

In some embodiments the antigen is selected from MAGE-A1 (e.g., MAGE-A1 according to accession number M77481), MAGE-A2, MAGE-A3, MAGE-A6 (e.g., MAGE-A6 according to accession number NM_005363), MAGE-C1, MAGE-C2, melan-A (e.g., melan-A according to accession number NM_00551 1), GP100 (e.g., GP100 according to accession number M77348), tyrosinase (e.g., tyrosinase according to accession number NM_000372), survivin (e.g., survivin according to accession number AF077350), CEA (e.g., CEA according to accession number NM_004363), Her-2/neu (e.g., Her-2/neu according to accession number M1 1 730), WT1 (e.g., WT1 according to accession number NM_000378), PRAME (e.g., PRAME according to accession number NM_0061 15), EGFRI (epidermal growth factor receptor 1) (e.g., EGFRI (epidermal growth factor receptor 1) according to accession number AF288738), MUC1, mucin-1 (e.g., mucin-1 according to accession number NM_002456), SEC61 G (e.g., SEC61 G according to accession number NM_014302), hTERT (e.g., hTERT accession number NM_198253), 5T4 (e.g., 5T4 according to accession number NM_006670), TRP-2 (e.g., TRP-2 according to accession number NM_001 922), STEAP1 (Six-transmembrane epithelial antigen of prostate 1), PSCA, PSA, PSMA, etc.

In some embodiments, the cancer antigen is selected from PCA, PSA, PSMA, STEAP, and optionally MUC-1, including fragments, variants, and derivatives thereof. In some embodiments, the cancer antigen selected from NY-ESO-1, MAGE-C1, MAGE-C2, survivin, 5T4, and optionally MUC-1, including fragments, variants, and derivatives thereof.

In some instances, cancer antigens encompass idiotypic antigens associated with a cancer or tumor disease, particularly lymphoma or a lymphoma associated disease, for example, wherein the idiotypic antigen is an immunoglobulin idiotype of a lymphoid blood cell or a T cell receptor idiotype of a lymphoid blood cell.

In some instances, the cancer antigen-specific T-cells are selected from one or more of chimeric antigen receptor (CAR)-modified T-cells (e.g., targeted against a cancer antigen), and T-cell Receptor (TCR)-modified T-cells, tumor infiltrating lymphocytes (TILs), and peptide-induced T-cells.

The skilled artisan will appreciate that the various cancer immunotherapy agents described herein can be combined with any one or more of the various HRS polypeptides described herein, and used according to any one or more of the methods or compositions described herein.

Chemotherapeutic Agents. Certain embodiments employ one or more chemotherapeutic agents, for example, small molecule chemotherapeutic agents. Non-limiting examples of chemotherapeutic agents include alkylating agents, anti-metabolites, cytotoxic antibiotics, topoisomerase inhibitors (type 1 or type II), an anti-microtubule agents, among others.

Examples of alkylating agents include nitrogen mustards (e.g., mechlorethamine, cyclophosphamide, mustine, melphalan, chlorambucil, ifosfamide, and busulfan), nitrosoureas (e.g., N-Nitroso-N-methylurea (MNU), carmustine (BCNU), lomustine (CCNU), semustine (MeCCNU), fotemustine, and streptozotocin), tetrazines (e.g., dacarbazine, mitozolomide, and temozolomide), aziridines (e.g., thiotepa, mytomycin, and diaziquone (AZQ)), cisplatins and derivatives thereof (e.g., carboplatin and oxaliplatin), and non-classical alkylating agents (optionally procarbazine and hexamethylmelamine).

Examples of anti-metabolites include anti-folates (e.g., methotrexate and pemetrexed), fluoropyrimidines (e.g., 5-fluorouracil and capecitabine), deoxynucleoside analogues (e.g., ancitabine, enocitabine, cytarabine, gemcitabine, decitabine, azacitidine, fludarabine, nelarabine, cladribine, clofarabine, fludarabine, and pentostatin), and thiopurines (e.g., thioguanine and mercaptopurine);

Examples of cytotoxic antibiotics include anthracyclines (e.g., doxorubicin, daunorubicin, epirubicin, idarubicin, pirarubicin, aclarubicin, and mitoxantrone), bleomycins, mitomycin C, mitoxantrone, and actinomycin. Examples of topoisomerase inhibitors include camptothecin, irinotecan, topotecan, etoposide, doxorubicin, mitoxantrone, teniposide, novobiocin, merbarone, and aclarubicin.

Examples of anti-microtubule agents include taxanes (e.g., paclitaxel and docetaxel) and vinca alkaloids (e.g., vinblastine, vincristine, vindesine, vinorelbine).

The skilled artisan will appreciate that the various chemotherapeutic agents described herein can be combined with any one or more of the various HRS polypeptides described herein, and used according to any one or more of the methods or compositions described herein.

Hormonal Therapeutic Agents. Certain embodiments employ at least one hormonal therapeutic agent. General examples of hormonal therapeutic agents include hormonal agonists and hormonal antagonists. Particular examples of hormonal agonists include progestogen (progestin), corticosteroids (e.g., prednisolone, methylprednisolone, dexamethasone), insulin like growth factors, VEGF derived angiogenic and lymphangiogenic factors (e.g., VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, androgens, estrogens, and somatostatin analogs. Examples of hormonal antagonists include hormone synthesis inhibitors such as aromatase inhibitors and gonadotropin-releasing hormone (GnRH)s agonists (e.g., leuprolide, goserelin, triptorelin, histrelin) including analogs thereof. Also included are hormone receptor antagonist such as selective estrogen receptor modulators (SERMs; e.g., tamoxifen, raloxifene, toremifene) and anti-androgens (e.g., flutamide, bicalutamide, nilutamide).

Also included are hormonal pathway inhibitors such as antibodies directed against hormonal receptors. Examples include inhibitors of the the IGF receptor (e.g., IGF-IR1) such as cixutumumab, dalotuzumab, figitumumab, ganitumab, istiratumab, and robatumumab; inhibitors of the vascular endothelial growth factor receptors 1, 2 or 3 (VEGFR1, VEGFR2 or VEGFR3) such as alacizumab pegol, bevacizumab, icrucumab, ramucirumab; inhibitors of the TGF-beta receptors R1, R2, and R3 such as fresolimumab and metelimumab; inhibitors of c-Met such as naxitamab; inhibitors of the EGF receptor such as cetuximab, depatuxizumab mafodotin, futuximab, imgatuzumab, laprituximab emtansine, matuzumab, modotuximab, necitumumab, nimotuzumab, panitumumab, tomuzotuximab, and zalutumumab; inhibitors of the FGF receptor such as aprutumab ixadotin and bemarituzumab; and inhibitors of the PDGF receptor such as olaratumab and tovetumab.

The skilled artisan will appreciate that the various hormonal therapeutic agents described herein can be combined with any one or more of the various HRS polypeptides described herein, and used according to any one or more of the methods or compositions described herein.

Kinase Inhibitors. Certain embodiments employ at least one kinase inhibitor, including tyrosine kinase inhibitors and phosphoinositide 3 (PI3) kinase inhibitors. Examples of kinase inhibitors include, without limitation, adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib. Exemplary PI3 kinase inhibitors include alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147.

The skilled artisan will appreciate that the various kinase inhibitors described herein can be combined with any one or more of the various HRS polypeptides described herein, and used according to any one or more of the methods or compositions described herein.

### Methods of Use

Certain embodiments include methods of treating, ameliorating the symptoms of, and/or reducing the progression of, a disease or condition in a subject in need thereof, comprising administering to the subject a HRS polypeptide. In some instances, the HRS polypeptide specifically binds to a human neuropilin-2 (NRP2) polypeptide (see Table N1). In some instances, the HRS polypeptide interferes with binding of the human NRP2 polypeptide to a NRP2 ligand. In some instances, the HRS polypeptide mimics one or more signaling activities of the NRP2 ligand binding to the NRP2 polypeptide, for example, by acting as an agonist, partial agonists, antagonist, partial agonist, inverse agonist of the NRP2 ligand. Exemplary NRP2 ligands are provided in Table N2 and Table N3.

In certain embodiments, subject in need thereof has a NRP2-associated disease or condition. Exemplary NRP2-associated diseases and conditions include, without limitation, cancer and diseases or pathologies associated with cancer, including cancer cell growth, cancer initiation, cancer migration, cancer cell adhesion, cancer cell invasion, cancer cell chemoresistance, and cancer cell metastasis. Also included are diseases associated with inflammation and autoimmunity, including inflammatory lung diseases such as chronic hypersensitivity pneumonitis, pulmonary inflammation, and related inflammatory diseases. Also included are diseases associated with inappropriate immune cell activation or migration such as graft versus host disease (GVHD) and rheumatoid arthritis-associated interstitial lung disease (RA-ILD). Additional examples include diseases associated with lymphatic development, lymphangiogenesis, and lymphatic damage, including edema, lymphedema, secondary lymphedema, inappropriate fat absorption and deposition, excess fat deposition, and vascular permeability. Also included are diseases associated with infections, including latent infections, and diseases associated with allergic disorders/diseases and allergic responses, including chronic obstructive pulmonary disorder (COPD), neutrophilic asthma, antineutrophil cytoplasmic antibody (ANCA)-associated systemic vasculitis, systemic lupus erythematosus, rheumatoid arthritis, inflammasome-related disease(s), and skin-related neutrophil-mediated disease(s) such as pyoderma gangrenosum. Additional examples include diseases associated with granulomatous inflammatory diseases, including sarcoidosis and other pulmonary granulomatous diseased, and non-lung granulomas. Also included are fibrotic diseases such endometriosis, fibrosis, endothelial to mesenchymal transition (EMT), and wound healing, among others. Also included are diseases associated with inappropriate smooth muscle contractility and vascular smooth muscle cell migration and/or adhesion, and diseases associated with inappropriate autophagy, phagocytosis, and efferocytosis. Additional examples include neuronal diseases, including diseases associated with peripheral nervous system remodeling and pain perception. Also included are diseases associated with bone development and/or bone remodeling, and diseases associated with inappropriate migratory cell movement.

In some embodiments, the subject has, and/or is selected for treatment based on having, increased extracellular fluid (for example, circulating or serum) levels of a soluble NRP2 polypeptide (for example, selected from Table N1), either bound or free or a coding mRNA thereof, for example, relative to the levels of a healthy or matched control standard or reference population of subject(s). In some embodiments, the extracellular fluid levels of NRP2 are detected in serum, plasma, lymphatic fluid, interstitial fluid, and/or in specific tissue compartments relevant to the NRP2 associated disease, including for example, bronchoalveolar lavage (BALF) and synovial fluid. In some embodiments, the extracellular fluid levels of a soluble NRP2 polypeptide are about or at least about 10, 20, 30, 50, 100, 200, 300, 400, 500, 600, 700, 800, 900, 1000, 1100, 1200, 1300, 1400, 1500, 1600, 1700, 1800, 1900, 2000, 3000, 4000, 5000 pM of the soluble NRP2 polypeptide, or about 30-50, 50-100, 100-2000, 200-2000, 300-2000, 400-2000, 500-2000, 600-2000, 700-2000, 800-2000, 900-2000, 1000-2000, 2000-3000, 3000-4000, 4000-5000 pM of the soluble NRP2 polypeptide.

In some embodiments, the subject has, and/or is selected for treatment based on having, increased levels of a soluble NRP2 polypeptide (for example, selected from Table N1), either bound or free or a coding mRNA thereof, relative to a non-diseased control cell or tissue, for instance, a non-diseased control cell or tissue of the same type as the NRP2-associated disease cell or tissue. For instance, in some embodiments, the levels of the soluble NRP2 polypeptide (or a coding mRNA thereof) in the NRP2-associated disease cell or tissue are about or at least about 1.5, 2, 3, 4, 5, 6, 7, 8, 9, 10, 15, 20, 30, 40, 50, 60, 70, 80, 90, 100 or more times the levels of a non-diseased control cell or tissue. Some embodiments thus include methods of selecting a subject for treatment, comprising (i) determining extracellular fluid levels of a soluble NRP2 polypeptide and/or a coding mRNA thereof in the subject relative to a control or reference, and (ii) administering to the subject a therapeutic composition comprising at least one HRS polypeptide, as described herein, if the subject has increased levels of the soluble NRP2 polypeptide and/or a coding mRNA thereof relative to the control or reference.

In some embodiments, the subject has, and/or is selected for treatment based on having, increased extracellular fluid levels of an NRP2 ligand, or increased extracellular fluid levels of NRP2:NRP2 ligand complexes (optionally selected from Table N1 and Table N2), relative to a healthy or matched control standard or reference population of subject(s), for example, levels that are about or at least about 1.5, 2, 3, 4, 5, 6, 7, 8, 9, 10, 15, 20, 30, 40, 50, 60, 70, 80, 90, 100 or more times the levels of the control or reference. Certain embodiments therefore include methods of selecting a subject for treatment, comprising (i) determining extracellular fluid levels of NRP2:NRP2 ligand complexes in the subject relative to a control or reference, and (ii) administering to the subject a therapeutic composition comprising at least one HRS-polypeptide, as described herein, if the subject has increased levels of the NRP2:NRP2 ligand complexes relative to the control or reference.

In some embodiments, the subject has, and/or is selected for treatment based on having, increased extracellular fluid levels of HRS:NRP2 complexes (optionally selected from Table H1 and Table N1) relative to a healthy or matched control standard or reference population of subject(s), for example, levels that are about or at least about 1.5, 2, 3, 4, 5, 6, 7, 8, 9, 10, 15, 20, 30, 40, 50, 60, 70, 80, 90, 100 or more times the levels of the control or reference. Certain embodiments therefore include methods of selecting a subject for treatment, comprising (i) determining extracellular fluid levels of HRS:NRP2 complexes in the subject relative to a control or reference, and (ii) administering to the subject a therapeutic composition comprising at least one HRS-polypeptide, as described herein, if the subject has increased levels of the HRS:NRP2 complexes relative to the control or reference.

In some embodiments, the subject has, and/or is selected for treatment based on having, a single nucleotide polymorphism (SNP) in an NRP2 polypeptide or an NRP2 encoding polynucleotide from the subject.

In some embodiments, the subject has, and/or is selected for treatment based on having, a disease associated with increased levels or expression of NRP2a and/or NRP2b, or an altered ratio of NRP2a:NRP2b expression, relative to a healthy control or matched control standard or population of subject(s). In some embodiments, the levels of NRP2b are increased by about or at least about 10%, 20%, 30%, 40%, 50%, 100%, 200%, 300%, 400%, 500%, 600%, 700%, 800%, 900%, 1000% compared to a healthy control or matched control standard or population of subject(s).

In some embodiments, the healthy control or matched control standard or population of subject(s) comprises average ranges for age-matched samples of cancerous or non-cancerous cells or tissue of the same type as the cancer, which comprise specific characteristics such as drug resistance, metastatic potential, aggressiveness, genetic signature (optionally p53 mutation(s), PTEN deletion, IGFR expression), and/or expression patterns.

In some embodiments, the subject has, and/or is selected for treatment based on having, an infection. In some instances, the infection is a lymphedema-associated infection, for example, erysipelas, cellulitis, lymphangitis, and/or sepsis.

In some embodiments, the HRS polypeptide is administered in combination with a second agent, for example, an antimicrobial agent, an antifungal agent, and/or an antihelminthic agent. In some embodiments, the HRS polypeptide and the second agent are administered together as part of the same therapeutic composition. In some embodiments, the HRS polypeptide and the second agent are administered as separate therapeutic compositions. In some embodiments, the second agent is selected from one or more of aminoglycosides such as Amikacin, Gentamicin, Kanamycin, Neomycin, Netilmicin, Tobramycin, Paromomycin, Streptomycin, and Spectinomycin; carbapenems such as Ertapenem, Doripenem, Imipenem/Cilastatin, and Meropenem; cephalasporins such as Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Cefepime, Ceftaroline fosamil, and Ceftobiprole; glycopeptides such as Teicoplanin, Vancomycin, Telavancin, Dalbavancin, Oritavancin; lincosamines such as Clindamycin and Lincomycin; macrolides such as Azithromycin, Clarithromycin, Erythromycin, Roxithromycin, Telithromycin, and Spiramycin; penicillins such as Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; polypeptides such as Bacitracin, Colistin, and Polymyxin B; quinolones/fluoroquinolones such as Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; sulfonamides such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), and Sulfonamidochrysoidine; tetracyclines such as Demeclocycline, Doxycycline, Metacycline, Minocycline, Oxytetracycline, and Tetracycline; anti-mycobacterials such as Clofazimine, Dapsone, Capreomycin, Cycloserine, Ethambutol, Ethionamide, Isoniazid, Pyrazinamide, Rifampicin (Rifampin), Rifabutin, Rifapentine, and Streptomycin; Chloramphenicol; Metronidazole; Mupirocin; Tigecycline; Tinidazole; and antihelminthic agents such as Diethylcarbamazine and Albendazole.

### HRS Polypeptide Mediated Treatment of Lymphedema

Lymphedema is a chronic debilitating disease that in the United States and Western countries occurs most typically as a result of cancer surgery, obesity, congestive heart failure, hypertension, and/or peripheral vascular/venous disease. In setting of cancer treatment, lymphedema occurs as a result of iatrogenic injury to the lymphatic system-most commonly after lymph node dissection but also as a result of wide skin excisions and adjuvant therapy with radiation. (See, e.g. Purushotham et al., J. Clin. Oncol. 23:4312-4321, 2005; Szuba et al., Cancer 95:2260-2267, 2002; Tsai et al., Ann. Surg. Oncol. 16:1959-72, 2009).

It is estimated that as many as 1 in 3 patients who undergo lymph node dissection will go on to develop lymphedema and conservative estimates suggest that as many as 50,000 new patients are diagnosed annually. (See, e.g. DiSipio et al., Lancet Oncol. 14:500-515, 2013; Petrek et al., Cancer 83:2776-2781, 1998). Because lymphedema is a life-long disease, the number of affected individuals is increasing annually with current estimates ranging between 5-6 million Americans (Rockson et al., Ann. NY Acad. Sci. 1131:147-154, 2008) and over 200 million people world-wide. It is likely that this number will continue to increase in the future since the development of lymphedema is nearly linearly related with cancer survivorship, and because the prevalence of known risk factors for lymphedema, such as obesity and radiation treatment, is rising (see, e.g., Erickson et al., J. Natl. Cancer Inst. 93:96-111, 2001).

Secondary lymphedema may also take several years to develop, suggesting that its development is not solely dependent upon on lymphatic injury, but may also be precipitated by underlying chronic inflammatory conditions. The CD4⁺ cell response in lymphedema, similar to other fibroproliferative disorders, is characterized by a mixed Th1/Th2 cell population. (Avraham et al., FASEB J. 27:1114-1126, 2013). Naive CD4+ T cells, also known as T-helper or Th cells, patrol secondary lymphoid structures and, upon activation, differentiate along numerous distinct/overlapping cell types (e.g., Th1, Th2, Th17, T regulatory, etc.). The Th2 subset of cells plays a key role in regulation of responses to parasites and some autoimmune responses. These cells have also been implicated in the pathology of fibroproliferative diseases in a number of organ systems including the heart, lung, kidneys and skin. More recent studies have shown that the number of Th2 cells is increased in tissue biopsies obtained from patients with lymphedema and that inhibition of Th2 differentiation decreases the pathology of lymphedema in mouse models.

Depletion of CD4⁺ cells, or macrophages (but not other inflammatory cell types including CD8⁺ cells) or inhibition of Th2 differentiation (but not generalized inflammation or inhibition of interleukin-6) markedly decreases the degree of fibrosis, increases lymphangiogenesis and lymphatic fluid transport, and effectively treats established lymphedema in preclinical mouse models. (See, e.g. Avraham et al., FASEB J. 27:1114-1126, 2013; Zampell et al., PLoS ONE 7:e49940, 2012; Ghanta et al., Am. J. Physiol. Heart Circ. Physiol. 308:H1065-1077, 2015). These findings are supported by recent studies demonstrating that T cells potently inhibit lymphangiogenesis by producing anti-lymphangiogenic cytokines/growth factors, including interferon gamma (IFN-γ), interleukin (IL)-4, IL-13, and TGF-β1. (See, e.g. Kataru et al., Immunity 34:96-107, 2011; Shin et al., Nat. Commun. 6:6196, 2015; Shao et al., J. Interferon. Cytokine Res. 26:568-574, 2006; Oka et al., Blood 111:4571-4579, 2008, Corliss et al., Microcirculation 23(2) 95-121, 2016). Taken together, these findings suggest that infiltrating CD4⁺ cells and macrophages in lymphedematous tissues decrease lymphatic function through multiple mechanisms including induction of structural changes of lymphatic vessels secondary to tissue fibrosis and inhibition of collateral lymphatic vessel formation.

Lymphedema is disfiguring and debilitating; patients have chronic swelling of the affected extremity, recurrent infections, limited mobility, and decreased quality of life. (See e.g. Hayes et al., Cancer 118:2237-2249, 2012). In addition, once lymphedema develops it is usually progressive. Currently there is no known pharmacologic therapy that can halt progression or promote resolution of lymphedema. (See, e.g. Cormier et al., Ann. Surg. Oncol. 19:642-651, 2012). As a result, patients are required to wear tight, uncomfortable garments for the rest of their lives, in an effort to prevent lymphatic fluid buildup in the affected extremity, and to undergo intense and time consuming physical therapy treatments. (See e.g. Koul et al., Int. J. Radiat. Oncol. Biol. Phys., 67:841-846, 2007). In addition, despite on-going chronic care, some patients still have severe progression of their disease with increasing swelling and frequent infections in the lymphedematous limb. Development of targeted treatments for lymphedema is therefore an important goal and is an unmet biomedical need.

A direct role of NRP2 in regulating lymphatic remodeling in adults has been demonstrated both in animal knock systems which demonstrate enhanced edema after inflammatory challenge in NPR2 KO mice. (See, e.g. Mucka et al., Am. J. Path 186 (11) 2803-2812, 2016) as well as increased disease susceptibility to secondary lymphedema in subjects with SNPs in neuropilin-2. (See, e.g., Miaskowski et al., PLoS ONE 8(4) e60164, 2013). Additionally the expression of NRP2 on immune cells and regulation of that activity by HRS polypeptides, in addition to the potential modulation of NRP2 mediated effects on vascular biology, strongly suggest that HRS polypeptides represent a new and exciting therapeutic option for the treatment of lymphedema.

In some embodiments, the subject has, and/or is selected for treatment based on having, a Stage of lymphedema selected from Stage 1, Stage 2, Stage 3, Stage 4, Stage 5, Stage 6, and Stage 7, as illustrated below.

Stage 1, swelling increases during the day and typically disappears overnight as the patient lies flat in bed. Affected tissues are at the pitting stage, and when pressed by the fingertips, the affected area indents and reverses with elevation.

Stage 2, swelling is not reversible overnight, and does not disappear without proper management. Affected tissues have a spongy consistency and are considered non-pitting, and when pressed by the fingertips, the affected area bounces back without indentation. Fibrosis, found in Stage 2 lymphedema, marks the beginning of the hardening of the limbs and increasing size.

Stage 3, swelling is irreversible and the affected tissues (e.g., limbs) typically become increasingly large. Affected tissues are hard (fibrotic) and unresponsive.

Stage 4, the size and circumference of the affected tissues (e.g., limbs) become noticeably large. Bumps, lumps, and/or protusions (also called knobs) begin to appear on the skin.

Stage 5, the affected tissues (e.g., limbs) become grossly large. In some instances, one or more deep skin folds is prevalent at this stage.

Stage 6, knobs of small elongated or small rounded sizes cluster together, resulting in mossy-like shapes on the affected tissues (e.g., limbs). Mobility of the subject is significantly reduced.

Stage 7, the subject is handicapped, and is unable to independently perform daily routine activities such as walking, bathing, and cooking. Assistance from the family and health care system is needed.

In some embodiments, the subject has, and/or is selected for treatment based on having, a Grade of lymphedema selected from Grade 1 (mild edema), Grade 2 (moderate edema), Grade 3a (severe edema), Grade 3b (massive edema), and Grade 4 (gigantic edema), as illustrated below.

Grade 1 (mild edema) involves the distal parts such as a forearm and hand or a lower leg and foot. The difference in circumference is less than 4 cm and other tissue changes are not yet present.

Grade 2 (moderate edema) involves an entire limb or corresponding quadrant of the trunk. The difference in circumference is 4-6 cm. Tissue changes, such as pitting, are apparent. The subject may experience erysipelas, an infection of the upper dermis and superficial lymphatics, usually caused by beta-hemolytic group A Streptococcus bacteria.

Grade 3a (severe edema), lymphedema is present in one limb and its associated trunk quadrant. The circumferential difference is greater than 6 centimeters. Significant skin alterations, such as cornification or keratosis, cysts and/or fistulae, are present. Additionally, the subject may experience repeated attacks of erysipelas.

Grade 3b (massive edema) includes the same symptoms as grade 3a, except that two or more extremities are affected.

Grade 4 (gigantic edema), the affected extremities are huge, due to almost complete blockage of the lymph channels.

Certain embodiments therefore include methods of treating, ameliorating the symptoms of, and/or reducing the progression of, lymphedema in a subject in need thereof, comprising administering to the subject a HRS polypeptide or therapeutic composition provided herein.

### HRS Polypeptide Mediated Treatment of Fibrotic Diseases

Fibrotic diseases encompass a wide spectrum of clinical entities including systemic fibrotic diseases such as systemic sclerosis, sclerodermatous graft versus host disease, nephrogenic systemic fibrosis, lymphedema associated fibrosis, and IgG₄-associated sclerosing disease, as well as numerous organ-specific disorders including radiation-induced fibrosis, and cardiac, pulmonary, liver, and kidney fibrosis. Although their causative mechanisms are quite diverse, these diseases share the common feature of an uncontrolled and progressive accumulation of fibrous tissue macromolecules in affected organs leading to their dysfunction and ultimate failure. Numerous studies have identified myofibroblasts as the cells responsible for the establishment and progression of the fibrotic process. Tissue myofibroblasts in fibrotic diseases originate from several sources including quiescent tissue fibroblasts, circulating CD34+ fibrocytes, and the phenotypic conversion of various cell types including epithelial and endothelial cells into activated myofibroblasts.

Transforming growth factor beta-1 (TGF-(31) is a critical regulator of fibrosis in a variety of organ systems, acting via direct mechanisms to increase collagen production by fibroblasts and decrease turnover of matrix products. (See e.g. Willis et al., Am. J. Pathol. 166:1321-1332, 2005; Sakai et al., Am. J. Pathol. 184:2611-2617, 2014; Qi et al., Am. J. Physiol. Renal Physiol. 288:F800-F809, 2005; Bonniaud et al., J. Immunol. 173:2099-2108, 2004). Moreover NRP2 plays a direct role in regulating TGF-β1 mediated EMT, directly leading to fibrosis (see, e.g., Grandclement et al., PLoS ONE 6(7) e20444, 2011) as well as mediating EMT, or endo-EMT in fibroblasts, myofibroblasts and endothelial cells to promote fibrosis formation (See, e.g., Pardali et al., Int. J. Mol. Sci. 18 2157 2017). In addition, TGF-β1 is a key regulator of inflammatory responses and is thought to regulate fibrosis indirectly by modulating chronic inflammation. (Pesce et al., PLoS Pathog. 5:e1000371, 2009).

Moreover TGF-β1 is markedly increased in lymphedematous tissues, both clinically and in mouse models of lymphedema. Inhibition of TGF-β1 using immunotherapy significantly accelerates lymphatic regeneration, decreases fibrosis, decreases inflammation, and improves lymphatic function in the mouse tail model. (See, e.g. Avraham et al., Plast. Reconstr. Surg. 124:438-450, 2009; Clavin et al., Am. J. Physiol. Heart Circ. Physiol. 295:H2113-H2127, 2008; Avraham et al., Am. J. Pathol. 177:3202-3214, 2010). Inhibition of fibrotic responses preserves the capacity of the lymphatic system to transport interstitial fluid and inflammatory cells.

Recent studies have shown that CD4⁺ cells and macrophages play a crucial role in the regulation of fibrosis in both clinical and animal models of lymphedema. (See, e.g. Ogata et al., J. Invest. Derm. 136 706-714, 2016, Avraham et al., Am. J. Pathol. 177:3202-3214, 2010; Avraham et al., FASEB J. 27:1114-1126, 2013; Zampell et al., Am. J. Physiol. Cell Physiol. 302:C392-C404, 2012; Zampell et al., PLoS ONE 7:e49940, 2012). For example, it has been found that clinical lymphedema biopsy specimens and animal models of lymphedema are infiltrated by CD4⁺ cells, and that the number of these cells correlates with the degree of fibrosis and clinical severity of disease. (See, e.g. Avraham et al., FASEB J. 27:1114-1126, 2013). Patients with late stage lymphedema had significantly more infiltrating T cells in general, specifically more CD4⁺ cells, than those with early stage disease. Improvements in clinical symptoms of lymphedema after lymphovenous bypass, a procedure in which obstructed lymphatics are shunted to the venous circulation, is associated with decreased tissue fibrosis and decreased CD4⁺ cell infiltration. (Torrisi, et al., Lymphat. Res. Biol. 13:46-53, 2015).

Fibrosis is also a hallmark of many autoimmune disease including chronic graft-versus-host disease (GVHD). Particularly for example associated with allogeneic hematopoietic stem cell transplantation (allo-HSCT) which is the main curative treatment for many hematological malignancies. Its anti-tumor activity relies in large part on immune-mediated graft-versus-tumor effects (GvT effects). However, donor immune cells contained in the graft can also attack healthy host tissues causing graft-versus-host disease (GVHD). GVHD can be divided into two syndromes, acute GVHD, historically defined as a GVHD reaction occurring within the first 100 days after allo-SCT and chronic GVHD (cGVHD), that generally occurs beyond day 100. While cGVHD has been associated with graft-versus-tumor effects, it is also a major cause of morbidity/mortality in long-term transplant recipients.

Sclerodermatous cGVHD (scl-cGVHD) is one of the most severe form of cGVHD and develops in approximately 20% of cGVHD patients. Although scl-cGVHD shares common features with systemic fibrosis, the two syndromes differ both in terms of pathology (scl-cGVHD usually begins in the superficial layer of the skin and then extents to deeper layers of the skin while the opposite is generally true in systemic sclerosis), and in terms of clinical symptoms, with clinical features such as Raynaud's syndrome, pulmonary hypertension and cardiac dysfunction being frequently observed in patients with systemic sclerosis but infrequently in scl-cGVHD patients.

Given the role of NRP2 in modulating both immune cell activation and migration, and TGF-β1-mediated EMT to promote fibrosis formation, as well as, the ability of HRS polypeptides to modulate these processes, it is clear that HRS polypeptides represent a new and exciting therapeutic option for the treatment of fibrosis, both in the context of lymphedema, and in other fibrotic diseases and disorders.

Certain embodiments therefore include methods of treating, ameliorating the symptoms of, and/or reducing the progression of, fibrosis in a subject in need thereof, comprising administering to the subject a HRS polypeptide or therapeutic composition provided herein.

### HRS Polypeptide Mediated Regulation of Lymphangiogenesis

The lymphatic system consists of networks of interconnected capillaries, collecting vessels and lymph nodes that absorb, collect and transport the fluid and protein filtered from the blood vascular system. This system provides a critical homeostatic function: in humans, lymphatic vessels return >4 liters of fluid and a substantial amount of protein per day back into the great veins of the neck.

Lymphatic vascular dysfunction (lymphedema) results in the accumulation of excess fluid (edema) in the interstitium. Although lymphedema is typically not life-threatening, it has serious health consequences, including pain, immobility, fibrosis, inflammation, adipose tissue accumulation, and tissue damage. Because the lymphatic system is also a critical component of immune responses, lymphedema is typically accompanied by an increased risk of infection and other immune system problems.

Lymphangiogenesis is the formation of new lymphatic vessels from preexisting lymphatic vessels and is associated with diverse pathological conditions including metastatic dissemination, graft rejection (e.g. cornea, kidney and heart), type 2 diabetes, obesity, hypertension, as well as lymphedema (See, e.g. Alitalo, K., et al. Nature 438:946-953, 2005; Karaman, S., et al. J Clin Invest 124:922-928, 2014; Kim, H., et al., J Clin Invest 124:936-942, 2014; Maby-El Hajjami, H., et al., Histochem Cell Biol 130:1063-107, 2008; Machnik, A., et al., Nat Med 15:545-552; Mortimer, P. S., et al. 2014. J Clin Invest 124:915-921; Skobe, M., et al. 2009. Nat Med 15:993-994).

Lymphatic vessel invasion in and around a primary tumor compared to invasion of blood vessels is a prognostic marker of the aggressiveness of various types of cancers. Growth of lymphatic vessels is also involved in graft rejection (Dietrich, T., et al., J Immunol 184:535-539, 2010, Hall, F. T., et al., Arch Otolaryngol Head Neck Surg 129:716-719, 2003.; Maula, S. M., et al., Cancer Res 63:1920-1926, 2003; Miyata, Y., et al., J Urol 176:348-353, 2006; Saad, R. S., et al., Mod Pathol 19:1317-1323, 2006; Schoppmann, S. F., et al., Ann Surg 240:306-312, 2004; Zeng, Y., et al., Prostate 65:222-230, 2005).

Despite the well-established significance of lymphatics in the pathogenesis of numerous diseases, there has been little progress in the development of anti-lymphangiogenic agents compared to the abundance of anti-angiogenic agents that have entered clinical trials. Therefore, the development of additional lymphangiogenesis inhibitors is of interest for the treatment of a range of conditions, including, lymphedema and cancer metastasis.

Anti-lymphangiogenic agents are useful, for example, for treatment of debilitating diseases of the eye, where the growth of lymphatic vessels is the major reason of corneal graft rejection, and also a major contributor to neovascularization that is associated with age related macular degeneration (see, for example, Dietrich et al., J Immunol 184:535-539, 2010). In particular, penetrating keratoplasty is the most common form of solid tissue transplantation, with approximately 40,000 corneal transplantations performed each year in the United States. The success rate of penetrating keratoplasty is as high as 90% for uncomplicated first grafts performed in avascular low-risk beds. However, the rejection rate of the corneal grafts placed in high-risk vascularized host beds is extremely high (70% to 90%). Thus, the development of safe and targeted new regimens to inhibit lymphangiogenesis is needed to promote graft survival and reduce or inhibit neovascularization.

Anti-lymphangiogenesis drugs are useful also for treatment of dry eye disease. Significant upregulation of prolymphangiogenic factors (e.g. VEGF-C, VEGF-D, and VEGFR-3) and selective growth of lymphatic vessels without concurrent growth of blood vessels has been demonstrated in corneas with dry eye disease (Goyal, S., et al., Arch Ophthalmol 128:819-824, 2010). Dry eye disease is an immune-mediated disorder affecting about 5 million Americans. It severely impacts the vision-related quality of life and the symptoms can be debilitating. The current therapeutic options for dry eye disease are limited, mostly palliative, and expensive. Therefore, development of lymphangiogenesis inhibitors is of therapeutic value for treatment of dry eye disease.

It is currently accepted that metastases are responsible for the vast majority, estimated at 90%, of deaths from solid tumors (Gupta and Massague, Cell 127, 679-695, 2006). The complex process of metastasis involves a series of distinct steps including detachment of tumor cells from the primary tumor, intravasation of tumor cells into lymphatic or blood vessels, and extravasation and growth of tumor cells in secondary sites. Analysis of regional lymph nodes in many tumor types suggests that the lymphatic vasculature is an important route for the dissemination of human cancers. Furthermore, in almost all carcinomas, the presence of tumor cells in lymph nodes is the most important adverse prognostic factor. While it was previously thought that such metastases exclusively involved passage of malignant cells along pre-existing lymphatic vessels near tumors, recent experimental studies and clinicopathological reports (See, e.g., Achen et al., Br J Cancer 94, 1355-1360, 2006 and Nathanson, Cancer 98,413-423, 2003) suggest that lymphangiogenesis can be induced by solid tumors and can promote tumor spread. These and other recent studies suggest targeting lymphatics and lymphangiogenesis may be a useful therapeutic strategy to restrict the development of cancer metastasis, which would have a significant benefit for many patients.

Accordingly, there is a need for methods and compositions that inhibit the activities of pro-lymphangiogenic factors and for methods that prevent or treat graft rejection, dry-eye disease tumor metastasis, lymphedema and other inflammatory conditions.

Given the role of HRS polypeptides in modulating the binding and activity of NRP2 ligands interacting with NRP2, such HRS polypeptides potentially represent powerful new tools for the development of both pro and anti-lymphangiogenic therapies. Such differential effects may be mediated for example, via the uses of different HRS polypeptide compositions, differential dosing, differential treatment duration, or the appropriate use of additional co-factors, such as, for example, VEGF-C, or Semaphorins 3F and/or 3G.

Certain embodiments therefore include methods of modulating (e.g., increasing, reducing) lymphangiogenesis in a subject in need thereof, comprising administering to the subject a HRS polypeptide or therapeutic composition provided herein. Some embodiments include methods of treating, ameliorating the symptoms of, and/or reducing the progression of, lymphangiogenesis and/or neovascularization in a subject in need thereof, comprising administering to the subject a HRS polypeptide or therapeutic composition provided herein. Some embodiments include methods of treating, ameliorating the symptoms of, and/or promoting the progression of, or restoring lymphangiogenesis in a subject in need thereof, comprising administering to the subject a HRS polypeptide or therapeutic composition provided herein.

### HRS Polypeptide Regulated Modulation of Smooth Muscle Contractility

Reduced smooth muscle (SM) contractility in the bladder can stem from numerous etiologies including obstruction secondary to benign prostatic hyperplasia (BPH), posterior urethral valves, diabetes mellitus, multiple sclerosis, spinal cord injury, or idiopathic causes. (See, e.g., Drake et al., Nat Rev Urol. 11(8):454-464, 2014). In conditions such as BPH or posterior urethral valves, the bladder contracts against an obstructed outlet. The initial response is adaptive, involving a compensatory phase of SM hypertrophy that enables increased force generation to overcome the increased outlet resistance. When the demand outstrips the adaptive capability of the bladder, contractile performance becomes less efficient, residual volumes increase, and the bladder remodels, ultimately leading to a loss of detrusor contractility as the bladder decompensates. (See, e.g., Zderic S A, et al., J Cell Mol Med. 16(2):203-217, 2012). The prevalence of underactive detrusor function is reported to be as high as 48% in adults (Osman N I, et al., Eur Urol; 65(2):389-398, 2014). Furthermore, existing pharmacological treatments for restoration of SM contraction such as muscarinic agonists or cholinesterase inhibitors have shown limited efficacy and adverse effects (Barendrecht et al., BJU Int. 99(4):749-752, 2007).

Recent studies have identified bladder smooth muscle as a major site of Nrp2 expression, demonstrated the inhibition of RhoA and cytoskeletal stiffness in primary bladder smooth muscle cells treated with the NRP2 ligand, SEMA3F; and observed increased contractility of bladder SM strips from mice with ubiquitous or smooth muscle-specific deletion of Nrp2 in vivo, when compared with tissues from Nrp2-intact littermate controls. (See, e.g. Bielenberg et al., Am. J. Pathol. 181 548-559, 2012; Vasquez et al., JCI Insight 2(3) e90617, 2017).

Collectively, these findings suggest that downregulation of Nrp2 is an important component of the compensatory response to obstruction in experimental animals and in humans with obstruction, and suggest that Nrp2 may represent a novel pharmacological target for maintenance or restoration of detrusor contractility in the decompensated bladder.

Furthermore, recent studies have shown that targeting Nrp2 in bladders undergoing decompensation has the potential to restore contractility in spite of ongoing obstruction. (Vasquez et al., JCI Insight 2(3) e90617, 2017). These findings argue that the Np2 axis represents a potentially novel pharmacologic target for restoration of SM contractility and provide an important platform for the development of HRS polypeptide based modulators of Nrp2 function.

Pharmacological management of diminished detrusor contractility to date has focused on stimulation of parasympathetic activity to enhance bladder contractility and reduction of outflow resistance to facilitate bladder emptying (Chancellor et al., Urology 72(5) 966-967, 2008). However, analysis of 10 randomized clinical trials of parasympathomimetic drugs in patients with poorly contractile bladders, revealed either a worsening of symptoms or a lack of significant improvement (Barendrecht et al., BJU Int. 99(4) 749-752, 2007).

The increase in contractility following Nrp2 deletion in the decompensating bladder suggests that Nrp2 may be a novel target to mitigate reduced detrusor contractility under conditions of chronic obstruction. Given the role of HRS polypeptides in modulating the binding and activity of NRP2 ligands interacting with NRP2, such HRS polypeptides potentially represent powerful new tools for the development of new therapies to modulate smooth muscle contractility, including for example, the treatment of reduced smooth muscle (SM) contractility in the bladder.

Certain embodiments therefore include methods of modulating (e.g., increasing, reducing) smooth muscle contractility in a subject in need thereof, comprising administering to the subject a HRS polypeptide or therapeutic composition provided herein. Certain embodiments include treating, ameliorating the symptoms of, and/or reducing the progression of, reduced smooth muscle contractility in a subject in need thereof, comprising administering to the subject a HRS polypeptide or therapeutic composition provided herein.

### HRS Polypeptide Regulated Modulation of Sarcoidosis and Related Granulomatous Inflammatory Diseases

Sarcoidosis is a multisystem granulomatous inflammatory disease which is typically characterized by the formation of small, granular inflammatory lesions or granulomas (e.g., non-caseating granulomas) in a variety of organs, and/or the presence of immune responses (e.g., presence of CD4⁺ T lymphocytes and macrophages) in affected tissues or organs. Granulomatous inflammation may be attributed to the accumulation of monocytes, macrophages, a pronounced Th1 response and activated T-lymphocytes, with elevated production of TNFα, IL-2, IL-12, IFNγIL-1, IL-6 or IL-15.

Sarcoidosis can be systemic (e.g., systemic sarcoidosis) or local (e.g., localized sarcoid-like reactions). Granulomas can appear in almost any organ, and most often appear in the lungs or the lymph nodes. Other common sites include the liver, spleen, skin and eyes. The involvement of a specific organ may be mild or severe, self-limited or chronic, and limited or wide-ranging in extent. Symptoms usually appear gradually but can occasionally appear suddenly. The common symptoms experienced by sarcoidosis patients comprise fatigue, shortness of breath, cough that will not go away, skin lesions or skin rashes on face, arms, or shins, inflammation of the eyes, weight loss, night sweats, dyspnea, cough, chest discomfort, crackles. malaise, weakness, anorexia, weight loss, or fever. Other symptoms include, for example, enlarged lymph glands (armpit lump), enlarged liver, enlarged spleen, dry mouth or nosebleed. Symptoms of different types of sarcoidosis are described below.

The clinical course generally varies. In some embodiments, sarcoidosis can be asymptomatic. In some embodiments, a tissue or organ can remained inflamed or become scarred or fibrotic if one or more granulomas in the tissue or organ do not heal. In some embodiments, sarcoidosis can result in a debilitating chronic condition that may lead to death (e.g., irreversible pulmonary fibrosis).

In certain embodiments, said sarcoidosis is one or more of systemic sarcoidosis, cutaneous sarcoidosis, Lofgren's syndrome, neurosarcoidosis, pulmonary sarcoidosis, cardiac sarcoidosis, ocular sarcoidosis, hepatic sarcoidosis, musculoskeletal sarcoidosis, renal sarcoidosis, or sarcoidosis with the involvement of other organs or tissues.

Systemic sarcoidosis—is sarcoidosis with multiple organ involvement. In some embodiments, provided herein is a method of treating a subject having systemic sarcoidosis, comprising administering to the subject a HRS polypeptide, wherein said administration results in the detectable reduction of progression, detectable lessening of worsening, and/or detectable improvement, of one or more symptoms of said systemic sarcoidosis. In some embodiments, said one or more symptoms of systemic sarcoidosis comprise non-specific general symptoms such as weight loss, fatigue, loss of appetite, fever, chills, night sweats, formation of granulomas, fatigue, aches, pains or arthritis.

In some embodiments, systemic sarcoidosis can present with specific symptoms related to a particular organ affected (e.g., dry eyes, swelling of the knees, blurry vision, shortness of breath, cough, skin lesions such as rashes etc.). In specific embodiments, said specific symptom is one or more of a pulmonary, pulmonary lymphatic, musculoskeletal, hepatic, joint, hematologic, dermatologic, ocular, psychiatric, neurological, renal, splenic, neurologic, nasal sinus, cardiac, bone, oral, gastric, intestinal, endocrine, pleural or reproductive symptom.

Cutaneous sarcoidosis—is a complication of sarcoidosis with skin involvement. In some embodiments, provided herein is a method of treating a subject having cutaneous sarcoidosis, comprising administering to the subject an HRS polypeptide, wherein said administration results in the detectable reduction of progression, detectable lessening of worsening, and/or detectable improvement, of one or more symptoms of said cutaneous sarcoidosis.

In certain embodiments, the cutaneous sarcoidosis comprises annular sarcoidosis, erythrodermic sarcoidosis, ichthyosiform sarcoidosis, hypopigmented sarcoidosis, morpheaform sarcoidosis, mucosal sarcoidosis, papular sarcoid, scar sarcoid, subcutaneous sarcoidosis and ulcerative sarcoidosis. In some embodiments, the one or more symptoms of cutaneous sarcoidosis comprise a variety of skin lesions or conditions, either specific or non-specific (e.g., similar to several other skin conditions). Exemplary skin lesions or conditions associated with cutaneous sarcoidosis comprise papules (e.g., granulomatous rosacea, acne or benign appendageal tumors), skin plaques (e.g., psoriasis, lichen planus, nummular eczema, discoid lupus erythematosus, granuloma annulare, cutaneous T-cell lymphoma, Kaposi's sarcoma or secondary syphilis), lupus pernio (e.g., scar or discoid lupus erythematosus), erythema nodosum (e.g., raised, red, firm skin sores, cellulitis, furunculosis or other inflammatory panniculitis), maculopapular eruptions, nodular lesions deeper in the skin or infiltration of old scars. Other skin symptoms include, for example, skin rashes, old scars become more raised, skin lesions or hair loss.

Lofgren's syndrome—represents an acute presentation of systemic sarcoidosis, which is typically characterized by the triad of erythema nodosum, bilateral hilar denopathy and arthritis or arthralgias. It may also be accompanied by fever. In some embodiments, provided herein is a method of treating a subject having Lofgren's syndrome, comprising administering to the subject a HRS polypeptide, wherein said administration results in the detectable reduction of progression, detectable lessening of worsening, and/or detectable improvement, of one or more symptoms of said Lofgren's syndrome. In specific embodiments, the one or more symptoms comprise erythema nodosum, bilateral hilar denopathy, arthritis, arthralgias or fever.

Neurosarcoidosis or neurosarcoid—refers to sarcoidosis in which inflammation and abnormal deposits occur in the brain, spinal cord, and any other areas of the nervous system. In some embodiments, provided herein is a method of treating a subject having neurosarcoidosis, comprising administering to the subject a HRS polypeptide, wherein said administration results in the detectable reduction of progression, detectable lessening of worsening, and/or detectable improvement, of one or more symptoms of said neurosarcoidosis. Neurosarcoidosis may affect any part of the nervous system, for instance, nerves to the muscles of the face (cranial nerve VII), which may lead to symptoms of facial weakness (e.g., facial palsy), nerves in the eye or nerves that control taste, smell, or hearing.

In some embodiments, the symptoms of neurosarcoidosis comprise changes in menstrual periods, excessive tiredness (e.g., fatigue), headache, visual changes, retinopathy, radicular pain, loss of bowel or bladder control, carpal tunnel syndrome, and/or paraplegia, excessive thirst or high urine output. In some embodiments, the symptoms of neurosarcoidosis comprise confusion, disorientation, decreased hearing, dementia or delirium, dizziness or vertigo (e.g., abnormal sensation of movement), double vision or other vision problems, facial palsy (weakness, drooping), headache, loss of sense of smell or taste, abnormal tastes, psychiatric disturbances, seizures or speech impairment, muscle weakness or sensory losses, or in some occasions, hypopituiarism. In specific embodiments, the symptoms of neurosarcoidosis comprise formation of granulomas in the nervous system (e.g., brain, spinal cord, or facial and optic nerves), headache, confusion, malaise or facial paralysis.

Pulmonary sarcoidosis—refers to sarcoidosis that affects pulmonary tissues or organs (e.g., lungs). In some embodiments, provided herein is a method of treating a subject having pulmonary sarcoidosis, comprising administering to the subject a HRS polypeptide, wherein said administration results in the detectable reduction of progression, detectable lessening of worsening, and/or detectable improvement, of one or more symptoms of said pulmonary sarcoidosis. The symptoms of pulmonary sarcoidosis usually involve lung and/or chest symptoms, which can be determined by, for examples, lung gallium (Ga.) scan, chest X-ray, pulmonary function tests, exercise pulse oximetry, CT scan of chest, PET scan, CT-guided biopsy, mediastinoscopy, open lung biopsy or bronchoscopy with biopsy.

In certain embodiments, the symptoms of pulmonary sarcoidosis comprise granulomas in alveolar septa, bronchiolar, and bronchial walls, shortness of breath, cough, loss of lung volume and abnormal lung stiffness, abnormal or deteriorating lung function, decrease in lung volume, decreased compliance, scarring of lung tissue, or bleeding from the lung tissue. Other symptoms include, for example, limited amount of air drawn into the lungs, higher than normal expiratory flow ratios, decreased vital capacity (full breath in, to full breath out), increased FEA₁/PVC ratio, obstructive lung changes, which can cause a decrease in the amount of air that can be exhaled, or enlarged lymph nodes in the chest, which can compress airways or when internal inflammation or nodules impede airflow, pulmonary hypertension, or pulmonary failure.

Without being bound by any theory, the Scadding criteria are the measures most commonly used for disease staging of patients who have pulmonary sarcoidosis. Briefly, the radiographic evidence for each stage can be described as follows: Stage I: bilateral hilar and/or mediastinal lymphadenopathy (enlarged lymph nodes); Stage II: bilateral hilar and/or mediastinal lymphadenopathy; evidence of pulmonary infiltrates; Stage III: alterations in the parenchymal tissue; no lymph node enlargement; and Stage IV: Evidence of pulmonary fibrosis. Thus, provided herein is a method of treating a subject having pulmonary sarcoidosis or disease, disorder or condition caused by, or relating to pulmonary sarcoidosis, comprising administering to the subject a HRS polypeptide, wherein said administration results in the detectable reduction of progression, detectable lessening of worsening, and/or detectable improvement, of one or more symptoms of said pulmonary sarcoidosis as determined by the Scadding criteria.

In some embodiments, pulmonary sarcoidosis can develop into pulmonary fibrosis (e.g., irreversible pulmonary fibrosis), which can distort the structure of the lungs and impair breathing or bronchiectasis, a lung disease characterized by destruction and widening of the large airways. Thus, in a specific embodiment, said disease or disorder is pulmonary fibrosis or bronchietasis. In a more specific embodiment, provided herein is a method of treating a subject having pulmonary fibrosis (e.g., irreversible pulmonary fibrosis) caused by, or relating to sarcoidosis, comprising administering to the subject a HRS polypeptide, wherein said administration results in the detectable reduction of progression, detectable lessening of worsening, and/or detectable improvement, of one or more symptoms of said pulmonary fibrosis (e.g., irreversible pulmonary fibrosis). In another specific embodiment, provided herein is a method of treating a subject having bronchiectasis caused by, or relating to sarcoidosis, comprising administering to the subject a HRS polypeptide, wherein said administration results in the detectable reduction of progression, detectable lessening of worsening, and/or detectable improvement, of one or more symptoms of said bronchiectasis.

In some embodiments, sarcoidosis involves pulmonary lymphatic system such as hilar or mediastinal involvement, as determined by chest x-ray, and present with symptoms of nontender peripheral or cervical lymphadenopathy.

Cardiac sarcoidosis—refers to sarcoidosis with myocardial involvement. In some embodiments, provided herein is a method of treating a subject having cardiac sarcoidosis, comprising administering to the subject a HRS polypeptide, wherein said administration results in the detectable reduction of progression, detectable lessening of worsening, and/or detectable improvement, of one or more symptoms of said cardiac sarcoidosis.

In some embodiments, the one or more symptoms of cardiac sarcoidosis are similar to the symptoms of a disease or disorder of the circulatory system, for example, myocardial infarction, cardiomyopathy, aneurysm, angina, aortic stenosis, aortitis, arrhythmias, arteriosclerosis, arteritis, asymmetric septal hypertrophy (ASH), atherosclerosis, atrial fibrillation and flutter, bacterial endocarditis, Barlow's Syndrome (mitral valve prolapse), bradycardia, Buerger's Disease (thromboangiitis obliterans), cardiomegaly, carditis, carotid artery disease, coarctation of the aorta, congenital heart defects, congestive heart failure, coronary artery disease, Eisenmenger's Syndrome, embolism, endocarditis, erythromelalgia, fibrillation, fibromuscular dysplasia, heart block, heart murmur, hypertension, hypotension, idiopathic infantile arterial calcification, Kawasaki Disease (mucocutaneous lymph node syndrome, mucocutaneous lymph node disease, infantile polyarteritis), metabolic syndrome, microvascular angina, myocarditis, paroxysmal atrial tachycardia (PAT), periarteritis nodosa (polyarteritis, polyarteritis nodosa), pericarditis, peripheral vascular disease, critical limb ischemia, phlebitis, pulmonary valve stenosis (pulmonic stenosis), Raynaud's Disease, renal artery stenosis, renovascular hypertension, rheumatic heart disease, diabetic vasculopathy, septal defects, silent ischemia, syndrome X, tachycardia, Takayasu's Arteritis, Tetralogy of Fallot, transposition of the great vessels, tricuspid atresia, truncus arteriosus, valvular heart disease, varicose ulcers, varicose veins, vasculitis, ventricular septal defect, Wolff-Parkinson-White Syndrome, endocardial cushion defect, acute rheumatic fever, acute rheumatic pericarditis, acute rheumatic endocarditis, acute rheumatic myocarditis, chronic rheumatic heart diseases, diseases of the mitral valve, mitral stenosis, rheumatic mitral insufficiency, diseases of aortic valve, diseases of other endocardial structures, ischemic heart disease (acute and subacute), angina pectoris, acute pulmonary heart disease, pulmonary embolism, chronic pulmonary heart disease, kyphoscoliotic heart disease, myocarditis, endocarditis, endomyocardial fibrosis, endocardial fibroelastosis, atrioventricular block, cardiac dysrhythmias, myocardial degeneration, cerebrovascular disease, a disease of arteries, arterioles and capillaries, or a disease of veins and lymphatic vessels. Thus, in certain embodiments, improvement in a subject having sarcoidosis or a sarcoidosis-related disease or disorder, wherein the subject is administered a HRS polypeptide or therapeutic composition provided herein, can be assessed or demonstrated by detectable improvement in one or more symptoms of said sarcoidosis or said sarcoidosis-related disease or disorder.

In certain embodiments, the method of treating comprises administering a HRS polypeptide to said subject in an amount and for a time sufficient for detectable improvement of one or more indicia of cardiac function, wherein said indicia of cardiac function are chest cardiac output (CO), cardiac index (CI), pulmonary artery wedge pressure (PAWP), cardiac index (CI), % fractional shortening (% FS), ejection fraction (EF), left ventricular ejection fraction (LVEF); left ventricular end diastolic diameter (LVEDD), left ventricular end systolic diameter (LVESD), contractility (dP/dt), a decrease in atrial or ventricular functioning, an increase in pumping efficiency, a decrease in the rate of loss of pumping efficiency, a decrease in loss of hemodynamic functioning, or decrease in complications associated with cardiomyopathy, as compared to the subject prior to administration of said HRS polypeptide.

Ocular Sarcoidosis—is sarcoidosis that affects the eye. In some embodiments, provided herein is a method of treating a subject having ocular sarcoidosis, comprising administering to the subject a HRS polypeptide, detectable lessening of worsening, and/or detectable improvement, of one or more symptoms of said ocular sarcoidosis. In certain embodiments, the one or more symptoms of ocular sarcoidosis comprise uveitis (e.g., granulomatous uvetis), uveoparotitis, retinal inflammation, loss of visual acuity, blindness red, watery eyes, iris nodules, retinochoroiditis, conjunctivitis, lacrimal gland involvement or proptosis.

Given the role of NRP2 in modulating both immune cell activation and migration, as well as, the ability of HRS polypeptides to modulate these processes, HRS polypeptides represent a new therapeutic option for the treatment of sarcoidosis and related granulomatous inflammatory diseases. Certain embodiments therefore include methods of treating, ameliorating the symptoms of, and/or reducing the progression of, sarcoidosis in a subject in need thereof, comprising administering to the subject a HRS polypeptide or therapeutic composition provided herein.

Sarcoidosis with Musculoskeletal, Hepatic, Hematologic, Psychiatric, Renal, Splenic, Nasal Sinus, Oral, Gastric or Intestinal, Endocrine, Pleural or Reproductive Involvement

In certain embodiments, sarcoidosis can involve muscle, hepatic, joint, hematologic, psychiatric, renal, splenic, nasal sinus, bone, oral gastric or intestinal, endocrine, pleural or reproductive system and present with respective symptoms. In some embodiments, provided herein is a method of treating a subject having sarcoidosis with musculoskeletal, hepatic, joint, hematologic, psychiatric, renal, splenic, nasal sinus, oral gastric or intestinal, endocrine, pleural or reproductive involvement, comprising administering to the subject a HRS polypeptide, wherein said administration results in the detectable reduction of progression, detectable lessening of worsening, and/or detectable improvement, of one or more symptoms of said sarcoidosis.

In some embodiments, sarcoidosis with musculoskeletal involvement can be asymptomatic with or without enzyme elevations, or present with symptoms of, for example, insidious or acute myopathy with muscle weakness, arthritis (e g, ankle, knee, wrist, and elbow arthritis), chronic arthritis with Jaccoud's deformities or dactylitis, periarthritis, arthralgia, osteolytic or cystic lesions, osteopenia or Lofgren's syndrome. In some embodiments, sarcoidosis with hepatic involvement can be aymptomatic, or present with one or more symptoms such as mild elevations in liver function test results, hypolucent lesions on CT scans with radiopaque dye, hepatomegaly, changes in the liver enzyme levels, liver diseases, fever, malaise, fatigue, cholestasis, cirrhosis, or with symptoms similar to granulomatous hepatitis.

In some embodiments, patients with hematologic sarcoidosis have one or more symptoms of lymphopenia, anemia of chronic disease, anemia due to granulomatous infiltration of bone marrow, pancytopenia, splenic sequestration, thrombocytopenia or leucopenia. In some embodiments, patients with sarcoidosis can exhibit psychiatric symptoms (e.g., depression). In some embodiments, sarcoidosis with renal involvement can present with symptoms of asymptomatic hypercalciuria, interstitial nephritis, chronic renal failure caused by nephrolithiasis, or nephrocalcinosis. In some embodiments, sarcoidosis with splenic involvement can be asymptomatic, or present with symptoms of pain, thrombocytopenia or as determined by x-ray or CT scan. In some embodiments, sarcoidosis with nasal sinus involvement can present with symptoms of sinus mucosa with symptoms similar to common allergic and infectious sinusitis, or upus pernio. In some embodiments, sarcoidosis with oral involvement can present with symptoms of asymptomatic parotid swelling, parotitis with xerostomia, Heerfordt's syndrome, uveitis, bilateral parotid swelling, facial palsy, chronic fever, oral lupus pernio, or disfigured hard palate, cheek, tongue, and gums. In some embodiments, symptoms of sarcoidosis with gastric or intestinal, endocrine, pleural or reproductive involvement comprise gastric granulomas, mesenteric lymphadenopathy, abdominal pain, panhypopituitarism, thyroid infiltration, secondary hypoparathyroidism, hypercalcemia, or lymphocytic exudative effusions.

Also included are methods of treating a subject having sarcoidosis with the involvement of one or more tissues or organs other than pulmonary tissues or organs, comprising administering to the subject a HRS polypeptide or composition provided herein, wherein said administration results in the detectable reduction of progression, detectable lessening of worsening, and/or detectable improvement, of one or more symptoms of said sarcoidosis. Also included are methods of treating a subject having sarcoidosis with the involvement of one or more tissues or organs other than the lungs, comprising administering to the a HRS polypeptide or composition provided herein, wherein said administration results in the detectable reduction of progression, detectable lessening of worsening, and/or detectable improvement, of one or more symptoms of said sarcoidosis.

### HRS Polypeptide Mediated Treatment of Cancer

In some embodiments, the NRP2-associated disease is a cancer, for example, a cancer that expresses or overexpresses NRP2. In some instances, the cancer displays NRP2-dependent growth, NRP2-dependent adhesion, NRP2-dependent migration, NRP2-dependent chemoresistance, and/or NRP2-dependent invasion. In some embodiments, the cancer is a primary cancer. In some embodiments, the cancer is a metastatic cancer, optionally a metastatic cancer that expresses NRP2a and/or NRP2b.

In some embodiments, the cancer is chemoresistant to a cancer therapy, for example, a cancer immunotherapy agent, a chemotherapeutic agent, a hormonal therapeutic agent, and/or a kinase inhibitor. In some embodiments, the treatment method comprises selecting a subject having a cancer that is chemoresistant to at least one cancer therapy prior to administering the HRS polypeptide. Exemplary cancer immunotherapy agents, chemotherapeutic agents, hormonal therapeutic agents, and kinase inhibitors are described herein.

In some embodiments, the HRS polypeptide modulates autophagy, phagocyte maturation, or efferocytosis in a cancer cell or a cancer associated macrophage. In specific embodiments, the HRS polypeptide modulates autophagy in a cancer cell.

In some embodiments, the cancer is selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, and stomach cancer.

In some embodiments, the metastatic cancer is selected from one or more of:

(a) a bladder cancer which has metastasized to the bone, liver, and/or lungs;

(b) a breast cancer which has metastasized to the bone, brain, liver, and/or lungs;

(c) a colorectal cancer which has metastasized to the liver, lungs, and/or peritoneum;

(d) a kidney cancer which has metastasized to the adrenal glands, bone, brain, liver, and/or lungs;

(e) a lung cancer which has metastasized to the adrenal glands, bone, brain, liver, and/or other lung sites;

(f) a melanoma which has metastasized to the bone, brain, liver, lung, and/or skin/muscle;

(g) a ovarian cancer which has metastasized to the liver, lung, and/or peritoneum;

(h) a pancreatic cancer which has metastasized to the liver, lung, and/or peritoneum;

(i) a prostate cancer which has metastasized to the adrenal glands, bone, liver, and/or lungs;

(j) a stomach cancer which has metastasized to the liver, lung, and/or peritoneum;

(l) a thyroid cancer which has metastasized to the bone, liver, and/or lungs; and

(m) a uterine cancer which has metastasized to the bone, liver, lung, peritoneum, and/or vagina.

Certain cancer therapies include combination therapies, as described herein. For instance, certain embodiments include administering to the subject at least one additional agent selected from one or more of a cancer immunotherapy agent, a chemotherapeutic agent, a hormonal therapeutic agent, and a kinase inhibitor. In some embodiments, the at least one HRS polypeptide and the at least one agent are administered separately, as separate compositions. In some embodiments, the at least one HRS polypeptide and the at least one agent are administered together as part of the same therapeutic composition.

In some embodiments, the cancer immunotherapy agent is selected from one or more of an immune checkpoint modulatory agent, a cancer vaccine, an oncolytic virus, a cytokine, and a cell-based immunotherapies. In some embodiments, the immune checkpoint modulatory agent is a polypeptide, optionally an antibody or antigen-binding fragment thereof or a ligand, or a small molecule. In some embodiments, the immune checkpoint modulatory agent comprises

(a) an antagonist of a inhibitory immune checkpoint molecule; or

(b) an agonist of a stimulatory immune checkpoint molecule. for instance, wherein the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736), optionally wherein the cancer is selected from one or more of colorectal cancer, melanoma, breast cancer, non-small-cell lung carcinoma, bladder cancer, and renal cell carcinoma;

the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab, optionally wherein the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer;

the PD-1 antagonist is pembrolizumab and the cancer is optionally selected from one or more of melanoma, non-small cell lung cancer, small cell lung cancer, head and neck cancer, and urothelial cancer;

the antagonist is a CTLA-4 antagonist optionally selected from one or more of an antibody or antigen-binding fragment or small molecule that specifically binds thereto, ipilimumab, tremelimumab, optionally wherein the cancer is selected from one or more of melanoma, prostate cancer, lung cancer, and bladder cancer;

the antagonist is an IDO antagonist optionally selected from one or more of an antibody or antigen-binding fragment or small molecule that specifically binds thereto, indoximod (NLG-8189), 1-methyl-tryptophan (IMT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor;

the antagonist is a TDO antagonist optionally selected from one or more of an antibody or antigen-binding fragment or small molecule that specifically binds thereto, 680C91, and LM10;

the antagonist is a TIM-3 antagonist optionally selected from one or more of an antibody or antigen-binding fragment or small molecule that specifically binds thereto;

the antagonist is a LAG-3 antagonist optionally selected from one or more of an antibody or antigen-binding fragment or small molecule that specifically binds thereto, and BMS-986016;

the antagonist is a VISTA antagonist optionally selected from one or more of an antibody or antigen-binding fragment or small molecule that specifically binds thereto;

the antagonist is a BTLA, CD160, and/or HVEM antagonist optionally selected from one or more of an antibody or antigen-binding fragment or small molecule that specifically binds thereto;

the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agonist is an OX40 agonist optionally selected from one or more of an antibody or antigen-binding fragment or small molecule or ligand that specifically binds thereto, OX86, Fc-OX40L, and GSK3174998;

the agonist is a CD40 agonist optionally selected from one or more of an antibody or antigen-binding fragment or small molecule or ligand that specifically binds thereto, CP-870,893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma;

the agonist is a GITR agonist optionally selected from one or more of an antibody or antigen-binding fragment or small molecule or ligand that specifically binds thereto, INCAGN01876, DTA-1, and MEDI1873;

the agonist is a CD137 agonist optionally selected from one or more of an antibody or antigen-binding fragment or small molecule or ligand that specifically binds thereto, utomilumab, and 4-1BB ligand;

the agonist is a CD27 agonist optionally selected from one or more of an antibody or antigen-binding fragment or small molecule or ligand that specifically binds thereto, varlilumab, and CDX-1127 (1F5);

the agonist is a CD28 agonist optionally selected from one or more of an antibody or antigen-binding fragment or small molecule or ligand that specifically binds thereto, and TAB08; and/or

the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In some embodiments, the cytokine selected from one or more of interferon (IFN)-a, IL-2, IL-12, IL-7, IL-21, and Granulocyte-macrophage colony-stimulating factor (GM-CSF).

In some embodiments, the cell-based immunotherapy agent comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

In some embodiments, the at least one chemotherapeutic agent is selected from one or more of an alkylating agent, an anti-metabolite, a cytotoxic antibiotic, a topoisomerase inhibitor (type 1 or type II), and an anti-microtubule agent.

In some embodiments, the alkylating agent is selected from one or more of nitrogen mustards (optionally mechlorethamine, cyclophosphamide, mustine, melphalan, chlorambucil, ifosfamide, and busulfan), nitrosoureas (optionally N-Nitroso-N-methylurea (MNU), carmustine (BCNU), lomustine (CCNU), semustine (MeCCNU), fotemustine, and streptozotocin), tetrazines (optionally dacarbazine, mitozolomide, and temozolomide), aziridines (optionally thiotepa, mytomycin, and diaziquone (AZQ)), cisplatins and derivatives thereof (optionally carboplatin and oxaliplatin), and non-classical alkylating agents (optionally procarbazine and hexamethylmelamine);

the anti-metabolite is selected from one or more of anti-folates (optionally methotrexate and pemetrexed), fluoropyrimidines (optionally 5-fluorouracil and capecitabine), deoxynucleoside analogues (optionally ancitabine, enocitabine, cytarabine, gemcitabine, decitabine, azacitidine, fludarabine, nelarabine, cladribine, clofarabine, fludarabine, and pentostatin), and thiopurines (optionally thioguanine and mercaptopurine);

the cytotoxic antibiotic is selected from one or more of anthracyclines (optionally doxorubicin, daunorubicin, epirubicin, idarubicin, pirarubicin, aclarubicin, and mitoxantrone), bleomycins, mitomycin C, mitoxantrone, and actinomycin;

the topoisomerase inhibitor is selected from one or more of camptothecin, irinotecan, topotecan, etoposide, doxorubicin, mitoxantrone, teniposide, novobiocin, merbarone, and aclarubicin; and/or

the anti-microtubule agent is selected from one or more of taxanes (optionally paclitaxel and docetaxel) and vinca alkaloids (optionally vinblastine, vincristine, vindesine, vinorelbine).

In some embodiments, the at least one hormonal therapeutic agent is a hormonal agonist or a hormonal antagonist. In some embodiments, the hormonal agonist is selected from one or more of a progestogen (progestin), a corticosteroid (optionally prednisolone, methylprednisolone, or dexamethasone), insulin like growth factors, VEGF derived angiogenic and lymphangiogenic factors (optionally VEGF-A, VEGF-A145, VEGF-A165, VEGF-C, VEGF-D, PIGF-2), fibroblast growth factor (FGF), galectin, hepatocyte growth factor (HGF), platelet derived growth factor (PDGF), transforming growth factor (TGF)-beta, an androgen, an estrogen, and a somatostatin analog. In some embodiments, the hormonal antagonist is selected from one or more of a hormone synthesis inhibitor, optionally an aromatase inhibitor or a gonadotropin-releasing hormone (GnRH) or an analog thereof, and a hormone receptor antagonist, optionally a selective estrogen receptor modulator (SERM) or an anti-androgen, or an antibody directed against a hormonal receptor, optionally cixutumumab, dalotuzumab, figitumumab, ganitumab, istiratumab, robatumumab, alacizumab pegol, bevacizumab, icrucumab, ramucirumab, fresolimumab, metelimumab, naxitamab, cetuximab, depatuxizumab mafodotin, futuximab, imgatuzumab, laprituximab emtansine, matuzumab, modotuximab, necitumumab, nimotuzumab, panitumumab, tomuzotuximab, zalutumumab, aprutumab ixadotin, bemarituzumab, olaratumab, or tovetumab.

In some embodiments, the kinase inhibitor is selected from one or more of adavosertib, afanitib, aflibercept, axitinib, bevacizumab, bosutinib, cabozantinib, cetuximab, cobimetinib, crizotinib, dasatinib, entrectinib, erdafitinib, erlotinib, fostamitinib, gefitinib, ibrutinib, imatinib, lapatinib, lenvatinib, mubritinib, nilotinib, panitumumab, pazopanib, pegaptanib, ponatinib, ranibizumab, regorafenib, ruxolitinib, sorafenib, sunitinib, SU6656, tofacitinib, trastuzumab, vandetanib, and vemuafenib. In some embodiments, the kinase inhibitor is a PI3 kinase inhibitor selected from one or more of alpelisib, buparlisib, copanlisib, CUDC-907, dactolisib, duvelisib, GNE-477, idelasib, IPI-549, LY294002, ME-401, perifosine, PI-103, pictilisib, PWT33597, RP6503, taselisib, umbralisib, voxtalisib, wortmannin, and XL147

For the treatment of cancer, in some instances, an HRS polypeptide enhances the immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to an untreated control. Exemplary immune responses include increasing or enhancing immune cell invasion of a solid tumor, and increasing the biological activity against the cancer. In certain embodiments, an HRS polypeptide enhances an adaptive immune response to the cancer, and in some embodiments, an HRS polypeptide enhances an innate immune response to the cancer. In some-instances, an HRS polypeptide directly or indirectly enhances a T-cell-mediated response to the cancer. In some-instances, an HRS polypeptide enhances a B-cell-mediated or antibody-mediated response to the cancer. In some-instances, an HRS polypeptide modulates a macrophage responses to the cancer. In some-instances, an HRS polypeptide modulates immune cell, or cancer autophagy. In some-instances, an HRS polypeptide modulates immune cell phagocytosis. In some-instances, an HRS polypeptide modulates cancer cell apoptosis. In some-instances, an HRS polypeptide modulates immune cell efferocytosis and/or cancer cell autophagy.

In some embodiments, an HRS polypeptide enhances macrophage responses to the cancer. In some embodiments, an HRS polypeptide inhibits macrophage responses to the cancer. In some embodiments, an HRS polypeptide enhances autophagy. In some embodiments, an HRS polypeptide inhibits autophagy. In some embodiments, an HRS polypeptide enhances phagocytosis. In some embodiments, an HRS polypeptide inhibits phagocytosis. In some embodiments, an HRS polypeptide enhances apoptosis. In some embodiments an HRS polypeptide inhibits apoptosis. In some embodiments, an HRS polypeptide enhances efferocytosis. In some embodiments, an HRS polypeptide inhibits efferocytosis.

In some-instances, an HRS polypeptide reduces cancer initiation, cancer cell migration, adhesion, or cancer cell metastasis by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control. In some-instances, HRS polypeptide reduces cancer mediated lymphangiogenesis by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control.

In some embodiments, an HRS polypeptide reduces the rate of in vitro growth of the cancer (for example, cancer cells isolated from a biopsy or other sample grown in vitro) by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control.

In some embodiments, an HRS polypeptide reduces the adhesiveness of the cancer (for example, cancer cells isolated from a biopsy or other sample grown in vitro) to a substrate by about or at least about, 5, 10, 15, 20, 25, 30, 35, 40, 45, 50, 60, 70, 80, 90, 100, 200, 300, 400, 500, 600, 700, 800, 900, 1000, 2000% or more relative to an untreated control. In some instances, the substrate comprises laminin.

In some embodiments, an HRS polypeptide reduces the invasiveness of the cancer (for example, cancer cells isolated from a biopsy or other sample grown in vitro) by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control.

In some embodiments, an HRS polypeptide inhibits the rate of migration or motility of the cancer or a migratory cell (for example, cancer or immune cells isolated from a biopsy or other sample grown in vitro) by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control.

In some embodiments, an HRS polypeptide inhibits the rate of autophagy or endosome maturation (for example, endosome acidification) of the cancer or associated immune cells by about or at least about 5, 10, 15, 20, 25, 30, 35, 40, 45, 50, 60, 70, 80, 90, 100, 200, 300, 400, 500, 600, 700, 800, 900, 1000, 2000% or more relative to an untreated control.

In some embodiments, an HRS polypeptide enhances the susceptibility of the cancer to an additional agent (for example, chemotherapeutic agent, hormonal therapeutic agent, and or kinase inhibitor) by about or at least about 5, 10, 15, 20, 25, 30, 35, 40, 45, 50, 60, 70, 80, 90, 100, 200, 300, 400, 500, 600, 700, 800, 900, 1000, 2000% or more relative to the additional agent alone. In some embodiments, an HRS polypeptide enhances an anti-tumor and/or immunostimulatory activity of a cancer immunotherapy agent by about or at least about 5, 10, 15, 20, 25, 30, 35, 40, 45, 50, 60, 70, 80, 90, 100, 200, 300, 400, 500, 600, 700, 800, 900, 1000, 2000% or more, relative to the cancer immunotherapy agent alone.

In some embodiments, the methods and therapeutic compositions described herein increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and therapeutic compositions described herein increase median survival time of a subject by 1 year, 2 years, 3 years, or longer. In some embodiments, the methods and therapeutic compositions described herein increase progression-free survival by 2 weeks, 3 weeks, 4 weeks, 5 weeks, 6 weeks, 7 weeks, 8 weeks, 9 weeks, 10 weeks or longer. In certain embodiments, the methods or therapeutic compositions described herein increase progression-free survival by 1 year, 2 years, 3 years, or longer.

In certain embodiments, the methods and therapeutic compositions described herein are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein are sufficient to result in stable disease. In certain embodiments, the methods and therapeutic compositions described herein are sufficient to result in clinically relevant reduction in symptoms of a particular disease indication known to the skilled clinician.

In some embodiments, an HRS polypeptide increases, complements, or otherwise enhances the anti-tumor and/or immunostimulatory activity of the cancer immunotherapy agent, relative to the cancer immunotherapy agent alone. In some embodiments, an HRS polypeptide enhances the anti-tumor and/or immunostimulatory activity of the cancer immunotherapy agent by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to the cancer immunotherapy agent alone.

### Pharmaceutical Compositions and Kits

Certain embodiments include pharmaceutical compositions, therapeutic compositions, and formulations suitable for the therapeutic delivery of the HRS polypeptides/expressible polynucleotides, and optionally one or more second agents, as described herein. Some embodiments therefore include pharmaceutically-acceptable compositions that comprise a therapeutically-effective amount of one or more of the HRS polypeptides/expressible polynucleotides, and optionally one or more second agents, as described herein, formulated together with one or more pharmaceutically-acceptable carriers and/or diluents.

In some embodiments, as noted above, the second agent as selected from antimicrobial agents, antifungal agents, and antihelminthic agents, including combinations thereof. In some embodiments, the antimicrobial agent, antifungal agent, and/or antihelminthic agent is selected from one or more of aminoglycosides such as Amikacin, Gentamicin, Kanamycin, Neomycin, Netilmicin, Tobramycin, Paromomycin, Streptomycin, and Spectinomycin; carbapenems such as Ertapenem, Doripenem, Imipenem/Cilastatin, and Meropenem; cephalasporins such as Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Cefepime, Ceftaroline fosamil, and Ceftobiprole; glycopeptides such as Teicoplanin, Vancomycin, Telavancin, Dalbavancin, Oritavancin; lincosamines such as Clindamycin and Lincomycin; macrolides such as Azithromycin, Clarithromycin, Erythromycin, Roxithromycin, Telithromycin, and Spiramycin; penicillins such as Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; polypeptides such as Bacitracin, Colistin, and Polymyxin B; quinolones/fluoroquinolones such as Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; sulfonamides such as Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole) (TMP-SMX), and Sulfonamidochrysoidine; tetracyclines such as Demeclocycline, Doxycycline, Metacycline, Minocycline, Oxytetracycline, and Tetracycline; anti-mycobacterials such as Clofazimine, Dapsone, Capreomycin, Cycloserine, Ethambutol, Ethionamide, Isoniazid, Pyrazinamide, Rifampicin (Rifampin), Rifabutin, Rifapentine, and Streptomycin; Chloramphenicol; Metronidazole; Mupirocin; Tigecycline; Tinidazole; and antihelminthic agents such as Diethylcarbamazine and Albendazole.

In some embodiments, the second agent is selected from a cancer immunotherapy agent, a chemotherapeutic agent, a hormonal therapeutic agent, and a kinase inhibitor, as described herein.

The compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) nasally; or (9) via bladder instillation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject.

Some examples of materials that can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Additional non-limiting examples of agents suitable for formulation with the HRS polypeptides/expressible polynucleotides and other agents include: PEG conjugated nucleic acids, phospholipid conjugated nucleic acids, nucleic acids containing lipophilic moieties, phosphorothioates, P-glycoprotein inhibitors (such as Pluronic P85) which can enhance entry of drugs into various tissues; biodegradable polymers, such as poly (DL-lactide-coglycolide) microspheres for sustained release delivery after implantation (Emerich, D F et al., 1999, Cell Transplant, 8, 47-58) Alkermes, Inc. Cambridge, Mass.; and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999).

Also included are compositions comprising surface-modified liposomes containing poly (ethylene glycol) lipids (PEG-modified, branched and unbranched or combinations thereof, or long-circulating liposomes or stealth liposomes). HRS polypeptides/expressible polynucleotides and/or other agents can also comprise covalently attached PEG molecules of various molecular weights. These formulations offer a method for increasing the accumulation of drugs in target tissues. Long-circulating liposomes are also likely to protect drugs from nuclease degradation to a greater extent compared to cationic liposomes, based on their ability to avoid accumulation in metabolically aggressive MPS tissues such as the liver and spleen.

Also included are compositions prepared for delivery as described in U.S. Pat. Nos. 6,692,911, 7,163,695 and 7,070,807. In this regard, certain embodiments include compositions comprising copolymers of lysine and histidine (HK) as described in U.S. Pat. Nos. 7,163,695, 7,070,807, and 6,692,911 either alone or in combination with PEG (e.g., branched or unbranched PEG or a mixture of both), in combination with PEG and a targeting moiety or any of the foregoing in combination with a crosslinking agent. Some embodiments provide HRS polypeptides/expressible polynucleotides and/or other agents in compositions comprising gluconic-acid-modified polyhistidine or gluconylated-polyhistidine/transferrin-polylysine. One skilled in the art will also recognize that amino acids with properties similar to His and Lys may be substituted within the composition.

Certain agents described herein may contain a basic functional group, such as amino or alkylamino, which is capable of forming pharmaceutically-acceptable salts with pharmaceutically-acceptable acids. The term “pharmaceutically-acceptable salts” in this respect, refers to the relatively non-toxic, inorganic and organic acid addition salts of an agent. These salts can be prepared in situ in the administration vehicle or the dosage form manufacturing process, or by separately reacting a purified agent in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

The pharmaceutically acceptable salts of the agents described herein include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain embodiments, the agents described herein contain one or more acidic functional groups and, thus, are capable of forming pharmaceutically acceptable salts with pharmaceutically acceptable bases. The term “pharmaceutically-acceptable salts” in these instances refers to the relatively non-toxic, inorganic and organic base addition salts of an agent. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of include those suitable for intravenous, intramuscular, oral, nasal, pulmonary, topical (including buccal and sublingual), rectal, vaginal, and/or parenteral administration. The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a composition or formulation comprises an excipient selected from cyclodextrins, celluloses, liposomes, micelle forming agents, e.g., bile acids, and polymeric carriers, e.g., polyesters and polyanhydrides; and an HRS polypeptide/expressible polynucleotide and any other agents.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an HRS polypeptide/expressible polynucleotide and/or other agent as an active ingredient. The compositions or agents may also be administered as a bolus, electuary, or paste.

In solid dosage forms for oral administration (capsules, tablets, pills, dragees, powders, granules, trouches and the like), the active ingredient may be mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (e.g., gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the HRS polypeptides/expressible polynucleotides and other agents include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations or dosage forms for the topical or transdermal administration of the HRS polypeptides/expressible polynucleotides and other agents include powders, sprays, ointments, pastes, creams, lotions, gels, solutions, patches and inhalants. The active HRS polypeptides/expressible polynucleotides and/or other agents may be mixed under sterile conditions with a pharmaceutically-acceptable carrier, and with any preservatives, buffers, or propellants which may be required. The ointments, pastes, creams and gels may contain, in addition to the HRS polypeptides/expressible polynucleotides and/or other agents, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of the HRS polypeptides/expressible polynucleotides and/or other agents to the body. Such dosage forms can be made by dissolving or dispersing the agent in the proper medium. Absorption enhancers can also be used to increase the flux of the agent across the skin. The rate of such flux can be controlled by either providing a rate controlling membrane or dispersing the agent in a polymer matrix or gel, among other methods known in the art.

Pharmaceutical compositions suitable for parenteral administration may comprise one or more HRS polypeptides/expressible polynucleotides and/or other agents in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility, among other methods known in the art. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms may be made by forming microencapsule matrices of the subject HRS polypeptides/expressible polynucleotides and/or other agents in biodegradable polymers such as polylactide-polyglycolide. Depending on the ratio of agent to polymer, and the nature of the particular polymer employed, the rate of release can be controlled. Examples of other biodegradable polymers include poly(orthoesters) and poly(anhydrides). Depot injectable formulations may also prepared by entrapping the drug in liposomes or microemulsions that are compatible with body tissues.

When the HRS polypeptides/expressible polynucleotides and/or other agents are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Regardless of the route of administration selected, the HRS polypeptides/expressible polynucleotides and/or other agents may be formulated into pharmaceutically-acceptable dosage forms by conventional methods known to those of skill in the art. Actual dosage levels of the active ingredients in the pharmaceutical compositions may be varied so as to obtain an amount of the active ingredient which is effective to achieve the desired therapeutic response for a particular patient, composition, and mode of administration, without being unacceptably toxic to the patient.

The selected dosage level will depend upon a variety of factors including the activity of the particular HRS polypeptides/expressible polynucleotides and/or other agents employed, or the ester, salt or amide thereof, the route of administration, the time of administration, the rate of excretion or metabolism of the particular agent being employed, the rate and extent of absorption, the duration of the treatment, other drugs, compounds and/or materials used in combination with the particular agent employed, the age, sex, weight, condition, general health and prior medical history of the patient being treated, and like factors well known in the medical arts.

A physician having ordinary skill in the art can readily determine and prescribe the effective amount of the pharmaceutical composition required. For example, the physician could start doses of the HRS polypeptides/expressible polynucleotides and/or other agents employed in the pharmaceutical composition at levels lower than that required in order to achieve the desired therapeutic effect and gradually increase the dosage until the desired effect is achieved. In general, a suitable daily dose of HRS polypeptides/expressible polynucleotides and/or other agents will be that amount of the compound which is the lowest dose effective to produce a therapeutic effect. Such an effective dose will generally depend upon the factors described herein. Generally, oral, intravenous, intramuscular, intracerebroventricular and subcutaneous doses of the HRS polypeptides/expressible polynucleotides and/or other agents for a subject or patient, when used for the indicated effects, will range from about 0.0001 to about 100 mg per dosage, or about about 0.0001 to about 100 mg per kilogram of body weight per dosage.

If desired, the effective daily dose of the active agent(s) may be administered as one, two, three, four, five, six or more sub-doses administered separately at appropriate intervals throughout the day or week, for example, in unit dosage forms. In certain situations, dosing is one administration per day. In certain situations, dosing is one, two, or three administration per week. In certain embodiments, dosing is one or more administration per every 2, 3, 4, 5, 6, 7, 8, 9, 10, 11, 12, 13, 14 days, or every 1, 2, 3, 4, 5, 6, 7, 8, 9, 10, 11, 12 weeks, or every 1, 2, 3, 4, 5, 6, 7, 8, 9, 10, 11, 12 months, as needed, to treat the desired condition.

HRS polypeptides/expressible polynucleotides and/or other agents can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres, as described herein and known in the art. In certain embodiments, microemulsification technology may be utilized to improve bioavailability of lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other benefits, microemulsification provides enhanced bioavailability by preferentially directing absorption to the lymphatic system instead of the circulatory system, which thereby bypasses the liver, and prevents destruction of the compounds in the hepatobiliary circulation.

In some embodiments, the compositions or formulations contain micelles which are formed from the HRS polypeptides/expressible polynucleotides and/or other agents and at least one amphiphilic carrier, in which the micelles have an average diameter of less than about 100 nm. Exemplary embodiments provide micelles having an average diameter less than about 50 nm, and even certain embodiments provide micelles having an average diameter less than about 30 nm, or even less than about 20 nm. While all suitable amphiphilic carriers are contemplated, the presently preferred carriers are generally those that have Generally-Recognized-as-Safe (GRAS) status, and that can both solubilize the active ingredient and microemulsify it at a later stage when the solution comes into a contact with a complex water phase (such as one found in human gastro-intestinal tract). Usually, amphiphilic ingredients that satisfy these requirements have HLB (hydrophilic to lipophilic balance) values of 2-20, and their structures contain straight chain aliphatic radicals in the range of C-6 to C-20. Examples are polyethylene-glycolized fatty glycerides and polyethylene glycols.

Examples of amphiphilic carriers include saturated and monounsaturated polyethyleneglycolyzed fatty acid glycerides, such as those obtained from fully or partially hydrogenated various vegetable oils. Such oils may advantageously consist of tri-, di-, and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers may be particularly useful, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

In certain embodiments, the delivery may occur by use of liposomes, nanocapsules, microparticles, microspheres, lipid particles, vesicles, and the like, for the introduction of the HRS polypeptides/expressible polynucleotides and/or other agents into suitable host cells. In particular, the compositions may be formulated for delivery either encapsulated in a lipid particle, a liposome, a vesicle, a nanosphere, a nanoparticle or the like. The formulation and use of such delivery vehicles can be carried out using known and conventional techniques.

Hydrophilic polymers suitable for use are those which are readily water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. In certain embodiments, polymers have a molecular weight of from about 100 or 120 daltons up to about 5,000 or 10,000 daltons, or from about 300 daltons to about 5,000 daltons. In some embodiments, the polymer is polyethyleneglycol having a molecular weight of from about 100 to about 5,000 daltons, or having a molecular weight of from about 300 to about 5,000 daltons. In certain embodiments, the polymer is polyethyleneglycol of 750 daltons (PEG(750)). Polymers may also be defined by the number of monomers therein.

Other hydrophilic polymers which may be suitable include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a composition or formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

Cyclodextrins are cyclic oligosaccharides, consisting of 6, 7 or 8 glucose units, designated by the Greek letter α, β, and γ, respectively. The glucose units are linked by α-1,4-glucosidic bonds. As a consequence of the chair conformation of the sugar units, all secondary hydroxyl groups (at C-2, C-3) are located on one side of the ring, while all the primary hydroxyl groups at C-6 are situated on the other side. As a result, the external faces are hydrophilic, making the cyclodextrins water-soluble. In contrast, the cavities of the cyclodextrins are hydrophobic, since they are lined by the hydrogen of atoms C-3 and C-5, and by ether-like oxygens. These matrices allow complexation with a variety of relatively hydrophobic compounds, including, for instance, steroid compounds such as 17α-estradiol (see, e.g., van Uden et al. Plant Cell Tiss. Org. Cult. 38:1-3-113 (1994)). The complexation takes place by Van der Waals interactions and by hydrogen bond formation. The physico-chemical properties of the cyclodextrin derivatives depend on the kind and the degree of substitution. For example, their solubility in water ranges from insoluble (e.g., triacetyl-beta-cyclodextrin) to 147% soluble (w/v) (G-2-beta-cyclodextrin). In addition, they are soluble in many organic solvents. The properties of the cyclodextrins enable the control over solubility of various formulation components by increasing or decreasing their solubility.

Numerous cyclodextrins and methods for their preparation have been described. For example, Parmeter (I), et al. (U.S. Pat. No. 3,453,259) and Gramera, et al. (U.S. Pat. No. 3,459,731) described electroneutral cyclodextrins. Other derivatives include cyclodextrins with cationic properties [Parmeter (II), U.S. Pat. No. 3,453,257], insoluble crosslinked cyclodextrins (Solms, U.S. Pat. No. 3,420,788), and cyclodextrins with anionic properties [Parmeter (III), U.S. Pat. No. 3,426,011]. Among the cyclodextrin derivatives with anionic properties, carboxylic acids, phosphorous acids, phosphinous acids, phosphonic acids, phosphoric acids, thiophosphonic acids, thiosulphinic acids, and sulfonic acids have been appended to the parent cyclodextrin [see, Parmeter (III), supra]. Furthermore, sulfoalkyl ether cyclodextrin derivatives have been described by Stella, et al. (U.S. Pat. No. 5,134,127).

Some embodiments relate to formulations comprising liposomes containing HRS polypeptides/expressible polynucleotides and/or other agents, where the liposome membrane is formulated to provide a liposome with increased carrying capacity. Alternatively or in addition, the active ingredients may be contained within, or adsorbed onto, the liposome bilayer of the liposome. The HRS polypeptides/expressible polynucleotides and/or other agents may be aggregated with a lipid surfactant and carried within the liposome's internal space; in these cases, the liposome membrane is formulated to resist the disruptive effects of the active agent-surfactant aggregate.

Liposomes consist of at least one lipid bilayer membrane enclosing an aqueous internal compartment. Liposomes may be characterized by membrane type and by size. Small unilamellar vesicles (SUVs) have a single membrane and typically range between 0.02 and 0.05 μm in diameter; large unilamellar vesicles (LUVS) are typically larger than 0.05 μm. Oligolamellar large vesicles and multilamellar vesicles have multiple, usually concentric, membrane layers and are typically larger than 0.1 μm. Liposomes with several nonconcentric membranes, i.e., several smaller vesicles contained within a larger vesicle, are termed multivesicular vesicles.

In some embodiments, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Liposomes maybe prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993. For example, liposomes may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art.

In another exemplary formulation procedure, the HRS polypeptides/expressible polynucleotides and/or other agents are first dispersed by sonication in a lysophosphatidylcholine or other low CMC surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques as are known in the art, and the resulting liposomes separated from the unencapsulated solution by standard column separation.

In one aspect, the liposomes are prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323 (Apr. 12, 1988). In certain embodiments, reagents such as DharmaFECT™ and Lipofectamine™ may be utilized to introduce polynucleotides or proteins into cells.

The release characteristics of a formulation depend on the encapsulating material, the concentration of encapsulated drug, and the presence of release modifiers. For example, release can be manipulated to be pH dependent, for example, using a pH sensitive coating that releases only at a low pH, as in the stomach, or a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore forming agents, which can increase water uptake or release of drug by diffusion from the capsule. Excipients which modify the solubility of the drug can also be used to control the release rate. Agents which enhance degradation of the matrix or release from the matrix can also be incorporated. They can be added to the drug, added as a separate phase (i.e., as particulates), or can be co-dissolved in the polymer phase depending on the compound. In most cases the amount should be between 0.1 and thirty percent (w/w polymer). Types of degradation enhancers include inorganic salts such as ammonium sulfate and ammonium chloride, organic acids such as citric acid, benzoic acid, and ascorbic acid, inorganic bases such as sodium carbonate, potassium carbonate, calcium carbonate, zinc carbonate, and zinc hydroxide, and organic bases such as protamine sulfate, spermine, choline, ethanolamine, diethanolamine, and triethanolamine and surfactants such as Tween™ and Pluronic™. Pore forming agents which add microstructure to the matrices (i.e., water soluble compounds such as inorganic salts and sugars) are added as particulates. The range is typically between one and thirty percent (w/w polymer).

Uptake can also be manipulated by altering residence time of the particles in the gut. This can be achieved, for example, by coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

The HRS polypeptides/expressible polynucleotides and/or other agents may be formulated to be contained within, or, adapted to release by a surgical or medical device or implant. In certain aspects, an implant may be coated or otherwise treated with an agent. For example, hydrogels, or other polymers, such as biocompatible and/or biodegradable polymers, may be used to coat an implant with the HRS polypeptides/expressible polynucleotides and/or other agents (i.e., the composition may be adapted for use with a medical device by using a hydrogel or other polymer). Polymers and copolymers for coating medical devices with an agent are well-known in the art. Examples of implants include, but are not limited to, stents, drug-eluting stents, sutures, prosthesis, vascular catheters, dialysis catheters, vascular grafts, prosthetic heart valves, cardiac pacemakers, implantable cardioverter defibrillators, IV needles, devices for bone setting and formation, such as pins, screws, plates, and other devices, and artificial tissue matrices for wound healing. In some embodiments such coatings would serve to prevent granuloma formation around the implant.

The HRS polypeptides/expressible polynucleotides and/or other agents may be administered in any convenient vehicle which is physiologically acceptable. Such a composition may include any of a variety of standard pharmaceutically acceptable carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration.

Also included are kits, for example, patient care kits, comprising one or more containers filled with one or more of the therapeutic compositions, HRS polypeptides/expressible polynucleotides and/or other agents described herein. In some embodiments, the kits include written instructions on how to use such compositions, for example, in the treatment of one or more diseases.

Certain embodiments therefore include a patient care kit, comprising: (a) a histidyl-tRNA synthetase (HRS) polypeptide, or an expressible polynucleotide that encodes the HRS polypeptide; and (b) a second agent, for example, an antimicrobial agent, an anti-fungal agent, an antihelminthic agent, a cancer immunotherapy agent, a chemotherapeutic agent, a hormonal therapeutic agent, and/or a kinase inhibitor, as described herein. In some kits, (a) and (b) are in separate compositions, and are optionally defined as described herein. In some kits, (a) and (b) are in the same composition, optionally as a therapeutic composition as described herein.

The kits and compositions described herein may also include a one or more additional therapeutic agents or other components suitable or desired for the indication being treated. An additional therapeutic agent may be contained in a second container, if desired. Examples of additional therapeutic agents include, but are not limited to anti-inflammatory agents, anticancer agents, antibacterial agents, antiviral agents, etc.

The kits herein can also include one or more syringes (e.g., injectable syringes) or other components necessary or desired to facilitate an intended mode of delivery (e.g., stents, implantable depots, etc.).

### EXAMPLES

### Materials and Methods

ELISA Assays

Assays to measure human and mouse endogenous HRS in circulation. ELISA detection assays were developed to quantitate the levels of mouse or human HRS in circulation using different capture and detection antibodies to enable selective measurement of full length HRS as well as the N-terminal region.

The Human N-terminal ELISA is designed to detect the N-terminal domain of human HRS (WHEP domain) utilizing capture and detection antibodies targeting this domain (approximately amino acids 1-60 of HRS).

ELISA assays were conducted using a 96 well Multi-Array plate coated with capture antibody, following standard Meso Scale Diagnostics ELISA protocols, and using the following reagents:

| | |
|---|---|
| Block buffer: | Casein (Thermo Scientific #37528) |
| Wash buffer: | PBST (0.05% Tween-20 in IX PBS; |
| | made in-house) |
| Diluent: | 1% BSA (diluted in PBS) and Casein |
| Capture antibody: | ATYR12H6, mouse monoclonal antibody |
| Capture antibody cone: | 1 μg/mL |
| Protein standard range: | 100-0.046 ng/mL |
| Detection antibody: | 1C8-b, biotinylated mouse monoclonal |
| | antibody, |
| Detection antibody cone: | 0.5 μg/mL |
| Secondary reagent: | Streptavidin SULFO-TAG, # R32AD-1, |
| | 500 μg/mL |
| Secondary reagent cone: | 1 μg/mL |
| Substrate: | MSD Read Buffer T (4X) with |
| | Surfactant #R92TC-1 |

The Human full-length HRS ELISA is designed to detect the multi-domain human HARS utilizing capture and detection antibodies targeting separate domains of the protein (WHEP (amino acids 1-60 of HRS) and catalytic domains (approximately amino acids 60-398 of HRS).

ELISA assays were conducted using a 96 well Multi-Array plate coated with capture antibody, following standard Meso Scale Diagnostics ELISA protocols, and using the following reagents:

| | |
|---|---|
| Block buffer: | Casein (Thermo Scientific #37528) |
| Wash buffer: | PBST (0.05% Tween-20 in IX PBS; made in-house) |
| Diluent: | 1% BSA (diluted in PBS) and Casein |
| Capture antibody: | ATYR12H6, mouse monoclonal antibody |
| Capture antibody cone: | 1 μg/mL |
| Protein standard range: | 100-0.046 ng/mL |
| Detection antibody: | ATYR13C8-b, biotinylated mouse monoclonal |
| | antibody |
| Detection antibody cone: | 0.5 μg/mL |
| Secondary reagent: | Streptavidin SULFO-TAG, # R32AD-1, |
| | 500 μg/mL |
| Secondary reagent cone: | 1 μg/mL |
| Substrate: | MSD Read Buffer T (4X) with |
| | Surfactant #R92TC-1 |

The Mouse N-terminal ELISA is designed to detect the N-terminal domain of mouse HRS (WHEP) utilizing capture and detection antibodies targeting this domain.

ELISA assays were conducted using a 96 well Multi-Array plate coated with capture antibody, following standard Meso Scale Diagnostics ELISA protocols, and using the following reagents:

| | |
|---|---|
| Block buffer: | Casein (Thermo Scientific #37528) |
| Wash buffer: | PBST (0.05% Tween-20 in IX PBS; |
| | made in-house) |
| Diluent: | 1% BSA (diluted in PBS) and Casein |
| Capture antibody: | ATYR13E9, mouse monoclonal antibody |
| Capture antibody cone: | 1 μg/mL |
| Protein standard range: | 100-0.046 ng/mL |
| Detection antibody: | lC8-b, biotinylated mouse monoclonal antibody, |
| Detection antibody cone: | 0.5 μg/mL |
| Secondary reagent: | Streptavidin SULFO-TAG, # R32AD-1, |
| | 500 μg/mL |
| Secondary reagent cone: | 1 μg/mL |
| Substrate: | MSD Read Buffer T (4X) with |
| | Surfactant #R92TC-1 |

The Mouse full-length HRS ELISA designed to detect the multi-domain mouse HARS utilizing capture and detection antibodies targeting separate domains of the protein (WHEP (approximately amino acids 1-60 of HRS) and catalytic domains (approximately amino acids 60-398 of HRS).

ELISA assays were conducted using a 96 well Multi-Array plate coated with capture antibody, following standard Meso Scale Diagnostics ELISA protocols, and using the following reagents:

| Block buffer: | Casein (Thermo Scientific #37528) | |
|---|---|---|
| Wash buffer: | PBST (0.05% Tween-20 in 1× PBS; | |
| | made in-house) | |
| Diluent: | 1% BSA (diluted in PBS) and Casein | |
| Capture antibody: | ATYR13E9, mouse monoclonal | |
| Capture antibody conc: | 1 | μg/mL |
| Protein standard range: | 100-0.046 | ng/mL |
| Detection antibody: | ATYR13C8-b, mouse monoclonal | |
| Detection antibody conc: | 0.5 | μg/mL |
| Secondary reagent: | Streptavidin SULFO-TAG, | |
| | # R32AD-1, 500 μg/mL | |
| Secondary reagent conc: | 1 | μg/mL |

| | |
|---|---|
| Substrate: | MSD Read Buffer T (4×) with |
| | Surfactant #R92TC-1 |

Assays to measure human endogenous Neuropilin 2 (NRP2, or NP2) in circulation. An ELISA detection assay was developed to quantitate the levels of human NRP2 in circulation using capture and detection antibodies to enable selective measurement of soluble NRP2. The human NRP2 ELISA was designed to detect soluble NRP2 utilizing a monoclonal capture antibody and a polyclonal detection antibody targeting NRP2. ELISA assays were conducted using a 96 well Multi-Array plate, following standard Meso Scale Diagnostics ELISA protocols, and using the following reagents:

| | |
|---|---|
| Block buffer: | Casein (Thermo Scientific #37528) |
| Wash buffer: | PBST (0.05% Tween-20 in 1× PBS; |
| | made in-house) |
| Diluent: | 1% BSA (diluted in PBS) and Casein |
| Capture antibody: | NRP2 mAb Cat. #MAB2215, R&D Systems |
| Capture antibody conc: | 2 μg/mL |
| Protein standard: | NRP2 Fc Cat. #2215-N2-025, R&D Systems |
| Protein standard range: | 100-0.046 ng/mL |
| Detection antibody: | NRP2 pAb Cat. #BAF2215, R&D Systems |
| Detection antibody conc: | 0.5 μg/mL |
| Secondary reagent: | Streptavidin SULFO-TAG, |
| | # R32AD-1, 500 μg/mL |
| Secondary reagent conc: | 1 μg/mL |
| Substrate: | MSD Read Buffer T (4×) with Surfactant |
| | #R92TC-1 |

Assays to measure human endogenous HRS & NRP-2 complexes in circulation. An ELISA detection assay was developed to measure levels of human HRS & NRP2 complexes in circulation using capture and detection antibodies specific to each protein partner. The human HRS & NRP-2 complex ELISA is designed to detect a complex of soluble NRP2 and HRS utilizing monoclonal and polyclonal antibodies specific for these two proteins. ELISA assays were conducted using a 96 well Multi-Array plate, following standard Meso Scale Diagnostics ELISA protocols, and using the following reagents:

| | |
|---|---|
| Block buffer: | Casein (Thermo Scientific #37528) |
| Wash buffer: | PBST (0.05% Tween-20 in 1× PBS; |
| | made in-house) |
| Diluent: | 1% BSA (diluted in PBS) and Casein |
| Capture antibodies: | NRP2 mAb Cat. #MAB22151, R&D Systems |
| | HRS C-terminal mAb Clone#ATYR13C8 |
| | HRS N-terminal mAb Clone #ATYR12H6 |
| Capture antibody conc: | 1 μg/mL |
| Detection antibodies: | NRP2 pAb Cat. #BAF2215, R&D Systems |
| | HRS C-terminal mAb Clone#13C8 |
| | HRS N-terminal mAb Clone #12H6 |
| Detection antibody conc: | 0.5 ug/mL |
| Secondary reagent: | Streptavidin SULFO-TAG, |
| | # R32AD-1, 500 μg/mL |
| Secondary reagent conc: | 1 μg/mL |
| Substrate: | MSD Read Buffer T (4×) with Surfactant |
| | #R92TC-1 |

Antibody characterization Studies. Surface plasmon resonance (SPR) methods were used to characterize the binding kinetics and affinities of the antibodies towards HRS proteins. SPR experiments were conducted on a Bio-Rad ProteOn XPR36 Protein Interaction Array instrument. HRS proteins were immobilized on different channels of a ProteOn GLC sensor chip through amine coupling. Each antibody at a series of different concentrations was flowed over the immobilized proteins. The sensor chip surface was regenerated between each antibody run to remove bound antibodies. The resulting sensograms were analyzed in the ProteOn Manager Software, and fitted globally to a bivalent analyte model to obtain on-rates (kₐ) and off-rates (k_{d}). The equilibrium dissociation constant (K_{D}) for each antibody-protein pair is the ratio of k_{d}/kₐ.

| | |
|---|---|
| Running buffer: | 1× PBS with 0.005% Tween-20 |
| Amine coupling: | ProteOn Amine Coupling Kit |
| | (Bio-Rad #1762410) |
| Ligand coupling buffer: | Sodium acetate pH 5.5 |
| Regeneration buffer: | 10 mM HCl |

Protein-Protein Interaction studies. Surface plasmon resonance (SPR) methods were used to demonstrate interactions between protein partners. SPR experiments were conducted on a Bio-Rad ProteOn XPR36 Protein Interaction Array instrument. Proteins were immobilized on different channels of ProteOn GLC sensor chips through amine coupling. Analyte proteins were flowed over the immobilized proteins. The sensor chip surface was regenerated between each analyte run to remove interacting proteins. Data was double referenced against an interspot (untreated chip surface) and a blank surface (activated and deactivated for amine coupling).

| | |
|---|---|
| Running buffer: | 50 mM HEPES, 300 mM NaCl, 5 mM CaCl₂, 0.005% |
| | Tween-20, pH 7.4 |
| Amine coupling: | ProteOn Amine Coupling Kit (Bio-Rad #1762410) |
| Ligand coupling buffer: | Sodium acetate (pH 4.0, 4.5, 5.0, 5.5 depending on pI of protein) |
| Regeneration buffer: | 10 mM glycine pH 2.0 |

Commercial proteins reagents (proteins are derived from human sequences unless noted):
NRP2-Fc (R&D Systems #2215-N2)
NRP1-His (R&D Systems #3870-N1)
Mouse PLXNA1-His (R&D Systems #4309-PA)
SEMA3C-Fc (R&D Systems #5570-S3)
Mouse SEMA3F-Fc (R&D Systems #3237-S3)
Mouse NRP2-Fc (R&D Systems #7988-N2)
Rat NRP2-Fc-His (R&D Systems #567-N2)
VEGF-C (R&D Systems #9199-VC/CF)
VEGF-A₁₆₅ (Peprotech #100-20)
VEGF-A₁₄₅ (R&D Systems #7626-VE-CF)
VEGF-A₁₂₁ (Peprotech #100-20A)
PlGF-2 (Peprotech #100-56)
Heparin (StemCell #07980)

Immunofluorescence Assays on Cultured Cells.

Reagents:
PE anti-human IgG Fc Antibody, clone HP6017, Biolegend cat 409304
Mouse Anti-HRS monoclonal antibody (1-96) Clone 1C8, Abnova cat H00003035-M01
Mouse IgG2a, Isotype control antibody (MOPC-173), Biolegend cat 400223
Recombinant Human VEGF-C Protein, R&D Systems, cat 2179-VC-025/CF
Formaldehyde, 16%, methanol free, Ultra Pure, Polysciences, cat 18814-10
Hoechst 33342, Trihydrochloride, Trihydrate, ThermoFisher Scientific, cat H1399
Fc-HRS (2-60) was prepared as described in PCT application PCT/US2014/029699
Gibco DMEM, High Glucose, ThermoFisher Scientific, cat 11965092
PolyJet™ In Vitro DNA Transfection Reagent, SignaGen, cat SL100688
Neuropilin 2 (NRP2) (NM_003872) Human ORF Clone, Origene, cat RG220706
Collagen Coating Solution, Cell Applications Cat 125-100
1×PBS containing 1% BSA, 0.9 mM CaCl₂ and 20 mM glucose
Binding buffer containing 1% normal mouse IgG (sigma cat 18765) and 2.5% (Human Fc Receptor Binding Inhibitor, ebioscience 14-9161-73).

Cell culture and transfection. HEK293T cells were cultured in DMEM containing 10% FBS and 1% Penicillin/Streptomycin. Cells were seeded in 6-well plates the night before transfection. 1 μg of plasmid DNA encoding a NRP2a-GFP fusion protein was pre-complexed with PolyJet transfection reagent according to the manufacturer's protocol and subsequently added to cells. Media was changed 16 hours-post transfection and transfected cells were passed to 96-well plates for staining.

Immunofluorescence assays on cultured cells. Binding and quantitation of Fc-HRS (2-60) to cell-expressed NRP2 was achieved using immunofluorescence microscopy. Fc-HRS (2-60) was pre-complexed for 1 hour at room temperature with PE-conjugated anti-Fc at a ratio of 2:1. HEK293T cells previously transfected with NRP2v2-GFP were passed the night before staining to 96 well Greiner Clear flat bottomed Microplates pre-coated with collagen coating solution. Supernatants were removed and cells were washed 1 time with binding buffer. Cells were then fixed with 50 μL of 3.7% methanol-free formaldehyde for 20 minutes at room temperature. Cells were washed 2× with binding buffer and then blocked with 100 μL of blocking buffer for 1 hour at room temperature. The cells were then washed one time with binding buffer and then incubated with 50 μL of staining complex overnight at 4 degrees Celsius. Cells were then washed 3 times with binding buffer, and then nuclei were counter stained with 2 μg/mL Hoechst diluted in DPBS for 10 minutes at room temperature. The Hoechst stain was replaced with 1×PBS and subsequently read on an IN Cell Analyzer 2200. 20× images were acquired and analyzed using In Cell Analyzer 1000 Workstation software. Segmentation of the cell mask was achieved using the GFP channel, and the average PE signal intensity was determined within this mask above a threshold intensity of 5000 (termed GFP Bright cells).

Stable NRP2 expressing cell pool generation. A plasmid (Origene Technologies Cat #RC220706) encoding the NRP2 variant 2 transcript NM_003872 fused to a Myc-DDK tag was purchased. The vector was PCR amplified using Q5 polymerase (New England Biolabs Cat #M0491) with the following primer pairs:

(SEQ ID NO: **)
5′-TGAGGATGACAAAGATTTGCAGCT-3′
(SEQ ID NO: **)
5′-ACCGCGGCCGGCCGTTTATGCCTCGGAGCAGCACTT-3′
(SEQ ID NO: **)
5′-AGTGCCAAGCAAGCAACTCAAA-3′
(SEQ ID NO: **)
5′-AAGTGCTGCTCCGAGGCATAAACGGCCGGCCGCGGT-3′

The resulting PCR products were then fused, cut with MfeI/AgeI (New England Biolabs Cat #R3589, R3552), and ligated into a vector fragment of RC220706 cut with the same enzymes. This vector, containing an untagged NRP2v2 transcript, was then linearized and re-suspended in 10 mM Tris-0.1 mM EDTA. Suspension Expi293 cells (ThermoFisher Cat #A14527), were grown in expression medium (ThermoFisher Cat #A1435101) at 37° C. and 8% CO₂. The linearized plasmid described above was transfected into Expi293 cells using an SF Cell Line 4D-Nucleofector® X Kit L (Lonza Cat #V4XC-2012) and standard protocol T-030 for suspension HEK293 cells. Cells were allowed to recover in static culture for 17 hours, transferred to suspension and recovered an additional 72 hours, and then were selected with 200-350 μg/mL G418 in 50 μg increments (ThermoFisher Cat #10131035). Cell densities and viabilities were monitored for a period of 3 weeks, with fresh media/antibiotic replacement every 2-3 days. Upon recovery of viabilities to >95%, stably transfected cell pools were re-suspended in freezing media and archived.

Flow Cytometry-Based Assay for Fc-HRS (2-60) Binding to NRP2-Expressing Cells
Immobilized TCEP Disulfide Reducing Gel (Thermo Scientific #77712)
PBS/EDTA (0.5 M EDTA in PBS)
EZ-Link™ Maleimide-PEG11-Biotin (Thermo Scientific #21911)
Spin Columns (Thermo Scientific #69705)
Zeba™ Spin Desalting Columns, 40K MWCO (Thermo Scientific #87770)
Pierce Biotin Quantitation Kit (Thermo Scientific #28005)
Streptavidin-PE (Thermo Scientific #12-4317-87)
Anti-NRP2-APC clone 257103 (R&D Systems #FAB22151A)
Propidium iodide solution (Miltenyi Biotec #130-093-233)

Biotinylation of Fc-HRS (2-60). Fc disulfide bonds in Fc-HRS (2-60) were reduced using TCEP gel equilibrated with PBS/EDTA, and the sample was separated using a spin column. Biotinylation was performed using maleimide-PEG11-biotin reagent with a 2 hour reaction at room temperature, and free reagent removed using a Zeba column. Degree of biotinylation was determined to be 3.35 biotins/molecule using the Pierce Biotin Quantitation kit according to the manufacturer's instructions.

Flow cytometry. Biotinylated Fc-HRS (2-60) was incubated for 1 hour on ice with streptavidin-PE at a 1:2 molar ratio to form a staining complex. The staining complex was then added to stably expressing Expi293-NRP2 cells along with titrated anti-HRS antibodies and incubated 30-60 min on ice. Final concentrations were 43.75 nM (biotinylated Fc-HRS (2-60)) and 87.5 nM (streptavidin-PE). Cells were pelleted and washed as described above, and stained with anti-NRP2-APC (1:20) and resuspended in FWB buffer along with 1 μg/mL propidium iodide for viability gating. Samples were acquired on a Cytoflex S flow cytometer (Beckman Coulter), and the percentage of streptavidin-PE+/NRP2+ cells in the viable singlet gate was determined.

Statistical analysis. Data are expressed as mean±SEM or as individual data points, except where noted. In experiments in which animals were euthanized due to tumor burden or body weight loss, the terminal tumor volume was carried forward for statistical analysis. In cases where an animal was found dead but did not have a large tumor (cause of mortality unknown), the animal's data were removed prior to statistical analysis. Significance of difference over time was tested with a 2-way repeated measures ANOVA followed by Dunnett's post-hoc test. Group comparisons were conducted using 1-way ANOVA (parametric or Kruskal Wallis, as noted in the figure legends). A p value≤0.05 was considered significant.

### Example 1

### Initial Receptor Identification Screen

To identify potential interacting partners of HRS, and related HRS polypeptides, the Retrogenix cell microarray screening technology (Retrogenix Ltd., High Peak Rd, United Kingdom) was used to evaluate binding of a HRS-Fc fusion protein construct ([Fc-HRS(2-60)] to a library of approximately 4500 membrane bound human proteins individually expressed in HEK293 cells.

In brief, HEK293 cells were plated onto glass cover slides which have been pre-treated by the application of discrete expression vectors to enable reverse transfection and expression of each of the 4500 membrane proteins, to create a cell microarray. Transfection efficiencies were assessed via ZsGreen1 expression and exceeded the minimum threshold for all of the library members screened.

Use of a smaller HRS fragment with a Fc tag provided for high sensitivity detection via the use of an AlexaFluor647 labeled anti-human IgG Fc antibody (AF647) as the detection reagent, and readily available controls to confirm specificity. The detection antibody was used at screening concentrations of 2, 5, and 20 μg/ml, as more fully described below.

Test Protein was screened at a concentration of 20 μg/ml using two different screening formats; either a sequential staining method, or a pre-incubation staining method. Sequential staining involved, in brief, the sequential addition to the test cells, of the test protein and detection reagents, while the pre-incubation staining method involved the pre-incubation of the test protein with the detection reagents (2:1 molar ratio of test protein to detection antibody) to pre-form higher avidity complexes prior to the addition to the test cells. A background screen was completed with test protein added to slides of fixed, untransfected HEK293 cells to confirm that the test protein did not bind to untransfected cells.

Primary hits (duplicate spots) were identified by analyzing fluorescence intensity in the AlexaFluor 647 and ZsGreen1 emission channels using the ImageQuant system, following standard fluorescent methodology. Confirmation Screens were run to evaluate any screening hits identified from the primary screen, using identical fixed slides treated with 20 μg/ml test protein, or positive or negative controls, using the sequential or pre-incubation methods (n=2 slides per treatment). Additionally all vectors encoding all hits, plus control vectors, were spotted in duplicate on new slides, and used to reverse transfect human HEK293 cells as before. All transfection efficiencies exceeded the minimum threshold Hits were categorized as specific, or non-specific (i.e. it also came up with at least one of the positive or negative controls), and if specific whether the hit was strong, medium or weak binding.

Confirmatory hits using both the sequential and pre-incubation staining methods are summarized in Table E1A and Table E1B below.

TABLE E1A
Sequential incubation confirmatory screening result summary

| Hit | | | | | | |
|---|---|---|---|---|---|---|
| No. | Gene ID: | Accession # | Fc-HRS | CTLA4-FC | RITUXIMAB | PBS |
| 1 | FCGR1A | | NON SPEC | NON SPEC | NON SPEC | NON SPEC |
| 2 | SLC13A3 | | INVERSE | NON SPEC | INVERSE | N/A |
| 3 | NRP2A | NM_003872.2 | WEAK/MED | N/A | N/A | N/A |
| 4 | IGHG3 | | STRONG | STRONG | STRONG | STRONG |
| 5 | FCGR2A | | NON SPEC | NON SPEC | NON SPEC | N/A |
| 6 | NRP2B | NM_201267.1 | WEAK/MED | N/A | N/A | N/A |
| 7 | MS4A1 | | N/A | N/A | STRONG | N/A |
| 8 | CD86 | | N/A | STRONG | N/A | N/A |
| 9 | EGFR | | N/A | N/A | N/A | N/A |
| 10 | SLC38A2 | | WEAK | N/A | N/A | N/A |
| 11 | SLC38A4 | | WEAK | N/A | N/A | N/A |
| 12 | COLEC12 | | WEAK | N/A | N/A | N/A |

TABLE E1B
Pre-incubation confirmatory screening result summary

| | | | Binding scoring | | |
|---|---|---|---|---|---|
| Hit | | | | CTLA4- | |
| No. | Gene ID: | Accession # | Fc-HRS | FC | RITUXIMAB |
| 1 | FCGR1A | | NON | NON | NON |
| | | | SPEC | SPEC | SPEC |
| 2 | SLC13A3 | | INVERSE | NON | INVERSE |
| | | | | SPEC | |
| 3 | NRP2 | NM_003872.2 | MED/ | N/A | N/A |
| | | | STRONG | | |
| 4 | IGHG3 | | NON | NON | NON |
| | | | SPEC | SPEC | SPEC |
| 5 | FCGR2A | | NON | NON | NON |
| | | | SPEC | SPEC | SPEC |
| 6 | NRP2 | NM_201267.1 | MED/ | N/A | N/A |
| | | | STRONG | | |
| 7 | MS4A1 | | N/A | N/A | STRONG |
| 8 | CD86 | | N/A | STRONG | N/A |
| 9 | EGFR | | N/A | N/A | N/A |
| 10 | SLC38A2 | | WEAK | N/A | N/A |
| 11 | SLC38A4 | | WEAK | N/A | N/A |
| 12 | COLEC12 | | WEAK | N/A | N/A |

Summary/Conclusions. After screening test protein (Fc-HRS) for binding against 4500+ human plasma membrane proteins expressed in human HEK293 cells, using two incubation approaches, two neuropilin 2 (NRP2) isoforms—(Neuropilin 2A and 2B) were identified as convincing and specific binding partners (using both incubation approaches). The sequential method also identified three, weak intensity hits: SLC38A2, SLC38A4 and COLEC12. These may also be of biological relevance to HRS polypeptides in general and in particular to those comprising the N-terminal domain (1-60) of HRS. Given the broad involvement of the Neuropilin 2 in a broad range of biological processes, including for example, immune activation, immune cell migration, cancer growth, motility and metastasis, lymphogenesis, epithelial-mesenchymal transition (EMT) and nerve fiber growth guidance, these results suggest that HRS, and related HRS polypeptides have the potential to play key regulatory roles in normal and pathophysiology.

### Example 2

### Confirmation of Binding Specificity by SPR Analysis and Identification and Use of Specific Epitopes

Studies were performed to confirm the binding specificity of Neuropilin 2 (NRP2) to Fc-HRS(2-60) using orthologous methods to those used in the large scale Retrogenix screening (Example 1). In a series of experiments, Fc-HRS(2-60) and related proteins were immobilized on SPR chips, and NRP2 and related proteins were flowed as analytes. Upon confirmation of the NRP2:Fc-HRS(2-60) interaction, the dependence on divalent cations was tested as NRP2 is known to have Ca²⁺ binding sites. The effects of previously characterized NRP2 ligands on the NRP2: Fc-HRS(2-60) interaction was also tested to determine if these known ligands have competitive effects on the Fc-HRS(2-60) interaction.

In another series of experiments, monoclonal antibodies (mAbs) which recognize Fc-HRS(2-60) were immobilized on SPR chips. Fc-HRS(2-60) and NRP2 were pre-incubated and injected over the mAb surfaces to determine if only Fc-HRS(2-60), or a larger NRP2: Fc-HRS(2-60) complex was capable of binding to the mAbs. Additionally, co-injection experiments were carried out in which sequential analyte injections of Fc-HRS(2-60) followed by NRP2 were performed. As the different mAbs bind to different epitopes on Fc-HRS(2-60), the ability of the mAbs to bind to an NRP2: Fc-HRS(2-60) complex as opposed to only binding free Fc-HRS(2-60) gives indications as to the interaction surface between the two proteins.

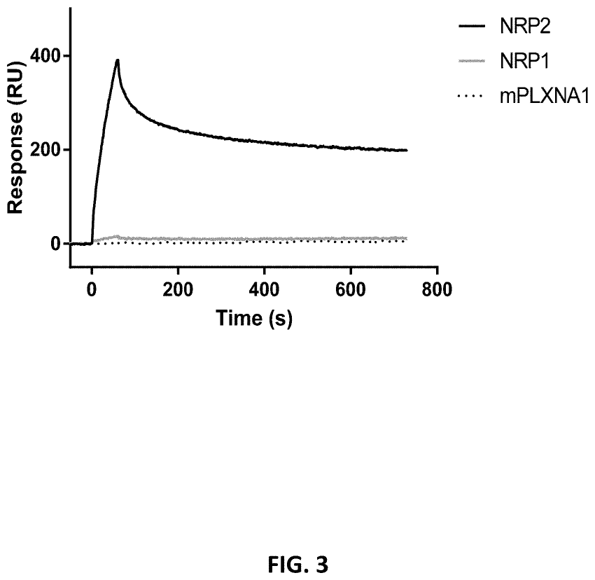

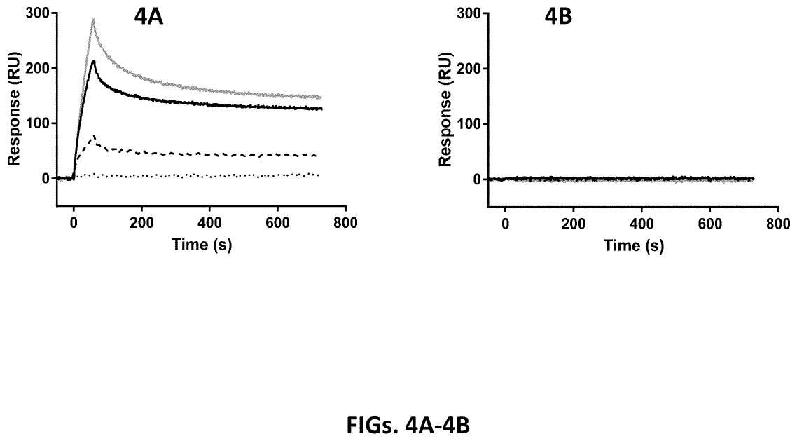

Results. NRP2 but not the closely related NRP1 protein, nor the mouse version of the Plexin A1 co-receptor bind to immobilized Fc-HRS(2-60) (FIG. 3). In addition to human NRP2, both mouse and rat NRP2 demonstrate binding to FC-HRS(2-60). However, none of these NRP2 forms bind to a truncated form of Fc-HRS(2-60) with a 49 amino acid deletion at the C-terminus the fusion protein ([Fc-HRS(2-11)] which deleted the majority of the WHEP domain (FIGS. 4A-3B).

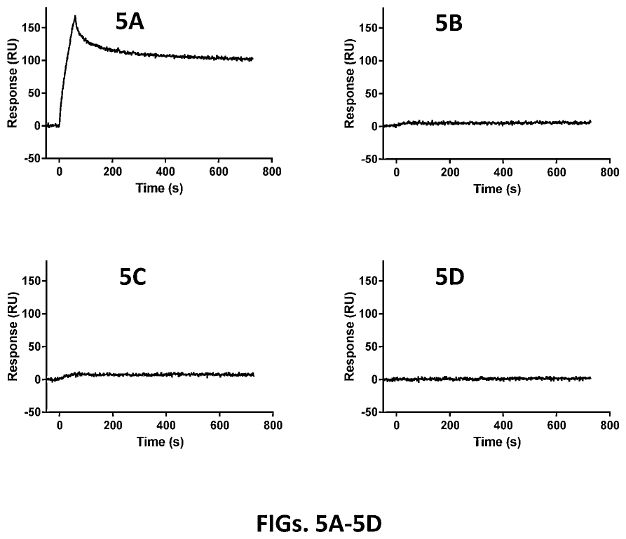

Fc-HRS(2-60) consists of a human IgG Fc region fused to the WHEP domain from histidyl-tRNA synthetase (HRS). Homologous WHEP domains are found in several other tRNA synthetases, including for example, WARS, GARS, MARS, and EPRS. While NRP2 binds to Fc-HRS(2-60), it does not bind to similar proteins consisting of Fc domain fused to the WHEP domain of either GARS or MARS (FIGS. 5A-4D). Additionally NRP2 does not bind to the WHEP domain of WARS with a V5/His tag, suggesting that this interaction with NRP-2 is specific to the HRS WHEP domain and not generally applicable to the other WHEP domains tested.

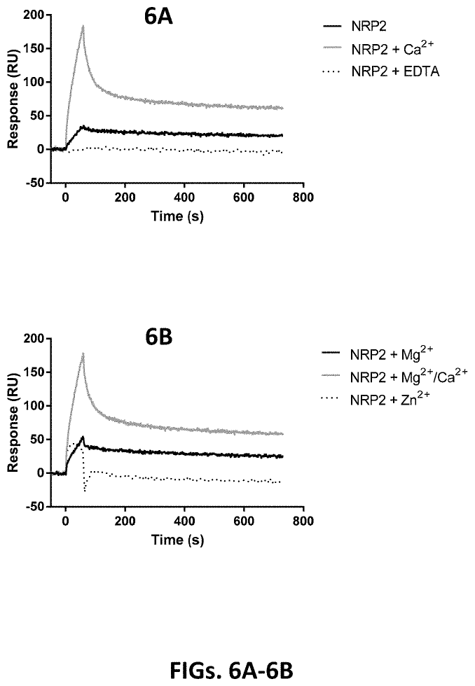

NRP2 is known to have calcium binding sites in its two CUB-domains (a1 and a2 domains). The running buffer of the SPR instrument was switched to a calcium-free buffer (50 mM HEPES, 300 mM NaCl, 0.005% Tween 20, pH 7.4), and CaCl₂, MgCl₂, ZnCl₂ or EDTA were added to the analytes prior to injection and flowed over immobilized Fc-HRS(2-60) (FIGS. 6A-5B). Slight binding was observed in the running buffer alone, while the addition of CaCl₂ greatly enhanced binding. Conversely, addition of ZnCl₂ or EDTA (which chelates divalent cations), resulted in no significant additional binding Additionally, MgCl₂ up to the concentrations tested, does not appear to have any significant effects on the binding. This result suggests the involvement of the a1 or a2 domains of NRP2 in the interaction with Fc-HRS(2-60) either directly or through maintenance of the conformation of the NRP-2 molecule.

A subset of the known ligands of NRP2 appears to compete binding of Fc-HRS(2-60) to NRP2. The VEGF family of ligands that bind NRP2 appear to prevent binding of Fc-HRS(2-60), while the SEMA family of ligands do not appear to compete binding under the conditions tested (Table E2). In the presence of either VEGF-C, VEGF-A₁₆₅, or P1GF-2/Heparin, binding of NRP2 to Fc-HRS(2-60) is reduced or ablated. Conversely, in the presence of VEGF-A145 (which has been reported to be an NRP2 ligand but does not bind NRP2 in our system) or VEGF-A₁₂₁ (which does not bind NRP2), binding of NRP2 to Fc-HRS(2-60), is unaffected. Although SEMA3C and mouse SEMA3F do bind to NRP2, the presence of either of these proteins also do not affect NRP2 binding to Fc-HRS(2-60) under the conditions tested. These results suggest that the Fc-HRS(2-60) binding site of NRP2 overlaps with the VEGF binding site, but not with the SEMA binding site of NRP2.

TABLE E2

| | Binding to NRP2 | Competes with Fc-HRS(2-60) for |
|---|---|---|
| Ligand | observed by SPR | NRP2 binding |
| VEGF-C | Yes | Yes |
| VEGF-A₁₆₅ | Yes | Yes |
| VEGF-A₁₄₅ | No | No |
| VEGF-A₁₂₁ | No | No |
| PlGf-2/Heparin | Not tested | Yes |
| SEMA3C | Yes | No |
| Mouse SEMA3F | Yes | No |

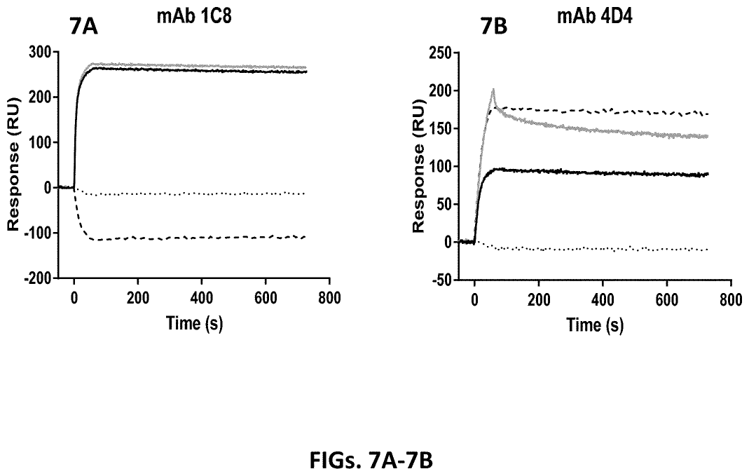

In another series of experiments, different monoclonal antibodies which recognize Fc-HRS(2-60) were immobilized on SPR chips. In FIGS. 7A-6B, the mAb clones 1C8 and 4D4 were immobilized on an SPR chip and then a mixture of Fc-HRS(2-60) and NRP2 which had been pre-incubated together was injected over the mAb surfaces.

Based on the resulting pattern of signal intensities it can be concluded that the monoclonal antibody clone 1C8 likely binds to Fc-HRS(2-60) at an epitope involved in NRP2 binding, because no larger complex binding is detected when the complex is passed over the detection surface. The lack of additional binding under these conditions suggests that the 1C8 antibody is capable of displacing Nrp-2 from the Fc-HRS(2-60):Nrp-2 complex.

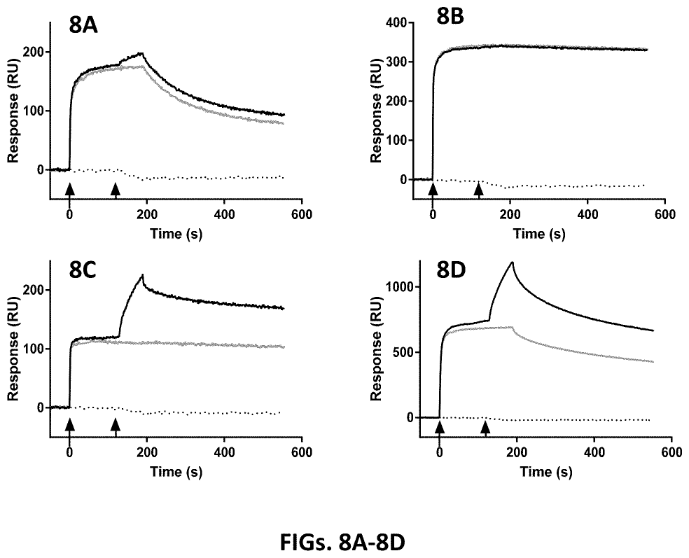

In contrast, when the monoclonal antibody clone 4D4 was attached to the detection surface, a significantly larger signal intensity was observed, suggesting that it was able to bind to the Fc-HRS(2-60) moiety, without displacing Nrp-2, from the Fc-HRS(2-60):Nrp-2. This indicates that NRP2 is able to bind Fc-HRS(2-60) in the presence of the 4D4 mAb and that they bind to non-overlapping regions of Fc-HRS(2-60). Additionally, co-injection experiments were carried out in which sequential analyte injections of Fc-HRS(2-60) followed by NRP2 were performed (FIGS. 8A-7D). In these experiments, Fc-HRS(2-60) that was bound to antibody clone ATYR4D4 or monoclonal antibody clone ATYR13E9 were able to further bind NRP2.

Fc-HRS(2-60) that was bound to monoclonal antibody clone ATYR12H6 showed only slight binding of NRP2, while Fc-HRS(2-60) bound to antibody clone ATYR1C8 showed no binding to NRP2. These data together indicate that antibody clone ATYR1C8 binding is able to block NRP2 binding to Fc-HRS(2-60), while antibody clone ATYR12H6 binding is able to partially block NRP2 binding, and antibody clones ATYR4D4 and ATYR13E9 are not able to block NRP2 binding to HRS.

### Example 3

### Confirmation of Binding to NRP2 Expressed in HEK293 Cells

To directly confirm direct binding of HRS to cells expressing recombinant Neuropilin 2a or 2b, Fc-HRS(2-60) was added to HEK293 cells which had been transfected with expression vectors encoding for either Neuropilin 2a or 2b, or as their respective fusion proteins with GFP, and detected via the use of fluorescently labelled anti-Fc-PE as described in the Materials and Methods.

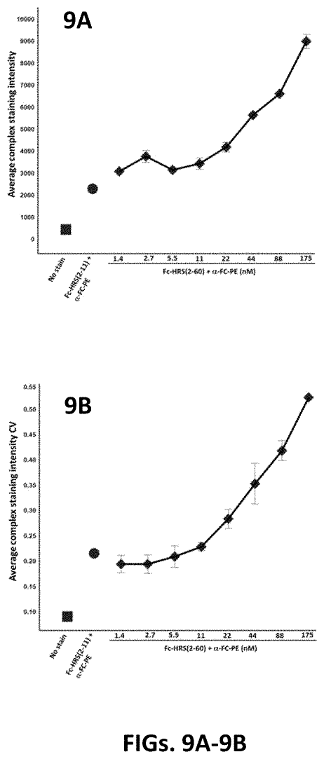

As shown in FIGS. 9A-8B, dose-dependent binding of Fc-HRS (2-60) to cell-expressed NRP2a was readily detectable under these conditions.

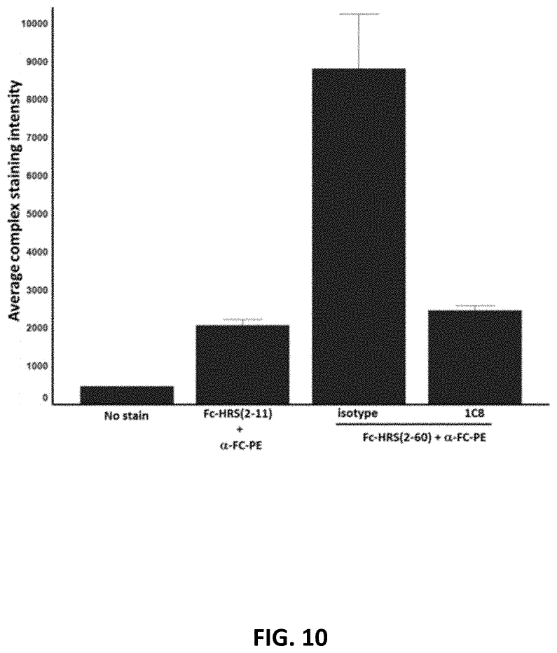

FIG. 10 shows that pre-incubation of Fc-HRS(2-60) with the blocking antibody clone 1C8, resulted in almost complete abolition of binding, demonstrating that the binding is specific for the epitope recognized by the anti-HRS antibody. Binding specificity was further confirmed through the use of the deleted control protein, Fc-HRS(2-11), which also showed negligible specific binding.

To determine the ability of anti-HRS antibodies to block binding of Fc-HRS (2-60) to NRP2, HEK293 cells were stably transfected with NRP2 and binding of biotinylated Fc-HRS (2-60) in the presence or absence of antibodies monitored by flow cytometry as described in the Materials and Methods.

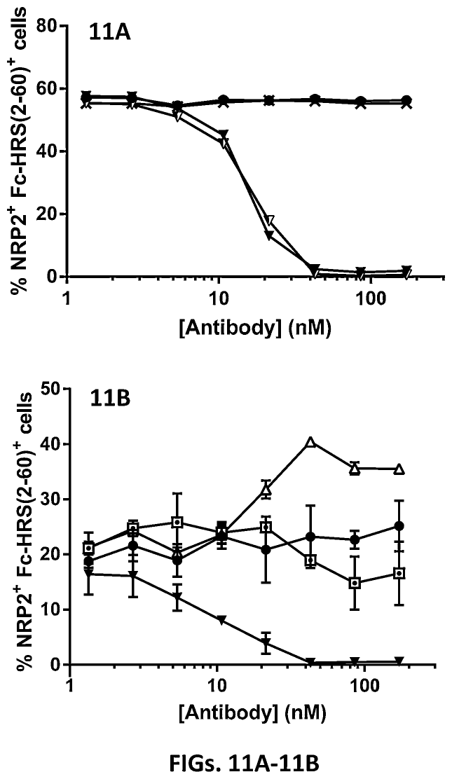

FIG. 11A-11B shows that antibodies from the KL31 series blocked binding of Fc-HRS to NRP2 in a concentration-dependent manner, whereas other antibodies tested did not demonstrate significant blocking characteristics in this assay.

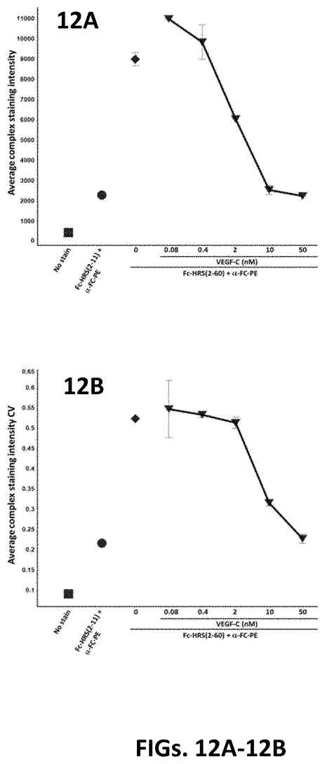

Functional interactions with other Neuropilin 2 interacting proteins was demonstrated via direct competition of Fc-HRS(2-60) by pre-incubation of cells expression NRP2 with VEGF-C (FIGS. 12A-12B).

These results confirm, and extend the Retrogenix screens and suggests that the interaction of HRS proteins such as wild type HRS, and HRS polypeptides comprising the N-terminal region play important, biologically relevant roles by binding to NRP2, and by interacting with its other natural ligands including VEGF-C.

### Example 4

### Circulating Levels of Soluble Neuropilin 2 (Nrp2) in Human Serum and Plasma

Serum & plasma samples from normal healthy volunteers (n=72) were tested for circulating levels of soluble NRP2. NRP2 levels were quantified with an internally developed human NRP2 ELISA (as described in the Materials and Methods).

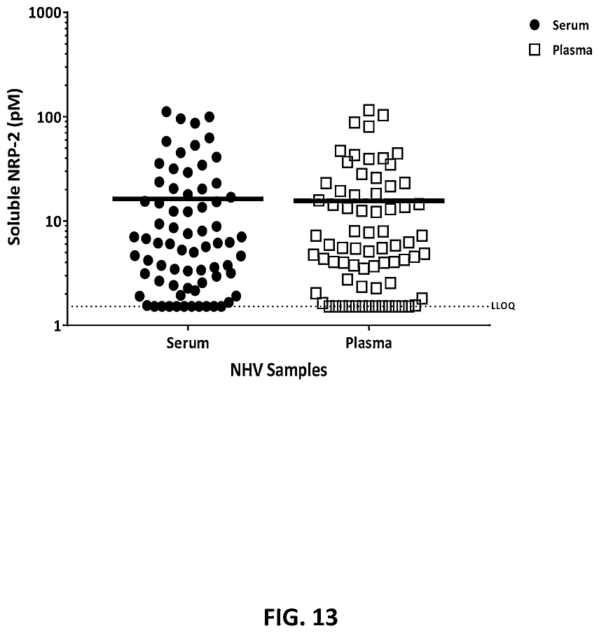

Summary of results. Analysis of circulating NRP2 in both serum and plasma revealed complimentary results in both matrices. Serums levels of NRP2 averaged 16.3 pM while mean plasma levels were 15.6 pM. Quantification revealed that 86% of the serum samples and 83% of the plasma samples were detectable and above the lower limit of quantitation for this assay (1.5 pM) (Table E3 and FIG. 13).

TABLE E3

| | Serum | Plasma |
|---|---|---|
| # of samples | 72 | 72 |
| Mean +/− SD (pM) | 16.3 +/− 24.3 | 15.6 +/− 23.3 |
| Median (pM) | 6.1 | 5.5 |
| Range (pM) | ≤1.5-111.6 | ≤1.5-115.3 |

### Example 5

### Comparison of Circulating HRS & NRP2 Levels

Circulating serum HRS levels from 72 normal healthy donors were rank ordered from lowest to highest levels. Matching serum NRP2 levels from the exact same donors were overlaid on the same axes.

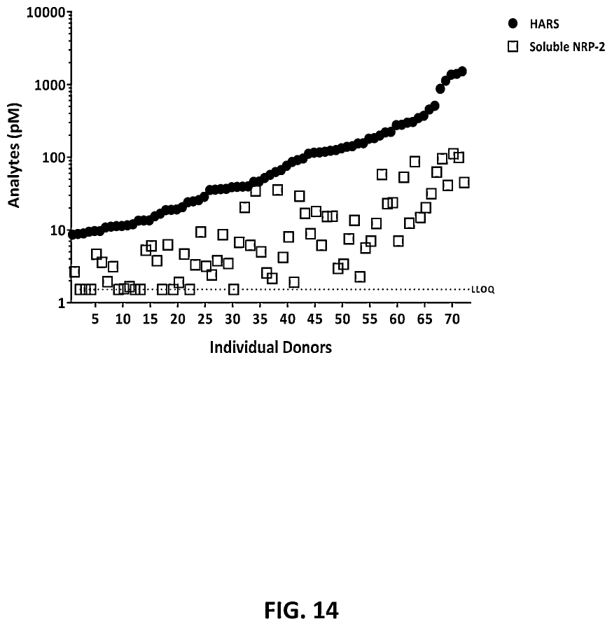

Summary of results. Human HRS levels from normal healthy donors spans nearly two logs (˜10 pM-1000 pM) in concentration. Similarly, soluble NRP2 levels also demonstrate a large distribution in circulating levels (˜1 pM-100 pM). Comparison of serum samples from normal healthy volunteers revealed a trend whereby people with low circulating HRS levels also have lower soluble NRP2 levels and conversely those individuals with higher HRS levels demonstrate higher circulating soluble NRP2 levels (see FIG. 14).

### Example 6

### N-Terminal HRS Assay Interference

Serum samples from normal healthy volunteers were assayed in two separate ELISAs to detect circulating levels of HRS. An assay designed to detect the full length version of HARS (HARS_FL) utilized an N-terminal capture antibody and a C-terminal detection antibody. The second assay was designed to exclusively detect the N-terminal portion of HRS (HARS_NT) with both capture and detection antibodies directed to the N-terminus. Accordingly, the FL-terminal assay, is unable to detect N-terminally truncated fragments of HRS, lacking the C-terminal epitope recognized by the C-terminal detection antibody. Conversely the N-terminal assay is susceptible to interference via the binding of other factors to the N-terminal domain of HRS, which compete with antibody binding.

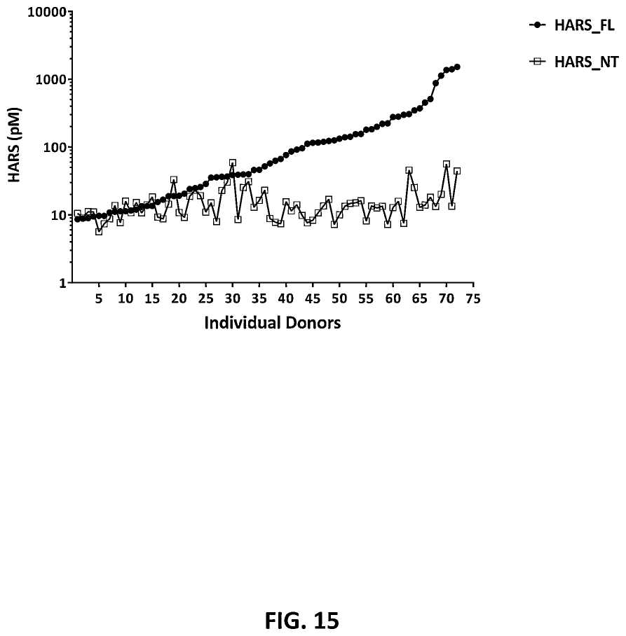

Summary of results. Individual healthy donor serum was assayed for HRS levels using both the full length and N-terminal ELISA formats. Samples with low levels of HRS detected via the full length ELISA HRS levels tended to correlate well with the N-terminal ELISA results (FIG. 15). However, in selected donors with relatively high levels of HRS detected via the FL-ELISA, it was also observed that the HRS levels detected via the N-terminal ELISA no longer showed a close correlation, but were in certain subjects significantly lower. Without being bound by any one specific explanation, it is believed that the significantly lower apparent HRS levels in the N-terminal assay is caused by the existence of an interfering substance which binds to the N-terminal domain of HRS, thereby blocking its detection in the N-terminal ELISA assay.

### Example 7

### Correlation of HRS N-Terminal Interference and Soluble NRP2

To further examine the relationship between HRS N-terminal assay interference and soluble NRP2 levels, circulating HRS and NRP-2 levels were analyzed in normal healthy volunteer serum samples. The difference in observed HRS levels between the full length ELISA and the N-terminal ELISA was calculated for each of the 72 healthy serum donor samples (N-terminal Interference Units). These same samples were additionally tested for circulating human NRP2 levels.

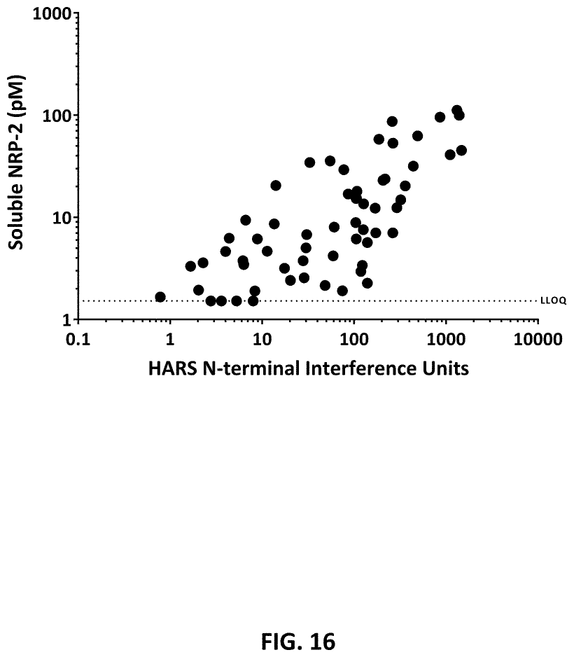

Summary of results. The interference observed between the two HRS assay formats was termed HARS N-terminal Interference Units (HARS_FL minus HARS_NT) and was plotted versus soluble NRP2 levels (FIG. 16). The resulting graph shows a clear trend for increased N-terminal interference and increased soluble NRP2 levels suggesting a potential role for soluble NRP2 to interfere with the detection of the N-terminus of HRS.

### Example 8

### Detection of HRS:NRP2 Soluble Complex in Normal Serum

In an attempt to observe an endogenous circulating HRS: NRP2 soluble complex in serum, several novel ELISA formats were utilized to capture this interaction. Normal healthy human serum was isolated from internal sources (#21949, #32565, #22447, #24098, #23024) or through commercial vendors (Sigma, CELLect). These healthy serum samples were analyzed for levels of N-terminal interference (data not shown) and categorized as either low N-terminal interference or high N-terminal interference and parsed accordingly. These 7 serum samples were assayed in multiple formats of a HRS:NRP-2 complex ELISA. These assays consisted of a capture antibody directed against HRS N-terminus (HARS_NT), HARS C-terminus (HARS_CT), or NRP2. The detection antibody in these assays was directed against the alternate protein in the complex (e.g., HRS detection antibody with a NRP2 capture antibody, and NRP2 detection antibody with a HRS capture antibody).

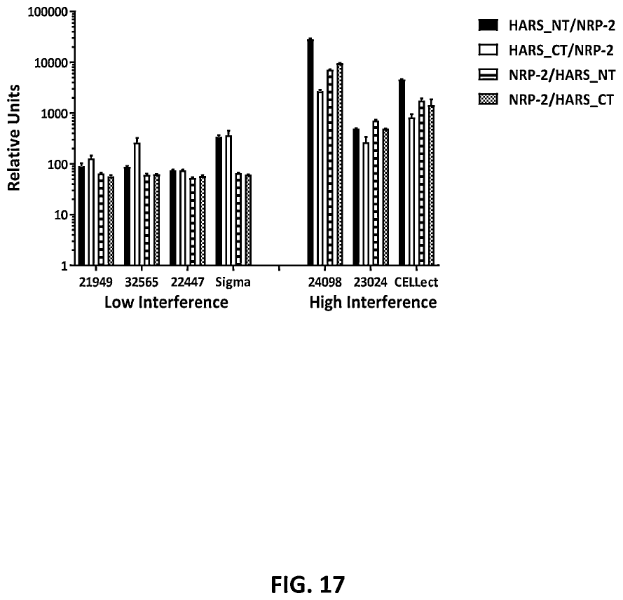

Summary of results. HRS:NRP2 complex ELISAs were tested with normal serum samples that had been previously identified as either having low or high N-terminal interference. All samples with low N-terminal HRS interference showed low signals in all formats of the HRS:NRP2 complex ELISAs (FIG. 17, left bar graphs). In contrast, serum samples identified as containing high N-terminal assay interference all showed elevated signals in HRS and NRP-2 complex ELISAs (FIG. 17, right bar graphs). These results were observed with multiple antibody pairings to both terminal ends of HRS, suggesting that the result is not the result of unanticipated antibody cross reactivity's between NRP2 and HRS.

### Example 9

### Confirmation of a HRS & NRP2 Soluble Complex in Normal Serum

To confirm the relationship between HRS N-terminal interference and the detection of an endogenous soluble HRS:NRP2 complex, the antibody reagents utilized to originally characterize the N-terminal interference observed in human serum were tested side by side in the HRS:NRP2 complex ELISA. Healthy normal serum samples from persons identified as low or high interference (as described above) were tested in a HRS:NRP2 complex ELISA consisting of an NRP2 capture antibody followed by detection with either a non-interfering HRS N-terminal antibody (HARS_NT) or an N-terminal HRS antibody that blocks the interaction (HRS blocking antibody).

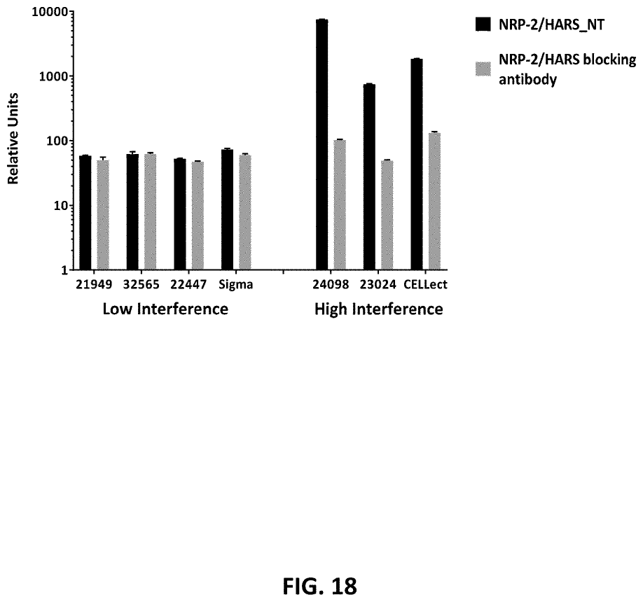

Summary of results. The results of the HRS:NRP2 complex ELISA show an increased signal between low and high interference samples when capturing soluble NRP2 and detecting with the HARS_NT antibody. However, when these same sample are tested in an assay format where the detection antibody against HRS is directed against the site where NRP2 is believed to bind, then the signal in this complex ELISA returns to the same levels as observed in samples without assay interference (FIG. 18). The results suggest that this blocking antibody is directed against the putative NRP2 binding site on the N-terminus of HRS.

### Example 10

### Evaluation of Activity of FC-HRS(2-60) in a Murine Model of Sclerodermatous Chronic Graft Versus Host Disease

This study was designed to investigate the in vivo potential of test compounds in a murine model of sclerodermatous chronic graft vs. host disease (scl cGvHD). A H-2^{d} minor histocompatibility antigen-mismatched model was employed. The model was prepared by allogenic transplantation of bone marrow cells and splenocytes of male donor B10.D2 mice into 8 week old female Balb/c mice which had been subject to whole body irradiation with 700 cGy 6 hours prior to transplantation.

Treatment Regimen. Fifty-six (56) Balb/c mice, (Janvier, Le Genest St. Isle, France, female, 8 weeks) were used as recipients in this study while B10.D2 mice (Jackson Laboratory, Bar Harbor, Me., USA, male) were euthanized to provide donor cells for allogenic transplantation. The animals were assigned to study groups of 8 mice, and housed in specific pathogen-free conditions with sterile food and water. The treatment regimens are shown in Table E4 below. In brief, animals received weekly intravenous injections of the Vehicle of Fc-HRS(2-60) (20 mM Histidine/125 mM NaCl/10 mM Methionine/3% Sucrose/0.02% PS20, pH 6.9) beginning on Day 7 post transplantation (groups 2 and 3) or Fc-HRS(2-60) (0.4 mg/kg) beginning on Day 7 (group 4) or Day 21 (group 6) post-transplantation. Daily oral dosing with nintedanib was included as a known comparator beginning on Day 7 (group 5) or Day 21 (group 7) post-transplantation. A syngeneic transplantation was included as an un-induced control (group 1). Termination for skin and lung harvest was performed on Day 21 (group 2) to provide baseline endpoints for groups that initiated treatment on Day 21 post-transplantation. Remaining groups were euthanized 8 weeks after donor cell injection. Tissues were examined histologically using common staining techniques and tissue homogenates were analyzed for hydroxyproline content as an index of collagen content. Kruskal-Wallis non-parametric ANOVA statistics followed by Dunn's multiple comparison test to Vehicle treatment (group 3) were conducted in GraphPad Prism.

TABLE E4
GROUP TREATMENTS

| | | Transplant | | | |
|---|---|---|---|---|---|
| Group | #Mice | (Day 0) | Treatment | Start | Termination |
| 1 | 8 | Balb/c (H-2^{d}) → | | | |
| | | Balb/c (H-2^{d}) | | | |
| 2 | 8 | B10.D2 (H-2^{d}) → | Vehicle | Day 7 | Day 21 |
| | | Balb/c (H-2^{d}) | IV, QW | | |
| 3 | 8 | B10.D2 (H-2^{d}) → | Vehicle | Day 7 | Week 8 |
| | | Balb/c (H-2^{d}) | IV, QW | | |
| 4 | 8 | B10.D2 (H-2^{d}) → | Fc-HRS(2-60) | Day 7 | Week 8 |
| | | Balb/c (H-2^{d}) | (0.4 mg/kg) | | |
| | | | IV, QW | | |
| 5 | 8 | B10.D2 (H-2^{d}) → | Nintedanib | Day 7 | Week 8 |
| | | Balb/c (H-2^{d}) | (60 mg/kg) | | |
| | | | PO, QD | | |
| 6 | 8 | B10.D2 (H-2^{d}) → | Fc-HRS(2-60) | Day 21 | Week 8 |
| | | Balb/c (H-2^{d}) | (0.4 mg/kg) | | |
| | | | IV, QW | | |
| 7 | 8 | B10.D2 (H-2^{d}) → | Nintedanib | Day 21 | Week 8 |
| | | Balb/c (H-2^{d}) | (60 mg/kg) | | |
| | | | PO, QD | | |

H-2d is a mouse MHC class I antigen
IV, intravenously;
QW, once a week;
PO, orally;
QD, once a day

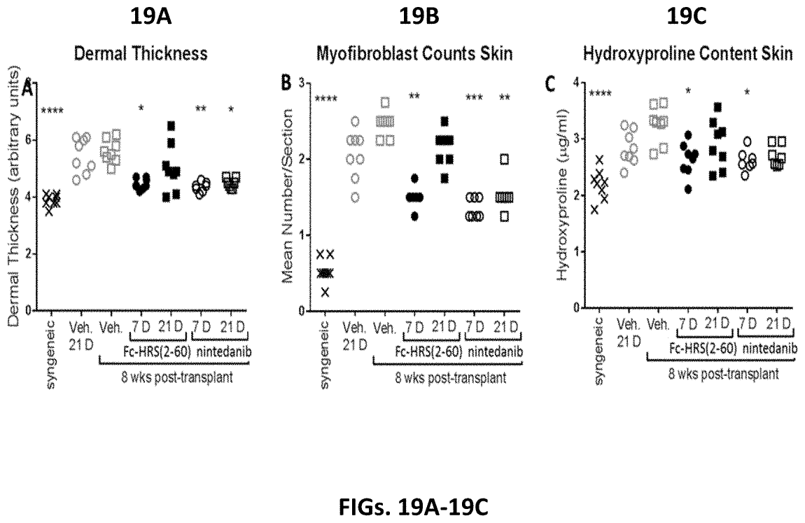

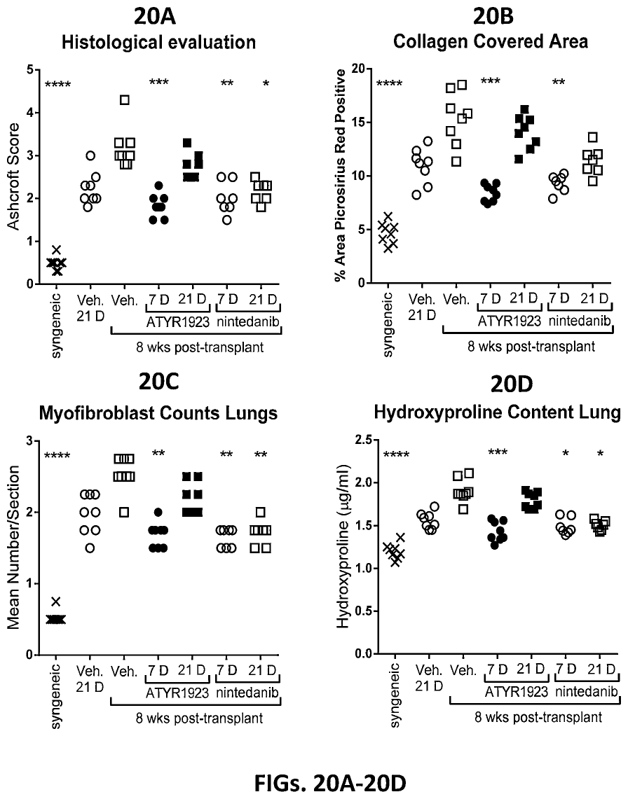

Summary of results. Two mice, one each in groups 5 and 7, died during the second week post-transplantation, presumably due to cGvHD inflammation. Data obtained are shown graphically in FIGS. 19 and 20. In animals that survived to scheduled necropsy, successful model induction was confirmed histologically in the skin by increased dermal thickness and in the lungs by increased fibrosis (both Ashcroft score and collagen stained area). The number of myofibroblasts and collagen content measures were also significantly increased in allogenic vs. syngeneic transplantation in both skin and lung at the 8 week termination time point, further substantiating successful induction of fibrosis in the model. Note that these measures were already increased in animals terminated 3 weeks post-transplantation and although all endpoints had trends for further increase in animals terminated 8 weeks post-transplantation, comparisons between these groups did not show statistically significant differences.

Intervention with nintedanib beginning 7 days post-transplantation significantly ameliorated all measures of fibrosis in both skin and lung as expected. Similarly, nintedanib significantly improved most endpoints measured when intervention was delayed until 21 days post-transplantation. The test compound, Fc-HRS(2-60), was surprisingly effective in this model in both the lung and in the skin when intervention was initiated 7 days post-transplantation. The degree to which Fc-HRS(2-60) was effective at the early intervention timepoint was particularly pronounced in lung where the moderate dose of Fc-HRS(2-60) tested had larger amplitude effects (i.e., lower p values) that the maximal dose of nintedanib tested. FC-HRS(2-60) (0.4 mg/kg) tended to improve fibrotic endpoints when initiated 21 days post-transplantation.

These results demonstrate that HRS polypeptides such as Fc-HRS(2-60) have clear potential for therapeutic impact on fibrotic processes in multiple organs in this murine model.

### Example 11

### Evaluation of Activity of FC-HRS(2-60) in a Murine Model of LPS-Induced Pulmonary Inflammation

This study was designed to investigate the in vivo potential of test compounds in a murine model of neutrophil accumulation in the lung induced by airway instillation of lipopolysaccharide (LPS).

Treatment Regimen. Sixty-four (64) C57BL/6 mice (Jackson Laboratory, Bar Harbor, Me., USA, 8 week old females) were assigned to study groups of 8 mice, and housed in specific pathogen-free conditions with sterile food and water.

The treatment regimens are shown in Table E5 below. In brief, animals received a single intravenous injection of the vehicle of Fc-HRS(2-60) (20 mM Histidine/125 mM NaCl/10 mM Methionine/3% Sucrose/0.02% PS20, pH 6.9), Fc-HRS(2-60) (1, 3, or 10 mg/kg), or HRS(2-60)-COMP (3 mg/kg). The following day, mice were anesthetized by isoflurane inhalation and, using a gavage needle and attached syringe, 50 μL of LPS (Sigma L3024, E. coli 0111-B4, 10 μg/mouse) formulated in PBS, or PBS alone, was delivered to the back of the oral soft palate for inhalation. Twenty-four hours later, animals received a lethal dose of ketamine/xylazine (˜300 and ˜30 mg/kg, respectively) and bronchoalveolar lavage was performed by inflating lungs with 0.8 mL PBS through a cannula placed in the trachea, withdrawing the fluid into a syringe, and recording the retrieved volume. Samples with a retrieved bronchoalveolar lavage volume at or above 0.4 mL were included in the analysis (see Table E5 for included number of samples per group). Cells obtained by bronchoalveolar lavage were collected by centrifugation at 300 g, 10 min at +4° C., washed once by PBS and centrifuged again as above, and resuspended in 100 μL 1×RBC Lysis Buffer (Thermo Fisher Scientific) in order to lyze red blood cells. After an incubation period of 2-3 min, 2 mL of PBS was added to neutralize the lysis buffer, the sample was again centrifuged as above, and the resulting cells were collected.

TABLE E5
GROUP TREATMENTS

| | # Samples | IV Treatment | | Inhaled |
|---|---|---|---|---|
| Group | included | Test article | Dose (mg/kg) | article |
| 1 | 8 | None | n/a | None |
| 2 | 7 | Vehicle | n/a | PBS |
| 3 | 6 | Vehicle | n/a | LPS |
| 4 | 6 | Fc-HRS(2-60) | 1 | LPS |
| 5 | 7 | Fc-HRS(2-60) | 3 | LPS |
| 6 | 7 | Fc-HRS(2-60) | 10 | LPS |
| 7 | 5 | HRS(2-60)-COMP | 3 | LPS |
| 8 | 8 | HRS(2-60)-COMP | 3 | PBS |

n/a, Not Appicable

Flow Cytometry Analysis. Flow wash buffer (FWB, 3% fetal bovine serum in PBS) was used for all subsequent staining and washing steps. Live/dead cells were distinguished by staining with 120 μL of Zombie Yellow (BioLegend) diluted 1:400 in FWB for 20 min in the dark at room temperature. An excess of FWB was then added, and the sample was centrifuged as above; these steps are henceforth collectively referred to as “washing”. Fc receptors were blocked by adding 25 μL of Mouse BD Fc Block (BD Biosciences) diluted 1:25 in FWB for 10 min in the dark at room temperature. NRP2 was detected by addition of 25 μL of rabbit anti-mouse NRP2 (Cell Signaling Technology) at 10 μg/mL in FWB for 1 h in the dark on ice, and cells were then washed (data not shown). Subsequently, cells were stained for specific surface markers using 50 μL of a mixture of the antibodies listed in Table E6, along with 50 μL of AF647-labelled goat-anti-rabbit secondary antibody diluted 1:200, all in FWB for 30 min in the dark on ice. Cells were then again washed, resuspended in 200 μL of FWB, and acquired on a CytoFLEX flow cytometer (Beckman Coulter).

TABLE E6
FLOW CYTOMETRY STAINING ANTIBODIES

| | Dilution in FWB | |
|---|---|---|
| Marker | (Staining Mixture) | Fluorophore* |
| Cd45 | 1:100 | BV421 |
| Cd11b | 1:200 | PE-Cy7 |
| SigLec-F | 1:200 | AF700 |
| Cd11c | 1:200 | APC-Cy7 |
| CX3CR1 | 1:100 | PE |
| GR1 | 1:200 | PerCP |
| Ly6G | 1:200 | FITC |

Conjugated antibodies were obtained from BD Biosciences or BioLegend.
*AF, AlexaFluor; APC, allophycocyanin; FITC, Fluorescein isothiocyanate; BV, Brilliant Violet; PE, phycoerythrin; PerCP, peridinin chlorphyll protein.

For gating strategy, the Cd45+CD11b+ population in single live cells was separated into SigLec-F+Cd11c+ cells (alveolar macrophages) and Siglec-F-Cd11c-cells (other). The latter population was then separated into CX3CR1+(monocytes) and CXC3CR1- (other), which was then separated into a GR1+Ly6G+ population (neutrophils) and a small percentage of GR1+Ly6G-cells that were not further defined.

Using GraphPad Prism 7, results were analyzed by one-way ANOVA followed by Dunn's multiple comparison test using the LPS/Vehicle group as the comparator. A p<0.05 was considered significant.

Preparation of HRS(2-60)-COMP fusion proteins. His tagged HRS(2-60)-COMP protein was designed virtually, and comprised schematically, amino acids 1-17 of the SPARC signal peptide, coupled to amino acids 2-60 of HARS, coupled by 2 copies of a (GGGGS) linker sequence (SEQ ID NO: 204), fused to the cartilage oligomeric matrix protein (COMP) pentameric domain, consisting of amino acids 28-73 of COMP fused to a GGGGS linker (SEQ ID NO:204), which was subsequently fused to a -Myc-His tag (EQKLISEEDLNMHTGHHHHHH) (SEQ ID NO:262). Reference sequences as follows were SPARC signal peptide (NP_003109.1), HARS(P12081.2), COMP(NP_000086.2).

The amino acid sequence was codon optimized for mammalian expression and synthesized by Genewiz LLC into a pUC57 vector, and subsequently sub-cloned into a CMV expression vector pNTC7485 (Nature Technologies Corporation) using flanking enzyme sites SalI and BglII. A negative control protein lacking the HARS (aa2-60) fragment was constructed by site directed mutagenesis of the previous plasmid using PCR primers 5′-GCTGGCAGAGCTCTGGCTGGAGGAGGCGGATCCGGA-3′ (SEQ ID NO: 263) and 5′-TCCGGATCCGCCTCCTCCAGCCAGAGCTCTGCCAGC-3′ (SEQ ID NO: 264). Proteins were produced transiently in Expi293 cells (ThermoFisher) using the Expifectamine transfection system, following the manufacturer's instructions. The over expressed recombinant proteins were then purified over a HisTrap FF column (GEHC), eluted via linear imidazole gradient, and dialysed into 1×PBS pH7.4.

Table H9 provides the full amino acid sequences of the SPARC-HRS(2-60)-COMP-MycHIS fusion protein; the mature, processed form of the HRS(2-60)-COMP-MycHIS fusion protein (with Myc-HIS tags); and the mature, processed form of the HRS(2-60)-COMP fusion protein (without Myc-HIS tags). The optimized (for Homo sapiens) nucleic acid sequence of SPARC-HRS(2-60)-COMP-MycHIS construct is provided in Table E7 below.

TABLE E7
Optimized nucleic acid sequence

| | | SEQ ID |
|---|---|---|
| Name | Sequence | NO: |
| SPARC- | ATGAGGGCCTGGATTTTCTTTCTGCTGTGCCTG | 200 |
| HRS(2-60)- | GCTGGCAGAGCTCTGGCTGCTGAGAGAGCCGCC | |
| COMP- | CTGGAGGAGCTGGTCAAGCTGCAGGGCGAGAGG | |
| MycHIS | GTGAGGGGCCTGAAGCAGCAGAAGGCCAGCGCC | |
| | GAGCTGATCGAGGAGGAGGTGGCCAAGCTGCTG | |
| | AAGCTGAAGGCCCAGCTCGGCCCTGACGAGAGC | |
| | AAGCAGAAGTTCGTGCTGAAGACACCCAAGGGA | |
| | GGAGGCGGATCCGGAGGAGGAGGAAGCAGCGAT | |
| | CTGGGCCCCCAGATGCTGAGGGAGCTGCAGGAG | |
| | ACAAACGCCGCCCTGCAGGACGTGAGAGAGCTG | |
| | CTGAGACAGCAGGTGAGGGAGATCACCTTCCTG | |
| | AAGAACACCGTGATGGAGTGCGACGCCTGTGGA | |
| | GGAGGAGGAGGCAGCGAGCAGAAGCTGATCAGC | |
| | GAGGAGGACCTGAACATGCACACCGGCCATCAC | |
| | CATCACCACCACTGA | |

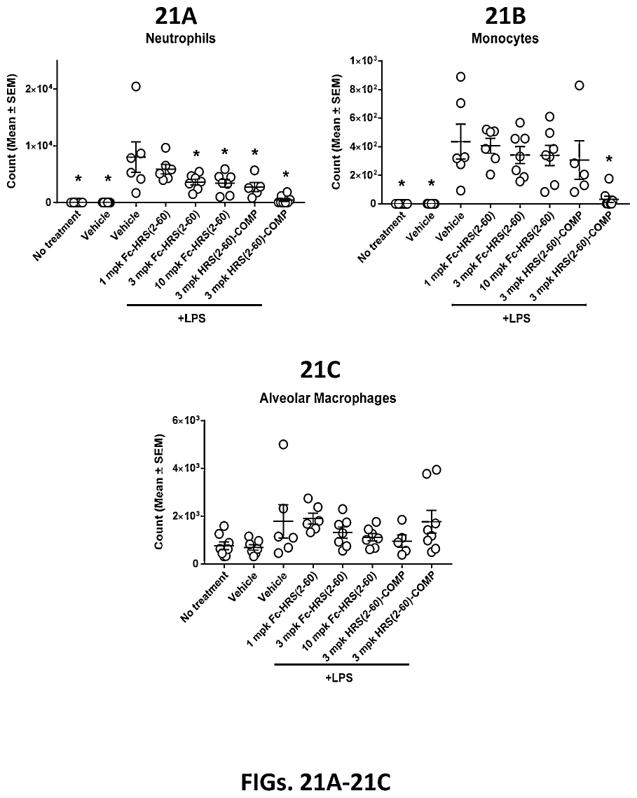

Summary of results. Data obtained from the samples where a sufficient bronchoalveolar lavage volume was collected are shown graphically in FIGS. 21A-21C. In non-treated, non-challenged animals, virtually no neutrophils or monocytes were detected in the airways, whereas alveolar macrophages could be detected as expected. None of the treatments (LPS or test agents) altered the number of alveolar macrophages retrieved by bronchoalveolar lavage. However, LPS inhalation led to robust airway accumulation of neutrophils and monocytes. Fc-HRS(2-60) significantly inhibited LPS-induced neutrophil infiltration when administered at 3 and 10 mg/kg (21A), whereas monocyte infiltration was not altered (21B). The pentamer of HRS(2-60), HRS(2-60)-COMP, also significantly reduced the number of airway neutrophils accumulating in response to LPS (21A).

These results demonstrate that HRS polypeptides such as Fc-HRS(2-60) and HRS(2-60)-COMP have clear potential to reduce neutrophilic inflammation of the airways, and have broad potential as therapeutics in a broad range of inflammatory conditions associated with the migration and/or activation of neutrophils and related immune cells.

### Example 12

### Evaluation of the Activity of FC-HRS(2-60) on Phagosome Maturation in Macrophages

This study was designed to investigate the in vivo potential of test compounds to modulate phagocytosis in murine bone marrow derived macrophages. Phagocytosis refers to the process by which a cell engulfs a solid particle to form an internal compartment known as a phagosome. The process is homologous to eating at the level of single-celled organisms; though in multicellular animals, the process has been adapted to eliminate debris and pathogens, as opposed to taking in fuel for cellular processes.

In the immune system, phagocytosis is a major mechanism used to remove pathogens and cell debris. For example, when a macrophage ingests a pathogenic microorganism, the pathogen becomes trapped in a phagosome which then fuses with a lysosome to form a phagolysosome. Central to phagocytosis is the maturation and acidification of the phagolysosome, a process which can be readily tracked with a pH sensitive dye, as described herein. Within the phagolysosome, enzymes digest the pathogen. Bacteria, dead tissue cells, and small mineral particles are all examples of objects that may be phagocytized.

Methods and protocol. C57BL/6J mice (8-12 weeks of age) (Jackson Laboratory, Bar Harbor, Me., USA) were housed in an animal facility under a 12-hr light/dark cycle and were given standard chow and water ad libitum. Bone-marrow-derived macrophages, were isolated from dissected tibiae and femoral bones after the tissue remaining on the bones was removed. The end of each bone was cut off, and the bone marrow was expelled. Cells from bone marrow were cultured for a total of 7 days with 50 ng/ml macrophage colony-stimulating factor, before confocal analysis. Compounds Fc-HRS(2-60), or control compound (N15-(Fc-HRS(2-15) comprising a deleted, non-functional WHEP domain), were added one day after plating of cells at a concentration of either 100 nM or 200 nM. Media was replaced every other day with fresh media including the respective concentrations of test compounds. After 6 days of culture, cells were washed twice with PBS and pulse labelled with 40 ug/mL of pHrodo™ Red E. coli BioParticles for 15 minutes. After incubation, cells were washed with PBS 3 times and media added back with M-CSF and either Fc-HRS(2-60), or N15 respectively. Cells were collected during the chase phase after 0, 45, 60 and 120 minutes chase. Cells were then washed with PBS, and then fixed with 4% paraformaldehyde and counter stained with Hoechst. Confocal images were captured in Zeiss 710 Confocal Laser Scanning Microscope equipped with 4 lasers and images were captured and analyzed using Zeiss Zen 2010 software. For quantification, the intensity of red fluorescence in each field was measured and the arithmetic mean was calculated and represented graphically.

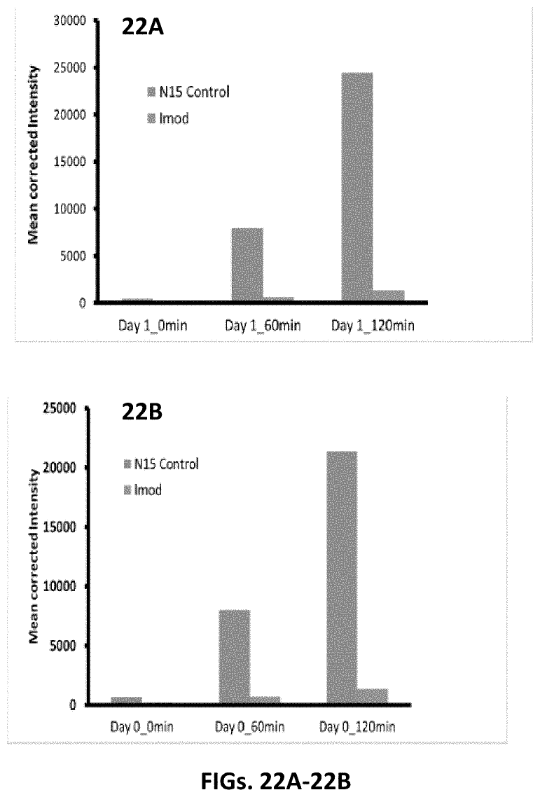

Summary of results. Incubation of bone marrow derived macrophages with either 100 nM or 200 nM Fc-HRS(2-60), but not the Fc control compound N15, for 5 days during monocyte differentiation resulted in a profound inhibition of phagocyte maturation, as revealed by the dramatically diminished spectral shift reported by the pH sensitive fluorescent dye pH Rhodo™ (see FIGS. 22A and 22B). Additional studies at shorter incubations, and different concentrations demonstrated a time and dose dependent inhibition of phagocyte maturation (data not shown). Similar results were also obtained with HRS(2-60)-COMP protein, comprising the pentameric COMP domain (data not shown), confirming that this was a specific effect mediated by the N-terminal region of histidyl t-RNA synthetase (HRS2-60). Bacterial uptake was not significantly impacted by incubation of test compounds (data not shown) by using bacterial particles labelled with a non pH sensitive dye. The profound inhibition of phagocyte acidification closely mimics the phenotype observed in macrophages (Roy et al., (2018) Cancer. Res. 78(19): 5600-5617) and cancer cells in which NRP2 has been knocked out, which also demonstrate endocytic processing blockage resulting in incomplete endosome acidification (Dutta et al., (2015) Cancer. Res. 76 418-28, Neuropilin-2 regulates endosome maturation and EGFR trafficking to support cancer cell pathobiology). These results are of particular significance in cancer treatment because endocytic activity is central to maintaining a metastatic phenotype (Lanzetti & Fiore (2008) Traffic 9, 2011-21 Endocytosis and cancer: an “insider” network with dangerous liaisons).

These results demonstrate that N terminal fragments of HRS, such as the HRS polypeptides Fc-HRS(2-60) and HRS(2-60)-COMP can act, inter alia, to functionally block NRP2's ability to mediate endosomal maturation. Because of the importance of endocytic activity to a broad range of cellular activities, including phagocytosis, efferocytosis, autophagy and receptor recycling, these results suggest that HRS polypeptides represent a fundamentally new pharmacological approach for modulating these activities, by interacting with NRP2. Accordingly such HRS polypeptides have potential utility in a broad range of NRP2 associated diseases, and in particular in the treatment of cancer, and modulation of macrophage function.

### Example 13

### Evaluation of the Activity of FC-HRS(2-60) on Efferocytosis in Macrophages

This study was designed to investigate the in vivo potential of test compounds to modulate efferocytosis in murine bone marrow derived macrophages. Efferocytosis refers to the process by which dying/dead cells (e.g. apoptotic or necrotic cells) are removed by phagocytic cells. During efferocytosis, the cell membrane of phagocytic cells engulfs the apoptotic cell, forming a large fluid-filled vesicle containing the dead cell. Efferocytosis can be performed not only by ‘professional’ phagocytic cells such as macrophages or dendritic cells, but also by many other cell types including epithelial cells, fibroblasts and other cells. To distinguish them from living cells, apoptotic cells carry specific ‘eat me’ signals, such as the presence of phosphatidyl serine (resulting from phospholipid flip-flop) or calreticulin on the outer leaflet of the cell membrane. Efferocytosis uses distinct signaling pathways for the initial recognition of apoptotic cells, but shares many mechanistic similarities to bacterial phagocytosis, endocytosis and autophagy, additionally previous studies have confirmed that NRP2 plays a key regulatory role in these processes (Dutta et al., (2015) Cancer. Res. 76 418-28 Neuropilin-2 regulates endosome maturation and EGFR trafficking to support cancer cell pathobiology). Like phagocytosis, acidification of the endosome, is a central aspect of endosome maturation, which can be readily monitored through the use of a pH sensitive probe, as described herein.

Methods and protocol. C57BL/6J mice (8-12 weeks of age) (Jackson Laboratory, Bar Harbor, Me., USA) were housed in an animal facility under a 12-hr light/dark cycle and were given standard chow and water ad libitum. Bone-marrow-derived macrophages, were isolated from dissected tibiae and femoral bones after the tissue remaining on the bones was removed. The end of each bone was cut off, and the bone marrow was expelled. Cells from bone marrow were cultured for a total of 7 days with 50 ng/ml macrophage colony-stimulating factor. Media was replaced every other day with fresh media supplemented with M-CSF. At day 5 of culture, the media was replaced with fresh media containing M-CSF and Compounds HRS(2-60)-COMP or control compound (COMP alone, at a concentration of 100 nM for 24 hours. At 6 day of culture, cells were washed twice with PBS and pulse labelled with apoptotic jurkat cells (prepared by treatment of jurkat cells with 50 uM etoposide for 12 hours—which had then been pre-loaded with pHrodo™ Red succinimidyl ester for 1 hour). After incubation, macrophages were washed with PBS 3 times and media added back with M-CSF along with either HRS (2-60)-COMP or control compound respectively. Cells were collected during the chase phase after 0, 2, 6, 8, and 10 hours chase. Macrophages were then washed with PBS, and then fixed with 4% paraformaldehyde and counter stained with Hoechst. Confocal images were captured in Zeiss 710 Confocal Laser Scanning Microscope equipped with 4 lasers and images were captured and analyzed using Zeiss Zen 2010 software. For quantification, the intensity of red fluorescence in each field were measured and the arithmetic mean was calculated and represented graphically.

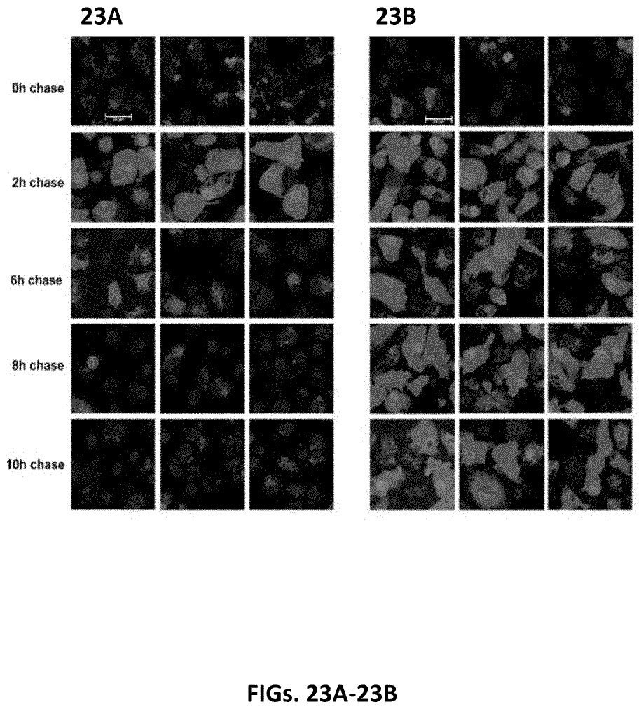

Summary of results. Incubation of bone marrow derived macrophages with 100 nM HRS(2-60)-COMP), but not the control compound COMP, for 5 days during monocyte differentiation resulted in a profound inhibition of efferocytosis, as revealed by the dramatically diminished spectral shift reported by the pH sensitive fluorescent dye pH Rhodo™ (FIGS. 23A and 23B). The profound inhibition of efferocytosis closely mimics the phenotype observed in macrophages in which NRP2 has been knocked out (Roy et al., (2018) Cancer. Res. 78(19): 5600-5617, Macrophage-Derived Neuropilin-2 Exhibits Novel Tumor-Promoting Functions).

These results demonstrate that N terminal fragments of HRS, such as the HRS polypeptides Fc-HRS(2-60) and HRS(2-60)-COMP have the ability, inter alia, to functionally block NRP2's ability to mediate endosomal maturation required for both efferocytosis and phagocytosis. Because of the importance of endocytic activity to a broad range of cellular activities, and diseases, these results suggest that HRS polypeptides represent a fundamentally new pharmacological approach for modulating NRP2 activity. Accordingly such HRS polypeptides have potential utility in a broad range of NRP2 associated diseases, and in particular in the treatment of cancer, and modulation of macrophage function.

### Example 14

### Evaluation of Activity of FC-HRS(2-60) in a P. Acnes-Induced Model of Pulmonary Granulomatous Disease

This study was designed to investigate the in vivo potential of test compounds in a murine model of sarcoid-like granulomatous disease. Granuloma formation and lung fibrosis was induced by sensitizing and challenging mice with heat-killed Propionibacterium acnes (P. acnes) over a four week period.

Treatment Regimen. 60 C57BL/6 mice separated into seven groups, housed under standard housing conditions, were used in this study. The P. acnes suspension needed to induce pulmonary granuloma formation was generated as follows: P. acnes (ATCC #6919) was grown in reinforced clostridial medium or agar for five days under anaerobic conditions at 37° C. until confluency. P. acnes colonies were washed twice with phosphate buffered saline and resuspended in PBS. The resulting P. acnes suspension was then heat-killed by autoclaving at 121° C. for 20 minutes. The protein concentration of the suspension was determined by Bradford method, and then maintained at −80° C. prior to use.

Treatment groups and treatment regimens are shown in Table E8 below. In brief, animals in group 1-2 were left untreated throughout the study. Animals in groups 3 and 6-7 received weekly intravenous injections of the Vehicle of Fc-HRS(2-60) (20 mM Histidine/125 mM NaCl/10 mM Methionine/3% Sucrose/0.02% PS20, pH 6.9) (group 3) or Fc-HRS(2-60) at 0.4 mg/kg (group 6) or 3 mg/kg (group 7). Group 4-5 mice were dosed with positive control Anti-mouse-TNF-α (group 5) or its vehicle control PBS (group 4) via IP route. On day 0 mice in groups 3-7 were first sensitized with 0.5 mg of P. acnes via an IP injection and then challenged with an intratracheal (IT) instillation of P. acnes on days 14 (0.1 mg) and 28 (0.025 mg).

TABLE E8
GROUP TREATMENTS

| | | P. acnes | | | |
|---|---|---|---|---|---|
| Group | #Mice | induction | Treatment | Start | Termination |
| 1 | 5 | No | Untreated | N/A | Day 42 |
| 2 | 5 | No (PBS) | Untreated | N/A | Day 42 |
| 3 | 10 | Yes (Day 0) | Vehicle, IV QW | Day 0 | Day 42 |
| 4 | 10 | Yes (Day 0) | PBS, IP TIW | Day 0 | Day 42 |
| 5 | 10 | Yes (Day 0) | Anti-mTNF-α, | Day 0 | Day 42 |
| | | | 500 μg, IP TIW | | |
| 6 | 10 | Yes (Day 0) | Fc-HRS(2-60), | Day 0 | Day 42 |
| | | | 0.4 mg/kg, IV QW | | |
| 7 | 10 | Yes (Day 0) | Fc-HRS(2-60), | Day 0 | Day 42 |
| | | | 3 mg/kg, IV QW | | |

IV, intravenous;
IP, intraperitoneal;
QW, once weekly;
TIW, thrice weekly

Sample Analysis. All mice were sacrificed for tissue collection and analysis on day 42 post disease induction. A portion of the lungs was designated for histopathological analysis. Paraffin-embedded sections were stained with H&E or Masson's Trichrome using standard histology procedures and scored for the extent of inflammation and fibrosis by a board-certified veterinary pathologist. In addition, harvested lung tissue was snap-frozen for later protein analysis. Frozen lung tissues were homogenized for 15 sec in 350 μl of ice-cold lysis buffer (150 mM NaCl, 50 mM TRIS pH 7.5, 1 mM EDTA, 0.5% Triton X-100, Protease Inhibitor Cocktail) using a hand-held homogenizer. The tissue samples were being kept on ice during the homogenization procedure. After a 20 min incubation period on ice the lysate was cleared by centrifugation at 13000 rpm at 4° C. for 10 min. The protein lysates were then analyzed via the MAGPIX Luminex instrument system using the following kit: MCYTOMAG-70K. One-way ANOVA with Dunn's multiple comparisons test to Vehicle treatment (group 3) was conducted in GraphPad Prism.

Summary of Results. A total of ten mice died throughout the study: One mouse died on day 10 and nine died after the first intratracheal dose on day 15. Two group 3 (Vehicle) animals were found dead on day 19, three group 4 (PBS) animals were found dead on day 10, 17 and 19, respectively. Another five mice were found dead in group 6 (Fc-HRS(2-60)); 0.4 mg/kg) on day 19. The mortality in the mice that died after the first IT challenge on day 14 was attributed to the initial intratracheal dosage of heat-killed P. acnes (0.1 mg). Based on these adverse events, the dose for the second IT challenge on day 28 was lowered to 0.025 mg and no additional mice were lost.

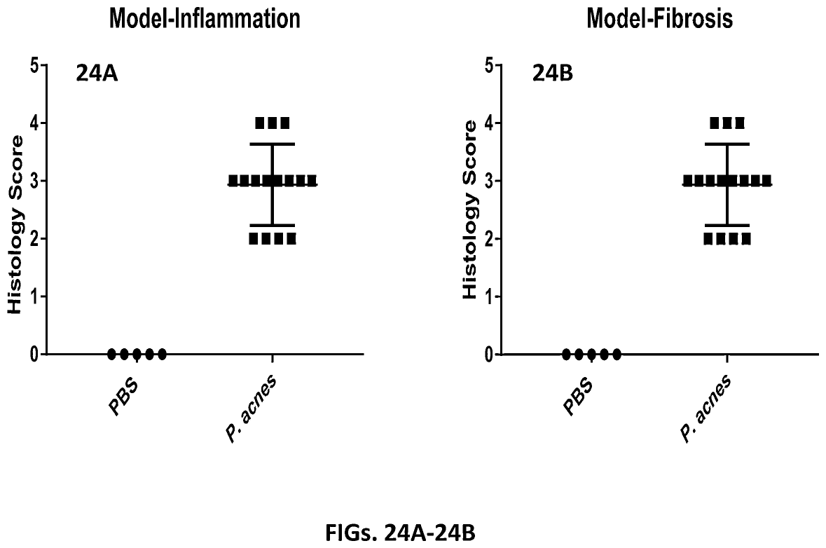

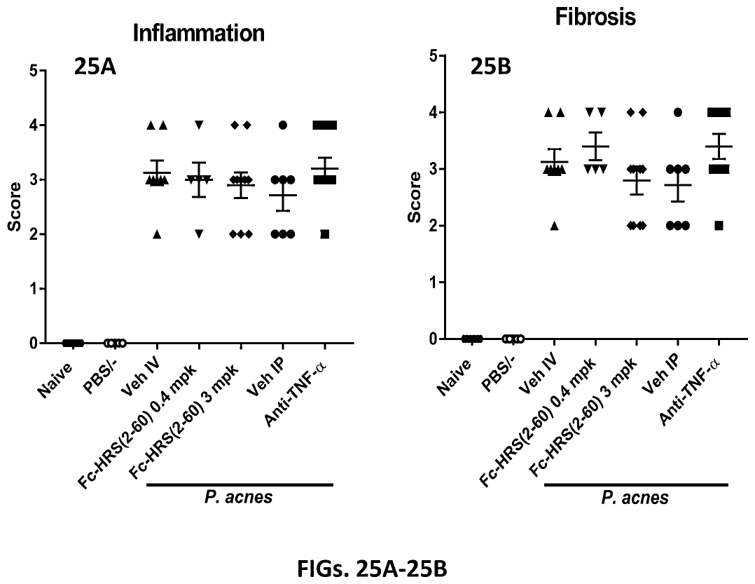

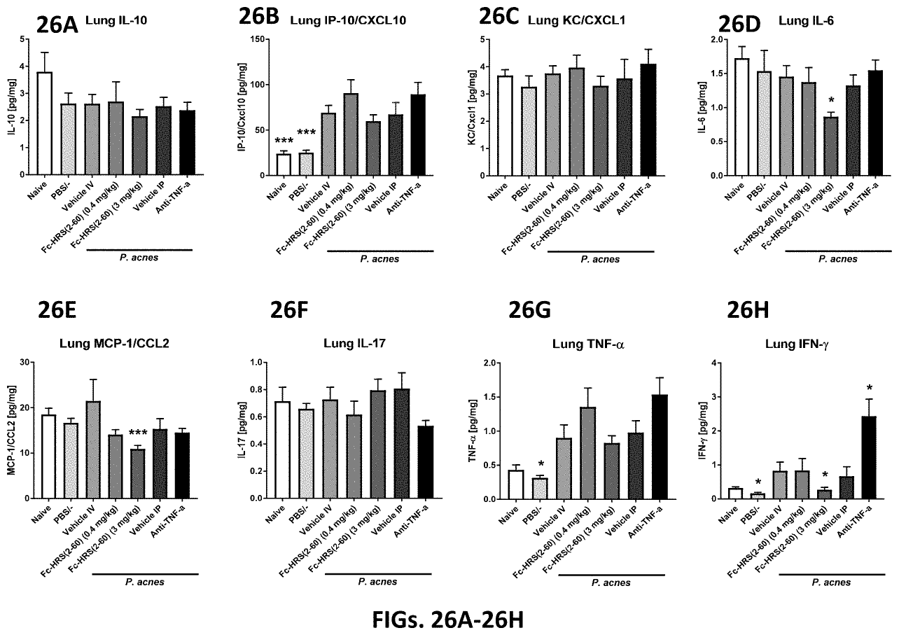

Successful model induction was confirmed histologically by presence of increased inflammation (H&E) and fibrosis (Masson's Trichrome) in mice that received P. acnes (group 3 and 4) as compared to mice that did not receive P. acnes (group 2) (see FIGS. 24A-24B). Although the presence of Fc-HRS(2-60) at 0.4 or 3 mg/kg did not lead to a statistically significant overall reduction in lung inflammation or fibrosis at study termination (see FIGS. 25A-25B), several profibrotic cytokines in the lung were reduced in response to Fc-HRS(2-60) treatment at 3 mg/kg (see FIGS. 26A-26H). Anti-TNF antibodies (the positive control) also failed to show significance in this study (data not shown), possibly indicating animal model variation in this study. Of the eight proteins analyzed, IL-6, MCP-1/CCL2 and IFN-γ were significantly reduced as compared to lungs of vehicle treated mice. The protein levels shown in FIGS. 26A-26H are normalized to total lung protein.

These results demonstrate that Fc-HRS(2-60) is a potent inhibitor of several pro-inflammatory lung proteins in this highly inflammatory model of pulmonary sarcoidosis. Controlling inflammation in sarcoidosis is key for managing flare-ups and inhibiting progression to fibrosis, suggesting that Fc-HRS(2-60) could be of potentially therapeutic value in sarcoidosis and other inflammatory lung diseases.

### Example 15

### Evaluation of Activity of FC-HRS(2-60) in a Murine Model of S. Rectivirgula-Induced Chronic Hypersensitivity Pneumonitis

This study was designed to investigate the in vivo potential of Fc-HRS(2-60) in a murine model of Saccharopolyspora rectivirgula (S. rectivirgula, S. rec)-induced chronic hypersensitivity pneumonitis (CHP).

Treatment Regimen. Sixty C57BL/6 female mice were assigned to study groups of five or ten mice, and housed under standard housing conditions. S. rectivirgula antigen required for disease induction was prepared as follows: S. rectivirgula was obtained from the American Type Cultures Collection (ATCC #15347), and cultivated in 1 L ATCC Medium 3 Broth at −52° C. in a shaking incubator for 4 days, centrifuged, and washed three times with water. The bacterial cell walls were disrupted with a homogenizer or sonication and the resulting S. rectivirgula antigen was then reconstituted in saline at a concentration of 1 mg/mL and kept at −80° C. until use.

The treatment regimens are shown in Table E9 below. In brief, animals in group 1-2 were left untreated throughout the study. Animals in groups 3 and 6-7 received weekly intravenous injections of the Vehicle of Fc-HRS(2-60) (20 mM Histidine/125 mM NaCl/10 mM Methionine/3% Sucrose/0.02% PS20, pH 6.9) (group 3) or Fc-HRS(2-60) at 0.4 mg/kg (group 6) or 3 mg/kg (group 7). Mice in group 5 were dosed with positive control CTLA-4-Fc (IP) and mice in group 4 with the corresponding vehicle control PBS (IP). The disease was induced in groups 2-7 via intranasal instillation of 25 μl S. rectivirgula antigen (1 mg/ml) or PBS on three consecutive days per week for three weeks.

TABLE E9
GROUP TREATMENTS

| | | S. rec | | | |
|---|---|---|---|---|---|
| Group | #Mice | induction | Treatment | Start | Termination |
| 1 | 5 | No | Untreated | N/A | Day 20 |
| 2 | 5 | No (PBS) | Untreated | N/A | Day 20 |
| 3 | 10 | Yes (Day 0) | Vehicle, IV QW | Day −1 | Day 20 |
| 4 | 10 | Yes (Day 0) | PBS, IP TIW | Day −1 | Day 20 |
| 5 | 10 | Yes (Day 0) | CTLA-4-Fc, 50 μg, | Day −1 | Day 20 |
| | | | IP TIW | | |
| 6 | 10 | Yes (Day 0) | Fc-HRS(2-60), | Day −1 | Day 20 |
| | | | 0.4 mg/kg, IV QW | | |
| 7 | 10 | Yes (Day 0) | Fc-HRS(2-60), | Day −1 | Day 20 |
| | | | 3 mg/kg, IV QW | | |

IV, intravenous;
IP, intraperitoneal;
QW, once weekly;
TIW, thrice weekly

Sample Analysis. All mice were sacrificed for tissue collection and analysis on day 20 post disease induction. A portion of the lungs was designated for histopathological analysis. Paraffin-embedded sections were stained with H&E for examination by a board-certified veterinary pathologist. Presence and severity of multifocal chronic pneumonia was scored using the accepted industry scoring system described in Mann, et al. (2012) ‘International Harmonization of Toxicologic Pathology Nomenclature: An Overview and Review of Basic Principles’, Toxicologic Pathology, 40(4_suppl), pp. 7S-13S. doi: 10.1177/0192623312438738). H&E stained lung sections were also analyzed using the HALO platform in order to quantify bronchus-associated lymphoid tissue (BALT). In addition, a portion of harvested lung tissue was snap-frozen for later protein analysis. Frozen lung tissues were homogenized for 15 s in 350 μl of ice-cold lysis buffer (150 mM NaCl, 50 mM TRIS pH 7.5, 1 mM EDTA, 0.5% Triton X-100, Protease Inhibitor Cocktail) using a hand-held homogenizer. The tissue samples were being kept on ice during the homogenization procedure. After a 20 min incubation period on ice the lysate was cleared by centrifugation at 13000 rpm at 4° C. for 10 min. The protein lysates were then analyzed via the MAGPIX Luminex instrument system using the following kits: MCYTOMAG-70K, MMMP3MAG-79K.

One-way ANOVA with Dunn's multiple comparisons test to vehicle treatment (group 3) was conducted in GraphPad Prism.

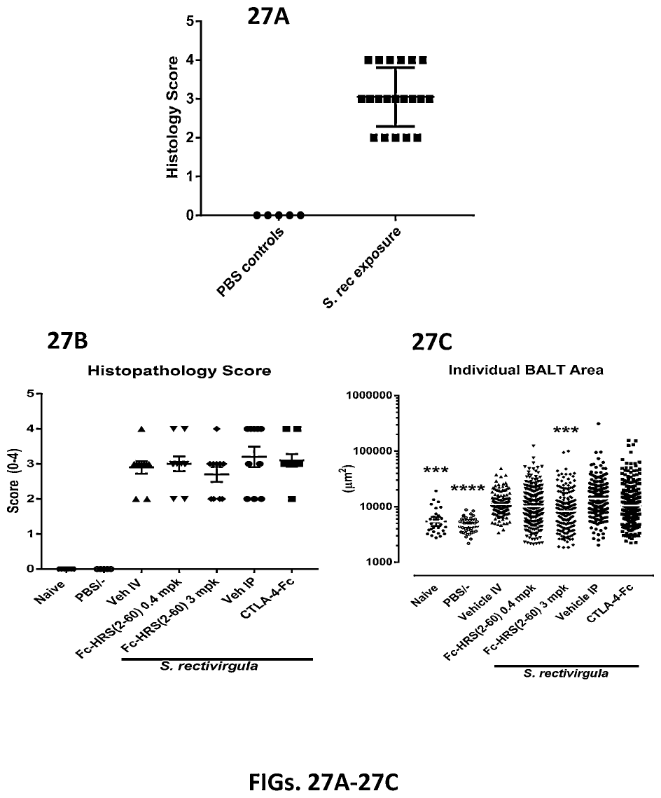

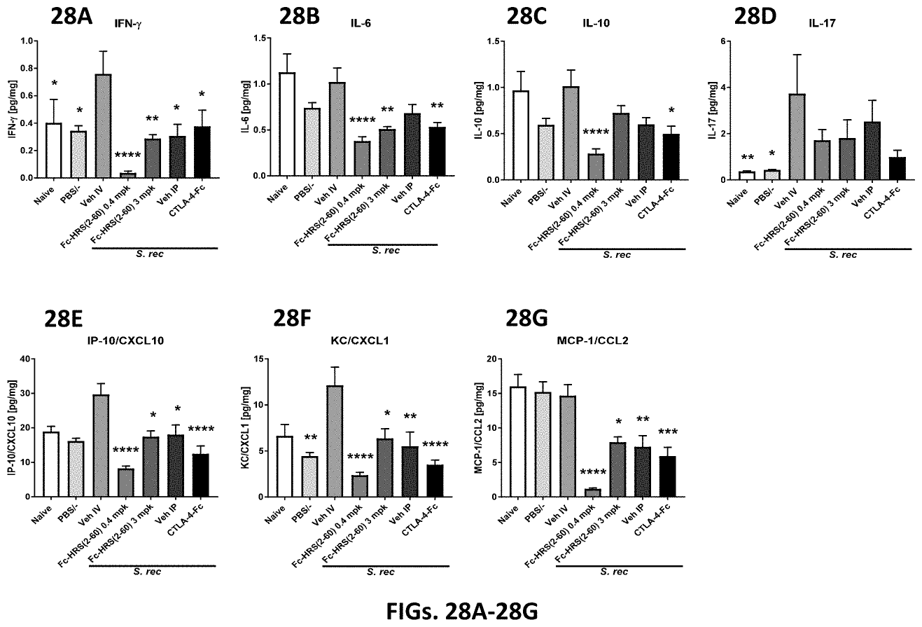

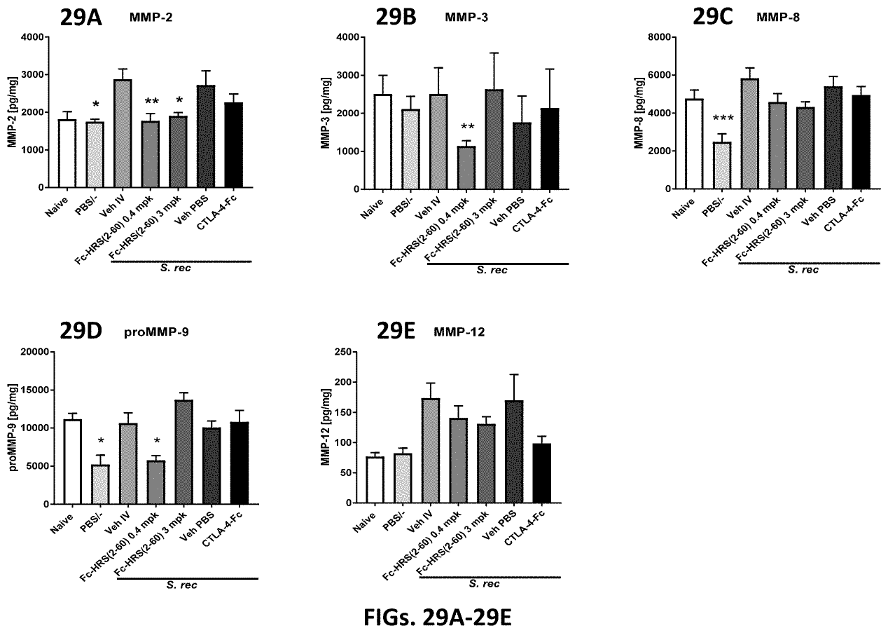

Summary of results. Chronic hypersensitivity pneumonitis was successfully induced with 25 μg intranasal challenges of S. rectivirgula administered on three consecutive days per week for three weeks. Mice exposed to S. rectivirgula in control groups (Group 3 and Group 4) had a robust and consistent multifocal chronic pneumonia compared to mice exposed to PBS in Group 2 (FIG. 27A). The mean histopathology scores as determined by a veterinary pathologist were similar among groups 3-7 (FIG. 27B). However, a following in-depth analysis of the H&E stained lung tissue sections using the HALO platform showed a reduction of individual BALT area in the Fc-HRS(2-60) 3 mg/kg group (FIG. 27C) suggesting anti-inflammatory activity in this model. Further, lung homogenates were analyzed for levels of pro-inflammatory and profibrotic proteins. Several pro-inflammatory cytokines and chemokines (see FIGS. 28A-28G) as well as matrix metalloproteases (MMPs) (see FIGS. 29A-29E) were significantly reduced in presence of Fc-HRS(2-60) at both 0.4 and 3 mg/kg. These results demonstrate that Fc-HRS(2-60) negatively regulates lung proteins that are implicated in the development of fibrosis. This broad anti-inflammatory effect may be translatable to human patients suffering of similar, highly inflammatory types of interstitial lung diseases.

### Example 16

### Evaluation of Activity of FC-HRS(2-60) in a Murine Model of RA-ILD

This study was designed to investigate the in vivo activity of Fc-HRS(2-60) in a transgenic mouse model (SKG) of Rheumatoid-Arthritis Interstitial Lung Disease (RA-ILD). SKG mice, which are genetically prone to develop autoimmune arthritis, develop severe chronic arthritis after a single intraperitoneal injection of zymosan, and approximately 20% of these SKG mice also develop ILD.

Treatment Regimen. 70 SKG/jcl female mice were assigned to seven study groups, and housed under standard housing conditions. The study groups are described in Table E10 below. In brief, animals in group 1 were left untreated throughout the study. Mice in groups 3 were dosed with positive control Anti-GM-CSF (BioXcell, # and mice in group 2 were dosed with the corresponding IgG control. Mice in groups 4-7 received weekly intravenous injections of the vehicle for Fc-HRS(2-60) (20 mM Histidine/125 mM NaCl/10 mM Methionine/3% Sucrose/0.02% PS20, pH 6.9) (group 4) or Fc-HRS(2-60) at 0.3 mg/kg (group 5), 1 mg/kg (group 6), or 3 mg/kg (group 7). Arthritis was induced in groups 2-7 on day 0 via IP administration of 5 mg zymosan (Sigma, #Z4250).

TABLE E10
GROUP TREATMENTS

| | | Disease | | | |
|---|---|---|---|---|---|
| | | induction | | | |
| Group | #Mice | (Day 0) | Treatment | Start | Termination |
| 1 | 10 | No | Untreated | N/A | Day 56 |
| 2 | 10 | Zymosan | Rat IgG2a, 100 μg, | Day −1 | Day 56 |
| | | | IP QW | | |
| 3 | 10 | Zymosan | Anti-GM-CSF, | Day −1 | Day 56 |
| | | | 100 μg, IP QW | | |
| 4 | 10 | Zymosan | Vehicle, IV QW | Day −1 | Day 56 |
| 5 | 10 | Zymosan | Fc-HRS(2-60), | Day −1 | Day 56 |
| | | | 0.3 mg/kg, IV QW | | |
| 6 | 10 | Zymosan | Fc-HRS(2-60), | Day −1 | Day 56 |
| | | | 1 mg/kg, IV QW | | |
| 7 | 10 | Zymosan | Fc-HRS(2-60), | Day −1 | Day 56 |
| | | | 3 mg/kg, IV QW | | |

IV, intravenous;
IP, intraperitoneal;
QW, once weekly

Clinical Scoring and Sample Analysis. Body weights and scoring for arthritis was conducted once a week. For assessing the extent of arthritis, each limb was scored separately and clinical points were assigned as follows: 0=no joint swelling, 0.1=swelling of one finger joint, 0.5=mild swelling of wrist and ankle, 0.75=moderate swelling of wrist and ankle and 1=severe swelling of wrist and ankle. At study termination on day 56, lungs were perfused with PBS, excised, and dissociated for flow cytometric analysis using Miltenyi's lung dissociation kit (#130-095-927).

Flow Cytometry Analysis. Flow wash buffer (FWB, 3% fetal bovine serum in PBS) was used for all subsequent staining and washing steps. Live/dead cells were distinguished by staining with 120 μL of Zombie Yellow (BioLegend) diluted 1:400 in FWB for 20 min in the dark at room temperature. An excess of FWB was then added, and the sample was centrifuged as above; these steps are henceforth collectively referred to as “washing”. Fc receptors were blocked by adding 25 μL of Mouse BD Fc Block (BD Biosciences) diluted 1:25 in FWB for 10 min in the dark at room temperature. Subsequently, cells were stained for specific surface markers using 50 μL of a mixture of the antibodies listed in Table E1l, all in FWB for 30 min in the dark on ice. Cells were then again washed, resuspended in 200 μL of FWB, and acquired on a CytoFLEX flow cytometer (Beckman Coulter). Only three of the study groups were analyzed by flow cytometry: group 1, group 4 and group 7.

TABLE E11
FLOW CYTOMETRY STAINING ANTIBODIES

| | Dilution in FWB | |
|---|---|---|
| Marker | (Staining Mixture) | Fluorophore* |
| CD3 | 1:200 | APC-Cy7 |
| CD4 | 1:200 | PerCpCy5.5 |
| CD19 | 1:200 | Alexa700 |
| CD8 | 1:200 | BV650 |
| F4/80 | 1:100 | BV510 |
| CD11b | 1:200 | PE-Cy7 |
| CD11c | 1:200 | FITC |
| NK1.1 | 1:100 | PE |
| GR-1 | 1:100 | BV421 |
| CD64 | 1:100 | PE/Dazzle594 |
| Live/Dead (Zombie Yellow) | 1:400 | BV570 |

Conjugated antibodies were obtained from BD Biosciences or BioLegend.

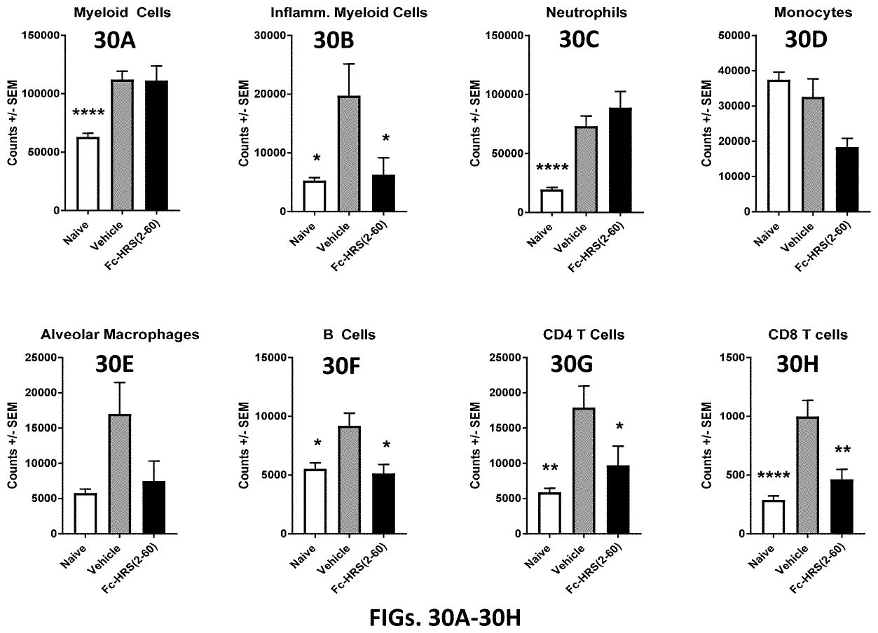

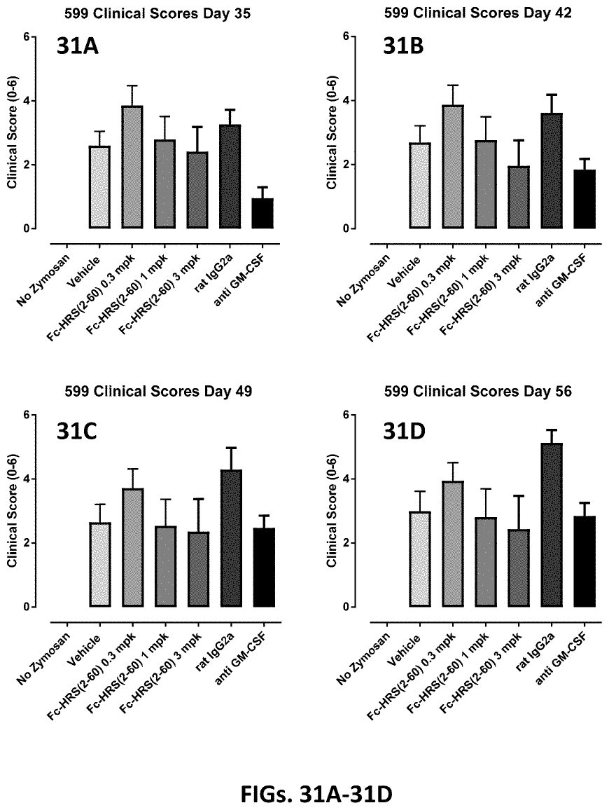

Summary of results. Inflammatory arthritis was successfully induced in SKG mice upon administration of 5 mg of zymosan, and flow cytometric analysis of lung single cell suspensions, demonstrated that Fc-HRS(2-60) can lower the number of specific immune cells in the lungs of SKG mice; most notably B cells and T cells (see FIGS. 30A-30H). Despite significant effects on immune cell infiltration, no significant effects of Fc-HRS(2-60) on overall clinical arthritis scores were observed in this experiment (see FIGS. 31A-31D). Both B and T cells are implicated in RA-ILD pathogenesis and the observed reduction of their numbers in the lung of diseased mice may provide an avenue for potential therapeutic use of Fc-HRS(2-60) in RA-ILD or other T-cell driven inflammatory diseases, either alone, or in combination with other agents.

1-108. (canceled)

109. A method for modulating autophagy, phagocytosis, or efferocytosis in a subject having a neuropilin-2 (NRP2) associated disease or condition, comprising
(a) selecting the subject for treatment based on having increased levels or expression of NRP2a and/or NRP2b relative to a healthy control or matched control standard or population of subjects; and
(b) administering to the subject a therapeutic composition comprising a histidyl-tRNA synthetase (HRS) polypeptide.

110. The method of claim 109, wherein the method comprises analyzing circulating levels of NRP2 in a serum or plasma sample from the subject.

111. The method of claim 109, wherein the disease is a cancer.

112. The method of claim 111, wherein the cancer is selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, and stomach cancer.

113. The method of claim 111, wherein the cancer expresses or overexpresses NRP2.

114. The method of claim 113, wherein the cancer displays NRP2-dependent growth, NRP2-dependent adhesion, NRP2-dependent migration, NRP2-dependent chemoresistance, and/or NRP2-dependent invasion.

115. The method claim 111, wherein the cancer is a primary cancer.

116. The method of claim 111, wherein the cancer is a metastatic cancer, optionally a metastatic cancer that expresses NRP2a and/or NRP2b.

117. The method of claim 111, wherein the cancer is chemoresistant to at least one cancer therapy selected from an immunotherapy agent, a chemotherapeutic agent, a hormonal therapeutic agent, and a kinase inhibitor.

118. The method of claim 117, wherein the method comprises selecting a subject having a cancer that is chemoresistant prior to administering the HRS polypeptide.

119. The method of claim 111, wherein the HRS polypeptide modulates autophagy, efferocytosis, or phagocyte maturation in a cancer cell or cancer-associated macrophage.

120. The method of claim 119, wherein the HRS polypeptide inhibits autophagy in the cancer cell or cancer-associated macrophage.

121. The method of claim 109, wherein the HRS polypeptide comprises an amino acid sequence that is at least 95%, 96%, 97%, 98%, 99%, or 100% identical to SEQ ID NO:156.

122. The method of claim 121, wherein the HRS polypeptide comprises or consists of the amino acid sequence set forth in SEQ ID NO: 156.

123. The method of claim 109, comprising administering to the subject at least one additional agent selected from one or more of a cancer immunotherapy agent, a chemotherapeutic agent, a hormonal therapeutic agent, and a kinase inhibitor.

124. The method of claim 123, wherein the cancer immunotherapy agent is selected from one or more of an immune checkpoint modulatory agent, a cancer vaccine, an oncolytic virus, a cytokine, and a cell-based immunotherapies.

125. The method of claim 124, wherein the immune checkpoint modulatory agent is a Programmed Death-Ligand 1 (PD-L1) and/or a Programmed Death 1 (PD-1) inhibitor.

126. The method of claim 123, wherein the at least one chemotherapeutic agent is selected from one or more of an alkylating agent, an anti-metabolite, a cytotoxic antibiotic, a topoisomerase inhibitor (type 1 or type II), and an anti-microtubule agent.

127. A method for treating an inflammatory lung disease in a subject in need thereof, comprising
(a) selecting the subject for treatment based on having increased levels or expression of NRP2a and/or NRP2b relative to a healthy control or matched control standard or population of subjects; and
(b) administering to the subject a therapeutic composition comprising a histidyl-tRNA synthetase (HRS) polypeptide.

128. The method of claim 127, wherein the inflammatory lung disease is selected from pulmonary sarcoidosis, RA-ILD, chronic hypersensitivity pneumonitis, pulmonary inflammation, pulmonary granulomatous disease, neutrophilic asthma, and pulmonary fibrosis.

129. The method of claim 127, wherein the subject has systemic sclerosis.

130. The method of claim 127, wherein the HRS polypeptide comprises an amino acid sequence that is at least 95%, 96%, 97%, 98%, 99%, or 100% identical to SEQ ID NO:156.

131. The method of claim 130, wherein the HRS polypeptide comprises or consists of the amino acid sequence set forth in SEQ ID NO: 156.

132. The method of claim 131, wherein the inflammatory lung disease is pulmonary sarcoidosis.
